SEQUENCE LISTING (145 sequences)

SEQ ID NO: 1 - DNA, 1575 bp, synthetic construct
  source 1..1575: mol_type = other DNA; note = MG153-22_cloned_into_untethered_plasmid
gctagcgccaccatgtacccctatgatgtacctgattatgcttctggcggcagcagcggcggctcaagcggaagtgaaacacctggaacttccgaaagcgctacgcctgaatcttctggtggatcttctggcggatcttccgcatgcatgcggcagaaatcagaacaacttgaattagccctggataataggggagaggctcccacctccagacgaagtggcgaagcaccgactacagcgcacgaagcagagaggtccggcgggggacaccggttgatggagcaggtcgtagctcgcgctaatgctttggccgccctgaggagggtgaaacaaaaccgggggagccctggcgtggacgggatgacagtcggcgagctgcctcaatatttagcaaaacactgggaaaccgtgcgggagcagctgctggccggatcttaccaaccagagcccgtacgtagacaggccatccccaaacctggcggaggtacccgagttctggggatcccgacagtgttggatcgcttcatccagcagtgcctcctgcaggtacttcagccaaggtttgaccccagtttttcagatcactcatacggattcaggccagggaggaacgctcacgatgcagtctgtgctgcacagcagtacatacaagccggtcgcagatgggtggctgacctggatcttgagaagttctttgatcgggtgaatcatgacgtgctcatggagcggctcgcgcggagaatcgctgatcggagggtgctgcggttgatacgacgttaccttgtcgctgggatcctgcatggaggtgttgttgtggagcgaagagaagggactccgcaggggggcccactgagccctctgctggccagtgtcctgctggacgaagtggatcgcgaacttgagaggcgagggctggcattcgcaagatatgctgatgatctcaacgtttactgcgggagtaggcgagcggcccatgacgcaatggccacattgaagaggctctttgccgccctccggttaaaagtaaacgaatccaagtccacggtggccagagtctgggaaaggaaatttctgggatattcattctgggtggcacccggtagggttatcagaccaaggatcgcaccggcagccctggctgtgatgaaagagcgggtgcgccgcatcacgcggcgcaccggcggtcgaagcttggaagctgtggcacaggagctgcgagaatatctcacagggtggaaagcatactttcggcttgccgggaaaccccgggtgttcagagacctggatgagtggacccgccacaggttgcgcgccgtccagctgaaacaatggaagagaggaaggaccgtctgtagagagttactggccagaggtgtccccgagagggaggccagagctgccgctgcccatgcaaggaggtggtgggcgatggctgctcacagtgcgttacagaccgccctgccaaactcccatttcgaccaattaggagtgccaaggttggcgggtcgtcctaggcctaagaagaaaagaaaggtgggtggttctcctaagaaaaagagaaaggtctgagaattc

SEQ ID NO: 2 - DNA, 1599 bp, synthetic construct
  source 1..1599: mol_type = other DNA; note = MG153-23_cloned_into_untethered_plasmid
gctagcgccaccatgtacccctatgatgtacctgattatgcttctggcggcagcagcggcggctcaagcggaagtgaaacacctggaacttccgaaagcgctacgcctgaatcttctggtggatcttctggcggatcttccgcatgcatgagggctgacgaggctgaggctcacgccagcgccgcgagcacaggaaagggcggcaggaaccttcctggtactgcagctggcgctgaagtgagagctgccgcaggtgggcgaacgaaaccggaggctctgaggcttatggaggctgccgttgagagatctaacatgctgggcgcctatgaaagagtggttaagaatcagggcgcccccggggttgatggcctgaccgtgactgagtttaaaccatggctgcaggcacactggcccaaaatcagacaagtgctgctcgcaggggaatacatgcccgccgccgtgagaaaggttgatatacccaaaccgcaggggggggtccggaccctgggcattccgacagtgctggatcggctcattcagcaggccctccaccaggtcctgcagcccctgttcgaaccagagttcagtgagtcttcctatggctttagacccggtcggaatgcccaccaggccgtcgaagccgcgcgctcttacgtcgctgaagggaaacgttgggtggttgatctggaaaaattttttgatcgagtgaaccacgacgtacttatggcccgggtggcccgcaaggttaaggatgagagagtgctgaaactgatccgcaggtatctcgaagcaggactgatggagggtggcatgaccagtgcacgaacagagggtacacctcagggtggccccttaagtcccctcttgagcaataccctgcttacagacttggatagagagctggagaaacgcggtcatcgtttctgtcgctatgctgacgattgtaatgtttatgtgggaagcaggagatctggtcagagggtcatggcaaagattaccgcgtttctggaacaaaggcttaagttacaggttaatgctgataaaagcgccgtggctagaccatggcaaaggaaattccttgggtacagcgtcacctggcaccggaaccccaaacttaaaattgctccatcctccagacagagactggctgagaagatacggcagaccctgagaggggcccgcggccagagcctcaggcaggtaatcgcacagctgaaccctatcctgaggggctgggttgcttactttagactgaccgaagaaaaaggcgtcctggaagaattagatgggtgggtcaggcgcaagctgagagccttgctttggagacattggaaacgcgggtacccaagagcacagaacttgatgagggctggtttaaggccagaaagagcttggcagtctgcgacaaatgggcacgggccttggtggaatgggggatcatctcatatgaatgctgcatgtccgaagagctggttcgatcacatgcagctcgtgtcacttttggctacccagagaagattttcccttgtaagccctaggcctaagaagaaaagaaaggtgggtggttctcctaagaaaaagagaaaggtctgagaattc

SEQ ID NO: 3 - DNA, 1611 bp, synthetic construct
  source 1..1611: mol_type = other DNA; note = MG153-24_cloned_into_untethered_plasmid
gctagcgccaccatgtacccctatgatgtacctgattatgcttctggcggcagcagcggcggctcaagcggaagtgaaacacctggaacttccgaaagcgctacgcctgaatcttctggtggatcttctggcggatcttccgcatgcatgaaggggggaaagcaaaagattagccaggacacatgtctccaagagtccagagccgaaccagagggttatgctgggggccagacctttatctggatgacagagaacaatctcacaaacgctaacaaaccagaatatgggcttctggagcaaatcctgtcccctacaaaccttaatagggcttataaaggagtgcgatccaatagggggtctggaggtattgataaaatggaggttgagtccctgaaggactatctggttgacaataaggagacgttaattcagtctatattggacggaaaatatagaccaaaccctgtgaggagggtggagattagcaaggagaagggcacacgcaagctcggtatacctacggtagtagacagggtcatccagcaggccattgctcaggttttgtcaccaacttacgaaaggcagttcagcgaaaattcctatgggttccgcccaggcagaaacgcccatcaggctctgaaccgctgtagggattacatcacagacggctacatatacgcagtagatatggatctcgagaaattctttgataccgtgaatcagtcaaaactcatagaggtcttgtccaggacagtgaaagatggccgtgtggtgtcacttattcacaaataccttgatgccggcgttgtgatcagaaataagttcgaagaaacagagatgggagttcctcaaggaagcccattgagccccgttctgtctaacattatgcttaacgaactggacaaagagctggaaaagagggggcacccattcgtgagatatgcagacgacctgattatattctgcaaatctaaaagaagcgccgaccggacgctggctaacaccgtgccctatatcgaaaacaagctgtttctgaaagtgaacagggagaaaactactacagcctatgtctctggaattaaattcctcggctactccttttatgtgtataaaggagaaggccggctgcgcgtgcacccgaaaagcattgctaagatgaaagaaaggattagaaaactgacttctcgttctaatggctggggctacgctcgccgcaaggaggcccttagacagtacatcacaggctgggtcaactatttcaaacttgcagatatgaaaaaactgttagttagcgttgacgagtggtacagacgacgcctgcgtctcgttatatggaagcagtggaaaagggttaggacgcgggggcggtatctgatgaaactggggatcgtgaagcatcaagcatgggagtttgccaatacacgcaaaggttactggcacactgccaaatcaccaatccttaacagatctgtaacgtccaaccggctcagacaagccgggtatgtattcttcgtggattactaccgggtagtgaatggaataaaccctaggcctaagaagaaaagaaaggtgggtggttctcctaagaaaaagagaaaggtctgagaattc

SEQ ID NO: 4 - DNA, 1026 bp, synthetic construct
  source 1..1026: mol_type = other DNA; note = MG160-7_cloned_into_untethered_plasmid
gctagcgccaccatgtacccctatgatgtacctgattatgcttctggcggcagcagcggcggctcaagcggaagtgaaacacctggaacttccgaaagcgctacgcctgaatcttctggtggatcttctggcggatcttccgcatgcatgcataacttcaagagtagattcgaactctccaagggcaaatgggtttacatccagatagaggacctcgccaaccatgccaaggaccacatcagacagattagggacctgtggacaccgccagagtactttttccaccttcaaaagggcggacatattgcagctctcaggcttcatacgcctaacgagtggtacggcaaggtcgatctgtctaagttctttaacaacataacccggcacagggtgacacgcagccttaaaagaattggctattctttccgagacgccgaagagttcgccgttgccagcactgtgcttgtgaatcacgcaaccagacgctatgtgctgccatacgggtttgtccagtctcccttgttggcgtccatcagcctcgacaaaagtgatcttggaaactgcctgaggcgccttcacgaagaaactgtcagcgtcagtgtatatgttgatgatatcgtggtatccgcagatagcgagcgagacgttgcagaggccctgtccaacatctatctggcagcgattaattcacggtttcctattaatgaagagaaaagtcgaggtcccagctcaacactgaatgcctttaatatcgaactggacatgcacgagttggagatcactgctaaacggtacgagaagatgtgtggtgaagtcctgatcaacgggaccggacaggtgtccgatgctattttgaattacgtgcagaccgtgaatcccgctcaggccgaacaaatgctccgggattttcctggcgtgttcgggacactgtcatcttccgcttctcgctatgacgcttcacctaggcctaagaagaaaagaaaggtgggtggttctcctaagaaaaagagaaaggtctgagaattc

SEQ ID NO: 5 - DNA, 5076 bp, synthetic construct
  source 1..5076: mol_type = other DNA; note = MG160-7_cloned_into_tethered_plasmid
atgaagaggaccgctgatggttccgaatttgaatctcctaagaagaaacgaaaggttgataagaaatatagtattgggttggatattggtacgaatagtgtaggatgggcggttattactgacgaatataaagtcccctctaagaagtttaaagtcctcggtaatacagataggcatagtattaagaagaatcttattggcgctctcttatttgattccggtgagactgctgaagctacaaggttaaagaggactgctcgccggcgctatacacgaaggaagaatcgcatatgttaccttcaggaaatatttagtaatgaaatggcaaaagtcgatgattcattcttccatcgacttgaggaatcatttctcgttgaggaagacaagaagcatgaaaggcatccaatctttggaaatatagtcgatgaagtagcttatcatgagaaatatccgactatttatcatttgcgaaagaagcttgtcgattctacagataaagcggatcttaggcttatatacctcgcactcgctcatatgattaaatttaggggtcattttctgattgaaggggatctcaatcctgataattccgatgtagataagctctttattcaacttgtacaaacatataatcaattatttgaagagaatcctattaatgcttctggagtcgatgcaaaagcgattttgagcgctcgcctttccaaaagtcgcagacttgagaacttgattgctcaactcccaggtgaaaagaagaacggactctttggtaatttgatcgcattgagtcttgggttgactccaaattttaaaagtaattttgatttggctgaagacgcaaagctccaattgtccaaagatacttatgatgatgatttggataatttattagcccaaattggagatcaatatgctgatctcttcctcgcagctaagaatctctcagatgcaattttgttgtctgatatacttcgcgtaaatactgaaattacaaaagctcctctctccgcaagcatgataaaacggtatgatgaacatcatcaagatcttactcttcttaaggccttagtacgccaacaacttccggagaaatataaggaaatcttctttgatcaatcaaagaatgggtatgcagggtatatagatggcggcgcgtcacaagaggaattttataaatttataaaaccgattcttgagaaaatggatggaacagaagagcttttggtcaaattaaatagggaagatttgttgaggaaacaaagaacctttgataatgggtctattccacatcaaatacatcttggcgaattgcacgcgatactcaggcgacaagaggacttctatccttttcttaaagataatcgcgagaaaattgaaaagattctcacgtttcggattccttattatgttggaccgttagcgcggggtaattcacggtttgcttggatgactaggaaatcagaagagactattacgccttggaattttgaagaggttgtcgataaaggagcctcagctcaatcttttattgaacgaatgactaattttgacaagaatctccctaatgaaaaggtcttgcctaaacatagtcttctctatgaatattttacagtctacaatgaattgacaaaggttaagtatgtcacagaaggtatgaggaaacctgcatttctgagtggggaacaaaagaaagcgattgtcgatctcctctttaaaacgaatagaaaggtcacggtaaaacaattaaaggaagattattttaagaagattgaatgttttgattcagtcgagatatctggggtcgaggacagatttaatgcaagcttgggtacctatcatgacttgctcaagatcattaaagataaagattttctcgataacgaagagaatgaagatatccttgaggacatagtccttactctcaccctcttcgaagatcgggaaatgattgaagagcgccttaagacatacgcacatctctttgatgataaggttatgaaacaactcaaacgccgacggtatacaggatggggaagacttagtcgcaaactcataaatggaataagggataaacaatcaggaaagaccattcttgactttcttaaatctgatggatttgcaaatcgcaatttcatgcaactcattcatgatgattccctcacattcaaggaagatatacaaaaggcacaagtcagtgggcaaggggactcccttcatgaacatatcgcaaaccttgctggttctcctgcaatcaagaagggaatactccaaactgtaaaagtcgtcgatgaacttgttaaggtaatgggacgccataaaccggaaaatattgtaatagagatggcgcgagaaaatcaaacgactcaaaagggccagaagaatagcagagaaaggatgaaacgcatagaggaagggataaaggaattggggtcccaaattttgaaggagcatccagtagagaatactcaattgcaaaatgagaaactctatctttattatctccaaaacggcagggacatgtatgtcgatcaagagttagatataaatcgccttagtgattatgacgtagatgcaattgtcccccaatccttccttaaagatgatagtattgataataaagtcttgactaggagtgataagaatcgcgggaaaagtgataatgtaccatctgaggaagtagtaaagaaaatgaagaattattggcgacaactcctcaatgcaaaattgatcacgcaacgaaaatttgataacttgacgaaagctgaacggggcggactctccgagcttgacaaagcagggtttattaaacgccaattggtcgagactagacaaattacgaaacatgttgctcaaattcttgatagcagaatgaataccaaatatgatgaaaacgataaattgattcgagaggtcaaggtaattacactcaaatcaaaattagtctctgactttaggaaagactttcaattctataaggtaagggaaattaataattatcatcatgcgcatgatgcatatttgaatgctgtggtcggcacagcgttgattaagaaatatcccaaattagagtcagaatttgtttatggagattataaagtctatgatgttaggaaaatgattgcaaagtctgagcaagagattgggaaagcgacagcaaaatatttcttttatagtaatattatgaacttctttaaaacggaaatcactctcgctaatggggaaattcgcaaaagaccccttattgaaactaatggtgagacaggcgaaattgtttgggacaaaggacgtgacttcgctacagtccgcaaggttctttctatgccacaggtaaacattgttaagaaaactgaagtccaaactggcggtttctccaaggaaagcattctccctaaacgcaatagtgacaaattgattgcaaggaagaaggattgggatccaaagaaatatggtgggtttgattcaccgacagttgcgtacagtgtccttgttgtagctaaggtagagaaaggaaaatccaagaagcttaaatcagttaaggaattattaggcattacaattatggagaggtccagttttgagaagaacccgatagatttcttagaggctaaagggtacaaagaggttaagaaagatcttattataaaacttccaaaatatagtctctttgaactggagaatggacgaaaacgaatgcttgcaagtgctggagagttacaaaagggcaatgagctcgctttaccaagcaagtacgtaaattttctctatcttgcatcccattacgaaaagttgaaaggtagtccagaagacaacgaacaaaagcaattattcgtagagcaacataaacattatcttgatgaaattattgaacagattagtgaattttcaaaacgcgttatactcgcagatgccaacttagataaggtactctctgcttataataaacatagggacaaacctatacgagaacaagcggaaaacattattcatttgttcacactcactaacctcggcgctccagctgcttttaaatatttcgatactacaatagatagaaaacgttatacttctacaaaggaagtcttagatgctacactcattcatcaaagtattacaggattatatgaaactcgtatagatctctcacaactcggcggggattctggcggcagcagcggcggctcaagcggaagtgaaacacctggaacttccgaaagcgctacgcctgaatcttctggtggatcttctggcggatcttccgcatgcatgcataacttcaagagtagattcgaactctccaagggcaaatgggtttacatccagatagaggacctcgccaaccatgccaaggaccacatcagacagattagggacctgtggacaccgccagagtactttttccaccttcaaaagggcggacatattgcagctctcaggcttcatacgcctaacgagtggtacggcaaggtcgatctgtctaagttctttaacaacataacccggcacagggtgacacgcagccttaaaagaattggctattctttccgagacgccgaagagttcgccgttgccagcactgtgcttgtgaatcacgcaaccagacgctatgtgctgccatacgggtttgtccagtctcccttgttggcgtccatcagcctcgacaaaagtgatcttggaaactgcctgaggcgccttcacgaagaaactgtcagcgtcagtgtatatgttgatgatatcgtggtatccgcagatagcgagcgagacgttgcagaggccctgtccaacatctatctggcagcgattaattcacggtttcctattaatgaagagaaaagtcgaggtcccagctcaacactgaatgcctttaatatcgaactggacatgcacgagttggagatcactgctaaacggtacgagaagatgtgtggtgaagtcctgatcaacgggaccggacaggtgtccgatgctattttgaattacgtgcagaccgtgaatcccgctcaggccgaacaaatgctccgggattttcctggcgtgttcgggacactgtcatcttccgcttctcgctatgacgcttca

SEQ ID NO: 6 - RNA, 125 nt, synthetic construct
  source 1..125: mol_type = other RNA; note = VEGFA_G_to_T_change_PBS2
  modified_base 1..3: mod_base = OTHER; note = 2'O-methyl ribonucleotide
  modified_base 1..4: mod_base = OTHER; note = phosphorothioate linkage
  modified_base 123..125: mod_base = OTHER; note = 2'-O-methyl ribonucleotide
  modified_base 122..125: mod_base = OTHER; note = phosphorothioate linkage
gattctgcaggccagatgagttttagagctagaaatagcaagttaaaataaggctagtccgttatcaacttgaaaaagtgggaccgagtcggtccaatgtgccatctggagcactcatctttttt

SEQ ID NO: 7 - RNA, 128 nt, synthetic construct
  source 1..128: mol_type = other RNA; note = VEGFA_G_to_T_change_PBS4
  modified_base 1..3: mod_base = OTHER; note = 2'O-methyl ribonucleotide
  modified_base 1..4: mod_base = OTHER; note = phosphorothioate linkage
  modified_base 126..128: mod_base = OTHER; note = 2'-O-methyl ribonucleotide
  modified_base 125..128: mod_base = OTHER; note = phosphorothioate linkage
gatgtctgcaggccagatgagttttagagctagaaatagcaagttaaaataaggctagtccgttatcaacttgaaaaagtggcaccgagtcggtgcaatgtgccatctggagcactcatctgtttttt

SEQ ID NO: 8 - RNA, 130 nt, synthetic construct
  source 1..130: mol_type = other RNA; note = VEGFA_G_to_T_change_PBS6
  modified_base 1..3: mod_base = OTHER; note = 2'O-methyl ribonucleotide
  modified_base 1..4: mod_base = OTHER; note = phosphorothioate linkage
  modified_base 128..130: mod_base = OTHER; note = 2'-O-methyl ribonucleotide
  modified_base 127..130: mod_base = OTHER; note = phosphorothioate linkage
gatgtctgcaggccagatgagttttagagctagaaatagcaagttaaaataaggctagtccgttatcaacttgaaaaagtggcaccgagtcggtgcaatgtgccatctggagcactcatctggctttttt

SEQ ID NO: 9 - RNA, 132 nt, synthetic construct
  source 1..132: mol_type = other RNA; note = VEGFA_G_to_T_change_PBS8
  modified_base 1..3: mod_base = OTHER; note = 2'O-methyl ribonucleotide
  modified_base 1..4: mod_base = OTHER; note = phosphorothioate linkage
  modified_base 130..132: mod_base = OTHER; note = 2'-O-methyl ribonucleotide
  modified_base 129..132: mod_base = OTHER; note = phosphorothioate linkage
gatgtctgcaggccagatgagttttagagctagaaatagcaagttaaaataaggctagtccgttatcaacttgaaaaagtggcaccgagtcggtgcaatgtgccatctggagcactcatctggccttttttt

SEQ ID NO: 10 - RNA, 134 nt, synthetic construct
  source 1..134: mol_type = other RNA; note = VEGFA_G_to_T_change_PBS10
  modified_base 1..3: mod_base = OTHER; note = 2'O-methyl ribonucleotide
  modified_base 1..4: mod_base = OTHER; note = phosphorothioate linkage
  modified_base 132..134: mod_base = OTHER; note = 2'-O-methyl ribonucleotide
  modified_base 131..134: mod_base = OTHER; note = phosphorothioate linkage
gatgtctgcaggccagatgagttttagagctagaaatagcaagttaaaataaggctagtccgttatcaacttgaaaaagtggcaccgagtcggtgcaatgtgccatctggagcactcatctggcctgctttttt

SEQ ID NO: 11 - RNA, 137 nt, synthetic construct
  source 1..137: mol_type = other RNA; note = VEGFA_G_to_T_change_PBS13
  modified_base 1..3: mod_base = OTHER; note = 2'O-methyl ribonucleotide
  modified_base 1..4: mod_base = OTHER; note = phosphorothioate linkage
  modified_base 135..137: mod_base = OTHER; note = 2'-O-methyl ribonucleotide
  modified_base 134..137: mod_base = OTHER; note = phosphorothioate linkage
gatgtctgcaggccagatgagttttagagctagaaatagcaagttaaaataaggctagtccgttatcaacttgaaaaagtggcaccgagtcggtgcaatgtgccatctggagcactcatctggcctgcagatttttt

SEQ ID NO: 12 - RNA, 140 nt, synthetic construct
  source 1..140: mol_type = other RNA; note = VEGFA_G_to_T_change_PBS16
  modified_base 1..3: mod_base = OTHER; note = 2'O-methyl ribonucleotide
  modified_base 1..4: mod_base = OTHER; note = phosphorothioate linkage
  modified_base 138..140: mod_base = OTHER; note = 2'-O-methyl ribonucleotide
  modified_base 137..140: mod_base = OTHER; note = phosphorothioate linkage
gatgtctgcaggccagatgagttttagagctagaaatagcaagttaaaataaggctagtccgttatcaacttgaaaaagtgggaccgagtcggtccaatgtgccatctggagcactcatctggcctgcagacattttttt

SEQ ID NO: 13 - RNA, 144 nt, synthetic construct
  source 1..144: mol_type = other RNA; note = VEGFA_G_to_T_change_PBS20
  modified_base 1..3: mod_base = OTHER; note = 2'O-methyl ribonucleotide
  modified_base 1..4: mod_base = OTHER; note = phosphorothioate linkage
  modified_base 142..144: mod_base = OTHER; note = 2'-O-methyl ribonucleotide
  modified_base 141..144: mod_base = OTHER; note = phosphorothioate linkage
gatgtctgcaggccagatgagttttagagctagaaatagcaagttaaaataaggctagtccgttatcaacttgaaaaagtggcaccgagtcggtgcaatgtgccatctggagcactcatctggcctgcagacatcaaatttttt

SEQ ID NO: 14 - RNA, 127 nt, synthetic construct
  source 1..127: mol_type = other RNA; note = VEGFA_deletion_change_PBS2
  modified_base 1..3: mod_base = OTHER; note = 2'O-methyl ribonucleotide
  modified_base 1..4: mod_base = OTHER; note = phosphorothioate linkage
  modified_base 125..127: mod_base = OTHER; note = 2'-O-methyl ribonucleotide
  modified_base 124..127: mod_base = OTHER; note = phosphorothioate linkage
gatgtctgcaggccagatgagttttagagctagaaatagcaagttaaaataaggctagtccgttatcaacttgaaaaagtggcaccgagtcggtgcgtgtgtccctctgacaatgtgctcttttttt

SEQ ID NO: 15 - RNA, 129 nt, synthetic construct
  source 1..129: mol_type = other RNA; note = VEGFA_deletion_change_PBS4
  modified_base 1..3: mod_base = OTHER; note = 2'O-methyl ribonucleotide
  modified_base 1..4: mod_base = OTHER; note = phosphorothioate linkage
  modified_base 127..129: mod_base = OTHER; note = 2'-O-methyl ribonucleotide
  modified_base 126..129: mod_base = OTHER; note = phosphorothioate linkage
gatgtctgcaggccagatgagttttagagctagaaatagcaagttaaaataaggctagtccgttatcaacttgaaaaagtggcaccgagtcggtgcgtgtgtccctctgacaatgtgctctgttttttt

SEQ ID NO: 16 - RNA, 131 nt, synthetic construct
  source 1..131: mol_type = other RNA; note = VEGFA_deletion_change_PBS6
  modified_base 1..3: mod_base = OTHER; note = 2'O-methyl ribonucleotide
  modified_base 1..4: mod_base = OTHER; note = phosphorothioate linkage
  modified_base 129..131: mod_base = OTHER; note = 2'-O-methyl ribonucleotide
  modified_base 128..131: mod_base = OTHER; note = phosphorothioate linkage
gatgtctgcaggccagatgagttttagagctagaaatagcaagttaaaataaggctagtccgttatcaacttgaaaaagtggcaccgagtcggtgcgtgtgtccctctgacaatgtgctctggcttttttt

SEQ ID NO: 17 - RNA, 133 nt, synthetic construct
  source 1..133: mol_type = other RNA; note = VEGFA_deletion_change_PBS8
  modified_base 1..3: mod_base = OTHER; note = 2'O-methyl ribonucleotide
  modified_base 1..4: mod_base = OTHER; note = phosphorothioate linkage
  modified_base 131..133: mod_base = OTHER; note = 2'-O-methyl ribonucleotide
  modified_base 130..133: mod_base = OTHER; note = phosphorothioate linkage
gatgtctgcaggccagatgagttttagagctagaaatagcaagttaaaataaggctagtccgttatcaacttgaaaaagtggcaccgagtcggtgcgtgtgtccctctgacaatgtgctctggcctttttttt

SEQ ID NO: 18 - RNA, 135 nt, synthetic construct
  source 1..135: mol_type = other RNA; note = VEGFA_deletion_change_PBS10
  modified_base 1..3: mod_base = OTHER; note = 2'O-methyl ribonucleotide
  modified_base 1..4: mod_base = OTHER; note = phosphorothioate linkage
  modified_base 133..135: mod_base = OTHER; note = 2'-O-methyl ribonucleotide
  modified_base 132..135: mod_base = OTHER; note = phosphorothioate linkage
gatgtctgcaggccagatgagttttagagctagaaatagcaagttaaaataaggctagtccgttatcaacttgaaaaagtggcaccgagtcggtgcgtgtgtccctctgacaatgtgctctggcctgcttttttt

SEQ ID NO: 19 - RNA, 138 nt, synthetic construct
  source 1..138: mol_type = other RNA; note = VEGFA_deletion_change_PBS13
  modified_base 1..3: mod_base = OTHER; note = 2'O-methyl ribonucleotide
  modified_base 1..4: mod_base = OTHER; note = phosphorothioate linkage
  modified_base 136..138: mod_base = OTHER; note = 2'-O-methyl ribonucleotide
  modified_base 135..138: mod_base = OTHER; note = phosphorothioate linkage
gatgtctgcaggccagatgagttttagagctagaaatagcaagttaaaataaggctagtccgttatcaacttgaaaaagtggcaccgagtcggtgcgtgtgtccctctgacaatgtgctctggcctgcagattttttt

SEQ ID NO: 20 - RNA, 141 nt, synthetic construct
  source 1..141: mol_type = other RNA; note = VEGFA_deletion_change_PBS16
  modified_base 1..3: mod_base = OTHER; note = 2'O-methyl ribonucleotide
  modified_base 1..4: mod_base = OTHER; note = phosphorothioate linkage
  modified_base 139..141: mod_base = OTHER; note = 2'-O-methyl ribonucleotide
  modified_base 138..141: mod_base = OTHER; note = phosphorothioate linkage
gatgtctgcaggccagatgagttttagagctagaaatagcaagttaaaataaggctagtccgttatcaacttgaaaaagtggcaccgagtcggtgcgtgtgtccctctgacaatgtgctctggcctgcagacatttttttt

SEQ ID NO: 21 - RNA, 145 nt, synthetic construct
  source 1..145: mol_type = other RNA; note = VEGFA_deletion_change_PBS20
  modified_base 1..3: mod_base = OTHER; note = 2'O-methyl ribonucleotide
  modified_base 1..4: mod_base = OTHER; note = phosphorothioate linkage
  modified_base 143..145: mod_base = OTHER; note = 2'-O-methyl ribonucleotide
  modified_base 142..145: mod_base = OTHER; note = phosphorothioate linkage
gatgtctgcaggccagatgagttttagagctagaaatagcaagttaaaataaggctagtccgttatcaacttgaaaaagtggcaccgagtcggtgcgtgtgtccctctgacaatgtgctctggcctgcagacatcaaattttttt

SEQ ID NO: 22 - RNA, 151 nt, synthetic construct
  source 1..151: mol_type = other RNA; note = VEGFA_insertion_change_PBS2
  modified_base 1..3: mod_base = OTHER; note = 2'O-methyl ribonucleotide
  modified_base 1..4: mod_base = OTHER; note = phosphorothioate linkage
  modified_base 149..151: mod_base = OTHER; note = 2'-O-methyl ribonucleotide
  modified_base 148..151: mod_base = OTHER; note = phosphorothioate linkage
gatgtctgcaggccagatgagttttagagctagaaatagcaagttaaaataaggctagtccgttatcaacttgaaaaagtggcaccgagtcggtgcaatgtgccatctggagcactcacttatcgtcgtcatccttgtaatctcttttttt

SEQ ID NO: 23 - RNA, 153 nt, synthetic construct
  source 1..153: mol_type = other RNA; note = VEGFA_insertion_change_PBS4
  modified_base 1..3: mod_base = OTHER; note = 2'O-methyl ribonucleotide
  modified_base 1..4: mod_base = OTHER; note = phosphorothioate linkage
  modified_base 151..153: mod_base = OTHER; note = 2'-O-methyl ribonucleotide
  modified_base 150..153: mod_base = OTHER; note = phosphorothioate linkage
gatgtctgcaggccagatgagttttagagctagaaatagcaagttaaaataaggctagtccgttatcaacttgaaaaagtggcaccgagtcggtgcaatgtgccatctggagcactcacttatcgtcgtcatccttgtaatctctgttttttt

SEQ ID NO: 24 - RNA, 155 nt, synthetic construct
  source 1..155: mol_type = other RNA; note = VEGFA_insertion_change_PBS6
  modified_base 1..3: mod_base = OTHER; note = 2'O-methyl ribonucleotide
  modified_base 1..4: mod_base = OTHER; note = phosphorothioate linkage
  modified_base 153..155: mod_base = OTHER; note = 2'-O-methyl ribonucleotide
  modified_base 152..155: mod_base = OTHER; note = phosphorothioate linkage
gatgtctgcaggccagatgagttttagagctagaaatagcaagttaaaataaggctagtccgttatcaacttgaaaaagtggcaccgagtcggtgcaatgtgccatctggagcactcacttatcgtcgtcatccttgtaatctctggcttttttt

SEQ ID NO: 25 - RNA, 157 nt, synthetic construct
  source 1..157: mol_type = other RNA; note = VEGFA_insertion_change_PBS8
  modified_base 1..3: mod_base = OTHER; note = 2'O-methyl ribonucleotide
  modified_base 1..4: mod_base = OTHER; note = phosphorothioate linkage
  modified_base 155..157: mod_base = OTHER; note = 2'-O-methyl ribonucleotide
  modified_base 154..157: mod_base = OTHER; note = phosphorothioate linkage
gatgtctgcaggccagatgagttttagagctagaaatagcaagttaaaataaggctagtccgttatcaacttgaaaaagtggcaccgagtcggtgcaatgtgccatctggagcactcacttatcgtcgtcatccttgtaatctctggcctttttttt

SEQ ID NO: 26 - RNA, 159 nt, synthetic construct
  source 1..159: mol_type = other RNA; note = VEGFA_insertion_change_PBS10
  modified_base 1..3: mod_base = OTHER; note = 2'O-methyl ribonucleotide
  modified_base 1..4: mod_base = OTHER; note = phosphorothioate linkage
  modified_base 157..159: mod_base = OTHER; note = 2'-O-methyl ribonucleotide
  modified_base 156..159: mod_base = OTHER; note = phosphorothioate linkage
gatgtctgcaggccagatgagttttagagctagaaatagcaagttaaaataaggctagtccgttatcaacttgaaaaagtggcaccgagtcggtgcaatgtgccatctggagcactcacttatcgtcgtcatccttgtaatctctggcctgcttttttt

SEQ ID NO: 27 - RNA, 162 nt, synthetic construct
  source 1..162: mol_type = other RNA; note = VEGFA_insertion_change_PBS13
  modified_base 1..3: mod_base = OTHER; note = 2'O-methyl ribonucleotide
  modified_base 1..4: mod_base = OTHER; note = phosphorothioate linkage
  modified_base 160..162: mod_base = OTHER; note = 2'-O-methyl ribonucleotide
  modified_base 159..162: mod_base = OTHER; note = phosphorothioate linkage
gatgtctgcaggccagatgagttttagagctagaaatagcaagttaaaataaggctagtccgttatcaacttgaaaaagtggcaccgagtcggtgcaatgtgccatctggagcactcacttatcgtcgtcatccttgtaatctctggcctgcagattttttt

SEQ ID NO: 28 - RNA, 165 nt, synthetic construct
  source 1..165: mol_type = other RNA; note = VEGFA_insertion_change_PBS16
  modified_base 1..3: mod_base = OTHER; note = 2'O-methyl ribonucleotide
  modified_base 1..4: mod_base = OTHER; note = phosphorothioate linkage
  modified_base 163..165: mod_base = OTHER; note = 2'-O-methyl ribonucleotide
  modified_base 162..165: mod_base = OTHER; note = phosphorothioate linkage
gatgtctgcaggccagatgagttttagagctagaaatagcaagttaaaataaggctagtccgttatcaacttgaaaaagtggcaccgagtcggtgcaatgtgccatctggagcactcacttatcgtcgtcatccttgtaatctctggcctgcagacatttttttt

SEQ ID NO: 29 - RNA, 169 nt, synthetic construct
  source 1..169: mol_type = other RNA; note = VEGFA_insertion_change_PBS20
  modified_base 1..3: mod_base = OTHER; note = 2'O-methyl ribonucleotide
  modified_base 1..4: mod_base = OTHER; note = phosphorothioate linkage
  modified_base 167..169: mod_base = OTHER; note = 2'-O-methyl ribonucleotide
  modified_base 166..169: mod_base = OTHER; note = phosphorothioate linkage
gatgtctgcaggccagatgagttttagagctagaaatagcaagttaaaataaggctagtccgttatcaacttgaaaaagtggcaccgagtcggtgcaatgtgccatctggagcactcacttatcgtcgtcatccttgtaatctctggcctgcagacatcaaattttttt

SEQ ID NO: 30 - DNA, 40 bp, synthetic construct
  source 1..40: mol_type = other DNA; note = VEGFA_forward_primer
gctcttccgatctnnnnnaaattcttctcccctgggaagc

SEQ ID NO: 31 - DNA, 40 bp, synthetic construct
  source 1..40: mol_type = other DNA; note = VEGFA_reverse_primer
gctcttccgatctnnnnntggaccccctatttctgacctc

SEQ ID NO: 32 - DNA, 230 bp, synthetic construct
  source 1..230: mol_type = other DNA; note = VEGFA_target_amplified
aaattcttctcccctgggaagcatccctggacacttcccaaaggaccccagtcactccagcctgttggctgccgctcactttgatgtctgcaggccagatgagggctccagatggcacattgtcagagggacacactgtggcccctgtgcccagccctgggctctctgtacatgaagcaactccagtcccaaatatgtagctgtttgggaggtcagaaatagggggtcca

SEQ ID NO: 33 - DNA, 105 bp, synthetic construct
  source 1..105: mol_type = other DNA; note = linker 1 for tethered RT
tctggcggcagcagcggcggctcaagcggaagtgaaacacctggaacttccgaaagcgctacgcctgaatcttctggtggatcttctggcggatcttccgcatgc

SEQ ID NO: 34 - DNA, 3652 bp, synthetic construct
  source 1..3652: mol_type = other DNA; note = MG3-6-mRNA-wild-type
aaaagccagctccagcaggcgctgctcactcctccccatcctctccctctgtccctctgtccctctgaccctgcactgtcccagcaccatggcccccaagaagaagcggaaagttaccggtggcggaggcagcagcaccgacatgaagaactaccgcatcggagtggacgtgggcgacagaagcgtgggactggccgccatcgaattcgacgacgacgggctgcccatccagaaactggcactggtgaccttccggcacgacggcggcctggaccccacgaagaacaagacaccgatgagcaggaaagagacgaggggaatcgcacgcagaacaatgaggatgaaccgagaaaggaagaggcgcctgagaaacctggacaacgtgctggaaaacctgggctacagcgtgcccgagggccccgagcccgaaacctacgaagcatggaccagccgggcactgctggccagcatcaagctggccagcgcagacgaactgaacgaacacctggtgcgagcggtgaggcacatggcacgccaccgaggatgggccaacccctggtggagcctggaccagctggaaaaggcaagccaggagcccagcgaaacattcgagatcatcctggcccgggccagggagctgttcggggagaaagtgcccgcaaaccccaccctgggcatgctgggagcactggccgcgaacaacgaagtgctgctgcgcccgagggacgagaagaagaggaagaccggatacgtgagagggacacccctgatgttcgcacaagtgcgacaaggcgaccaactggcagaactgaggcgaatctgcgaggtgcaagggatcgaagaccaatacgaggcactgcggctgggcgtgttcgaccacaaacacccctacgtgccaaaagagagagtgggaaaagacccgctgaaccccagcacaaaccgcacaatcagggcgagcctggagttccaagaattcagaatcctggacagcgtggccaacctgagagtgagaatcggcagcagggcgaaaagagagctgaccgaggccgaatacgacgcagccgtggaattcctgatggactacgcggacaaggagcagcccagctgggccgacgtggccgaaaagatcggcgtgcccggaaaccgactggtggcccccgtgctggaagacgtgcaacagaaaaccgcaccctacgacagaagcagcgcagcattcgagaaagcgatggggaagaagaccgaggcaagacaatggtgggagagcaccgacgacgaccaactgagaagcctgctgatcgcattcctggtggacgccacgaacgacacagaagaggccgccgccgaggcgggactgagcgaactgtacaagagctggcccgcagaagagagagaggccctgagcaacatcgacttcgagaaaggaagggtggcatacagccaagagaccctgagcaaactgagcgaatacatgcacgaatacagagtgggactgcacgaggcccgcaaagcagtgttcggggtggacgacacctggagaccaccactggacaaactggaggagccaacaggccaacccgcagtggacagagtgctgacaatcctgcgacggttcgtgctggactgcgagaggcaatggggccgccccagggccatcaccgtggaacacaccagaaccggcctgatggggccgacccagcgccagaaaatcctgaacgaacaaaagaagaaccgggccgacaacgagcgaatcagagacgagctgagggaaagcggggtggacaaccccagcagggccgaagtgcgaagacacctgatcgtgcaagaacaagagtgccaatgcctgtactgcggaaccatgatcaccacaaccacgagcgagctggaccacatcgtgccccgcgccggaggaggcagcagccgaagagaaaacctggccgcagtgtgcagggcctgcaacgcgaagaagaagcgcgagctgttctacgcctgggcaggcccggtgaaaagccaagaaacaatcgagcgcgtgcgccaactgaaagcattcaaagacagcaagaaggcgaaaatgttcaagaaccagatccggcgactgaaccaaaccgaggcagacgagccaatcgacgagagaagcctggccagcaccagctacgcagccgtggcagtgagggaacgcctggagcaacacttcaacgaagggctggccctggacgacaaaagccgggtggtgctggacgtctacgcgggagccgtgaccagggagagccgcagagcaggcgggatcgacgaaagaatcctgctgcggggcgagcgcgacaagaacagattcgacgtgcggcaccacgccgtggacgcggccgtgatgaccctgctgaaccgcagcgtggccctgaccctggagcaacgcagccaactgcgacgcacattctacgaacaaggcctggacaaactggaccggaaccaactgaaacccgaggaagactggcgagacttcacagggctggcccccgccagccaagagaagttcctggaatggcgaaaggcggcaaccatcctgggcgacctgctggccgaggcgatcgaggacgacagcatcgcagtggtgagccccctgagactgaggccacaaaacggcagcgtgcacctggagacaatcagcgccgtgaagaaacagacactgggaagcgactggcccgccgacgccgtgaaaaggatcgtggacccagagatctacctggccatgaaagacgcactgggcaagctgaaagaactgccagaggacagcgcacggagcctggagctgccggacggcaggttcgtggaggccgacgacgaagtgctgttcttccccgaaaacgccgccagcatcctgacccccagaggggtggccgaaatcggcggcagcatccaccacgcgcggctgtacggctggctgaccaagaaaggggagctgaaagtgggcatgctgagggtgtacggagcagagttcccctggctgatgagggaaagcggcagcagaaacgtgctgagcatgcccatccaccgcggcagccagagcttcagggacatgcaagacaccaccagaaaagccgtggaaagcggagaagccgtggaattcgcctggatcacacagaacgacgaactggaattcgacccggacgactacatcgcccacggcggaaaagacgagctgagacaattcctggggttcatgcccgagtgccgctggcgagtggacggcttcaagaagaactaccagatccgcatccgaccagccatgctgagccgcgagcagctgcccagcgacatccaacgcaggctggaaagcaaaacactgaccaagaacgagagcctgctgctgaaagcactggacaccgggctggtggtggcaatcggcggcctgctgcccctggaaacactgaaggtgatccggagaaacaacctgggcttcccacgatggcgcgggaacgggaacctgccgaccagcttcgaagtgcggagcagcgcactgagagcactgggcgtggaaggatctggcggaaaaagacctgccgccacaaagaaagccggacaggccaagaaaaagaagtgaccacacccccattcccccactccagatagaacttcagttatatctcacgtgtctggagtt

SEQ ID NO: 35 - DNA, 506 bp, synthetic construct
  source 1..506: mol_type = other DNA; note = AAVS1_target_amplified
ggacagcatgtttgctgcctccagggatcctgtgtccccgagctgggaccaccttatattcccagggccggttaatgtggctctggttctgggtacttttatctgtcccctccaccccacagtggggccactagggacaggattggtgacagaaaagcccccatccttaggcctcctccttcctagtctcctgatattcgtctaacccccacctcctgttaggcagattccttatctggtgacacacccccatttcctggagccatctctctccttgccagaacctctaaggtttgcttacgatggagccagagaggatcctgggagggagacttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaa

SEQ ID NO: 36 - DNA, 306 bp, synthetic construct
  source 1..306: mol_type = other DNA; note = B2M_target_amplified
gggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggc

SEQ ID NO: 37 - DNA, 430 bp, synthetic construct
  source 1..430: mol_type = other DNA; note = CD5_target_amplified
ggctgcctgggtcagggtcctctgggaagcccctgcagtgccccagaagggacgaagctcacaaggggcaaggcaggcagcccacggggcaggagggagctcaactgggcgtcctagggagagggcagtgaggggtgccagtggggaacccctcccagcctgacccccaccacacctttctgacccccagatttccaggcaaggctcacccgttccaactcgaagtgccagggccagctggaggtctacctcaaggacggatggcacatggtttgcagccagagctggggccggagctccaagcagtgggaggaccccagtcaagcgtcaaaagtctgccagcggctgaactgtggggtgcccttaagccttggccccttccttgtcacctacacacctcagagctcaatcatctgctacggacaactgg

SEQ ID NO: 38 - DNA, 393 bp, synthetic construct
  source 1..393: mol_type = other DNA; note = CD38_target_amplified
gggacaaaccctgctgccggctctctaggagagcccaactctgtcttggcgtcagtatcctggtcctgatcctcgtcgtggtgctcgcggtggtcgtcccgaggtggcgccagcagtggagcggtccgggcaccaccaagcgctttcccgagaccgtcctggcgcgatgcgtcaagtacactgaaattcatcctgagatgaggtgggttggcgactaaggcgcaccggtgggcactgcggggacagcagggccccgcgcgcagggaagccgcccggatcgcccggaaccgggcatcttccgtggcgggtcagccgagagcccgccgggtggtgctgagtagggagtcccgggctcggggctccgcgggccgctttcaggagcagctggccttg

SEQ ID NO: 39 - RNA, 129 nt, synthetic construct
  source 1..129: mol_type = other RNA; note = AAVS1_PBS_2
  modified_base 1..3: mod_base = OTHER; note = 2'O-methyl ribonucleotide
  modified_base 1..4: mod_base = OTHER; note = phosphorothioate linkage
  modified_base 127..129: mod_base = OTHER; note = 2'-O-methyl ribonucleotide
  modified_base 126..129: mod_base = OTHER; note = phosphorothioate linkage
ggctccatcgtaagcaaaccttgttgagaatcgaaagattcttaataaggcatccttccgatgctgacttctcaccgtccgttatccaataggagcgggcggtatggccagaccttctaaggttttttt

SEQ ID NO: 40 - RNA, 131 nt, synthetic construct
  source 1..131: mol_type = other RNA; note = AAVS1_PBS_4
  modified_base 1..3: mod_base = OTHER; note = 2'O-methyl ribonucleotide
  modified_base 1..4: mod_base = OTHER; note = phosphorothioate linkage
  modified_base 129..131: mod_base = OTHER; note = 2'-O-methyl ribonucleotide
  modified_base 128..131: mod_base = OTHER; note = phosphorothioate linkage
ggctccatcgtaagcaaaccttgttgagaatcgaaagattcttaataaggcatccttccgatgctgacttctcaccgtccgttatccaataggagcgggcggtatggccagaccttctaaggttttttttt

SEQ ID NO: 41 - RNA, 135 nt, synthetic construct
  source 1..135: mol_type = other RNA; note = AAVS1_PBS_8
  modified_base 1..3: mod_base = OTHER; note = 2'O-methyl ribonucleotide
  modified_base 1..4: mod_base = OTHER; note = phosphorothioate linkage
  modified_base 133..135: mod_base = OTHER; note = 2'-O-methyl ribonucleotide
  modified_base 132..135: mod_base = OTHER; note = phosphorothioate linkage
ggctccatcgtaagcaaaccttgttgagaatcgaaagattcttaataaggcatccttccgatgctgacttctcaccgtccgttatccaataggagcgggcggtatggccagaccttctaaggtttgctttttttt

SEQ ID NO: 42 - RNA, 137 nt, synthetic construct
  source 1..137: mol_type = other RNA; note = AAVS1_PBS_10
  modified_base 1..3: mod_base = OTHER; note = 2'O-methyl ribonucleotide
  modified_base 1..4: mod_base = OTHER; note = phosphorothioate linkage
  modified_base 135..137: mod_base = OTHER; note = 2'-O-methyl ribonucleotide
  modified_base 134..137: mod_base = OTHER; note = phosphorothioate linkage
ggctccatcgtaagcaaaccttgttgagaatcgaaagattcttaataaggcatccttccgatgctgacttctcaccgtccgttatccaataggagcgggcggtatggccagaccttctaaggtttgcttactttttt

SEQ ID NO: 43 - RNA, 140 nt, synthetic construct
  source 1..140: mol_type = other RNA; note = AAVS1_PBS_13
  modified_base 1..3: mod_base = OTHER; note = 2'O-methyl ribonucleotide
  modified_base 1..4: mod_base = OTHER; note = phosphorothioate linkage
  modified_base 138..140: mod_base = OTHER; note = 2'-O-methyl ribonucleotide
  modified_base 137..140: mod_base = OTHER; note = phosphorothioate linkage
ggctccatcgtaagcaaaccttgttgagaatcgaaagattcttaataaggcatccttccgatgctgacttctcaccgtccgttatccaataggagcgggcggtatggccagaccttctaaggtttgcttacgattttttt

SEQ ID NO: 44 - RNA, 143 nt, synthetic construct
  source 1..143: mol_type = other RNA; note = AAVS1_PBS_16
  modified_base 1..3: mod_base = OTHER; note = 2'O-methyl ribonucleotide
  modified_base 1..4: mod_base = OTHER; note = phosphorothioate linkage
  modified_base 141..143: mod_base = OTHER; note = 2'-O-methyl ribonucleotide
  modified_base 140..143: mod_base = OTHER; note = phosphorothioate linkage
ggctccatcgtaagcaaaccttgttgagaatcgaaagattcttaataaggcatccttccgatgctgacttctcaccgtccgttatccaataggagcgggcggtatggccagaccttctaaggtttgcttacgatggatttttt

SEQ ID NO: 45 - RNA, 147 nt, synthetic construct
  source 1..147: mol_type = other RNA; note = AAVS1_PBS_20
  modified_base 1..3: mod_base = OTHER; note = 2'O-methyl ribonucleotide
  modified_base 1..4: mod_base = OTHER; note = phosphorothioate linkage
  modified_base 145..147: mod_base = OTHER; note = 2'-O-methyl ribonucleotide
  modified_base 144..147: mod_base = OTHER; note = phosphorothioate linkage
ggctccatcgtaagcaaaccttgttgagaatcgaaagattcttaataaggcatccttccgatgctgacttctcaccgtccgttatccaataggagcgggcggtatggccagaccttctaaggtttgcttacgatggagccatttttt

SEQ ID NO: 46 - RNA, 112 nt, synthetic construct
  source 1..112: mol_type = other RNA; note = AAVS1_Guide
  modified_base 1..3: mod_base = OTHER; note = 2'O-methyl ribonucleotide
  modified_base 1..4: mod_base = OTHER; note = phosphorothioate linkage
  modified_base 110..112: mod_base = OTHER; note = 2'-O-methyl ribonucleotide
  modified_base 109..112: mod_base = OTHER; note = phosphorothioate linkage
ggctccatcgtaagcaaaccttgttgagaatcgaaagattcttaataaggcatccttccgatgctgacttctcaccgtccgttatccaataggagcgggcggtatgtttttt

SEQ ID NO: 47 - RNA, 129 nt, synthetic construct
  source 1..129: mol_type = other RNA; note = B2M_PBS_2
  modified_base 1..3: mod_base = OTHER; note = 2'O-methyl ribonucleotide
  modified_base 1..4: mod_base = OTHER; note = phosphorothioate linkage
  modified_base 127..129: mod_base = OTHER; note = 2'-O-methyl ribonucleotide
  modified_base 126..129: mod_base = OTHER; note = phosphorothioate linkage
cgtcagagcgccgaggttgggggttgagaatcgaaagattcttaataaggcatccttccgatgctgacttctcaccgtccgttatccaataggagcgggcggtatgagagaaacactcctccatttttt

SEQ ID NO: 48 - RNA, 131 nt, synthetic construct
  source 1..131: mol_type = other RNA; note = B2M_PBS_4
  modified_base 1..3: mod_base = OTHER; note = 2'O-methyl ribonucleotide
  modified_base 1..4: mod_base = OTHER; note = phosphorothioate linkage
  modified_base 129..131: mod_base = OTHER; note = 2'-O-methyl ribonucleotide
  modified_base 128..131: mod_base = OTHER; note = phosphorothioate linkage
cgtcagagcgccgaggttgggggttgagaatcgaaagattcttaataaggcatccttccgatgctgacttctcaccgtccgttatccaataggagcgggcggtatgagagaaacactcctccaactttttt

SEQ ID NO: 49 - RNA, 135 nt, synthetic construct
  source 1..135: mol_type = other RNA; note = B2M_PBS_8
  modified_base 1..3: mod_base = OTHER; note = 2'O-methyl ribonucleotide
  modified_base 1..4: mod_base = OTHER; note = phosphorothioate linkage
  modified_base 133..135: mod_base = OTHER; note = 2'-O-methyl ribonucleotide
  modified_base 132..135: mod_base = OTHER; note = phosphorothioate linkage
cgtcagagcgccgaggttgggggttgagaatcgaaagattcttaataaggcatccttccgatgctgacttctcaccgtccgttatccaataggagcgggcggtatgagagaaacactcctccaacctcgtttttt

SEQ ID NO: 50 - RNA, 137 nt, synthetic construct
  source 1..137: mol_type = other RNA; note = B2M_PBS_10
  modified_base 1..3: mod_base = OTHER; note = 2'O-methyl ribonucleotide
  modified_base 1..4: mod_base = OTHER; note = phosphorothioate linkage
  modified_base 135..137: mod_base = OTHER; note = 2'-O-methyl ribonucleotide
  modified_base 134..137: mod_base = OTHER; note = phosphorothioate linkage
cgtcagagcgccgaggttgggggttgagaatcgaaagattcttaataaggcatccttccgatgctgacttctcaccgtccgttatccaataggagcgggcggtatgagagaaacactcctccaacctcggctttttt

SEQ ID NO: 51 - RNA, 140 nt, synthetic construct
  source 1..140: mol_type = other RNA; note = B2M_PBS_13
  modified_base 1..3: mod_base = OTHER; note = 2'O-methyl ribonucleotide
  modified_base 1..4: mod_base = OTHER; note = phosphorothioate linkage
  modified_base 138..140: mod_base = OTHER; note = 2'-O-methyl ribonucleotide
  modified_base 137..140: mod_base = OTHER; note = phosphorothioate linkage
cgtcagagcgccgaggttgggggttgagaatcgaaagattcttaataaggcatccttccgatgctgacttctcaccgtccgttatccaataggagcgggcggtatgagagaaacactcctccaacctcggcgcttttttt

SEQ ID NO: 52 - RNA, 143 nt, synthetic construct
  source 1..143: mol_type = other RNA; note = B2M_PBS_16
  modified_base 1..3: mod_base = OTHER; note = 2'O-methyl ribonucleotide
  modified_base 1..4: mod_base = OTHER; note = phosphorothioate linkage
  modified_base 141..143: mod_base = OTHER; note = 2'-O-methyl ribonucleotide
  modified_base 140..143: mod_base = OTHER; note = phosphorothioate linkage
cgtcagagcgccgaggttgggggttgagaatcgaaagattcttaataaggcatccttccgatgctgacttctcaccgtccgttatccaataggagcgggcggtatgagagaaacactcctccaacctcggcgctctgtttttt

SEQ ID NO: 53 - RNA, 147 nt, synthetic construct
  source 1..147: mol_type = other RNA; note = B2M_PBS_20
  modified_base 1..3: mod_base = OTHER; note = 2'O-methyl ribonucleotide
  modified_base 1..4: mod_base = OTHER; note = phosphorothioate linkage
  modified_base 145..147: mod_base = OTHER; note = 2'-O-methyl ribonucleotide
  modified_base 144..147: mod_base = OTHER; note = phosphorothioate linkage
cgtcagagcgccgaggttgggggttgagaatcgaaagattcttaataaggcatccttccgatgctgacttctcaccgtccgttatccaataggagcgggcggtatgagagaaacactcctccaacctcggcgctctgacgctttttt

SEQ ID NO: 54 - RNA, 112 nt, synthetic construct
  source 1..112: mol_type = other RNA; note = B2M_Guide
  modified_base 1..3: mod_base = OTHER; note = 2'O-methyl ribonucleotide
  modified_base 1..4: mod_base = OTHER; note = phosphorothioate linkage
  modified_base 110..112: mod_base = OTHER; note = 2'-O-methyl ribonucleotide
  modified_base 109..112: mod_base = OTHER; note = phosphorothioate linkage
cgtcagagcgccgaggttgggggttgagaatcgaaagattcttaataaggcatccttccgatgctgacttctcaccgtccgttatccaataggagcgggcggtatgtttttt

SEQ ID NO: 55 - RNA, 129 nt, synthetic construct
  source 1..129: mol_type = other RNA; note = CD5_PBS_2
  modified_base 1..3: mod_base = OTHER; note = 2'O-methyl ribonucleotide
  modified_base 1..4: mod_base = OTHER; note = phosphorothioate linkage
  modified_base 127..129: mod_base = OTHER; note = 2'-O-methyl ribonucleotide
  modified_base 126..129: mod_base = OTHER; note = phosphorothioate linkage
ggtgagccttgcctggaaatctgttgagaatcgaaagattcttaataaggcatccttccgatgctgacttctcaccgtccgttatccaataggagcgggcggtatgtttctgaccgaaagatttttttt

SEQ ID NO: 56 - RNA, 131 nt, synthetic construct
  source 1..131: mol_type = other RNA; note = CD5_PBS_4
  modified_base 1..3: mod_base = OTHER; note = 2'O-methyl ribonucleotide
  modified_base 1..4: mod_base = OTHER; note = phosphorothioate linkage
  modified_base 129..131: mod_base = OTHER; note = 2'-O-methyl ribonucleotide
  modified_base 128..131: mod_base = OTHER; note = phosphorothioate linkage
ggtgagccttgcctggaaatctgttgagaatcgaaagattcttaataaggcatccttccgatgctgacttctcaccgtccgttatccaataggagcgggcggtatgtttctgaccgaaagatttctttttt

SEQ ID NO: 57 - RNA, 135 nt, synthetic construct
  source 1..135: mol_type = other RNA; note = CD5_PBS_8
  modified_base 1..3: mod_base = OTHER; note = 2'O-methyl ribonucleotide
  modified_base 1..4: mod_base = OTHER; note = phosphorothioate linkage
  modified_base 133..135: mod_base = OTHER; note = 2'-O-methyl ribonucleotide
  modified_base 132..135: mod_base = OTHER; note = phosphorothioate linkage
ggtgagccttgcctggaaatctgttgagaatcgaaagattcttaataaggcatccttccgatgctgacttctcaccgtccgttatccaataggagcgggcggtatgtttctgaccgaaagatttccaggtttttt

SEQ ID NO: 58 - RNA, 137 nt, synthetic construct
  source 1..137: mol_type = other RNA; note = CD5_PBS_10
  modified_base 1..3: mod_base = OTHER; note = 2'O-methyl ribonucleotide
  modified_base 1..4: mod_base = OTHER; note = phosphorothioate linkage
  modified_base 135..137: mod_base = OTHER; note = 2'-O-methyl ribonucleotide
  modified_base 134..137: mod_base = OTHER; note = phosphorothioate linkage
ggtgagccttgcctggaaatctgttgagaatcgaaagattcttaataaggcatccttccgatgctgacttctcaccgtccgttatccaataggagcgggcggtatgtttctgaccgaaagatttccaggcatttttt

SEQ ID NO: 59 - RNA, 140 nt, synthetic construct
  source 1..140: mol_type = other RNA; note = CD5_PBS_13
  modified_base 1..3: mod_base = OTHER; note = 2'O-methyl ribonucleotide
  modified_base 1..4: mod_base = OTHER; note = phosphorothioate linkage
  modified_base 138..140: mod_base = OTHER; note = 2'-O-methyl ribonucleotide
  modified_base 137..140: mod_base = OTHER; note = phosphorothioate linkage
ggtgagccttgcctggaaatctgttgagaatcgaaagattcttaataaggcatccttccgatgctgacttctcaccgtccgttatccaataggagcgggcggtatgtttctgaccgaaagatttccaggcaaggtttttt

SEQ ID NO: 60 - RNA, 143 nt, synthetic construct
  source 1..143: mol_type = other RNA; note = CD5_PBS_16
  modified_base 1..3: mod_base = OTHER; note = 2'O-methyl ribonucleotide
  modified_base 1..4: mod_base = OTHER; note = phosphorothioate linkage
  modified_base 141..143: mod_base = OTHER; note = 2'-O-methyl ribonucleotide
  modified_base 140..143: mod_base = OTHER; note = phosphorothioate linkage
ggtgagccttgcctggaaatctgttgagaatcgaaagattcttaataaggcatccttccgatgctgacttctcaccgtccgttatccaataggagcgggcggtatgtttctgaccgaaagatttccaggcaaggctctttttt

SEQ ID NO: 61 - RNA, 147 nt, synthetic construct
  source 1..147: mol_type = other RNA; note = CD5_PBS_20
  modified_base 1..3: mod_base = OTHER; note = 2'O-methyl ribonucleotide
  modified_base 1..4: mod_base = OTHER; note = phosphorothioate linkage
  modified_base 145..147: mod_base = OTHER; note = 2'-O-methyl ribonucleotide
  modified_base 144..147: mod_base = OTHER; note = phosphorothioate linkage
ggtgagccttgcctggaaatctgttgagaatcgaaagattcttaataaggcatccttccgatgctgacttctcaccgtccgttatccaataggagcgggcggtatgtttctgaccgaaagatttccaggcaaggctcaccctttttt

SEQ ID NO: 62 - RNA, 112 nt, synthetic construct
  source 1..112: mol_type = other RNA; note = CD5_Guide
  modified_base 1..3: mod_base = OTHER; note = 2'O-methyl ribonucleotide
  modified_base 1..4: mod_base = OTHER; note = phosphorothioate linkage
  modified_base 110..112: mod_base = OTHER; note = 2'-O-methyl ribonucleotide
  modified_base 109..112: mod_base = OTHER; note = phosphorothioate linkage
ggtgagccttgcctggaaatctgttgagaatcgaaagattcttaataaggcatccttccgatgctgacttctcaccgtccgttatccaataggagcgggcggtatgtttttt

SEQ ID NO: 63 - RNA, 129 nt, synthetic construct
  source 1..129: mol_type = other RNA; note = CD38_PBS_2
  modified_base 1..3: mod_base = OTHER; note = 2'O-methyl ribonucleotide
  modified_base 1..4: mod_base = OTHER; note = phosphorothioate linkage
  modified_base 127..129: mod_base = OTHER; note = 2'-O-methyl ribonucleotide
  modified_base 126..129: mod_base = OTHER; note = phosphorothioate linkage
ctgaaattcatcctgagatgaggttgagaatcgaaagattcttaataaggcatccttccgatgctgacttctcaccgtccgttatccaataggagcgggcggtatgtcgccaacacacctcattttttt

SEQ ID NO: 64 - RNA, 131 nt, synthetic construct
  source 1..131: mol_type = other RNA; note = CD38_PBS_4
  modified_base 1..3: mod_base = OTHER; note = 2'O-methyl ribonucleotide
  modified_base 1..4: mod_base = OTHER; note = phosphorothioate linkage
  modified_base 129..131: mod_base = OTHER; note = 2'-O-methyl ribonucleotide
  modified_base 128..131: mod_base = OTHER; note = phosphorothioate linkage
ctgaaattcatcctgagatgaggttgagaatcgaaagattcttaataaggcatccttccgatgctgacttctcaccgtccgttatccaataggagcgggcggtatgtcgccaacacacctcatcttttttt

SEQ ID NO: 65 - RNA, 135 nt, synthetic construct
  source 1..135: mol_type = other RNA; note = CD38_PBS_8
  modified_base 1..3: mod_base = OTHER; note = 2'O-methyl ribonucleotide
  modified_base 1..4: mod_base = OTHER; note = phosphorothioate linkage
  modified_base 133..135: mod_base = OTHER; note = 2'-O-methyl ribonucleotide
  modified_base 132..135: mod_base = OTHER; note = phosphorothioate linkage
ctgaaattcatcctgagatgaggttgagaatcgaaagattcttaataaggcatccttccgatgctgacttctcaccgtccgttatccaataggagcgggcggtatgtcgccaacacacctcatctcaggtttttt

SEQ ID NO: 66 - RNA, 137 nt, synthetic construct
  source 1..137: mol_type = other RNA; note = CD38_PBS_10
  modified_base 1..3: mod_base = OTHER; note = 2'O-methyl ribonucleotide
  modified_base 1..4: mod_base = OTHER; note = phosphorothioate linkage
  modified_base 135..137: mod_base = OTHER; note = 2'-O-methyl ribonucleotide
  modified_base 134..137: mod_base = OTHER; note = phosphorothioate linkage
ctgaaattcatcctgagatgaggttgagaatcgaaagattcttaataaggcatccttccgatgctgacttctcaccgtccgttatccaataggagcgggcggtatgtcgccaacacacctcatctcaggattttttt

SEQ ID NO: 67 - RNA, 140 nt, synthetic construct
  source 1..140: mol_type = other RNA; note = CD38_PBS_13
  modified_base 1..3: mod_base = OTHER; note = 2'O-methyl ribonucleotide
  modified_base 1..4: mod_base = OTHER; note = phosphorothioate linkage
  modified_base 138..140: mod_base = OTHER; note = 2'-O-methyl ribonucleotide
  modified_base 137..140: mod_base = OTHER; note = phosphorothioate linkage
ctgaaattcatcctgagatgaggttgagaatcgaaagattcttaataaggcatccttccgatgctgacttctcaccgtccgttatccaataggagcgggcggtatgtcgccaacacacctcatctcaggatgaatttttt

SEQ ID NO: 68 - RNA, 143 nt, synthetic construct
  source 1..143: mol_type = other RNA; note = CD38_PBS_16
  modified_base 1..3: mod_base = OTHER; note = 2'O-methyl ribonucleotide
  modified_base 1..4: mod_base = OTHER; note = phosphorothioate linkage
  modified_base 141..143: mod_base = OTHER; note = 2'-O-methyl ribonucleotide
  modified_base 140..143: mod_base = OTHER; note = phosphorothioate linkage
ctgaaattcatcctgagatgaggttgagaatcgaaagattcttaataaggcatccttccgatgctgacttctcaccgtccgttatccaataggagcgggcggtatgtcgccaacacacctcatctcaggatgaattttttttt

SEQ ID NO: 69 - RNA, 146 nt, synthetic construct
  source 1..146: mol_type = other RNA; note = CD38_PBS_20
  modified_base 1..3: mod_base = OTHER; note = 2'O-methyl ribonucleotide
  modified_base 1..4: mod_base = OTHER; note = phosphorothioate linkage
  modified_base 144..146: mod_base = OTHER; note = 2'-O-methyl ribonucleotide
  modified_base 143..146: mod_base = OTHER; note = phosphorothioate linkage
ctgaaattcatcctgagatgaggttgagaatcgaaagattcttaataaggcatccttccgatgctgacttctcaccgtccgttatccaataggagcgggcggtatgtcgccaacacacctcatctcaggatgaatttcagtttttt

SEQ ID NO: 70 - RNA, 112 nt, synthetic construct
  source 1..112: mol_type = other RNA; note = CD38_Guide
  modified_base 1..3: mod_base = OTHER; note = 2'O-methyl ribonucleotide
  modified_base 1..4: mod_base = OTHER; note = phosphorothioate linkage
  modified_base 110..112: mod_base = OTHER; note = 2'-O-methyl ribonucleotide
  modified_base 109..112: mod_base = OTHER; note = phosphorothioate linkage
ctgaaattcatcctgagatgaggttgagaatcgaaagattcttaataaggcatccttccgatgctgacttctcaccgtccgttatccaataggagcgggcggtatgtttttt

SEQ ID NO: 71 - DNA, 21 bp, synthetic construct
  source 1..21: mol_type = other DNA; note = AAVS1_forward_primer
ggacagcatgtttgctgcctc

SEQ ID NO: 72 - DNA, 20 bp, synthetic construct
  source 1..20: mol_type = other DNA; note = AAVS1_reverse_primer
ttccaaactgcttctcctct

SEQ ID NO: 73 - DNA, 20 bp, synthetic construct
  source 1..20: mol_type = other DNA; note = B2M_forward_primer
gggctagtccagggctggat

SEQ ID NO: 74 - DNA, 24 bp, synthetic construct
  source 1..24: mol_type = other DNA; note = B2M_reverse_primer
gccaagcattctacaaacgtcgcg

SEQ ID NO: 75 - DNA, 20 bp, synthetic construct
  source 1..20: mol_type = other DNA; note = CD5_forward_primer
ggctgcctgggtcagggtcc

SEQ ID NO: 76 - DNA, 21 bp, synthetic construct
  source 1..21: mol_type = other DNA; note = CD5_reverse_primer
ccagttgtccgtagcagatga

SEQ ID NO: 77 - DNA, 20 bp, synthetic construct
  source 1..20: mol_type = other DNA; note = CD38_forward_primer
gggacaaaccctgctgccgg

SEQ ID NO: 78 - DNA, 21 bp, synthetic construct
  source 1..21: mol_type = other DNA; note = CD38_reverse_primer
caaggccagctgctcctgaaa

SEQ ID NO: 79 - RNA, 113 nt, synthetic construct
  source 1..113: mol_type = other RNA; note = VEGFA_Guide
  modified_base 1..3: mod_base = OTHER; note = 2'O-methyl ribonucleotide
  modified_base 1..4: mod_base = OTHER; note = phosphorothioate linkage
  modified_base 111..113: mod_base = OTHER; note = 2'-O-methyl ribonucleotide
  modified_base 110..113: mod_base = OTHER; note = phosphorothioate linkage
ctttgatgtctgcaggccagatgttgagaatcgaaagattcttaataaggcatccttccgatgctgacttctcaccgtccgttatccaataggagcgggcggtatgttttttt

SEQ ID NO: 80 - DNA, 2931 bp, synthetic construct
  source 1..2931: mol_type = other DNA; note = MCP-RT-GFP-Normalintron-MS2
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagctaaaaccacaaaggccaccatggactacaaagacgatgacgacaagtacccctatgatgtacctgattatgctcctaagaagaaaagaaaggtggccagcaatttcacacagttcgtccttgtagacaacggcggcaccggggatgttacagtggccccgagcaatttcgcaaacggcgtagccgagtggatctcctctaatagcagaagccaggcttataaggttacatgctctgtaaggcagtcttccgcacaaaatagaaagtacacaatcaaggtcgaagtgcccaaggttgctacacagaccgttggtggagaggagttgccagtggctgggtggcgctcctacctgaatatggagttaacaatccccattttcgctaccaattcagactgtgaactcattgtcaaagctatgcaaggtcttcttaaggatggtaacccaattccaagtgcaattgctgctaattcaggcatttattctggcggcagcagcggcggctcaagcggaagtgaaacacctggaacttccgaaagcgctacgcctgaatcttctggtggatcttctggcggatcttccgcatgcgtcgacgatatccctaagaagaaaagaaaggtgtgactagctggccggccgtgcgttttattcctgtctttttattgccgatccccgcgatcgcttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctaaggtgagtccaggagatgtttcagcactgttgcctttagtctcgaggcaacttagacaactgagtattgatctgagcacagcagggtgtgagctgtttgaagatactggggttgggggtgaagaaactgcagaggactaactgggctgagacccagtggcaatgttttagggcctaaggaatgcctctgaaaatctagatggacaactttgactttgagaaaagagaggtggaaatgaggaaaatgacttttctttattagatttcggtagaaagaactttcacctttcccctatttttgttattcgttttaaaacatctatctggaggcaggacaagtatggtcgttaaaaagatgcaggcagaaggcatatattggctcagtcaaagtgggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtatgtgtatgtgtatgtgtatgtgtatgtgtacgcgcacgtgtgtttgtgtgtgtgtgagagcgtgtgtttcttttaacgttttcagcctacagcatacagggttcatggtggcaagaagataacaagatttaaattatggccagtgactagtgctgcaagaagaacaactacctgcatttaatgggaaagcaaaatctcaggctttgagggaagttaacataggcttgattctgggtggaagctgggtgtgtagttatctggaggccaggctggagctctcagctcactatgggttcatctttattgtctcctttcatctcaacggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggccctgcaggctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggatcaattgccgacccctccccccaacttctcggggactgtgggcgatgtgcgctctgcccgatgagggctccagatggcatttagagctaggccaacatgaggattacccatgtctgcagggcctagcaagttaaaataaggctagtccgttatcaacttggccaggactgaggattacccagtctatctgcagggccaagtaaaaaaaaaaggaagggaaaaggggaattc

SEQ ID NO: 81 - DNA, 2163 bp, synthetic construct
  source 1..2163: mol_type = other DNA; note = MCP-RT-GFP-Chimericintron-MS2
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagctaaaaccacaaaggccaccatggactacaaagacgatgacgacaagtacccctatgatgtacctgattatgctcctaagaagaaaagaaaggtggccagcaatttcacacagttcgtccttgtagacaacggcggcaccggggatgttacagtggccccgagcaatttcgcaaacggcgtagccgagtggatctcctctaatagcagaagccaggcttataaggttacatgctctgtaaggcagtcttccgcacaaaatagaaagtacacaatcaaggtcgaagtgcccaaggttgctacacagaccgttggtggagaggagttgccagtggctgggtggcgctcctacctgaatatggagttaacaatccccattttcgctaccaattcagactgtgaactcattgtcaaagctatgcaaggtcttcttaaggatggtaacccaattccaagtgcaattgctgctaattcaggcatttattctggcggcagcagcggcggctcaagcggaagtgaaacacctggaacttccgaaagcgctacgcctgaatcttctggtggatcttctggcggatcttccgcatgcgtcgacgatatccctaagaagaaaagaaaggtgtgactagctggccggccgtgcgttttattcctgtctttttattgccgatccccgcgatcgcttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccaggtaagtatcaaggttacaagacaggtttaaggagaccaatagaaactgggcttgtcgagacagagaagactcttgcgtttctgataggcacctattggtcttactgacatccactttgcctttctctccacagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggccctgcaggctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggatcaattgccgacccctccccccaacttctcggggactgtgggcgatgtgcgctctgcccgatgagggctccagatggcatttagagctaggccaacatgaggattacccatgtctgcagggcctagcaagttaaaataaggctagtccgttatcaacttggccaggactgaggattacccagtctatctgcagggccaagtaaaaaaaaaaggaagggaaaaggggaattc

SEQ ID NO: 82 - protein, 35 aa, synthetic construct
  source 1..35: note = linker 1 for tethered RT
SGGSSGGSSGSETPGTSESATPESSGGSSGGSSAC

SEQ ID NO: 83 - protein, 14 aa, synthetic construct
  source 1..14: note = linker 2 for tethered RT
KLGGGAPAVGGGPK

SEQ ID NO: 84 - protein, 15 aa, synthetic construct
  source 1..15: note = linker 3 for tethered RT
GGGGSGGGGSGGGGS

SEQ ID NO: 85 - protein, 25 aa, synthetic construct
  source 1..25: note = linker 4 for tethered RT
GGGGSGGGGSEAAAKGGGGSGGGGS

SEQ ID NO: 86 - protein, 30 aa, synthetic construct
  source 1..30: note = linker 5 for tethered RT
GGGGSGGGGSEAAAKEAAAKGGGGSGGGGS

SEQ ID NO: 87 - protein, 16 aa, synthetic construct
  source 1..16: note = linker 6 for tethered RT
SGSETPGTSESATPES

SEQ ID NO: 88 - protein, 793 aa, synthetic construct
  source 1..793: note = MG140-29 retrotransposition protein
MDGVFAVHNVDNLTDHEPIVLQLSLKTDVLQNSEIVHVPRVSWRKASHDQVRNYKNVLSSNLKSIYLPVKTLLCNDIHCSNPIHFEDINRYSISIHDACISAARDTIPVTCRRGSRGRIPGWTERVKPFRERSLFWHNIWVDCGRPRDGHVAAAMRRTRAAYHLQIKLVRKNEDAVVREKVADSVIHDRGRNFWSEIKKIRGTKTRPPGTVDGLTDTPSIAALFADKYRDLYTSVPYDKDDMQRIIDVVDSKLSCTNFSGDCTFYATEVREAIGHMKRGKNDGSNELLSDHLINANDDLIIHIACLFSTFSVHGAVPANFHSSVILPIPKSRNASAASSNNYRGIALSSLLGKILDYIILERYRDTLSSCEYQFGFKRSSSTNLCTMVLKVTLNYYHDNSTNVFCCFLDATKAFDRVNYCKLFRLLLTRDLPPHIIRLLINIYVNNNVCVSWCNVKSDTFRALNGVKQGAVLSPILYCVYVDNLLQLLEKAGVGCHIGFQFLGALAYADDLVLIAPTASAMRTMLSVCDNFARDFDVIFNASKSKWLAVIPSNRSFMSSMINDCLFEVGGNPIDFVDKFVHLGHIISSNLTDTADVESRRSSFIAEVNNMSCFFSKLDVFTRFSLFRSYCTSYYGSELWKLNCPSIDSFYTSWRRALRTVWRLPFRAHSFLLPLISNVLPFRDEICRRSINFMRSCLFHNSYLVRFISYNSIMFGTKSSFLRHNAYYCASRLGCGLDSIIYGNVCVLAEVFSNEMFADAGMLLELLLLRDGVLHCGLNIDEISTLIIHLCTND

SEQ ID NO: 89 - protein, 795 aa, synthetic construct
  source 1..795: note = MG140-30 retrotransposition protein
MILSIDNTSDHDPIILRLSLDIKYVSVCNRTSSSRVSWVKASDRDIRNYQYNLASNLQHVTIPSVALLCKDVNCSNFAHRCQLSRYLTDISDACLAAGEASIPHTCSRHSGKRIPGWSEKVEPLRQRSLFWHSMWVECGRPRSGVVADCMRRARASYHYAIRAVRRNEQNIIRERVADALLRDPSRDFWTEVKKIRNSKSGRAVIVDGCSDAPSISQLFASKYRHLYTSVPYQHNDLQSIVSDVESRISFDGDCFIGSQEVMAALSKLKLHKNDGDLGLASDYFINSDPALSVHIALLFTGIVIHGFVPSNLLSSTIVPIPKKSNVNATDSDNYRGIALSSVLGKIFDNVILVKYSDKLSTCNLQFGFKRNSSTHMCTMVLKETISYYVNNNSSVFCTFLDASKAFDRVHYCKLFRLLLGRGLPVCILRVLIQLYVGHSIRVTWAGLVSSCFTALNGVKQGGVLSPVLFCIYVDELLIRLAESGVGCYIGFSFAGALAYADDIVLIAPTPSAMRKLLAICDTYATEYNILFNAQKSNFIAFVPSSRHFLHKAMTNCVFRIGGVQIEHVFSYTHLGHIITSRLDDADDILHRRSSYIGQVNNVVCYFDSLSWTVKLGLHKSYCSSIFGCELWALDSVRDIEKFCVAWRKGLRRVLSLPRAAHSHLLPLLSNSLPVYDEICKRSAKFIVSCQCSDNILVRAVVNYAIAARSKSVLGRNVMLLCRRFHLSFDDFVSGRLLLSGDIFVSHYLNSLSEAQLQSVCFALELLCLREQSFKLNNNMRLNADEISDYLAAVLC

SEQ ID NO: 90 - protein, 1034 aa, synthetic construct
  source 1..1034: note = MG140-31 retrotransposition protein
MLLFIRSVMENSNSVLDNLQLTICSYNCRGFNGFKKDYISGLLESAQIDVLLLQEHWLSDAQLNALNNVGANYLNFGVSGFDTSAVLGGRPYGGCAVLWRSDLLLQVQPLVVSSRRLSAVIFSTDNWSLILINVYMPYEGDEIKTDEFIDLLSIIEDLVLSNSASHVIVGGDFNVEFNRNRMHTALLNSFCDNTGLSPVIQHSSCNIDYTYNFNMSRFNILDHFLLSGTLFDVCVTSAYVLHDIDNTSHHDPIILRLSLDIKYVSVCNRTSSSRVSWVKASDRDIRNYQYNLASNLQHVTIPSVALLCKDVNCSNFAHRCQLSRYLTDISDACLAAGEASIPHTCSRHSGKRIPGWSEKVEPLRQRSLFWHSMWVECGRPRSGVVADCMRRARASYHYAIRAVRRNEQNIIRERVADALLRDPSRDFWTEVKKIRNSKSGRAVIVDGCSDAPSISQLFASKYRHLYTSVPYQHNDLQSIVSDVESRISFDGDCFIGSQEVMAALSKLKLHKNDGDLGLASDYFINSDPALSVHIALLFTGIVIHGFVPSNLLSSTIVPIPKKSNVNATDSDNYRGIALSSVLGKIFDNVILVKYSDKLSTCNLQFGFKRNSSTHMCTMVLKETISYYVNNNSSVFCTFLDASKAFDRVHYCKLFRLLLGRGLPVCILRVLIQLYVGHSIRVTWAGLVSSCFTALNGVKQGGVLSPVLFCIYVDELLIRLAESGVGCYIGFSFAGALAYADDIVLIAPTPSAMRKLLAICDTYATEYNILFNAQKSNFIAFVPSSRHFLHKAMTNCVFRIGGVQIEHVFSYTHLGHIITSRLDDADDILHRRSSYIGQVNNVVCYFDSLSWTVKLGLHKSYCSSIFGCELWALDSVRDIEKFCVAWRKGLRRVLSLPRAAHSHLLPLLSNSLPVYDEICKRSAKFIVSCQCSDNILVRAVVNYAIAARSKSVLGRNVMLLCRRFHLSFDDFVSGRLLLSGDIFVSHYLNSLSEAQLQSVCFALELLCLREQSFKLNNNMRLNADEISDYLAAVLC

SEQ ID NO: 91 - protein, 976 aa, synthetic construct
  source 1..976: note = MG140-32 retrotransposition protein
MVQNTELADCINITLQTNDSRFIVTSYNMHGFNQGLEGTKEMINKLYPDVIALQEHWLTPTNLDRLGEVSNDYFFIGSSSMNDVVTAGPLFGRPFGGTAILINNKLANATVNVACNDKFTAVLISECLVISAYMPCAGTCDRMSLYSAIISEIQALIEFYPQYKLILCADLNVELDAPSSVADLVNNFICRNNLRRTELANPTASRFTYMHESLQAASYIDYIISSDALDSIAFNVLDLDINLSDHLPIMCVFMCKMSACYSDPSQSVKSKSHVDDVSYFRWDHAALHLYYEQTRVMMEPILDKLNICVNSVCDEDAFIDHSKLEDIYSSVVAALTTSANLCIPKIKHNFFKFWWNQELNELKAAAVTSARAWQQVGKPKHGSVYHKYQKDKLLYKKCIRENQKQESVSFTNELHDALLRKTGKDFWKTWNAKVGVKRKCIEQVDGIVDGATIACNFAKHFEKICQPATATFNTTMKAKFEEMRSMYYTPFCDKTVEFDVQLIGKIISNMSCGKAAGLDELSAEHLKHCHPIVIIILCKLFNLFVHVGYLPLSFGTSYTVPIPKQNGRSHALTVNDFRGISISPVISKIFEHAIFARFGDYFSTSDHQFGFKESLSCSHAIYCVRNVIDHYVKRGSTVNICTVDISKAFDTVNHFVLFIKLMERKLPVQLLDLFVLWFSMSETCVRWGAHDSYFFKLKAGVRQGGVLSPYFFAVSVDDVVDKIAACNAGCYINNLCSAIFLYADDIILLSPSVCGLQRLLGICEAAITELDMKINASKTVCTRVGPRFDSACVGPNLTSGGQLNWVKTCRYLGVHFAAGRSFNCSFEEAKKKFYFSFNSVYSKLGRFASEEVILNLLNVKCVSAMLYATEACPVLSRHKHSLDFVITRVFMKILRTGSPQVVAECQKYFGFLPVSYRIDIRSARFLERFSSSVNSFCIAFGDRARKQLSDIVCKYNISDGLSSGVLRTVINRIYFQ

SEQ ID NO: 92 - protein, 970 aa, synthetic construct
  source 1..970: note = MG140-33 retrotransposition protein
MTITNDAAQLVVISYNLHGLNQGLPGIREFMTELKPDVIMVQEHWLTSDNLNKLSDISDEYFVIGSSAMDARVSAGPLFGRPFGGTAVFIKNKYINVTVNLVTSERYVVIQLCDWLLINVYLPCIGTSNRILLYSDTLCELQSIIRAHPECNCLVGGDFNTDLNDTRSHTANTVVNGFIAGCNLHRCDCLFPTRLKHTYVNDSMNCYSTIDYMLSSNPEKIVAFNVLDIDLNLSDHLPIISICVFDCNCKMNNPKPTSTENVTHFRWDHAPLQDYYEHTRLGLQPILADLDELIGNKLSYSNVDFLCQVDCIYNRVVSVLQQCSHAYVPKHKKNFYKFWWNQELDELKDKAIASCKMWKDAGKPRHGAIHAKYRQDKLLYKKRIRQERVQETSSFTNELHDALLNKSGRDFWKKWNSKFENKSNKILQVGGTSDTATILNNFAKHFEQVCVPFNATRNEELKSRYKEMRLNYSESSVINDTTVFDVQLVENLLTNMKNGKAGGLDELTSEHLKFCHPVVVIILCKLFNLFVINGHIPDSFGVSYTVPIPKSDGRSRSMTVDDFRGISISPVISKLFELCVLDRYSDYLQTSDHQFGFKKQLGCRHAIFSVRSVIEQYISNGSTVNLCALDLSKAFDRMNQYALFIKLMERRFPVKILTILEQWFSIAETCVRWGSEFSYFFSLLAGVRQGGVLSPVLFAIFIDGIVNRVNATNVGCYNSTVCVSIFLYADDILLLSPTVTGLQTLLTVCENELCELDMRLNVNKSVCMRFGARFKAHCANLVSVQGGALQWASSCRYLGVYFVSGRVFRCCFHSAKCNFFRAFNSVYSKIGCCASEEVILSLLKSKCLPYILYGVEACPVLQRDKHSFDFTLTRTLMKLFMTSSPVIVNECQTQFNLLPLRYQIDIRTVKFLNQYIISRNSICMLYKSHAQSVLDTIFGVYGNNVSSLHDLHNIINEHFYDFNPSTLGYN

SEQ ID NO: 93 - protein, 1020 aa, synthetic construct
  source 1..1020: note = MG140-34 retrotransposition protein
MYNLRITSYNCRSLSALKRDFVKTLLVSCDILFLQEHWLSDDQLTLLGGLDEGFTFTGVSGFGNKEILSGRPYGGCAILWRSALKFSVDFLSVNSRRVSAIKLSNDCCNLLLINVYMPFEDSDCNVNEFFDVLFDIEYLLVNNVDCHYLIGGDFNVDLSRNTVHTALLRSFSDNNGLLYASDFDGANFDFTYQFNMSRFSLIDNFMLSSFLFHNMLQRADVVHDVNNLSDHEPVTIELCMSVTHVDIQRSTVPPLQKVSWTTAAESHVIDYREELVDRLKTVVMPIDSLTCCDRRCSVAKHRSDIAEYANNIADVCIKAGLTTIPLMKPGHRATPGFSEHVKPARDKSMFWHQLWLECGRPRTGHVADCMRRTRAAYHYALRSVKRRRDEITQERFATALLHNNSRNFWSEVKKIRANRMTYNGVVDGHSDATDIVRIFGERYRDLYTSVPYDECNMHEINDTIDEHISDDDDAAFVVSLRDVSDAIAHIKHYKNDVDNMLTSDHFINAPSVLHVHISILFSAMLMHGCVPNLLMYSSIRPIPKGHNLSTCDSNNYRGIAISSIFNKIFDNVVLIKYRHLLSTCDLQFGFKKKHSTQMCTMVLKETLSYYLSNRSNVFCTFLDATKAFDRINYCKLFNLLLCRSLPYCIIRVLLSLYTNNYVYVSWVGSNSSSFRACNGVKQGGVLSPVLFCLYMDGLLNKLSHAGVGCYMGEMFVGALAYADDIVLISPTPSAMNNMLSICDEFSIEYNVLFNPLKSKCMYFYPKSRSTLLLHRYNVCDLQFSINGKAIEFVDSYKHLGHVICSDMTDDIDISEKRGVFIGQANNIICYFAKLSSAIKYRLFTSYCTSFFGCELWRISNDSLDSMCTAWRRAIRRIWSLPYTAHGRFLPVLCNSYNIFDQFCVRILNFIRRCLSDQSSLLVRSIATQALMFHHARSPLGYNFIYCARRYCFSLTDFVNYNNFVSNVEKLNCTQNDDDTIANCRLLRELIDLRDGVLHLSDDVFLTNSELSFMINCVSTL

SEQ ID NO: 94 - protein, 1020 aa, synthetic construct
  source 1..1020: note = MG140-35 retrotransposition protein
MYNLRITSYNCRSLSALKRDFVKTLLVSCDILFLQEHWLSDDQLTLLGGLDEGFTFTGVSGFGNKEILSGRPYGGCAILWRSALKFSVDFLSVNSRRVSAIKLSNDCCNLLLINVYMPFEDSDCNVNEFFDVLFDIEYLLVNNVDCHYLIGGDFNVDLSRNTVHTALLRSFSDNNGLLYASDFDGANFDFTYQFNMSRFSLIDNFMLSSFLFHNMLQRADVVHDVNNLSDHEPVTIELCMSVTHVDIQRSTVPPLQKVSWTTAAESHVIDYREELVDRLKTVVMPIDSLTCCDRRCSVARHRSDIAEYANNIADVCIKAGLTTIPLMKPGHRATPGFSEHVKPARDKSMFWHQLWLECGRPRTGHVADCMRRTRAAYHYALRSVKRRRDEITQERFATALLHNNSRNFWSEVKKIRANRMTYNGVVDGHSDATDIVRIFGERYRDLYTSVPYDECNMHEINDTIDEHISDDDDAAFVVSLRDVSDAIAHIKHYKNDVDNMLTSDHFINAPSVLHVHISILFSAMLMHGCVPNLLMYSSIRPIPKGHNLSTCDSNNYRGIAISSIFNKIFDNVVLIKYRHLLSTCDLQFGFKKKHSTQMCTMVLKETLSYYLSNRSNVFCTFLDATKAFDRINYCKLFNLLLCRSLPYCIIRVLLSLYTNNYVYVSWVGSNSSSFRACNGVKQGGVLSPVLFCLYMDGLLNKLSHAGVGCYMGEMFVGALAYADDIVLISPTPSAMNNMLSICDEFSIEYNVLFNPLKSKCMYFYPKSRSTLLLHRYNVCDLQFSINGKAIEFVDSYKHLGHVICSDMTDDIDISEKRGVFIGQANNIICYFAKLSSAIKYRLFTSYCTSFFGCELWRISNDSLDSMCTAWRRAIRRIWSLPYTAHGRFLPVLCNSYNIFDQFCVRILNFIRRCLSDQSSLLVRSIATQALMFHHARSPLGYNFIYCARRYCFSLTDFVNYNNFVSNVEKLNCTQNDDDTIANCRLLRELIDLRDGVLHLSDDVFLTNSELSFMINCVSTL

SEQ ID NO: 95 - protein, 977 aa, synthetic construct
  source 1..977: note = MG140-36 retrotransposition protein
MVQNTELADCINITLQTNDSRFIVTSYNMHGFNQGLEGTKEMINKLYPDVIALQEHWLTPTNLDRLGEVSNDYFFIGSSSMNDVVTAGPLFGRPFGGTAILINNKLANATVNVACNDKFTAVLISECLVISAYMPCAGTCDRMSLYSAIISEIQALIEFYPQYKLILCADLNVELDAPSSVADLVNNFICRNNLRRTELANPTASRFTYMHESLQAASYIDYIISSDALDSIAFNVLDLDINLSDHLPIMCVFMCKMSACYSDPSQSVKSKSHVDDVSYFRWDRAALHLYYEQTRVMMEPILDKLNICVNSVCDEDAFIDHSKLEDIYSSVVAALTTSANLCIPKIKHNFFKFWWNQELNELKAAAVTSARAWQQVGKPKHGSVYHKYQKDKLLYKKCIRENQKQESVSFTNELHDALLRKTGKDFWKTWNAKVGVKRKCIEQVDGIVDGATIACNFAKHFEKICQPATATFNTTMKAKFEEMRSMYYTPFCDKTVEFDVQLIGKIISNMSCGKAAGLDELSAEHLKHCHPIVIIILCKLFNLFVHVGYLPLSFGTSYTVPIPKQNGRSHALTVNDFRGISISPVISKIFEHAIFARFGDYFSTSDHQFGFKESLSCSHAIYCVRNVIDHYVKRGSTVNICTVDISKAFDTVNHFVLFIKLMERKLPVQLLDLFVLWFSMSETCVRWGAHDSYFFKLKAGVRQGGVLSPYFFAVSVDDVVDKIAACNAGCYINNLCSAIFLYADDIILLSPSVCGLQRLLGICEAAITELDMKINASKTVCTRVGPRFDSACVGPNLTSGGQLNWVKTCRYLGVHFAAGRSFNCSFEEAKKKFYFSFNSVYSKLGRFASEEVILNLLNVKCVSAMLYATEACPVLSRHKHSLDFVITRVFMKILRTGSPQVVAECQKYFGFLPVSYRIDIRSARFLERFSSSVNSFCIAFGDRARKQLSDILCKYNIPDGLSSGVLRTIINRIFFNS

SEQ ID NO: 96 - protein, 969 aa, synthetic construct
  source 1..969: note = MG140-37 retrotransposition protein
MTITNDAAQLVVISYNLHGLNQGLPGIREFMTELKPDVIMVQEHWLTSDNLNKLSDISDEYFVIGSSAMDARVSAGPLFGRPFGGTAVFIKNKYINVTVNLVTSERYVVIQLCDWLLINVYLPCIGTSNRILLYSDTLCELQSIIRAHPECNCLVGGDFNTDLNDTRSHTANTVVNGFIAGCNLHRCDCLFPTRLKHTYVNDSMNCYSTIDYMLSSNPEKIVAFNVLDIDLNLSDHLPIITVCVFDSNCKLNNPKPTSTEDVTHFRWDHAPLQDYYEHTRLGLQPILADLDELIGNKLYSNVDFLCQVDCIYNRVVSVLQQCSHAYVPKHKKNFYKFWWNQELDELKDKAIASCKMWKDAGKPRHGAIHAKYRQDKLLYKKRIRQERVQETSSFTNELHDALLNKSGRDFWKKWNSKFENKSNKILQVGGTSDTATILNNFAKHFEQVCVPFNATRNEELKSRYKEMRLNYSESSVINDTTVFDVQLVENLLTNMKNGKAGGLDELTSEHLKFCHPVVVIILCKLFNLFVINGHIPDSFGVSYTVPIPKSDGRSRSMTVDDFRGISISPVISKLFELCVLDRYSDYLQTSDHQFGFKKQLGCRHAIFSVRSVIEQYISNGSTVNLCALDLSKAFDRMNQYALFIKLMERRFPVKILTILEQWFSIAETCVRWGSEFSYFFSLLAGVRQGGVLSPVLFAIFIDGIVNRVNATNVGCYNSTVCVSIFLYADDILLLSPTVTGLQTLLTVCENELCELDMRLNVNKSVCMRFGARFKAHCANLVSVQGGALQWASSCRYLGVYFVSGRVFRCCFHSAKCNFFRAFNSVYSKIGCCASEEVILSLLKSKCLPYILYGVEACPVLQRDKHSFDFTLTRTLMKLFMTSSPVIVNECQTQFNLLPLRYQIDIRTVKFLNQYIISRNSICMLYKSHAQSVLDTIFGVYGNNVSSLHDLHNIINEHFYDFNPSTLGYN

SEQ ID NO: 97 - protein, 1025 aa, synthetic construct
  source 1..1025: note = MG140-38 retrotransposition protein
MVILLLMASYTNAILSIASFNCRGFNQIKKQYICNLLGKCNFLFLQEHWLSERQLGILGNIQSGVLYSGISGFDSSEVLAGRPFGGCAILWHSDVLINVTSVETNSNRICAVLVTTESWKLILINVYLPHEGDDIKSDEFVHCLYLVEDIIGKHSDCHVVVGGDFNVDFNRNWNHTKILNSFCDSNELLSACRSNSNIDYTYNFSMERFSILDHFLLSSTLHNTCIEKISVMHDVDNISDHDPIFLKLKLDVKYIGFSSRIFAPRTSWVKATENDLNRFRHTLSDNLKCITTPTSLLLCHDMKCTDACHHNAIAEYATAISEACLLAAGTCIPHTSNRCTDRRVPGWTERVEPLREKSIFWHKLWMECGRPRDGHVADCMRRTRAGYHYAIRQVRKDEELIVKQRIAEALARDPSRDFWTEIKRIRGNKAGLSRIVDGCIDEASISKLFADKYKCLYASVPYDTVEMQNIQDIVDSQLAASDFFDDSYIINYQDVSDAIFKLNAHKNDGNLGISTDHFLHGGSDLHMHIAFLLTSIVVHGSVPSEFVSSTVIPIPKKPNVNATQSDNYRGIALSSCFCKILDNIILAKNADRLSTSDLQFGFKRKSSTHMCTMVLKETLSYYVSNNSCTFCTFLDATKAFDRVNYCKLFHLLIKRGLPASVIRILIVMYTGHCVRVAWAGLASSFFSAVNGVKQGGVLSPILFCIYMDDLLVKLHKSGVGCYIGTAFVGALAYADDIVLLAPSPSAMRKLLSVCEFYALDFDISFNAGKSNFILVMPNSNQRLRTQMNNCTFSIGGAPVVKVNSYLHLGHIINNQLNDDDDVMYRRNCFIGQANNVLCYFNKLDMCVRIKLFKNFCSSMYGCELWSTDNVEVFCVAWRKALRRVLNLPYDTHSYLLPLLTDTLPVFEEICRRSAKFILKCFNSSSTLVKYVTRHAIDIARYNSNVGKNALFCCNYFKWQLYDFVNGSVSLNRNSFLTFCLNRLSGCEVDNAGSLYEALLVREGNLFVESFTRDDIELIIDAMSRC

SEQ ID NO: 98 - protein, 831 aa, synthetic construct
  source 1..831: note = MG140-39 retrotransposition protein
MANCNGTIISYNMHGFNQGSELLKSYCANSSVDFLLIQEHWLSPDALHKIDDIAQDYFCFSVSSMTAVLESGPLRGRPFGGLSILVKNMHRQFCSVVALNERYIAVQYNDILIIDVYFPCVSSLNHKDETIDLLVQLDTLVNQSVVKDIIIGGDFNCNLEIESWSSKVICDFMEDNSLSSCNKLAVNVIGAEYTYSNEVLGHYSYLDYFVVSNSLTSSVMSLEICSDDFNLSDHSPVCIEVCNIISDCETTVLCSNKGGKSTKIPDTCYQNRWDHADIVAYYELTRLGLQPILAYLNDCSNSFPPQSAIDYKRYMRACIDLAYNDAVSVLVDAAKNTVPRFKANALKHWWDQELSELKEKAFASHKLWIEAGKPRNGCIFDIRKADKYKYKCLIKRKQLEVRDSITNDLHDALLLKDADHFWKVWKSKFPAKRPNKYALIEGNSDPNIISNTFCNYFSEICTAHPSVNASSNNIFLNRFDGYIGDLNQSTKNIISIDLIEDFILSMKRGKAAGLDSLTIEHIQFAHPAIICILKLLFNYMLEFGIVPEGFRNGLVIPLPKEDSIKKNVKLENFRCITISPVISKIFEHCLMRLFAKYLNSDDAQLGFKKKCGCSHAIYCVKQVVDYYVRGGSTVNVCTLDISKAFDKVNLFVLLCKLMDRNIPNYVINVLYDWFSNNYITVKWLNILSSRCPVNSGVRQGGVLSPVLFAIYVDDILVKLRKSRLGCTIQGLSVNAYMYADDLIILSGSVTDLQKLITLSIEELKCIHLSINPKKCFCMRVGKRFKVNCNNVVVDNYSIQWSSEIRYLGVYLTAGHVLKFNLDYGKKSSIAA

SEQ ID NO: 99 - protein, 926 aa, synthetic construct
  source 1..926: note = MG140-40 retrotransposition protein
MHGFNQGFTYVNEICNDQAYDIIFIQEHWLYPSTLHKILEISDNYIGFGTSAMQSELEARFLRGRPYGGTAILINKKFKTYCIDSCVFKRVVSVLLGDFLFINVYLPCNDGSAANVNTLGEILANISNIIDAHDVNCIIFGGDLNTDITKVSLHSNMILNFMKDYDLTVCMKHIFGTCDVQFVDTFVCENLNASSCIDFLCMSNSLSDNVSRYKVIDAYNNHSDHLPVSIDVCLPVKCVLYACMNTVCDDVQVHSEHASKCTRSKAKDSRLRWDLGNIQMYYDLTCAHLYPIYECITQVESNMYMYDEQDRSSRIDELYNKLVSTLHNCTKPAIPLVKANTLKHWWTTELNQLKSKSLTSHNIWLDAGKPLRGEIYQSKRHDKLSYKLAIKTAKKEADAAVSDALHTNLISKSSVNFWKTWKSKVCNKVKTCVSVEGCNTDEQAAAKFSDYFSRATSPNSQEYSSAKQNEFNRKLLQYRKVEYANDITAELVALAVAKMQDGKAAGFDNIYVEHIRHSHPLIFSLLAKLFNLMLRAGIVPTLFGNGVVIPIPKENTHKKTHPVDNFRGITLSPILSKVFEHCLLQLMSKYLATSENQFGFKSNTGCTQAVYAVRKVTEYYVANESTMNLCFLDISKGFDKVNNYELMMKLMKRRTPSYFIQILNHWFSISQCTVRWNGVLSAMYRLEAGVRQGGVLSPVLFSVYVDDMLCKLKSMGCSYKALHLSAFMYADDLVLLSPSVSAMQTMLNVCNCELKLLDLKLNVKKSKAVRIGKRFKNNTVNLNIEGQAICWSSEAKYLGVVIVSAPRFKCSFDAAKAKFYRSANSILAKVGHKQNVTVTLHLIATIALPTLTYCIEALHLNKSEVSSLSHPWQSCLFKLFRTFDAEIIKNCCELLNYKTISELYNERVAKFARNLKICNNHVLPLL

SEQ ID NO: 100 - protein, 995 aa, synthetic construct
  source 1..995: note = MG140-41 retrotransposition protein
MHGFNQGHSVISDFCNPKNVNSMDIICIQEHWLSSTNLSKLANFSNSYFMYGISAMENAEKAGLIKGRPYGGVCILLRKELCKCVKFSKCLERLVCLVIDGYIFINCYFPSIRSDDDLNTVYLLFSEIDDILRDFPGNRVILGGDFNVNIVNKAKYADIFIKKLQNLNLMFCNDIYNSAINFTYCHESLQNYSYIDYFAVSNFMIKDIIDFDILDLGNNISDHNPIVIKVKCQLSDVNVSSKASNYKEFFRWDHANISNYCNLTRTYLQSILEYVNDVSDYLEMHACCYSCSDILKAGKERAYADLNLSASYQTEPCKSCSGNYDIARRCIDSSYNKIVNVLNFSAEKTVPVRKANFYKFWWDDELSQLKYNSIQAHETWVKLNKPKQGIAFDNKVKAKTEYKLAIRQKQSLERDGITNDLHESLLAKSTNVFWKSWNSKFGQKSKVPLVIESSQDNQVISDKFAGYFASINSSNFPSNKNRHKDVFLCKFKDYKGDNWSYRDKINTFMLDYCIKESKLGKSPGADGITIEHLLYSHPMLICLLSKLFNLMLRFSYVPDKFAECIVIPVPKLQNVSKLMKCEDFRPISLSPIISKIFEKCILKLVEEYFVTSDRQFGFKKRLGCTHAISAVKSTVDFFTENGSTVNMCALDLSKAFDKINHFILFNKLMKSNMPLRILLLIICWYSKLVNVVRWNGSLSNVFKTANGIRQGGILSPFLFNIYVDDILFSLKRSKLGCYVGNMCCNSFMYADDLLLLAISVTQLKQMIIMCTNLFEEIDMRLNITKTNCIRVGNRHLSTVIPMVIGDIVVNWKQEMRYLGVCFVSAKKLKLNIIFAKQKFFKSLNSIFSKVGSNASPEVILSLFNSFCVPVLLYGLGPFNLSLNLKTRNSLIKAYDSVFYKTFKTFDNEVTRYCQLMFRVMPLTFKLDLLTINFMLSSQKCDNEIIKFFSHRSFKINMDKLLYKYNIVIDDMYKAKEALFDNFKVKVESNIALGLV

SEQ ID NO: 101 - protein, 1438 aa, synthetic construct
  source 1..1438: note = MG140-42 retrotransposition protein
MTETSHAPVLDQTCTRYQRHNVHEQAMQFVPPNRVVFSFFGPCDIVCHRGAFLSVTSYCKTLSLLQRAHFTRHRNKATGQPFLLLSVYKQQDLQTLTQQGLVPFTQTCNEALSLRGCRVAPSGRVALPYMMYPQGDHRNQPVHNQETHTPLTPGLDANPFAALSVEQQTCGASTSRTSLIVGSMNVNGLFTQTMSSKHQDVCAFVSSHTVDICCLQETWCDMSETEYSSMLASCGYTSFGVSGYSTRTRTGGGTGILVATSISHLAHMLHWESQKHTQTTWIKLNSTQYKARKTAVGSVYLRPVSHTRPGDAERYVTELIALRDDITYLSEHNFDVVLCGDFNARIGNSQVSPHVPQHNEQTRNTQGDRLVTLLEQCNMFVMHNHTSFSPTCMHAGGSSVVDYCITNSSCRARCSDAAINFAGPCVSDHALLTMSLSHATRTRHRKTRRAHAKLWNRRAMHDEQTIAQFHATLETPLTQLSDFIDASTLSPQEATDTFTSKLTAALRDAGATCFGTHQVRPHRAQWWNKDYANLRHECWNLYETHRRSGSEADRAAYEEKRAAKNKMKRNLKRAYVKAQAGHVSKLCTPGLVSKTSWGAAKRLLKLVTRGSSATEHTLPTVIHDGAPTDDLEAVMQVFSSHYHTEMNPVPSATFCEATDTLVKDKLHAWRQAEPEHINNMDDEFTITELDRALGSMHNWKAADHDGMIIELLRAGGPALQLVVLKILNFCWTHETIPTNWKLGTIISLYKAGKKENPSNYRGITLLSVVRKLFCTLLRSRLQDNISLHESQAAFRANRGCMDHVHTLARIVRAANRKDIPVYAFFLDIRKAYDTVWRDGLIYKLLQKGVTGRLGRVISQVLTDTQSRVRFQHRESAYFPLTLGVGQGDPLSTILFDVFIDDLLEELHARPVHHCIPVDSPHLDRIADLTYADDVNALSLTPEGLQGHIDTIDTWLFRWRSQPNVSKSKTMVFNPPQGVPPTVFTMRGSNLDTVQCFKYLGVFFQSNGSWTDHTQHVRTQMNKAVGMWRPVLRCHYLPVAARLRIIYAFVYAPALYGAEVWIAPKSELEKLDTICKSAIRTVFGLHQFDCREEILFADTGLLPVSSLINAAKLCWFTKLLNMPESRFPTAVEAVTLPGDTQRGRVAGGDFGTRIADITTEIRRYAHMKLVMQDPLEHAPHRRRRPIRVSKRLSHTKPDAHLYETITYSSLTRERVKALLWKVYMEKCFERRGRKDGVTGEWMRSVIKHELGSLAPFLHSVECGLVRVLMSARSRLSPVLCRPPQDKHREYSQAAGAFTTYISRAPIGAVLPSPPVAHCVRHVSAVAELVRELLAQPGPYGCGSSVSWSDIAVVLVVGIDGVGGAPGVLGVVARPSPLSELARVVAFCGASPRLRHHVAWMSRSVPVAGVVNVPVDMAMLQRTKAGRQVAPQGKHGAHSS

SEQ ID NO: 102 - protein, 972 aa, synthetic construct
  source 1..972: note = MG140-43 retrotransposition protein
MAISYTSNNSVRICSFNMHGFNNGVSMTKILCNSFDVILLQEHWLLPSNLSKLGDISHDFTHHSVSAMTTKISEGILYGRPFGGVSILYRNSLTKNISIIDADKEEGRYVTIKLKCVNNEFITVTNVYFPTICAYSDYIVNTSSIMAYLDNLFANEVSCHHVVAGDFNFEYRNDNIGFDLFRSLAVDNNLICCDDLHLNQNIKFTYKHETLPQQSWLDHFFVSAGLTSSIVYCDTIDNGSNLSDHLPICCTINVSLSDSNCTPKLSKVYRDRWDKADLIKYYYQSGIHLQSLTAPPHVTQCSTHCQKAQHLQDINAYYERIIQALKASAIGCVPRMPVNCLKHWWNDDLTRLKNLSIDMHNLWRQVGSPRNGIINEARLKAKLDYKQAIRQAMLDCENKDADIINNKFNQKDSRNFWKCWGAKYRKKVNNTACIDGCTDNSTIANKFKAYFQNTYVDSTCDVNACMEFDKLMSDHSHLQHNDIVEEIRIEDIEKCIELLKPLKSAGHDDVAPEHLIHSHPSLCMHLKLLFSMMLNHNYVPDSFGIGIIIPVVKDKRGNLNSVENYRPITLSPIISKVFESFVLNRFAKFMTCDPLQFGFQRSVGCNNALFAIRQVIQYFNDRDSNVMVASLDACKAFDRVNHFKLFSTLLQRKLPLHIIKVLINWYCKLMVQVRWNNSLSDLFHVKSGVRQGGILSPALFNVYIDCVINKLRASQLGCHIGSLYIAVVLFADDILLLSSSFMELQRMIDLCVESGDEIGLKFNAAKSHCMIIGPHKVLVKPDMMMGNSPTAWSETIKYLGVYIQSDKKFTVDLSLVRRKFFASVNCILRNAAFTSDIVKLELVEKQCFPILLYGLQSFDLKSSVIANVNAWVNSVYRKIFGYHKWESVKECIYLLGRLDVFHTIKLRRINFLKNIQKCNNEVARSLFNYVLHTREFQSCCTLPNNSVINISKSCDNVRKAVFNSFQSKVVGH

SEQ ID NO: 103 - protein, 957 aa, synthetic construct
  source 1..957: note = MG140-44 retrotransposition protein
MASQTLDTQTANNVCNSVSVMSYNMHGFNQGYSYLNDICMKQTYQVILIQEHWLYPATLHKLANISEHYSFYGASAMQTALDAGFIRGRPYGGTGILLHKDIAKYCVESCAFERVVGVLLGDFLFVNAYFPCWDGSVINLDAANELLANLSNVLDSYQAKHVVFGGDFNANLTKNSVYSNMILEFMSEHQLDICRKVLFGSSNVIFSDTYVNEALNASSCIDFICISSGLSNNVAQYDVHDVFNNHADHLPVSLKLCLPVSSILYNCIASGSSACMFNEAKSRDTQSCDSKGNKLRWDKGNTQLYSDLTYAQLFPIYETLHSIDVESSAYTAQEHRSLINETYTQVVSALHYAANVSIPAMASHTLKHWWCSDLSELKKKSMLSHNEWINAGKPHAGRIHTAKQQSKLQYKRAIKHAKASAENSVSDELHRNLCSRNNVKFWKTWKNKIKRPGTEKLYVEGCNTDAKAAELLADYFNKATSPNSNEYNNSKRVEFEKSFASYAIQANDIDISAGLVESAALKMTAGKAAGIDNISIEHVHYCHIVIYSLLAKLFNLMLCFSCVPDAFGYGVTTPIPKEESHKKIHPVENFRGITLSPVLSKLFEHCLLSVFSDFLQTSDNQFGFKRSTGCTHAIYALRKVTEFFICNESTVNMCFLDISKGFDKVNNFELLLKLMKRKAPSCFIKLLHDWFSISYGSVKWNTSRSDWYKIGAGVRQGGVLSPILFAVYVDAMLEKVKTMGCQYKSLCTGAFMYADDLVLLSPSIYELQHMIALCRNELHSLDLKLNVKKSKALRIGKRYKCKALPLMIDGQAVHWSNEARYLGIVIRSACKFKCNFDPAKVKFYKAANTILAKLGNKCNVTVTLYLVAAVALPPLIYGIEALTLNSSELNSLNHPWNNCLGKMFNTFDKELIKNCCDILGYESCQNVYVKKVEKFLRNMKFIDNAILSALNTEQVNV

SEQ ID NO: 104 - protein, 976 aa, synthetic construct
  source 1..976: note = MG148-12 reverse transcriptase
MANCNGTIISYNMHGFNQGSELLKSYCANSSVDFLLIQEHWLSPDALHKIDDIAQDYFCFSVSSMTAVLESGPLRGRPFGGLSILVKNMHRQFCSVVALNERYIAVQYNDILIIDVYFPCVSSLNHKDETIDLLVQLDTLVNQSVVKDIIIGGDFNCNLEIESWSSKVICDFMEDNSLSSCNKLAVNVIGAEYTYSNEVLGHYSYLDYFVVSNSLTSSVMSLEICSDDFNLSDHSPVCIEVCNIISDCETTVLCSNKGGKSTKIPDTCYQNRWDHADIVAYYELTRLGLQPILAYLNDCSNSFPPQSAIDYKRYMRACIDLAYNDAVSVLVDAAKNTVPRFKANALKHWWDQELSELKEKAFASHKLWIEAGKPRNGCIFDIRKADKYKYKCLIKRKQLEVRDSITNDLHDALLLKDADHFWKVWKSKFPAKRPNKYALIEGNSDPNIISNTFCNYFSEICTAHPSVNASSNNIFLNRFDGYIGDLNQSTKNIISIDLIEDFILSMKRGKAAGLDSLTIEHIQFAHPAIICILKLLFNYMLEFGIVPEGFRNGLVIPLPKEDSIKKNVKLENFRCITISPVISKIFEHCLMRLFAKYLNSDDAQLGFKKKCGCSHAIYCVKQVVDYYVRGGSTVNVCTLDISKAFDKVNLFVLLCKLMDRNIPNYVINVLYDWFSNNYITVKWLNILSSRCPVNSGVRQGGVLSPVLFAIYVDDILVKLRKSRLGCTIQGLSVNAYMYADDLIILSGSVTDLQKLITLSIEELKCIHLSINPKKCFCMRVGKRFKVNCNNVVVDNYSIQWSSEIRYLGVYLTAGHVLKFNLDYGKKSSIAALNSIMSKTGNKAIDIVLSLTQSYCIPILLYAVESMCLTTTERQRLGSSFNKLYNKLFSTFDTQTIAYCQYYTGYLPLDYVIDLRCWNFLQKLSISNNLVLNKLFRLNGNNTIDSLCLKYNCEFKNYPSLKVSMWEKFKFVYSLCP

SEQ ID NO: 105 - protein, 950 aa, synthetic construct
  source 1..950: note = MG148-38 reverse transcriptase
MRGFFQGLVVVNDLISGYQSPDVILLQEHWLTPANMNLFDEKITTHFVVGKSAMSDRVSAGPLVGRPYGGAAILIRNELRADTECVFCSDRFAVVRICNLLIMSVYLPCAATAARMFIVEDLLQEIWSLRLKYSECSVVIGGDFNADLNKQNDVSNFINSFLTTCLLIRCDSKFLSRQQSTYVNESLGQSSTIDFFVCDVVDDIIDYCVIDPDVNFSDHLPVAVRCKWSRTDNLKRSKSSNSSKVKHLRWDHGDLLSYYSSTMSRLYPLYEYLIRIEGWLVEPSNAVQRETVIRLVDHVYDQLVVALRESANSYIPKHAKKFFKFWWNQELDALKHNAIASSSVWKNAGKPRGGALYLQYKRDKLLYKKRLREEQKAEAACYSNDLHDALLRKSGQDFWKCWNSKFERRSNKVVQVDGITDSAVIADKFAEYFESVCRPFNADRNNLIKSKYNELRSTYTGTPIVEEQWFNVELLSKLVDSMSKGKAAGLDELSSEHLKYSHPVVVCILSKLFNLFVYYSHIPASFGRSFTVPIPKHDGRTHALHVDDFRGISISPVISKLFEMAILDRFSVFFSTSDHQFGFKKNLSCRHAIYCVRSVIDNFVLHGSTVNVCALDLSKAFDRMNHYALFIKLMERSFPCELLAILETWFYISVSCVKWNDNLSSFFVLTAGVRQGGVLSPYLFAIFIDDLVYKVKSLNVGCYISLTCAAIFMYADDILLLSPTVDGLQQLLHVCENELEQLDMKLNVNKSVCIRVGPRFNADCAELRSRNGAVLKWTDSCRYLGVYFVRGRTLKCSFSNAKSRFFRAFNAVFGKVGRAASEETVIELIRAKCIPILLYATEACPFFSRDKQSLEFTVTRLFMKIFQTGSPAVVRDCQRSFNFLPIEMQVIIRTSRFLQAFVATQNSLCSLFQRSASCQLNDIFSKYDRVQTASQLANRIHHTFAMCDSV

SEQ ID NO: 106 - protein, 486 aa, synthetic construct
  source 1..486: note = MG148-40 reverse transcriptase
MHGFTPDCMLLSTVVPIQKGKNVNVTDSANYRGISLSSIFAKLFDLLILQRYSDCLCLSDQQFGFRAKRSTDMCSMVLKESISYYVNNGSSVYCTFIDATKAFDRVEYCKLFRQLLSRGLPPVIIRIMLNLYVGHVTRVEWNGIRSRNFSVFNGVKQGGIVSPILFCIYLDGLLQSLATSGVGCYIGTIFVGAMAYADDLVLLAPSANAMRLMLRNCDAFANEYNIRFNANKSKCLFCSARRHSRCNIGAQPVFYIGGNPIEIVDHWPHLGHIISSHLDDEQDIIQRRNAMAGQINNVLNYFVGLDCFVKQKLLTTYCYSLYGSVLWDLRHLCIDSVCTTWRRGLRRVWGLPHNTHSNLLPLLSCSLPVYDELCKRFVLFAQKCLMSDSSLVSSVATYAFIYGRSDSVFGRNVSKCCLRFNVANDDFLSLNKKFMFNNYCSNLEQDTMSTVNLLLELICVRDGLFIMPMFADAAELIEIIRLLCTV

SEQ ID NO: 107 - protein, 802 aa, synthetic construct
  source 1..802: note = MG148-41 reverse transcriptase
MTITNDAAQLVVISYNLHGLNQGLPGIREFMTELKPDVIMVQEHWLTSDNLNKLSDISDEYFVIGSSAMDARVSAGPLFGRPFGGTAVFIKNKYINVTVNLVTSERYVVIQLCDWLLINVYLPCIGTSNRILLYSDTLCELQSIIRAHPECNCLVGGDFNTDLNDTRSHTANTVVNGFIAGCNLNLSDHLPIITVCVFDSNCKLNNPKPTSTEDVTHFRWDHAPLQDYYEHTRLGLQPILADLDELIGNKLSYSNVDFLCQVDCIYNRVVSVLQQCSHAYVPKHKKNFYKFWWNQELDELKDKAIASCKMWKDAGKPRHGAIHAKYRQDKLLYKKRIRQERVQETSSFTNELHDALLNKSGRDFWKKWNSKFENKSNKILQVGGTSDTATILNNFAKHFEQVCVPFNATRNEELKSRYKEMRLNYSESSVINDTTVFDVQLVENLLTNMKNGKAGGLDELTSEHLKFCHPVVVIILCKLFNLFVINGHIPDSFGVSYTVPIPKSDGRSRSMTVDDFRGISISPVISKLFELCVLDRYSDYLQTSDHQFGFKKQLGCRHAIFSVRSVIEQYISNGSTVNLCALDLSKAFDRMNQYALFIKLMERRFPVKILTILEQWFSIAETCVRWGSEFSYFFSLLAGVRQGGVLSPVLFAIFIDGIVNRVNATNVGCYNSTVCVSIFLYADDILLLSPTVTGLQTLLTVCENELCELDMRLNVNKSVCMRFGARFKSHCANLVSVQGGALQWASSCRYLGVYFVSGRVFRCCFHSAKCNFFRAFNSVYSKIGCCASEEVILSLLKSKCLP

SEQ ID NO: 108 - protein, 950 aa, synthetic construct
  source 1..950: note = MG148-42 reverse transcriptase
MRGFFQGLVVVNDLISGYQSPDVILLQEHWLTPANMNLFDEKITTHFVVGKSAMSDRVSAGPLVGRPYGGAAILIRNELRADTECVFCSDRFAVVRICNLLIMSVYLPCAGTADRMFIVEDLLQEIWSLRLKYSECSVVIGGDFNADLNKQNDVSNFINSFLTTCLLIRCDSKFLSRQQSTYVNESLGQSSTIDFFVCDVVDDIIDYCVIDPDVNFSDHLPVAVRCKWSRTDNLKRSKSSNSSKVKHLRWDHGDLLSYYSSTMSRLYPLYEYLIKFEGFLVEPTNAVQRETVIRLVDHVYDQLVVALRESANSYIPKHAKKFFKFWWNQELDALKHNAIASSSVWKNAGKPRGGALYLQYKRDKLLYKKRLREEQKAEAACYSNDLHDALLRKSGQDFWKCWNSKFERRSNKVVQVDGITDSAVIADKFAEYFESVCRPFNADRNNLIKSKYNELRSTYTGTPIVEEQWFNVELLSKLVDSMSKGKAAGLDELSSEHLKYSHPVVVCILSKLFNLFVYYSHIPASFGRSFTVPIPKHDGRTHALHVDDFRGISISPVISKLFEMAILDRFSVFFSTSDHQFGFKKNLSCRHAIYCVRSVIDNFVLHGSTVNVCALDLSKAFDRMNHYALFIKLMERSFPCELLAILETWFYISVSCVKWNDNLSSFFVLTAGVRQGGVLSPYLFAIFIDDLVYKVKSLNVGCYISLTCAAIFMYADDILLLSPTVDGLQQLLHVCENELEQLDMKLNVNKSVCIRVGPRFNADCAELRSRNGAVLKWTDSCRYLGVYFVRGRTLKCSFSNAKSRFFRAFNAVFGKVGRAASEETVIELIRAKCIPILLYATEACPFFSRDKQSLEFTVTRLFMKIFQTGSPAVVRDCQRSFNFLPIEMQVIIRTSRFLQAFVATQNSLCSLFQRSASCQLNDIFSKYDRVQTASQLANRIHHTFAMCDSV

SEQ ID NO: 109 - protein, 807 aa, synthetic construct
  source 1..807: note = MG148-43 reverse transcriptase
MESAQIDVLLLQEHWLSDAQLNALNNVGANYLNFGVSGFDTSAVLGGRPYGGCAVLWRSDLLLQVQPLVVSSRRLSAVIFSTDNWSLILINVYMPYEGDEIKTDEFIDLLSIIEDLVLSNSASHVIVGGDFNVEFNRNRMHTALLNSFCDNTGLSPVIQHSSCNIDYTYNFNMSRFNILDHFLLSGTLFDVCVTSAYVVHDIDNTSDHDPIILRLSLDIKYVSVCNRTSSSRVSWVKASDRDIRNYQYNLASNLQHVTIPSVALLCKDVNCSNFAHRCQLSRYLTDISDACLAAGEASIPHTCSRHSGKRIPGWSEKVEPLRQRSLFWHSMWVECGRPRSGVVADCMRRARASYHYAIRAVRRNEQNIIRERVADALLRDPSRDFWTEVKKIRNSKSGRAVIVDGCSDAPSISQLFASKYRHLYTSVPYQHNDLQSIVSDVESRISFDGDCFIGSQEVMAALSKLKLHKNDGDLGLASDYFINSDPALSVHIALLFTGIVIHGFVPSNLLSSTIVPIPKKSNVNATDSDNYRGIALSSVLGKIFDNVILVKYSDKLSTCNLQFGFKRNSSTHMCTMVLKETISYYVNNNSSVFCTFLDASKAFDRVHYCKLFRLLLGRGLPVCILRVLIQLYVGHSIRVTWAGLVSSCFTALNGVKQGGVLSPVLFCIYVDELLIRLAESGVGCYIGFSFAGALAYADDIVLIAPTPSAMRKLLAICDTYATEYNILFNAQKSNFIAFVPSSRHFLHKAMTNCVFRIGGVQIEHVFSYTHLGHIITSRLDDADDILHRRSSYIGQVNNVVCYFDSLS

SEQ ID NO: 110 - protein, 357 aa, synthetic construct
  source 1..357: note = MG148-44 reverse transcriptase
GSARCPSIGQPTSRVEPWRLTREGNPTASNVVDGCTTPDSISQLFAAKYKELYTSVPYSADDMSSLNSDINALMSSGFCAGCFVNANEVFAAVKELKLHKNDGNTGLTSDHVKCALPDLSVHIALLLSGVLSHGSIPKDLLLSTVIPIPKNKNGNLADSSNYRGIALSSVFCKIFDKILLARYNDKFVTSELQFGFKAGRSTHMCSMLLKESMLYYKNNNSLVYCAFLDATKAFDRVNYCKLFRLLIKRGLPPPILRALLNFYVGHTVRVSWFGSVSSYFLALNGMKQGGVISPLLFCVYIDDLLCALEKSGVGCYIGLHFTGALAYADDIVLLAPSPTALRRLLSICDVYAAEYCI

SEQ ID NO: 111 - protein, 969 aa, synthetic construct
  source 1..969: note = MG148-45 reverse transcriptase
MGFQAQTAGGMNNTAIMTSDQNCTSLSALTFNMHGFNQGSVLLKEVCEKQIYDLIFIQEHWLYPSNISKLLEISPHYIGHGISAMESAVDAGFIKGRPYGGCAILVNNKYKNMHADVCTFERVVAILLGDVLLINVYCPCENGSTEGFDALNEILANIANILDTYKAQYVVIGGDFNTDLSKDTTHSVLIKEFLDEYGIKYCKSEMFGSVNIQFERTFACETTGAGSCIDFFCVSDNICNNVMRYKVLDMYSNHSDHRPILCQFCVPVQSSLHQYVSSGCVNFVNERANANVHASKQSAESEQALRWDLGNVQLYNDLTYQYLYPIYDRLMNYDNMHSVNETNINEGKEAACVFIEQSYNDLTNALHHCAANSIPRVQRNAIKHWWNSELTELKKCSITSHNNWLESGKPRSGAVFALKQQDKLRYKQAIKKVKNEADAAISDELHAKLCAKKTTSFWKTWKNKVSNKAKKQISVEGCNTDEMAVCSLAEHFRKATVPNCEIFNNEKKTEFVHKLSEYSVNSSPTAVTAEIVYISLSKVHKGKAPGIDNITAEHIGNSHVVIYSLLAKLFNMMLEYGHVPVSFTNGIIVPIPKDENNKNKYPVDNFRGITLSPVLSKIFEHCLLNTFGDYLLTCDNQFGFKTKVGCSHAVYAVRKITDYFVSGESTVNLCFLDISKGFDKVNKYELLLKLMNRRIPVCFIELLNYWLSMSISTVKWNGFFSQPYSLEAGVRQGGVLSPLLFAVYVNDMLCKLRKMGCSYKWLHFGAIMYADDLVLLAPSVYELQCMIRVCQEQLKLLDLRLNFKKSKALRIGKRYKHKAAQLQLNGEKIPWGTEAKYLGMVIQSAARFKCSFDQAKVKFYRGANAILSKLSNKSNVTLTLYLVSTIALPTLTYGIECLSLNKTEIKSLDYPWYKCFCRVFQTFDAGIIQLCQAILGYKSIKELYENRCQMFLEKIKIYGNFMLREASEL

SEQ ID NO: 112 - protein, 955 aa, synthetic construct
  source 1..955: note = MG148-46 reverse transcriptase
MNSYSSTTLSAAESSCTTLSITTFNMHGFNQGSTLLIDVCYKETYDVIFIQEHWLYPHNIRKLLDISSKYTGYGISAMQSVVDSGFVKGRPFGGCAILIHNKYINLHVDCSVFERVVAVNVGDFLLTNVYFPCENGSAEGFDTLNEILANVSNILDTYKAPYAIIGGDFNTNLTKRSIHANTVNDFLNAHNMSYCKAKVFGSVEISFEDTFACDKTGASSCIDFVCVSNCLMHNVSKYEVLDVYNNHSDHKPLFCQLCVPVSSTLHQFVASGCRKLMNGNAGDSKCSNDSNCALRWDLGNVQLYTTLTSERLYPIYEKLLNYRYVVDEHNPHESKQLASQRIDKLYNELILALHECAATCIPQVKRNAIKHWWNSELNELKTCSIKSHKSWLEAHKPKSGALYLAKQQAKLRYKQAIKRTRLAADASISDALHVNLTTSSTEKFWKTWKTKVCKKNKQNMLLEGCSTDDIAVCKLADFFSKTTAPNSDEYNVKKKSEFLEQITDYSGQTNYDTINAELVYNSMSKLKKGKSPGIDNITVEHICNSHVVIFALLAKLFNLMLEVRYVPAIFGNGIIVPIPKEDTGKKVHPVDNFRGITLSPVLSKLFEHCLKCIFAEFMSTCDNQFAFKNNMGCSHAIYSVRKVTEYFVMGDSTVNLCFLDISKGFDKVNKYELLLKLIKRNVPVCFIELMHHWFDISKSTVKWNGHFSQSYELKAGVRQGGVLSPLLFAVYVDDMLRKISKMGCTYNWLHLGAIMYADDLVLIASSVYELQCMLRVCQAELNLIDLKLNVKKSKAIRIGKRSKLKAASLKLSEGHIAWSSEAKYLGIVVQSAARFKCNFDHVKIKFYRGANAILSKLSSKANVTVTLHLISSIALPSLTYGLECLALNKTELKSLHYPWHKCLCRIFQTFDEQIINLCQAILGYKSVKDLYEIRACMFLSRVKKCDNFLLRALAA

SEQ ID NO: 113 - protein, 454 aa, synthetic construct
  source 1..454: note = MG153-22 reverse transcriptase
MRQKSEQLELALDNRGEAPTSRRSGEAPTTAHEAERSGGGHRLMEQVVARANALAALRRVKQNRGSPGVDGMTVGELPQYLAKHWETVREQLLAGSYQPEPVRRQAIPKPGGGTRVLGIPTVLDRFIQQCLLQVLQPRFDPSFSDHSYGFRPGRNAHDAVCAAQQYIQAGRRWVADLDLEKFFDRVNHDVLMERLARRIADRRVLRLIRRYLVAGILHGGVVVERREGTPQGGPLSPLLASVLLDEVDRELERRGLAFARYADDLNVYCGSRRAAHDAMATLKRLFAALRLKVNESKSTVARVWERKFLGYSFWVAPGRVIRPRIAPAALAVMKERVRRITRRTGGRSLEAVAQELREYLTGWKAYFRLAGKPRVFRDLDEWTRHRLRAVQLKQWKRGRTVCRELLARGVPEREARAAAAHARRWWAMAAHSALQTALPNSHFDQLGVPRLAGR

SEQ ID NO: 114 - protein, 462 aa, synthetic construct
  source 1..462: note = MG153-23 reverse transcriptase
MRADEAEAHASAASTGKGGRNLPGTAAGAEVRAAAGGRTKPEALRLMEAAVERSNMLGAYERVVKNQGAPGVDGLTVTEFKPWLQAHWPKIRQVLLAGEYMPAAVRKVDIPKPQGGVRTLGIPTVLDRLIQQALHQVLQPLFEPEFSESSYGFRPGRNAHQAVEAARSYVAEGKRWVVDLEKFFDRVNHDVLMARVARKVKDERVLKLIRRYLEAGLMEGGMTSARTEGTPQGGPLSPLLSNTLLTDLDRELEKRGHRFCRYADDCNVYVGSRRSGQRVMAKITAFLEQRLKLQVNADKSAVARPWQRKFLGYSVTWHRNPKLKIAPSSRQRLAEKIRQTLRGARGQSLRQVIAQLNPILRGWVAYFRLTEEKGVLEELDGWVRRKLRALLWRHWKRGYPRAQNLMRAGLRPERAWQSATNGHGPWWNGGSSHMNAACPKSWFDHMQLVSLLATQRRFSLVS

SEQ ID NO: 115 - protein, 466 aa, synthetic construct
  source 1..466: note = MG153-24 reverse transcriptase
MKGGKQKISQDTCLQESRAEPEGYAGGQTFIWMTENNLTNANKPEYGLLEQILSPTNLNRAYKGVRSNRGSGGIDKMEVESLKDYLVDNKETLIQSILDGKYRPNPVRRVEISKEKGTRKLGIPTVVDRVIQQAIAQVLSPTYERQFSENSYGFRPGRNAHQALNRCRDYITDGYIYAVDMDLEKFFDTVNQSKLIEVLSRTVKDGRVVSLIHKYLDAGVVIRNKFEETEMGVPQGSPLSPVLSNIMLNELDKELEKRGHPFVRYADDLIIFCKSKRSADRTLANTVPYIENKLFLKVNREKTTTAYVSGIKFLGYSFYVYKGEGRLRVHPKSIAKMKERIRKLTSRSNGWGYARRKEALRQYITGWVNYFKLADMKKLLVSVDEWYRRRLRLVIWKQWKRVRTRGRYLMKLGIVKHQAWEFANTRKGYWHTAKSPILNRSVTSNRLRQAGYVFFVDYYRVVNGIN

SEQ ID NO: 116 - protein, 271 aa, synthetic construct
  source 1..271: note = MG160-7 reverse transcriptase
MHNFKSRFELSKGKWVYIQIEDLANHAKDHIRQIRDLWTPPEYFFHLQKGGHIAALRLHTPNEWYGKVDLSKFFNNITRHRVTRSLKRIGYSFRDAEEFAVASTVLVNHATRRYVLPYGFVQSPLLASISLDKSDLGNCLRRLHEETVSVSVYVDDIVVSADSERDVAEALSNIYLAAINSRFPINEEKSRGPSSTLNAFNIELDMHELEITAKRYEKMCGEVLINGTGQVSDAILNYVQTVNPAQAEQMLRDFPGVFGTLSSSASRYDAS

SEQ ID NO: 117 - protein, 1134 aa, synthetic construct
  source 1..1134: note = nMG3-6_D13A
MSTDMKNYRIGVAVGDRSVGLAAIEFDDDGLPIQKLALVTFRHDGGLDPTKNKTPMSRKETRGIARRTMRMNRERKRRLRNLDNVLENLGYSVPEGPEPETYEAWTSRALLASIKLASADELNEHLVRAVRHMARHRGWANPWWSLDQLEKASQEPSETFEIILARARELFGEKVPANPTLGMLGALAANNEVLLRPRDEKKRKTGYVRGTPLMFAQVRQGDQLAELRRICEVQGIEDQYEALRLGVFDHKHPYVPKERVGKDPLNPSTNRTIRASLEFQEFRILDSVANLRVRIGSRAKRELTEAEYDAAVEFLMDYADKEQPSWADVAEKIGVPGNRLVAPVLEDVQQKTAPYDRSSAAFEKAMGKKTEARQWWESTDDDQLRSLLIAFLVDATNDTEEAAAEAGLSELYKSWPAEEREALSNIDFEKGRVAYSQETLSKLSEYMHEYRVGLHEARKAVFGVDDTWRPPLDKLEEPTGQPAVDRVLTILRRFVLDCERQWGRPRAITVEHTRTGLMGPTQRQKILNEQKKNRADNERIRDELRESGVDNPSRAEVRRHLIVQEQECQCLYCGTMITTTTSELDHIVPRAGGGSSRRENLAAVCRACNAKKKRELFYAWAGPVKSQETIERVRQLKAFKDSKKAKMFKNQIRRLNQTEADEPIDERSLASTSYAAVAVRERLEQHFNEGLALDDKSRVVLDVYAGAVTRESRRAGGIDERILLRGERDKNRFDVRHHAVDAAVMTLLNRSVALTLEQRSQLRRTFYEQGLDKLDRNQLKPEEDWRDFTGLAPASQEKFLEWRKAATILGDLLAEAIEDDSIAVVSPLRLRPQNGSVHLETISAVKKQTLGSDWPADAVKRIVDPEIYLAMKDALGKLKELPEDSARSLELPDGRFVEADDEVLFFPENAASILTPRGVAEIGGSIHHARLYGWLTKKGELKVGMLRVYGAEFPWLMRESGSRNVLSMPIHRGSQSFRDMQDTTRKAVESGEAVEFAWITQNDELEFDPDDYIAHGGKDELRQFLGFMPECRWRVDGFKKNYQIRIRPAMLSREQLPSDIQRRLESKTLTKNESLLLKALDTGLVVAIGGLLPLETLKVIRRNNLGFPRWRGNGNLPTSFEVRSSALRALGVEG

SEQ ID NO: 118 - protein, 1134 aa, synthetic construct
  source 1..1134: note = nMG3-6_H586A
MSTDMKNYRIGVDVGDRSVGLAAIEFDDDGLPIQKLALVTFRHDGGLDPTKNKTPMSRKETRGIARRTMRMNRERKRRLRNLDNVLENLGYSVPEGPEPETYEAWTSRALLASIKLASADELNEHLVRAVRHMARHRGWANPWWSLDQLEKASQEPSETFEIILARARELFGEKVPANPTLGMLGALAANNEVLLRPRDEKKRKTGYVRGTPLMFAQVRQGDQLAELRRICEVQGIEDQYEALRLGVFDHKHPYVPKERVGKDPLNPSTNRTIRASLEFQEFRILDSVANLRVRIGSRAKRELTEAEYDAAVEFLMDYADKEQPSWADVAEKIGVPGNRLVAPVLEDVQQKTAPYDRSSAAFEKAMGKKTEARQWWESTDDDQLRSLLIAFLVDATNDTEEAAAEAGLSELYKSWPAEEREALSNIDFEKGRVAYSQETLSKLSEYMHEYRVGLHEARKAVFGVDDTWRPPLDKLEEPTGQPAVDRVLTILRRFVLDCERQWGRPRAITVEHTRTGLMGPTQRQKILNEQKKNRADNERIRDELRESGVDNPSRAEVRRHLIVQEQECQCLYCGTMITTTTSELDAIVPRAGGGSSRRENLAAVCRACNAKKKRELFYAWAGPVKSQETIERVRQLKAFKDSKKAKMFKNQIRRLNQTEADEPIDERSLASTSYAAVAVRERLEQHFNEGLALDDKSRVVLDVYAGAVTRESRRAGGIDERILLRGERDKNRFDVRHHAVDAAVMTLLNRSVALTLEQRSQLRRTFYEQGLDKLDRNQLKPEEDWRDFTGLAPASQEKFLEWRKAATILGDLLAEAIEDDSIAVVSPLRLRPQNGSVHLETISAVKKQTLGSDWPADAVKRIVDPEIYLAMKDALGKLKELPEDSARSLELPDGRFVEADDEVLFFPENAASILTPRGVAEIGGSIHHARLYGWLTKKGELKVGMLRVYGAEFPWLMRESGSRNVLSMPIHRGSQSFRDMQDTTRKAVESGEAVEFAWITQNDELEFDPDDYIAHGGKDELRQFLGFMPECRWRVDGFKKNYQIRIRPAMLSREQLPSDIQRRLESKTLTKNESLLLKALDTGLVVAIGGLLPLETLKVIRRNNLGFPRWRGNGNLPTSFEVRSSALRALGVEG

SEQ ID NO: 119 - protein, 1134 aa, synthetic construct
  source 1..1134: note = nMG3-6_N609A
MSTDMKNYRIGVDVGDRSVGLAAIEFDDDGLPIQKLALVTFRHDGGLDPTKNKTPMSRKETRGIARRTMRMNRERKRRLRNLDNVLENLGYSVPEGPEPETYEAWTSRALLASIKLASADELNEHLVRAVRHMARHRGWANPWWSLDQLEKASQEPSETFEIILARARELFGEKVPANPTLGMLGALAANNEVLLRPRDEKKRKTGYVRGTPLMFAQVRQGDQLAELRRICEVQGIEDQYEALRLGVFDHKHPYVPKERVGKDPLNPSTNRTIRASLEFQEFRILDSVANLRVRIGSRAKRELTEAEYDAAVEFLMDYADKEQPSWADVAEKIGVPGNRLVAPVLEDVQQKTAPYDRSSAAFEKAMGKKTEARQWWESTDDDQLRSLLIAFLVDATNDTEEAAAEAGLSELYKSWPAEEREALSNIDFEKGRVAYSQETLSKLSEYMHEYRVGLHEARKAVFGVDDTWRPPLDKLEEPTGQPAVDRVLTILRRFVLDCERQWGRPRAITVEHTRTGLMGPTQRQKILNEQKKNRADNERIRDELRESGVDNPSRAEVRRHLIVQEQECQCLYCGTMITTTTSELDHIVPRAGGGSSRRENLAAVCRACAAKKKRELFYAWAGPVKSQETIERVRQLKAFKDSKKAKMFKNQIRRLNQTEADEPIDERSLASTSYAAVAVRERLEQHFNEGLALDDKSRVVLDVYAGAVTRESRRAGGIDERILLRGERDKNRFDVRHHAVDAAVMTLLNRSVALTLEQRSQLRRTFYEQGLDKLDRNQLKPEEDWRDFTGLAPASQEKFLEWRKAATILGDLLAEAIEDDSIAVVSPLRLRPQNGSVHLETISAVKKQTLGSDWPADAVKRIVDPEIYLAMKDALGKLKELPEDSARSLELPDGRFVEADDEVLFFPENAASILTPRGVAEIGGSIHHARLYGWLTKKGELKVGMLRVYGAEFPWLMRESGSRNVLSMPIHRGSQSFRDMQDTTRKAVESGEAVEFAWITQNDELEFDPDDYIAHGGKDELRQFLGFMPECRWRVDGFKKNYQIRIRPAMLSREQLPSDIQRRLESKTLTKNESLLLKALDTGLVVAIGGLLPLETLKVIRRNNLGFPRWRGNGNLPTSFEVRSSALRALGVEG

SEQ ID NO: 120 - protein, 7 aa, synthetic construct
PKKKRKV

SEQ ID NO: 121 - protein, 16 aa, synthetic construct
KRPAATKKAGQAKKKK

SEQ ID NO: 122 - protein, 9 aa, synthetic construct
PAAKRVKLD

SEQ ID NO: 123 - protein, 11 aa, synthetic construct
RQRRNELKRSP

SEQ ID NO: 124 - protein, 38 aa, synthetic construct
NQSSNFGPMKGGNFGGRSSGPYGGGGQYFAKPRNQGGY

SEQ ID NO: 125 - protein, 42 aa, synthetic construct
RMRIZFKNKGKDTAELRRRRVEVSVELRKAKKDEQILKRRNV

SEQ ID NO: 126 - protein, 8 aa, synthetic construct
VSRKRPRP

SEQ ID NO: 127 - protein, 8 aa, synthetic construct
PPKKARED

SEQ ID NO: 128 - protein, 8 aa, synthetic construct
PQPKKKPL

SEQ ID NO: 129 - protein, 12 aa, synthetic construct
SALIKKKKKMAP

SEQ ID NO: 130 - protein, 5 aa, synthetic construct
DRLRR

SEQ ID NO: 131 - protein, 7 aa, synthetic construct
PKQKKRK

SEQ ID NO: 132 - protein, 10 aa, synthetic construct
RKLKKKIKKL

SEQ ID NO: 133 - protein, 10 aa, synthetic construct
REKKKFLKRR

SEQ ID NO: 134 - protein, 20 aa, synthetic construct
KRKGDEVDGVDEVAKKKSKK

SEQ ID NO: 135 - protein, 17 aa, synthetic construct
RKCLQAGMNLEARKTKK

SEQ ID NO: 136 - protein, 1134 aa, synthetic construct
  source 1..1134: note = MG3-6
MSTDMKNYRIGVDVGDRSVGLAAIEFDDDGLPIQKLALVTFRHDGGLDPTKNKTPMSRKETRGIARRTMRMNRERKRRLRNLDNVLENLGYSVPEGPEPETYEAWTSRALLASIKLASADELNEHLVRAVRHMARHRGWANPWWSLDQLEKASQEPSETFEIILARARELFGEKVPANPTLGMLGALAANNEVLLRPRDEKKRKTGYVRGTPLMFAQVRQGDQLAELRRICEVQGIEDQYEALRLGVFDHKHPYVPKERVGKDPLNPSTNRTIRASLEFQEFRILDSVANLRVRIGSRAKRELTEAEYDAAVEFLMDYADKEQPSWADVAEKIGVPGNRLVAPVLEDVQQKTAPYDRSSAAFEKAMGKKTEARQWWESTDDDQLRSLLIAFLVDATNDTEEAAAEAGLSELYKSWPAEEREALSNIDFEKGRVAYSQETLSKLSEYMHEYRVGLHEARKAVFGVDDTWRPPLDKLEEPTGQPAVDRVLTILRRFVLDCERQWGRPRAITVEHTRTGLMGPTQRQKILNEQKKNRADNERIRDELRESGVDNPSRAEVRRHLIVQEQECQCLYCGTMITTTTSELDHIVPRAGGGSSRRENLAAVCRACNAKKKRELFYAWAGPVKSQETIERVRQLKAFKDSKKAKMFKNQIRRLNQTEADEPIDERSLASTSYAAVAVRERLEQHFNEGLALDDKSRVVLDVYAGAVTRESRRAGGIDERILLRGERDKNRFDVRHHAVDAAVMTLLNRSVALTLEQRSQLRRTFYEQGLDKLDRNQLKPEEDWRDFTGLAPASQEKFLEWRKAATILGDLLAEAIEDDSIAVVSPLRLRPQNGSVHLETISAVKKQTLGSDWPADAVKRIVDPEIYLAMKDALGKLKELPEDSARSLELPDGRFVEADDEVLFFPENAASILTPRGVAEIGGSIHHARLYGWLTKKGELKVGMLRVYGAEFPWLMRESGSRNVLSMPIHRGSQSFRDMQDTTRKAVESGEAVEFAWITQNDELEFDPDDYIAHGGKDELRQFLGFMPECRWRVDGFKKNYQIRIRPAMLSREQLPSDIQRRLESKTLTKNESLLLKALDTGLVVAIGGLLPLETLKVIRRNNLGFPRWRGNGNLPTSFEVRSSALRALGVEG

SEQ ID NO: 137 - protein, 1280 aa, synthetic construct
  source 1..1280: note = MG29-1
MFNNFIKKYSLQKTLRFELKPVGETADYIEDFKSEYLKDTVLKDEQRAKDYQEIKTLIDDYHREYIEECLREPVDKKTGEILDFTQDLEDAFSYYQKLKENPTENRVGWEKEQESLRKKLVTSFVGNDGLFKKEFITRDLPEWLQKKGLWGEYKDTVENFKKFTTYFSGFHENRKNMYTAEAQSTAIANRLMNDNLPKFFNNYLAYQTIKEKHPDLVFRLDDALLQAAGVEHLDEAFQPRYFSRLFAQSGITAFNELIGGRTTENGEKIQGLNEQINLYRQQNPEKAKGFPRFMPLFKQILSDRETHSFLPDAFENDKELLQALRDYVDAATSEEGMISQLNKAMNQFVTADLKRVYIKSAALTSLSQELFHFFGVISDAIAWYAEKRLSPKKAQESFLKQEVYAIEELNQAVVGYIDQLEDQSELQQLLVDLPDPQKPVSSFILTHWQKSQEPLQAVIAKVEPLFELEELSKNKRAPKHDKDQGGEGFQQVDAIKNMLDAFMEVSHAIKPLYLVKGRKAIDMPDVDTGFYADFAEAYSAYEQVTVSLYNKTRNHLSKKPFSKDKIKINFDAPTLLNGWDLNKESDNKSIILRKDGNFYLAIMHPKHTKVFDCYSASEAAGKCYEKMNYKLLSGANKMLPKVFFSKKGIETFSPPQEILDLYKNNEHKKGATFKLESCHKLIDFFKRNIPKYKVHPTDNFGWDVFGFHFSPTSSYGDLSGFYREVEAQGYKLWFSDVSEAYINKCVEEGKLFLFQIYNKDFSPNSTGKPNLHTLYWKGLFEPENLKDVVLKLNGEAEIFYRKHSIKHEDKTIHRAKDPIANKNADNPKKQSVFDYDIIKDKRYTQDKFFFHVPISLNFKSQGVVRFNDKINGLLAAQDDVHVIGIDRGERHLLYYTVVNGKGEVVEQGSLNQVATDQGYVVDYQQKLHAKEKERDQARKNWSTIENIKELKAGYLSQVVHKLAQLIVKHNAIVCLEDLNFGFKRGRFKVEKQVYQKFEKALIDKLNYLVFKERGATQAGGYLNAYQLAAPFESFEKLGKQTGILYYVRSDYTSKIDPATGFVDFLKPKYESMAKSKVFFESFERIQWNQAKGYFEFEFDYKKMCPSRKFGDYRTRWVVCTFGDTRYQNRRNKSSGQWETETIDVTAQLKALFAAYGITYNQEDNIKDAIAAVKYTKFYKQLYWLLRLTLSLRHSVTGTDEDFILSPVADENGVFFDSRKATDKQPKDADANGAYHIALKGLWNLQQIRQHDWNVEKPKKLNLAMKNEEWFGFAQKKKFRA

SEQ ID NO: 138 - protein, 245 aa, synthetic construct
  source 1..245: note = MG160-62 reverse transcriptase
MAVRFSTFDYKYQKNAKWYFAPSKKGEEIGGDLKRQIEAAHRFDTTYFHLHKGGHVAALHEHRSHRLFCRVDIENFFYRIARNRVARALRNIGIARAEHYAKWSTVKDPFGEFNYVLPYGFVQSPILATLVLDQSGVGDFIRAIPPAIGRSIYMDDIVLSGDDEVQLQQYFKGLTNELERSNFRLNPSKIRAPAKTMDVFNCDLETGQSVVQTKRKEKFFAKARSPASIEAFEEYCERVEHGNSI

SEQ ID NO: 139 - protein, 225 aa, synthetic construct
  source 1..225: note = MG160-366 reverse transcriptase
MPSDDGRAVGYELKALVEAKFTPDPFLYHLAKGGHVAAIHIHRRQKYFSRLDFANFFYSIARNRVARALREIGLARAEYYAKWSCVKNPYERPSYALPYGFVQSPLLASLVLAKSEVGTFLRELSERIVVSVYVDDIALSGNNRRVLQRVHRELRRIAVTSNFTINEAKSVELGETVELFNCDLLHQKTIVTEARRREFYAIARSAASETAFENYCAAIAVGNVA

SEQ ID NO: 140 - protein, 4 aa, synthetic construct
YADD

SEQ ID NO: 141 - protein, 4 aa, synthetic construct
YIDD

SEQ ID NO: 142 - protein, 4 aa, synthetic construct
FADD

SEQ ID NO: 143 - protein, 4 aa, synthetic construct
FVDD

SEQ ID NO: 144 - protein, 4 aa, synthetic construct
FIDD

SEQ ID NO: 145 - protein, 4 aa, synthetic construct
FLDD

(19) United States
(12) Patent Application Publication
(10) Pub. No.: US 2026/0146263 A1
(43) Pub. Date: May 28, 2026

(54) GENE EDITING SYSTEMS COMPRISING REVERSE TRANSCRIPTASES

(71) Applicant: Metagenomi, Inc., Emeryville, CA (US)

(72) Inventors: Brian C. THOMAS, Emeryville, CA (US); Christopher BROWN, Emeryville, CA (US); Daniela S.A. GOLTSMAN, Emeryville, CA (US); Sourab KULKARNI, Emeryville, CA (US); Sarah LAPERRIERE, Emeryville, CA (US); Leanna MONTELEONE, Emeryville, CA (US); Morayma TEMOCHE-DIAZ, Emeryville, CA (US); Anu THOMAS, Emeryville, CA (US)

(21) Appl. No.: 19/122,156

(22) PCT Filed: Oct. 18, 2023
(86) PCT No.: PCT/US2023/077217

§ 371 Date: Apr. 17, 2025

Related U.S. Application Data
(60) Provisional application No. 63/380,195, filed on Oct. 19, 2022.
(60) Provisional application No. 63/386,659, filed on Dec. 8, 2022.

(51) Int. Cl. C12N 15/90; C12N 9/12; C12N 9/22; C12N 15/11

(52) U.S. Cl. CPC C12N 15/907; C12N 9/1276; C12N 9/226; C12N 15/11; C12Y 207/07049; C12N 2310/20; C12N 2800/90

(57) ABSTRACT

The disclosure relates generally to gene editing systems comprising reverse transcriptases and fusion proteins of reverse transcriptases with nickases or nucleases, methods of making such reverse transcriptases and fusion proteins, and methods of using such reverse transcriptases and fusion proteins for site directed genome editing in cells.

### CROSS-REFERENCE

This application claims the benefit of and priority to U.S. Provisional Patent Application No. 63/380,195 filed Oct. 19, 2022 and U.S. Provisional Patent Application No. 63/386,659 filed Dec. 8, 2022, each of which is incorporated by reference in its entirety herein.

### BRIEF SUMMARY

The disclosure is based, in part, upon the development of a gene editing system comprising a reverse transcriptase, a nuclease or nickase, and a guide RNA or pegRNA.

Described herein are fusion proteins comprising a nickase linked to a reverse transcriptase using a linker, wherein the reverse transcriptase comprises at least about 80% sequence identity to any one of SEQ ID NOs: 88-116 and 138-139.

Described herein are fusion proteins comprising a nuclease linked to a reverse transcriptase using a linker, wherein the reverse transcriptase comprises at least about 80% sequence identity to any one of SEQ ID NOs: 88-116 and 138-139.

Described herein are fusion proteins comprising a catalytically dead nuclease linked to a reverse transcriptase using a linker, wherein the reverse transcriptase comprises at least about 80% sequence identity to any one of SEQ ID NOs: 88-116 and 138-139.

Described herein are gene editing systems, comprising a) a nickase; b) a guide nucleic acid configured to form a complex with the nickase and to hybridize to a target nucleic acid sequence; and c) a reverse transcriptase having at least about 80% sequence identity to any one of SEQ ID NOs: 88-116 and 138-139 and configured to form a complex with the nickase. In some embodiments, the gene editing system further comprises a nucleic acid template. In some embodiments, the nickase is a modified endonuclease. In some embodiments, the modified endonuclease is a Type II CRISPR endonuclease. In some embodiments, the modified endonuclease is a Type V CRISPR endonuclease. In some embodiments, the Type II CRISPR endonuclease or the Type V CRISPR endonuclease has nickase activity. In some embodiments, the modified endonuclease is selected from the group consisting of spCas9 (H840A), spCas9 (D10A), nMG3-6 (D13A), nMG3-6 (H586A), nMG3-6 (N609A), Cas12a, and MG29-1. In some embodiments, the modified endonuclease comprises at least about 80% sequence identity to any one of SEQ ID NOs: 117-119.

In some embodiments, the nickase and the reverse transcriptase are linked. In some embodiments, the nickase and the reverse transcriptase are linked by a linker. In some embodiments, the linker comprises at least 10, 20, or 30 amino acids. In some embodiments, the linker comprises about 30-35 amino acids. In some embodiments, the linker comprises about 30 amino acids. In some embodiments, the linker comprises at least 80% sequence identity to SEQ ID NO: 33. In some embodiments, the linker comprises at least 80% sequence identity to any one of SEQ ID NOs: 82-87. In some embodiments, the nickase and the reverse transcriptase are not linked. In some embodiments, the guide nucleic acid comprises a spacer sequence and a crRNA. In some embodiments, the guide nucleic acid further comprises a reverse transcriptase template (RTT). In some embodiments, a base in the RTT comprises a bulky modification selected from the group of complex sugars, or complex amino groups, and/or other modifications compatible with RNA. In some embodiments, the guide nucleic acid further comprises a primer binding site. In some embodiments, the primer binding site is on a 3′ end of the guide nucleic acid. In some embodiments, the primer binding site comprises at least 2, 4, 6, 8, 10, 13, 16, 20, 24, 28, 32, 36, 40, 45, 50, 55, 60, or 65 nucleotides. In some embodiments, the gene editing system further comprises a transposase, integrase, or homing endonuclease. In some embodiments, the gene editing system further comprises a retrotransposon. In some embodiments, the reverse transcriptase comprises a processivity of at least about 2-fold more than Moloney Murine Leukemia Virus (MMLV) reverse transcriptase. In some embodiments, the reverse transcriptase comprises a processivity of at least about 2-fold less than Moloney Murine Leukemia Virus (MMLV) reverse transcriptase. In some embodiments, the reverse transcriptase comprises an error rate of less than about 2.5%, 2.0%, 1.5%, 1%, 0.5%, 0.25%, 0.10%, or 0.05%. In some embodiments, the reverse transcriptase comprises an error rate of less than about 2.5%, 2.0%, 1.5%, 1%, 0.5%, 0.25%, 0.10%, or 0.05% as compared to Moloney Murine Leukemia Virus (MMLV) reverse transcriptase.

Described herein are gene editing systems, comprising a) a nuclease; b) a guide nucleic acid configured to form a complex with the nuclease and to hybridize to a target nucleic acid sequence; and c) a reverse transcriptase having at least about 80% sequence identity to any one of SEQ ID NOs: 88-116 and 138-139 and configured to form a complex with the nickase. In some embodiments, the gene editing system further comprises a nucleic acid template. In some embodiments, the nuclease is a double strand nuclease. In some embodiments, the nuclease is a Type II CRISPR endonuclease. In some embodiments, the CRISPR endonuclease is Cas9. In some embodiments, the Cas9 is catalytically dead Cas9 (dCas9). In some embodiments, the nuclease and the reverse transcriptase are linked. In some embodiments, the nuclease and the reverse transcriptase are linked by a linker. In some embodiments, the linker comprises at least 10, 20, or 30 amino acids. In some embodiments, the linker comprises about 30-35 amino acids. In some embodiments, the linker comprises about 30 amino acids. In some embodiments, the linker comprises at least 80% sequence identity to SEQ ID NO: 33. In some embodiments, the linker comprises at least 80% sequence identity to any one of SEQ ID NOs: 82-87. In some embodiments, the nuclease and the reverse transcriptase are not linked. In some embodiments, the guide nucleic acid further comprises a primer binding site. In some embodiments, the primer binding site is on a 3′ end of the guide nucleic acid. In some embodiments, the primer binding site comprises at least 2, 4, 6, 8, 10, 13, 16, 20, 24, 28, 32, 36, 40, 45, 50, 55, 60, or 65 nucleotides. In some embodiments, the gene editing system further comprises a transposase, integrase, or homing endonuclease. In some embodiments, the gene editing system further comprises comprising a retrotransposon. In some embodiments, the reverse transcriptase comprises a processivity of at least about 2-fold more than Moloney Murine Leukemia Virus (MMLV) reverse transcriptase. In some embodiments, the reverse transcriptase comprises a processivity of at least about 2-fold less than Moloney Murine Leukemia Virus (MMLV) reverse transcriptase. In some embodiments, the reverse transcriptase comprises an error rate of less than about 2.5%, 2.0%, 1.5%, 1%, 0.5%, 0.25%, 0.10%, or 0.05%. In some embodiments, the reverse transcriptase comprises an error rate of less than about 2.5%, 2.0%, 1.5%, 1%, 0.5%, 0.25%, 0.10%, or 0.05% as compared to Moloney Murine Leukemia Virus (MMLV) reverse transcriptase.

Described herein are gene editing systems, comprising a) a nickase, b) a guide nucleic acid configured to form a complex with the nickase and to hybridize to a target nucleic acid sequence; and c) a reverse transcriptase configured to form a complex with the nickase, the reverse transcriptase having a X₁X₂DD motif, wherein X₁ is F or Y, and wherein when X₁ is Y, X₂ is A, R, N, D, C, E, Q, G, H, I, L, K, M, F, P, S, T, V, W, or Y. In some embodiments, the X₂ is A or I. In some embodiments, the X₁X₂DD motif is YADD (SEQ ID NO: 140) or YIDD (SEQ ID NO: 141). In some embodiments, the X₁X₂DD motif is FADD (SEQ ID NO: 142), FVDD (SEQ ID NO: 143), FIDD (SEQ ID NO: 144), or FLDD (SEQ ID NO: 145). In some embodiments, the reverse transcriptase has at least about 80% sequence identity to any one of SEQ ID NOs: 88-116 and 138-139.

In Described herein are gene editing systems, comprising a) a nuclease; b) a guide nucleic acid configured to form a complex with the nuclease and to hybridize to a target nucleic acid sequence; and c) a reverse transcriptase configured to form a complex with the nuclease, the reverse transcriptase having a X₁X₂DD motif, wherein X₁ is F or Y, and wherein when X₁ is Y, X₂ is A, R, N, D, C, E, Q, G, H, I, L, K, M, F, P, S, T, V, W, or Y. In some embodiments, the X₂ is A or I. In some embodiments, the X₁X₂DD motif is YADD (SEQ ID NO: 140) or YIDD (SEQ ID NO: 141). In some embodiments, the X₁X₂DD motif is FADD (SEQ ID NO: 142), FVDD (SEQ ID NO: 143), FIDD (SEQ ID NO: 144), or FLDD (SEQ ID NO: 145). In some embodiments, the reverse transcriptase has at least about 80% sequence identity to any one of SEQ ID NOs: 88-116 and 138-139.

Described herein are isolated reverse transcriptases having at least about 80% sequence identity to any one of SEQ ID NOs: 88-116 and 138-139.

Described herein are nucleic acids encoding for a fusion protein or a gene editing system as described above. In some embodiments, the nucleic acid is a DNA or an RNA. In some embodiments, the RNA is an mRNA. In some embodiments, nucleic acid is comprised in a vector. In some embodiments, the nucleic acid or the vector comprising the nucleic acid is comprised in an adeno-associated virus or a lipid nanoparticle. In some embodiments, the nucleic acid or the vector comprising the nucleic acid is comprised in a cell. In some embodiments, the cell is a human cell.

Described herein are methods for modifying a double- and/or single-stranded nucleic acid, comprising contacting a cell using a fusion protein or a gene editing system as described above.

Described herein are methods for modifying a double- and/or single-stranded nucleic acid in a cell comprising a) providing a cell with a guide nucleic acid to bind to a target strand of the nucleic acid; b) providing the cell with a nuclease or nickase to cleave the nucleic acid at a location of binding of the guide nucleic acid; c) providing the cell with a reverse transcriptase to synthesize a modification in the target strand of the nucleic acid at a location of cleavage by the nickase and/or nuclease. In some embodiments, the reverse transcriptase has at least about 80% sequence identity to any one of SEQ ID NOs: 88-116 and 138-139. In some embodiments, the modification is an insertion, deletion, or mutation. In some embodiments, the method further comprises providing an RNA or DNA template to the cell. In some embodiments, the nucleic acid is a genome or a vector. In some embodiments, the method further comprises providing the cell with a transposase, integrase, or homing endonuclease. In some embodiments, the method further comprises providing the cell with a retrotransposon.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1B are bar graphs showing the G-to-T conversion editing percentage of untethered reverse transcriptase (RT) candidates from the MG153 family. Candidates MG153-23 (FIG. 1A) and MG153-24 (FIG. 1B) were tested with eight different primer binding site (PBS) nucleotides of varying length (PBS lengths of 2, 4, 6, 8, 10, 13, 16, and 20 nucleotides) in HEK293T cells. The graph further shows untreated samples and wild-type MMLV1 as control.

FIG. 2 is a bar graph showing the G-to-T conversion editing percentage of an untethered reverse transcriptase (RT) candidate from the MG160 family. Candidate MG160-7 was tested with eight different primer binding site (PBS) nucleotides of varying length (PBS lengths of 2, 4, 6, 8, 10, 13, 16, and 20 nucleotides) in HEK293T cells. The graph further shows untreated samples and wild-type MMLV1 as control.

FIG. 3 is a bar graph showing the G-to-T conversion editing percentage of an RT candidate from the MG160 family tethered to spCas9(H840A). The MG160-7 candidate is shown with untreated samples and MMLV1 and MMVL2 as controls.

### BRIEF DESCRIPTION OF THE SEQUENCE LISTING

The Sequence Listing filed herewith provides exemplary polynucleotide and polypeptide sequences for use in methods, compositions, and systems according to the disclosure. Below are exemplary descriptions of sequences therein.

SEQ ID NOs: 1-3 show the full-length nucleic acid sequences of untethered MG153 family reverse transcriptases suitable for the gene editing systems described herein.

SEQ ID NO: 4 shows the full-length nucleic acid sequence of an untethered MG160 family reverse transcriptase suitable for the gene editing systems described herein.

SEQ ID NO: 5 shows the full-length nucleic acid sequence of a tethered MG160 family reverse transcriptase suitable for the gene editing systems described herein.

SEQ ID NOs: 6-13 show the RNA sequences of chemically modified guide RNAs with a single point mutation (VEGFA spacer G to T) with PBS of different lengths suitable for the gene editing systems described herein.

SEQ ID NOs: 14-21 show the RNA sequences of chemically modified guide RNAs with a single deletion (VEGFA spacer deletion change) with PBS of different lengths suitable for the gene editing systems described herein.

SEQ ID NOs: 22-29 show the RNA sequences of chemically modified guide RNAs with a single insertion (VEGFA spacer single insertion) with PBS of different lengths suitable for the gene editing systems described herein.

SEQ ID NOs: 30-31 show the sequences of primers suitable for conducting site-directed editing in the VEGFA site.

SEQ ID NO: 32 shows the nucleic acid sequence of the VEGFA target site.

SEQ ID NO: 33 shows the nucleic acid sequence of an exemplary RT-nickase linker.

SEQ ID NO: 34 shows the nucleic acid sequence of an MG3 effector nuclease suitable for the gene editing systems described herein.

SEQ ID NOs: 35-38 show the nucleic acid sequences of the endogenous targets AAVS1, B2M, CD5, and CD38.

SEQ ID NOs: 39-70 show the RNA sequences of chemically modified guide RNAs with spacers targeting AAVS1, B2M, CD5, and CD38 with PBS of different lengths suitable for the gene editing systems described herein.

SEQ ID NOs: 71-78 show the sequences of primers suitable for conducting site-directed editing in the AAVS1, B2M, CD5, and CD38 site.

SEQ ID NO: 79 shows the RNA sequence of a chemically modified guide RNA with a spacer targeting VEGFA.

SEQ ID NOs: 80-81 show the sequences of two retrotransposition assay reporters.

SEQ ID NOs: 82-87 show the amino acid sequences of exemplary RT-nickase linkers.

SEQ ID NOs: 88-103 show the amino acid sequences of MG140 family retrotransposition proteins suitable for the gene editing systems described herein.

SEQ ID NOs: 104-112 show the amino acid sequences of MG148 family reverse transcriptase proteins suitable for the gene editing systems described herein.

SEQ ID NOs: 113-115 show the amino acid sequences of MG153 family reverse transcriptase proteins suitable for the gene editing systems described herein.

SEQ ID NO: 116 shows the amino acid sequence of an MG160 family reverse transcriptase protein suitable for the gene editing systems described herein.

SEQ ID NOs: 117-119 show the amino acid sequences of MG3-6 nucleases suitable for the gene editing systems described herein.

SEQ ID NOs: 120-135 show nuclear localization signals (NLS) suitable for the gene editing systems described herein.

SEQ ID NO: 136 shows the amino acid sequence of an MG3-6 nuclease suitable for the gene editing systems described herein.

SEQ ID NO: 137 shows the amino acid sequence of an MG29-1 nuclease suitable for the gene editing systems described herein.

SEQ ID NOs: 138-139 show the amino acid sequences of MG160 family reverse transcriptase proteins suitable for the gene editing systems described herein.

### DETAILED DESCRIPTION

Site-directed gene editing systems are powerful tools for site-directed genome engineering in cells. Programmable nucleases such as Clustered Regularly Interspaced Short Palindromic Repeats (CRISPR) nucleases have been used recently for diverse DNA manipulation and gene editing applications. CRISPR nucleases can be used with or without a repair template to introduce site-directed insertions and deletions (indels) or varying length as well as point mutations. Single nucleotide point (SNP) mutations, deletions, and insertions represent over 80% of disease-causing mutations. However, not all of these mutations can be accurately repaired with the available gene editing systems. Clinical genome editing applications with a higher efficiency and fidelity of the system are needed.

Additionally, the repair or insertion of longer pieces of DNA has remained challenging, and a safe and efficient way of targeted integration of large templates into a genome, for example for gene therapies or engineered cell therapies, is lacking. To date, lentiviruses or adeno-associated viruses (AAV) in combination with a CRISPR nuclease are used to insert large pieces of DNA, for example whole genes. However, lentiviral-mediated integration lacks the targetability feature, as integration occurs mostly randomly in open chromatin. AAV-mediated delivery has a limited cargo capacity and is not available for all cell types. A safe and efficient targeted genome editing system that allows for large template integration is needed.

The present disclosure is based, in part, upon the development of a gene editing system comprising a reverse transcriptase, a nuclease or nickase, and a guide RNA or pegRNA. The gene editing system can be used to introduce site-directed insertions, deletions, and mutations in the genome of cells. Furthermore, it is contemplated that the gene editing system can be used in combination with a nucleic acid template to facilitate site-directed insertions into the genome of a cell, as well as for large template integration.

### Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. The section headings used herein are for organizational purposes only and are not to be construed as limiting the subject matter described.

The practice of some methods disclosed herein employ, unless otherwise indicated, techniques of immunology, biochemistry, chemistry, molecular biology, microbiology, cell biology, genomics, and recombinant DNA. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)).

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within one or more than one standard deviation, per the practice in the art. Alternatively, “about” can mean a range of up to 20%, up to 15%, up to 10%, up to 5%, or up to 1% of a given value.

The term “nucleotide,” as used herein, refers to a base-sugar-phosphate combination. Contemplated nucleotides include naturally occurring nucleotides and synthetic nucleotides. Nucleotides are monomeric units of a nucleic acid sequence (e.g., deoxyribonucleic acid (DNA) and ribonucleic acid (RNA)). The term nucleotide includes ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives include, for example, [αS]dATP, 7-deaza-dGTP and 7-deaza-dATP, and nucleotide derivatives that confer nuclease resistance on the nucleic acid molecule containing them. The term nucleotide as used herein encompasses dideoxyribonucleoside triphosphates (ddNTPs) and their derivatives. Illustrative examples of ddNTPs include, but are not limited to, ddATP, ddCTP, ddGTP, ddITP, and ddTTP. A nucleotide may be unlabeled or detectably labeled, such as using moieties comprising optically detectable moieties (e.g., fluorophores) or quantum dots. Detectable labels include, for example, radioactive isotopes, fluorescent labels, chemiluminescent labels, bioluminescent labels, and enzyme labels. Fluorescent labels of nucleotides include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif; FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, IL; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. The term nucleotide encompasses chemically modified nucleotides. An exemplary chemically-modified nucleotide is biotin-dNTP. Non-limiting examples of biotinylated dNTPs include, biotin-dATP (e.g., bio-N6-ddATP, biotin-14-dATP), biotin-dCTP (e.g., biotin-1 l-dCTP, biotin-14-dCTP), and biotin-dUTP (e.g., biotin-11-dUTP, biotin-16-dUTP, biotin-20-dUTP).

The terms “polynucleotide,” “oligonucleotide,” and “nucleic acid” are used interchangeably to refer to a polymeric form of nucleotides of any length, either deoxyribonucleotides or ribonucleotides, or analogs thereof, either in single-, double-, or multi-stranded form. Contemplated polynucleotides include a gene or fragment thereof. Exemplary polynucleotides include, but are not limited to, DNA, RNA, coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, cell-free polynucleotides including cell-free DNA (cfDNA) and cell-free RNA (cfRNA), nucleic acid probes, and primers. In a polynucleotide when referring to a T, a T means U (Uracil) in RNA and T (Thymine) in DNA. A polynucleotide can be exogenous or endogenous to a cell and/or exist in a cell-free environment. The term polynucleotide encompasses modified polynucleotides (e.g., altered backbone, sugar, or nucleobase). If present, modifications to the nucleotide structure are imparted before or after assembly of the polymer. Non-limiting examples of modifications include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol-containing nucleotides, biotin-linked nucleotides, fluorescent base analogs, CpG islands, methyl-7-guanosine, methylated nucleotides, inosine, thiouridine, pseudouridine, dihydrouridine, queuosine, and wyosine. The sequence of nucleotides may be interrupted by non-nucleotide components.

The terms “transfection” or “transfected” refer to introduction of a polynucleotide into a cell by non-viral or viral-based methods. The polynucleotides may be gene sequences encoding complete proteins or functional portions thereof. See, e.g., Sambrook et al., 1989, Molecular Cloning: A Laboratory Manual, 18.1-18.88.

The terms “peptide,” “polypeptide,” and “protein” are used interchangeably herein to refer to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer is interrupted by non-amino acids. The terms include amino acid chains of any length, including full length proteins, and proteins with or without secondary or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, refer to natural and non-natural amino acids, including, but not limited to, modified amino acids. Modified amino acids include amino acids that have been chemically modified to include a group or a chemical moiety not naturally present on the amino acid. The term “amino acid” includes both D-amino acids and L-amino acids.

As used herein, the “non-native” refers to a nucleic acid or polypeptide sequence that is non-naturally occurring. Non-native refers to a non-naturally occurring nucleic acid or polypeptide sequence that comprises modifications such as mutations, insertions, or deletions. The term non-native encompasses fusion nucleic acids or polypeptides that encodes or exhibits an activity (e.g., enzymatic activity, methyltransferase activity, acetyltransferase activity, kinase activity, ubiquitinating activity, etc.) of the nucleic acid or polypeptide sequence to which the non-native sequence is fused. A non-native nucleic acid or polypeptide sequence includes those linked to a naturally-occurring nucleic acid or polypeptide sequence (or a variant thereof) by genetic engineering to generate a chimeric nucleic acid or polypeptide sequence encoding a chimeric nucleic acid or polypeptide.

The term “promoter”, as used herein, refers to the regulatory DNA region which controls transcription or expression of a polynucleotide (e.g., a gene) and which may be located adjacent to or overlapping a nucleotide or region of nucleotides at which RNA transcription is initiated. A promoter may contain specific DNA sequences which bind protein factors, often referred to as transcription factors, which facilitate binding of RNA polymerase to the DNA leading to gene transcription. Eukaryotic basal promoters typically, though not necessarily, contain a TATA-box and/or a CAAT box.

The term “expression”, as used herein, refers to the process by which a nucleic acid sequence or a polynucleotide is transcribed from a DNA template (such as into mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, the term expression includes splicing of the mRNA in a eukaryotic cell.

As used herein, “operably linked”, “operable linkage”, “operatively linked”, or grammatical equivalents thereof refer to an arrangement of genetic elements, e.g., a promoter, an enhancer, a polyadenylation sequence, etc., wherein an operation (e.g., movement or activation) of a first genetic element has some effect on the second genetic element. The effect on the second genetic element can be, but need not be, of the same type as operation of the first genetic element. For example, two genetic elements are operably linked if movement of the first element causes an activation of the second element. For instance, a regulatory element, which may comprise promoter and/or enhancer sequences, is operatively linked to a coding region if the regulatory element helps initiate transcription of the coding sequence. There may be intervening residues between the regulatory element and coding region so long as this functional relationship is maintained.

A “vector” as used herein, refers to a macromolecule or association of macromolecules that comprises or associates with a polynucleotide and which mediates delivery of the polynucleotide to a cell. Examples of vectors include nucleic-based vectors (e.g., plasmids and viral vectors) and liposomes. An exemplary nucleic-acid based vector comprises genetic elements, e.g., regulatory elements, operatively linked to a gene to facilitate expression of the gene in a target.

As used herein, “expression cassette” and “nucleic acid cassette” are used interchangeably to refer to a component of a vector comprising a combination of nucleic acid sequences or elements (e.g., therapeutic gene, promoter, and a terminator) that are expressed together or are operably linked for expression. The terms encompass an expression cassette including a combination of regulatory elements and a gene or genes to which they are operably linked for expression.

A “functional fragment” of a DNA or protein sequence refers to a fragment that retains a biological activity (either functional or structural) that is substantially similar to a biological activity of the full-length DNA or protein sequence. A biological activity of a DNA sequence includes its ability to influence expression in a manner attributed to the full-length sequence.

The terms “engineered,” “synthetic,” and “artificial” are used interchangeably herein to refer to an object that has been modified by human intervention. For example, the terms refer to a polynucleotide or polypeptide that is non-naturally occurring. An engineered peptide has, but does not require, low sequence identity (e.g., less than 50% sequence identity, less than 25% sequence identity, less than 10% sequence identity, less than 5% sequence identity, less than 1% sequence identity) to a naturally occurring human protein. For example, VPR and VP64 domains are synthetic transactivation domains. Non-limiting examples include the following: a nucleic acid modified by changing its sequence to a sequence that does not occur in nature; a nucleic acid modified by ligating it to a nucleic acid that it does not associate with in nature such that the ligated product possesses a function not present in the original nucleic acid; an engineered nucleic acid synthesized in vitro with a sequence that does not exist in nature; a protein modified by changing its amino acid sequence to a sequence that does not exist in nature; an engineered protein acquiring a new function or property. An “engineered” system comprises at least one engineered component.

As used herein, a “guide nucleic acid” or “guide polynucleotide” refers to a nucleic acid that may hybridize to a target nucleic acid and thereby directs an associated nuclease to the target nucleic acid. A guide nucleic acid is, but is not limited to, RNA (guide RNA or gRNA), DNA, or a mixture of RNA and DNA. A guide nucleic acid can include a crRNA or a tracrRNA or a combination of both. The term guide nucleic acid encompasses an engineered guide nucleic acid and a programmable guide nucleic acid to specifically bind to the target nucleic acid. A portion of the target nucleic acid may be complementary to a portion of the guide nucleic acid. The strand of a double-stranded target polynucleotide that is complementary to and hybridizes with the guide nucleic acid is the complementary strand. The strand of the double-stranded target polynucleotide that is complementary to the complementary strand, and therefore is not complementary to the guide nucleic acid is called noncomplementary strand. A guide nucleic acid having a polynucleotide chain is a “single guide nucleic acid.” A guide nucleic acid having two polynucleotide chains is a “double guide nucleic acid.” If not otherwise specified, the term “guide nucleic acid” is inclusive, referring to both single guide nucleic acids and double guide nucleic acids. A guide nucleic acid may comprise a segment referred to as a “nucleic acid-targeting segment” or a “nucleic acid-targeting sequence,” or a “spacer.” A nucleic acid-targeting segment can include a sub-segment referred to as a “protein binding segment” or “protein binding sequence” or “Cas protein binding segment.”

The term “tracrRNA” or “tracr sequence” means trans-activating CRISPR RNA. tracrRNA interacts with the CRISPR (cr) RNA to form a guide nucleic acid (e.g., guide RNA or gRNA) that may hybridize to a target nucleic acid and thereby directs an associated nuclease to the target nucleic acid.

As used herein, the term “RuvC_III domain” refers to a third discontinuous segment of a RuvC endonuclease domain (the RuvC nuclease domain being comprised of three discontiguous segments, RuvC_I, RuvC_II, and RuvC_III). A RuvC domain or segments thereof can generally be identified by alignment to documented domain sequences, structural alignment to proteins with annotated domains, or by comparison to Hidden Markov Models (HMMs) built based on documented domain sequences (e.g., Pfam HMM PF18541 for RuvC_III).

As used herein, the term “HNH domain” refers to an endonuclease domain having characteristic histidine and asparagine residues. An HNH domain can generally be identified by alignment to documented domain sequences, structural alignment to proteins with annotated domains, or by comparison to Hidden Markov Models (HMMs) built based on documented domain sequences (e.g., Pfam HMM PF01844 for domain HNH).

As used herein, the term “transposon” refers to mobile elements that move in and out of genomes carrying “cargo DNA” with them. These transposons can differ on the type of nucleic acid to transpose, the type of repeat at the ends of the transposon, the type of cargo to be carried, or by the mode of transposition (i.e., self-repair or host-repair).

As used herein, the term “transposase” or “transposases” refers to an enzyme that binds to the end of a transposon and catalyzes its movement to another part of the genome. Types of movement include a cut and paste mechanism and a replicative transposition mechanism.

As used herein, the term “Tn7” or “Tn7-like transposase” refers to a family of transposases comprising three main components: a heteromeric transposase (TnsA and/or TnsB) alongside a regulator protein (TnsC). In addition to the TnsABC transposition proteins, Tn7 elements can encode dedicated target site-selection proteins, TnsD and TnsE. In conjunction with TnsABC, the sequence-specific DNA-binding protein TnsD directs transposition into a conserved site referred to as the “Tn7 attachment site,” attTn7. TnsD is a member of a large family of proteins that also includes TniQ. TniQ has been shown to target transposition into resolution sites of plasmids.

As used herein, the terms “gene editing” and “genome editing” can be used interchangeably. Gene editing or genome editing means to change the nucleic acid sequence of a gene or a genome. Genome editing can include, for example, insertions, deletions, and mutations. Genome editing can be performed by a gene editing system, for example a nuclease, a reverse transcriptase, a recombinase, or a base editor.

As used herein, the term “recombinase” refers to a site-specific enzyme that mediates the recombination of DNA between recombinase recognition sequences, which results in the excision, integration, inversion, or exchange (e.g., translocation) of DNA fragments between the recombinase recognition sequences.

As used herein, the term “recombine,” or “recombination,” in the context of a nucleic acid modification (e.g., a genomic modification), refers to the process by which two or more nucleic acid molecules, or two or more regions of a single nucleic acid molecule, are modified by the action of a recombinase protein. Recombination can result in, inter alia, the insertion, inversion, excision, or translocation of a nucleic acid sequence, e.g., in or between one or more nucleic acid molecules.

As used herein, the term “complex” refers to a joining of at least two components. The two components may each retain the properties/activities they had prior to forming the complex or gain properties as a result of forming the complex. The joining includes, but is not limited to, covalent bonding, non-covalent bonding (i.e., hydrogen bonding, ionic interactions, Van der Waals interactions, and hydrophobic bond), use of a linker, fusion, or any other suitable method. Contemplated components of the complex include polynucleotides, polypeptides, or combinations thereof. For example, a complex comprises an endonuclease and a guide polynucleotide.

The term “sequence identity” or “percent identity” in the context of two or more nucleic acids or polypeptide sequences, refers to two (e.g., in a pairwise alignment) or more (e.g., in a multiple sequence alignment) sequences that are the same or have a specified percentage of amino acid residues or nucleotides that are the same, when compared and aligned for maximum correspondence over a local or global comparison window, as measured using a sequence comparison algorithm. Suitable sequence comparison algorithms for polypeptide sequences include, e.g., BLASTP using parameters of a wordlength (W) of 3, an expectation (E) of 10, and the BLOSUM62 scoring matrix setting gap costs at existence of 11, extension of 1, and using a conditional compositional score matrix adjustment for polypeptide sequences longer than 30 residues; BLASTP using parameters of a wordlength (W) of 2, an expectation (E) of 1000000, and the PAM30 scoring matrix setting gap costs at 9 to open gaps and 1 to extend gaps for sequences of less than 30 residues (these are the default parameters for BLASTP in the BLAST suite available at https://blast.ncbi.nlm.nih.gov); CLUSTALW with the Smith-Waterman homology search algorithm parameters with a match of 2, a mismatch of −1, and a gap of −1; MUSCLE with default parameters; MAFFT with parameters of a retree of 2 and max iterations of 1000; Novafold with default parameters; HMMER hmmalign with default parameters.

The term “optimally aligned” in the context of two or more nucleic acids or polypeptide sequences, refers to two (e.g., in a pairwise alignment) or more (e.g., in a multiple sequence alignment) sequences that have been aligned to maximal correspondence of amino acids residues or nucleotides, for example, as determined by the alignment producing a highest or “optimized” percent identity score.

Included in the current disclosure are variants of any of the enzymes described herein with one or more conservative amino acid substitutions. Such conservative substitutions can be made in the amino acid sequence of a polypeptide without disrupting the three-dimensional structure or function of the polypeptide. Conservative substitutions can be accomplished by substituting amino acids with similar hydrophobicity, polarity, and R chain length for one another. Additionally, or alternatively, by comparing aligned sequences of homologous proteins from different species, conservative substitutions can be identified by locating amino acid residues that have been mutated between species (e.g., non-conserved residues) without altering the basic functions of the encoded proteins. Such conservatively substituted variants include variants with at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of the reverse transcriptases protein sequences described herein (e.g., MG140, MG148, MG153, or MG160), family reverse transcriptases or retrotransposases described herein, or any other family reverse transcriptases or retrotransposases described herein). In some embodiments, such conservatively substituted variants are functional variants. Such functional variants can encompass sequences with substitutions such that the activity of one or more critical active site residues are not disrupted.

Also included in the current disclosure are variants of any of the enzymes described herein with substitution of one or more catalytic residues to decrease or eliminate activity of the enzyme (e.g. decreased-activity variants). In some embodiments, a decreased activity variant as a protein described herein comprises a disrupting substitution of at least one, at least two, or all three catalytic residues (for example a programmable nuclease MG3 family nickase with a D13A mutation, a H586A mutation, or a N609A mutation).

Conservative substitution tables providing functionally similar amino acids are available from a variety of references (see, for e.g., Creighton, Proteins: Structures and Molecular Properties (W H Freeman & Co.; 2nd edition (December 1993)). The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)

### Gene Editing Systems

Described herein are gene editing systems, comprising: a) a nickase; b) a guide nucleic acid (e.g., pegRNA or other guide RNA) configured to form a complex with the nickase and to hybridize to a target nucleic acid sequence; and c) a reverse transcriptase having at least about 80% sequence identity to any one of SEQ ID NOs: 88-116 and 138-139 and configured to form a complex with the nickase. Further described herein are gene editing systems, comprising: a) a nuclease; b) a guide nucleic acid (e.g., pegRNA or other guide RNA) configured to form a complex with the nuclease and to hybridize to a target nucleic acid sequence; and c) a reverse transcriptase having at least about 80% sequence identity to any one of SEQ ID NOs: 88-116 and 138-139 and configured to form a complex with the nuclease. Further described herein are gene editing systems, comprising: a) a nickase; b) a guide nucleic acid (e.g., pegRNA) configured to form a complex with the nickase and to hybridize to a target nucleic acid sequence; and c) a reverse transcriptase configured to form a complex with the nickase, the reverse transcriptase having a X₁X₂DD motif, wherein X₁ is F or Y, and wherein when X₁ is Y, X₂ is A, R, N, D, C, E, Q, G, H, I, L, K, M, F, P, S, T, V, W, or Y. Further described herein are gene editing systems, comprising: a) a nuclease; b) a guide nucleic acid (e.g., pegRNA) configured to form a complex with the nuclease and to hybridize to a target nucleic acid sequence; and c) a reverse transcriptase configured to form a complex with the nuclease, the reverse transcriptase having a X₁X₂DD motif, wherein X₁ is F or Y, and wherein when X₁ is Y, X₂ is A, R, N, D, C, E, Q, G, H, I, L, K, M, F, P, S, T, V, W, or Y.

Gene editing systems as described herein, in some embodiments, comprising a nickase, a nuclease, a reverse transcriptase, or combinations thereof are capable of introduction of site-directed insertions, deletions, and mutations. In some embodiments, the nickase, the nuclease, the reverse transcriptase, or combinations thereof are capable of integration of polynucleotides of large sizes. In some embodiments, the integrated polynucleotide comprises a size of at least about 1 kilobase (kb), 2 kb, 3 kb, 4 kb, 5 kb, 6 kb, 7 kb, 8 kb, 9 kb, 10 kb, or more than 10 kb.

### Reverse Transcriptases

Reverse transcription is the translation of an RNA template into a complementary DNA. Reverse transcription is performed by enzymes termed reverse transcriptases (RT) that are enzymes with RNA-dependent DNA polymerase activity that create the complementary DNA (cDNA) strand from a RNA template. Some of the RT enzymes also have DNA-dependent DNA polymerase activity to create a double-stranded dsDNA. Reverse transcriptases can be of viral origin (for example HIV, hepatitis B, Moloney murine leukemia virus (MMLV), or avian myeloblastosis virus (AMV)) or bacterial origin (for example group II introns, retrons/retron-like RTs, diversity-generating retroelements (DGRs), Abi-like RTs, CRISPR-associated RTs, and group 1-like RTs (G2L)). Reverse transcriptases of eukaryotic origin comprise the telomerase reverse transcriptase that maintains the telomeres of eukaryotic chromosomes. Reverse transcription allows the introduction of site-directed insertions, deletions, and mutations into the cDNA by encoding them in the RNA template.

In some embodiments, the reverse transcriptase is a viral, prokaryotic, or eukaryotic reverse transcriptase. In some embodiments, the reverse transcriptase comprises a sequence of SEQ ID NOs: 88-116 and 138-139, a variant thereof, or a functional fragment thereof. In some embodiments, the reverse transcriptase comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to any one of SEQ ID NOs: 88-116 and 138-139, a variant thereof, or a functional fragment thereof. In some embodiments, the reverse transcriptase comprises a sequence having at least about 70% identity to any one of SEQ ID NOs: 88-116 and 138-139. In some embodiments, the reverse transcriptase comprises a sequence having at least about 75% identity to any one of SEQ ID NOs: 88-116 and 138-139. In some embodiments, the reverse transcriptase comprises a sequence having at least about 80% identity to any one of SEQ ID NOs: 88-116 and 138-139. In some embodiments, the reverse transcriptase comprises a sequence having at least about 85% identity to any one of SEQ ID NOs: 88-116 and 138-139. In some embodiments, the reverse transcriptase comprises a sequence having at least about 90% identity to any one of SEQ ID NOs: 88-116 and 138-139. In some embodiments, the reverse transcriptase comprises a sequence having at least about 95% identity to any one of SEQ ID NOs: 88-116 and 138-139. In some embodiments, the reverse transcriptase comprises a sequence having at least about 96% identity to any one of SEQ ID NOs: 88-116 and 138-139. In some embodiments, the reverse transcriptase comprises a sequence having at least about 97% identity to any one of SEQ ID NOs: 88-116 and 138-139. In some embodiments, the reverse transcriptase comprises a sequence having at least about 98% identity to any one of SEQ ID NOs: 88-116 and 138-139. In some embodiments, the reverse transcriptase comprises a sequence having at least about 99% identity to any one of SEQ ID NOs: 88-116 and 138-139. In some embodiments, the reverse transcriptase comprises a sequence having 100% identity to any one of SEQ ID NOs: 88-116 and 138-139.

In some embodiments, the reverse transcriptase is a MG140, MG148, MG153 or MG160 family reverse transcriptase. In some embodiments, the reverse transcriptase comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to any one of the MG140, MG148, MG153, or MG160 family reverse transcriptase or retrotransposase. In some embodiments, the reverse transcriptase comprises a sequence with at least 80% sequence identity to any one of MG140, MG148, MG153, or MG160 family reverse transcriptase or retrotransposase or a variant thereof.

In some embodiments, the reverse transcriptase is encoded by a nucleic acid sequence having at least 80% sequence identity with the nucleic acid sequence of any one of SEQ ID NOs: 1-5. In some embodiments, the reverse transcriptase is encoded by a nucleic acid sequence having at least 85% sequence identity with the nucleic acid sequence of any one of SEQ ID NOs: 1-5. In some embodiments, the reverse transcriptase is encoded by a nucleic acid sequence having at least 90% sequence identity with the nucleic acid sequence of any one of SEQ ID NOs: 1-5. In some embodiments, the reverse transcriptase is encoded by a nucleic acid sequence having at least 95% sequence identity with the nucleic acid sequence of any one of SEQ ID NOs: 1-5. In some embodiments, the reverse transcriptase is encoded by a nucleic acid sequence having at least 96% sequence identity with the nucleic acid sequence of any one of SEQ ID NOs: 1-5. In some embodiments, the reverse transcriptase is encoded by a nucleic acid sequence having at least 97% sequence identity with the nucleic acid sequence of any one of SEQ ID NOs: 1-5. In some embodiments, the reverse transcriptase is encoded by a nucleic acid sequence having at least 98% sequence identity with the nucleic acid sequence of any one of SEQ ID NOs: 1-5. In some embodiments, the reverse transcriptase is encoded by a nucleic acid sequence having at least 99% sequence identity with the nucleic acid sequence of any one of SEQ ID NOs: 1-5. In some embodiments, the reverse transcriptase is encoded by a nucleic acid sequence of any one of SEQ ID NOs: 1-5.

Reverse transcriptases typically have an active site core tetrad motif of the amino acid sequence XXDD. In some embodiments, the reverse transcriptase has an active site tetrad motif of X₁X₂DD wherein X₁ is F or Y, and wherein when X₁ is Y, X₂ is A, R, N, D, C, E, Q, G, H, I, L, K, M, F, P, S, T, V, W, or Y. In some embodiments, X₂ is A or I. In some embodiments, the X₁X₂DD motif is YADD (SEQ ID NO: 140) or YIDD (SEQ ID NO: 141). In some embodiments, the X₁X₂DD motif is FADD (SEQ ID NO: 142), FVDD (SEQ ID NO: 143), FIDD (SEQ ID NO: 144), or FLDD (SEQ ID NO: 145). In some embodiments, the reverse transcriptase is isolated. In some embodiments, the reverse transcriptase is a MG140, MG148, MG153, and MG160 family reverse transcriptase or retrotransposase and the X₁X₂DD motif is YADD (SEQ ID NO: 140) or YIDD (SEQ ID NO: 141). In some embodiments, the reverse transcriptase is isolated. In some embodiments, the reverse transcriptase is a MG140, MG148, MG153, or MG160 family reverse transcriptase or retrotransposase and the X₁X₂DD motif is FADD (SEQ ID NO: 142), FVDD (SEQ ID NO: 143), FIDD (SEQ ID NO: 144), or FLDD (SEQ ID NO: 145).

In some embodiments, the reverse transcriptase is smaller than 300 amino acids. In some embodiments, the reverse transcriptase is smaller than 250 amino acids. In some embodiments, the reverse transcriptase comprises at least about 50, 75, 100, 125, 150, 175, 200, 225, 250, 275, 300, or more than 300 amino acids. In some embodiments, the reverse transcriptase comprises a range of about 50 to about 300, about 75 to about 300, about 100 to about 300, about 125 to about 300, about 150 to about 300, about 175 to about 300, about 200 to about 300, about 225 to about 300, about 250 to about 300, about 275 to about 300, about 100 to about 300, about 125 to about 300, about 150 to about 300, about 175 to about 300, about 200 to about 300, about 225 to about 300, about 250 to about 300, or about 275 to about 300 amino acids.

In some embodiments, the reverse transcriptase comprises a processivity of at least about 2-fold more than Moloney Murine Leukemia Virus (MMLV) reverse transcriptase. In some embodiments, the reverse transcriptase comprises a processivity of at least about 2-fold less than Moloney Murine Leukemia Virus (MMLV) reverse transcriptase. In some embodiments, the reverse transcriptase comprises an error rate of less than about 2.5%, 2.0%, 1.5%, 1%, 0.5%, 0.25%, 0.10%, or 0.05%. In some embodiments, the reverse transcriptase comprises an error rate of less than about 2.5%, 2.0%, 1.5%, 1%, 0.5%, 0.25%, 0.10%, or 0.05% as compared to Moloney Murine Leukemia Virus (MMLV) reverse transcriptase. Methods to measure reverse transcriptase processivity are known in the art or are described herein, for example in Example 2.

In some embodiments, the reverse transcriptase is targetable. Targetable reverse transcriptases are engineered ribonucleoprotein complexes that act as tools for genome editing in cells and organisms. In some embodiments, targetable reverse transcriptases are created by fusing a reverse transcriptase and a site-directed CRISPR nuclease variant that nicks the non-targeting strand of dsDNA, such that a guide RNA or pegRNA comprising a primer binding site (PBS) sequence can find and hybridize with its complementary target sequence to prime the reverse transcriptase reaction using a reverse transcriptase template (RTT) as the template. Two DNA flaps are produced, one containing the desired change encoded in the RTT, and the other with the original sequence; post-equilibration, the change is incorporated into the genomic DNA when the DNA flap with the desired edit is repaired by the cellular host repair machinery.

In some embodiments, the gene editing system comprises a reverse transcriptase described herein and a nickase. In some embodiments, the gene editing system comprises a reverse transcriptase described herein and a nuclease. In some embodiments, the gene editing system comprises a reverse transcriptase described herein and a modified nuclease. In some embodiments, the gene editing system is programmable. In some embodiments, the modified nuclease is a site-directed nickase.

In some embodiments, the reverse transcriptase and the nuclease or nickase are linked or tethered. In some embodiments, the gene editing system comprises a fusion protein of a reverse transcriptase and a nuclease or nickase. In some embodiments, the gene editing system comprises a fusion protein comprising a nickase linked to a reverse transcriptase using a linker, wherein the reverse transcriptase comprises at least about 80% sequence identity to any one of SEQ ID NOs: 88-116 and 138-139. In some embodiments, the gene editing system comprises a fusion protein comprising a nuclease linked to a reverse transcriptase using a linker, wherein the reverse transcriptase comprises at least about 80% sequence identity to any one of SEQ ID NOs: 88-116 and 138-139. In some embodiments, the gene editing system comprises a fusion protein comprising a catalytically dead nuclease linked to a reverse transcriptase using a linker, wherein the reverse transcriptase comprises at least about 80% sequence identity to any one of SEQ ID NOs: 88-116 and 138-139.

In some embodiments, the reverse transcriptase and the nuclease or nickase is linked or fused using a linker. In some embodiments, the linker comprises at least 10, 20, or 30 amino acids. In some embodiments, the linker comprises about 30-35 amino acids. In some embodiments, the linker comprises about 30 amino acids.

In some embodiments, the linker comprises at least 80% sequence identity to SEQ ID NO: 33. In some embodiments, the linker comprises a sequence having at least about 85% identity to SEQ ID NO: 33. In some embodiments, the linker comprises a sequence having at least about 90% identity to SEQ ID NO: 33. In some embodiments, the linker comprises a sequence having at least about 91% identity to SEQ ID NO: 33. In some embodiments, the linker comprises a sequence having at least about 92% identity to SEQ ID NO: 33. In some embodiments, the linker comprises a sequence having at least about 93% identity to SEQ ID NO: 33. In some embodiments, the linker comprises a sequence having at least about 94% identity to SEQ ID NO: 33. In some embodiments, the linker comprises a sequence having at least about 95% identity to SEQ ID NO: 33. In some embodiments, the linker comprises a sequence having at least about 96% identity to SEQ ID NO: 33. In some embodiments, the linker comprises a sequence having at least about 97% identity to SEQ ID NO: 33. In some embodiments, the linker comprises a sequence having at least about 98% identity to SEQ ID NO: 33. In some embodiments, the linker comprises a sequence having at least about 99% identity to SEQ ID NO: 33. In some embodiments, the linker comprises a sequence having 100% identity to SEQ ID NO: 33.

Suitable linkers are known in the art and comprise, for example, any one of SEQ ID NOs: 82-87. In some embodiments, the linker comprises at least 80% sequence identity to any one of SEQ ID NOs: 82-87. In some embodiments, linkers joining any of the enzymes or domains described herein comprise one or multiple copies of a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SGGSSGGSSGSETPGTSESATPESSGGSSGGSSAC (SEQ ID NO: 82), KLGGGAPAVGGGPK(SEQ ID NO: 83), (GGGGS)3(SEQ ID NO: 84), (GGGGS)2EAAAK(GGGGS)2 (SEQ ID NO: 85), (GGGGS)2(EAAAK)2(GGGGS)2 (SEQ ID NO: 86), or SGSETPGTSESATPES (SEQ ID NO: 87), or any other linker sequence described herein. In some embodiments, the linker comprises a sequence having at least about 85% identity to any one of SEQ ID NOs: 82-87. In some embodiments, the linker comprises a sequence having at least about 90% identity to any one of SEQ ID NOs: 82-87. In some embodiments, the linker comprises a sequence having at least about 91% identity to any one of SEQ ID NOs: 82-87. In some embodiments, the linker comprises a sequence having at least about 92% identity to any one of SEQ ID NOs: 82-87. In some embodiments, the linker comprises a sequence having at least about 93% identity to any one of SEQ ID NOs: 82-87. In some embodiments, the linker comprises a sequence having at least about 94% identity to any one of SEQ ID NOs: 82-87. In some embodiments, the linker comprises a sequence having at least about 95% identity to any one of SEQ ID NOs: 82-87. In some embodiments, the linker comprises a sequence having at least about 96% identity to any one of SEQ ID NOs: 82-87. In some embodiments, the linker comprises a sequence having at least about 97% identity to any one of SEQ ID NOs: 82-87. In some embodiments, the linker comprises a sequence having at least about 98% identity to any one of SEQ ID NOs: 82-87. In some embodiments, the linker comprises a sequence having at least about 99% identity to any one of SEQ ID NOs: 82-87. In some embodiments, the linker comprises a sequence having 100% identity to any one of SEQ ID NOs: 82-87.

In some embodiments, the nickase or nuclease and the reverse transcriptase are not linked.

In some embodiments, the reverse transcriptase, nuclease, nickase, or fusion protein described herein comprises one or more nuclear localization sequences (NLSs) proximal to an N- or C-terminus of the reverse transcriptase, nuclease, nickase, or fusion protein.

In some embodiments, the NLS comprises any of the sequences in Table 1 below, or a combination thereof.

TABLE 1
Example NLS Sequences

| | NLS amino | SEQ |
|---|---|---|
| | acid | ID |
| Source | sequence | NO: |
| SV40 | PKKKRKV | 120 |
| nucleoplasmin | KRPAATKKAGQAK | 121 |
| bipartite NLS | KKK | |
| c-myc NLS | PAAKRVKLD | 122 |
| c-myc NLS | RQRRNELKRSP | 123 |
| hRNPA1 M9 NLS | NQSSNFGPMKGGNFGG | 124 |
| | RSSGPYGGGGQYFA | |
| | KPRNQGGY | |
| Importin-alpha | RMRIZFKNKGKDTAEL | 125 |
| IBB domain | RRRRVEVSVELRKAK | |
| | KDEQILKRRNV | |
| Myoma T protein | VSRKRPRP | 126 |
| Myoma T protein | PPKKARED | 127 |
| p53 | PQPKKKPL | 128 |
| mouse c-abl IV | SALIKKKKKMAP | 129 |
| influenza virus NS1 | DRLRR | 130 |
| influenza virus NS1 | PKQKKRK | 131 |
| Hepatitis virus | RKLKKKIKKL | 132 |
| delta antigen | | |
| mouse Mx1 protein | REKKKFLKRR | 133 |
| human poly(ADP-ribose) | KRKGDEVDGVDEV | 134 |
| polymerase | AKKKSKK | |
| steroid hormone receptor | RKCLQAGMNLEAR | 135 |
| (human) glucocorticoid | KTKK | |

In some embodiments, the reverse transcriptase comprises a tag. In some embodiments, the nuclease comprises a tag. In some embodiments, the nickase comprises a tag. In some embodiments, the fusion protein comprises a tag. In some embodiments, the tag is an affinity tag. Exemplary affinity tags include, but are not limited to, His-tag, a Flag tag, a Myc-tag, an MBP-tag, and a GST-tag.

In some embodiments, the reverse transcriptase comprises a protease cleavage site. In some embodiments, the nuclease comprises a protease cleavage site. In some embodiments, the nickase comprises a protease cleavage site. In some embodiments, the fusion protein comprises a protease cleavage site. Exemplary protease cleavage sites include, but are not limited to, a TEV site, a C3 site, a Factor Xa site, and an Enterokinase site.

In some embodiments, the gene editing system comprises a) a nickase; b) a guide nucleic acid (e.g., pegRNA or other guide RNA); and c) a reverse transcriptase having at least about 80% sequence identity to any one of SEQ ID NOs: 88-116 and 138-139.

In some embodiments, the gene editing system comprises a) a nuclease; b) a guide nucleic acid (e.g., pegRNA or other guide RNA); and c) a reverse transcriptase having at least about 80% sequence identity to any one of SEQ ID NOs: 88-116 and 138-139.

In some embodiments, the gene editing system comprises a) a nickase b) a guide nucleic acid (e.g., pegRNA); and c) a reverse transcriptase having a X₁X₂DD motif, wherein X₁ is F or Y, and wherein when X₁ is Y, X₂ is A, R, N, D, C, E, Q, G, H, I, L, K, M, F, P, S, T, V, W, or Y.

In some embodiments, the gene editing system comprises a) a nuclease; b) a guide nucleic acid (e.g., pegRNA); and c) a reverse transcriptase having a X₁X₂DD motif, wherein X₁ is F or Y, and wherein when X₁ is Y, X₂ is A, R, N, D, C, E, Q, G, H, I, L, K, M, F, P, S, T, V, W, or Y. In some embodiments, X₂ is A or I. In some embodiments, the X₁X₂DD motif is YADD (SEQ ID NO: 140) or YIDD (SEQ ID NO: 141). In some embodiments, the X₁X₂DD motif is FADD (SEQ ID NO: 142), FVDD (SEQ ID NO: 143), FIDD (SEQ ID NO: 144), or FLDD (SEQ ID NO: 145). In some embodiments, the reverse transcriptase has at least about 80% sequence identity to any one of SEQ ID NOs: 88-116 and 138-139.

In some embodiments, the nuclease is configured to cleave one strand of a double-stranded target deoxyribonucleic acid (nickase). In some embodiment, the nickase or nuclease is a CRISPR nuclease described herein. In some embodiments, the nickase or nuclease is encoded by a nucleic acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 34 or a variant thereof. In some embodiments, the nickase or nuclease is encoded by a nucleic acid sequence having at least about 70% identity to SEQ ID NO: 34. In some embodiments, the nickase or nuclease is encoded by a nucleic acid sequence having at least about 75% identity to SEQ ID NO: 34. In some embodiments, the nickase or nuclease is encoded by a nucleic acid sequence having at least about 80% identity to SEQ ID NO: 34. In some embodiments, the nickase or nuclease is encoded by a nucleic acid sequence having at least about 85% identity to SEQ ID NO: 34. In some embodiments, the nickase or nuclease is encoded by a nucleic acid sequence having at least about 90% identity to SEQ ID NO: 34. In some embodiments, the nickase or nuclease is encoded by a nucleic acid sequence having at least about 95% identity to SEQ ID NO: 34. In some embodiments, the nickase or nuclease is encoded by a nucleic acid sequence having at least about 96% identity to SEQ ID NO: 34. In some embodiments, the nickase or nuclease is encoded by a nucleic acid sequence having at least about 97% identity to SEQ ID NO: 34. In some embodiments, the nickase or nuclease is encoded by a nucleic acid sequence having at least about 98% identity to SEQ ID NO: 34. In some embodiments, the nickase or nuclease is encoded by a nucleic acid sequence having at least about 99% identity to SEQ ID NO: 34. In some embodiments, the nickase or nuclease is encoded by a nucleic acid sequence having 100% identity to SEQ ID NO: 34.

In some embodiments, the system further comprises a source of Mg=+.

In some embodiments, the nuclease is a modified endonuclease. In some embodiments, the modified endonuclease is a Type II CRISPR endonuclease or a Type V CRISPR endonuclease. In some embodiments, the Type II or Type V CRISPR endonuclease comprises double-stranded cutting activity, nickase activity, or can be catalytically dead. In some embodiments, the CRISPR nuclease has a modification in the HNH domain or in the RuvC domain.

In some embodiments, the modified endonuclease comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NOs: 117-119 or a variant thereof. In some embodiments, the modified endonuclease comprises at least about 80% sequence identity to any one of SEQ ID NOs: 117-119. In some embodiments, the modified endonuclease comprises a sequence having at least about 70% identity to any one of SEQ ID NOs: 117-119. In some embodiments, the modified endonuclease comprises a sequence having at least about 75% identity to any one of SEQ ID NOs: 117-119. In some embodiments, the modified endonuclease comprises a sequence having at least about 80% identity to any one of SEQ ID NOs: 117-119. In some embodiments, the modified endonuclease comprises a sequence having at least about 85% identity to any one of SEQ ID NOs: 117-119. In some embodiments, the modified endonuclease comprises a sequence having at least about 90% identity to any one of SEQ ID NOs: 117-119. In some embodiments, the modified endonuclease comprises a sequence having at least about 95% identity to any one of SEQ ID NOs: 117-119. In some embodiments, the modified endonuclease comprises a sequence having at least about 96% identity to any one of SEQ ID NOs: 117-119. In some embodiments, the modified endonuclease comprises a sequence having at least about 97% identity to any one of SEQ ID NOs: 117-119. In some embodiments, the modified endonuclease comprises a sequence having at least about 98% identity to any one of SEQ ID NOs: 117-119. In some embodiments, the modified endonuclease comprises a sequence having at least about 99% identity to any one of SEQ ID NOs: 117-119. In some embodiments, the modified endonuclease comprises a sequence having 100% identity to any one of SEQ ID NOs: 117-119.

In some embodiments, the modified endonuclease is selected from the group consisting of: spCas9 (H840A), spCas9 (D10A), nMG3-6 (D13A), nMG3-6 (H586A), nMG3-6 (N609A), Cas12a, and MG29-1.

In some embodiments, the gene editing system comprises a nucleic acid template. The nucleic acid template can be an RNA or a DNA. The nucleic acid template can be 10, 20, 30, 40, 50, 60, 70, 80, 90, 100, 110, 120, 130, 140, 150, 160, 170, 180, 190, or 200 bases long. The nucleic acid template can be 100, 200, 300, 400, 500, 600, 700, 800, 900, or 1000 bases long. In some embodiments, the nucleic acid template has a homology region that is homologous to a site in the genome. In some embodiments, the homology region is 10, 20, 30, 40, 50, 60, 70, 80, 90, 100, 110, 120, 130, 140, 150, 160, 170, 180, 190, or 200 bases long.

In some embodiments, the gene editing system further comprises a transposase, an integrase, or a homing endonuclease. In some embodiments, the transposase is transposase (Tnp) Tn5, Sleeping Beauty transposase, or a Tn7 transposon. In some embodiments, the gene editing system comprises an enzyme with transposase activity. Additional enzymes with transposase activity include, but are not limited to, retrons and IS200/IS605 transposons

In some embodiments, the gene editing system further comprises a retrotransposon of the disclosure. In some embodiments, the retrotransposon is a MG140 family retrotransposon. In some embodiments, the retrotransposon comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NOs: 88-103, or a variant thereof.

### CRISPR Nucleases

Described herein, in some embodiments, are nickases or endonucleases, wherein the nickase or endonuclease is a CRISPR nuclease. In some embodiments, the CRISPR nuclease is a modified nuclease.

CRISPR systems are RNA-directed nuclease complexes that have been described to function as an adaptive immune system in microbes. In their natural context, CRISPR systems occur in CRISPR (clustered regularly interspaced short palindromic repeats) operons or loci, which generally comprise two parts: (i) an array of short repetitive sequences (30-40 bp) separated by equally short spacer sequences, which encode the RNA-based targeting element; and (ii) ORFs encoding the nuclease polypeptide directed by the RNA-based targeting element alongside accessory proteins/enzymes. Efficient nuclease targeting of a particular target nucleic acid sequence generally requires both (i) complementary hybridization between the first 6-8 nucleic acids of the target (the target seed) and the crRNA guide; and (ii) the presence of a protospacer-adjacent motif (PAM) sequence within a defined vicinity of the target seed (the PAM usually being a sequence not commonly represented within the host genome). Depending on the exact function and organization of the system, CRISPR systems are commonly organized into 2 classes, 5 types, and 16 subtypes based on shared functional characteristics and evolutionary similarity.

Class I CRISPR systems have large, multi-subunit effector complexes, and comprise Types I, III, and IV. Class II CRISPR systems generally have single-polypeptide multidomain nuclease effectors, and comprise Types II, V, and VI.

Type II CRISPR systems are considered the simplest in terms of components. In Type II CRISPR systems, the processing of the CRISPR array into mature crRNAs does not require the presence of a special endonuclease subunit, but rather a small trans-encoded crRNA (tracrRNA) with a region complementary to the array repeat sequence; the tracrRNA interacts with both its corresponding effector nuclease (e.g. Cas9) and the repeat sequence to form a precursor dsRNA structure, which is cleaved by endogenous RNAse III to generate a mature effector enzyme loaded with both tracrRNA and crRNA. Type II nucleases are known as DNA nucleases. Type II effectors generally exhibit a structure consisting of a RuvC-like endonuclease domain that adopts the RNase H fold with an unrelated HNH nuclease domain inserted within the folds of the RuvC-like nuclease domain. The RuvC-like domain is responsible for the cleavage of the target (e.g., crRNA complementary) DNA strand, while the HNH domain is responsible for cleavage of the displaced DNA strand. Exemplary CRISPR Cas9 proteins include, but are not limited to, Cas9 from Streptococcus pyogenes (UniProtKB—Q99ZW2 (CAS9 STRP1)), Streptococcus thermophilus (UniProtKB—G3ECR1 (CAS9 STRTR)), Staphylococcus aureus (UniProtKB—J7RUA5 (CAS9 STAAU), Campylobacter jejuni (UniProtKB—Q0P897 (CAS9 CAMJE)), Campylobacter lari (UniProtKB—A0A0A8HTA3 (A0A0A8HTA3 CAMLA), and Helicobacter canadensis (UniProtKB—C5ZYI3 (C5ZYI3 9HELI)), Francisella tularensis subsp. Novicida (UniProtKB—A0Q5Y3 (CAS9_FRATN). Additional Type II nucleases are described in International Patent Application Publication WO 2021/226363, WO 2022/159758, and WO 2022/056324.

Type V CRISPR systems are characterized by a nuclease effector (e.g. Cas12) structure similar to that of Type II effectors, comprising a RuvC-like domain. Similar to Type II, most (but not all) Type V CRISPR systems use a tracrRNA to process pre-crRNAs into mature crRNAs; however, unlike Type II systems which requires RNAse Ill to cleave the pre-crRNA into multiple crRNAs, Type V systems are capable of using the effector nuclease itself to cleave pre-crRNAs. Like Type-II CRISPR systems, Type V CRISPR systems are known as DNA nucleases. Unlike Type II CRISPR systems, some Type V enzymes (e.g., Cas12a) appear to have a robust single-stranded nonspecific deoxyribonuclease activity that is activated by the first crRNA-directed cleavage of a double-stranded target sequence.

In some embodiments, the nuclease or nickase is a CRISPR nuclease. In some embodiments, the CRISPR nuclease is a Class 2 Type II SpCas9 or a Class 2 Type V-A Cas12a (previously Cpf1). In some embodiments, the Type V-A nuclease has a guide RNA of 42-44 nucleotides compared with approximately 100 nt for SpCas9. In some embodiments, the Type V-A nuclease results in staggered cut sites. In some embodiments, the Type V-A nuclease results in staggered cut sites to facilitate directed repair pathways, such as microhomology-dependent targeted integration (MITI).

The most commonly used Type V-A enzymes require a 5′ protospacer adjacent motif (PAM) next to the chosen target site: 5′-TTTV-3′ for Lachnospiraceae bacterium ND2006 LbCas12a and Acidaminococcus sp. AsCas12a; and 5′-TTV-3′ for Francisella novicida FnCas12a. In some embodiments the PAM sequence is YTV, YYN, or TTN. Additional Type II nucleases are described in International Patent Application Publication WO 2021/226363.

In some embodiments, the nickase is a modified nuclease. In some embodiments, the modified endonuclease is a Type H CRISPR endonuclease. In some embodiments, the modified endonuclease is a Type II CRISPR endonuclease or a Type V endonuclease. In some embodiments, the Type II CRISPR endonuclease or the Type V endonuclease has nickase activity.

In some embodiments, the modified endonuclease is selected from the group consisting of: spCas9 (H840A), spCas9 (D10A), nMG3-6 (D13A), nMG3-6 (H586A), nMG3-6 (N609A), Cas12a, and MG29-1. In some embodiments, the nuclease comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to any one of SEQ ID NOs: 117-119 or a variant thereof. In some embodiments, the modified endonuclease comprises a sequence having at least about 70% identity to any one of SEQ ID NOs: 117-119. In some embodiments, the modified endonuclease comprises a sequence having at least about 75% identity to any one of SEQ ID NOs: 117-119. In some embodiments, the modified endonuclease comprises a sequence having at least about 80% identity to any one of SEQ ID NOs: 117-119. In some embodiments, the modified endonuclease comprises a sequence having at least about 85% identity to any one of SEQ ID NOs: 117-119. In some embodiments, the modified endonuclease comprises a sequence having at least about 90% identity to any one of SEQ ID NOs: 117-119. In some embodiments, the modified endonuclease comprises a sequence having at least about 95% identity to any one of SEQ ID NOs: 117-119. In some embodiments, the modified endonuclease comprises a sequence having at least about 96% identity to any one of SEQ ID NOs: 117-119. In some embodiments, the modified endonuclease comprises a sequence having at least about 97% identity to any one of SEQ ID NOs: 117-119. In some embodiments, the modified endonuclease comprises a sequence having at least about 98% identity to any one of SEQ ID NOs: 117-119. In some embodiments, the modified endonuclease comprises a sequence having at least about 99% identity to any one of SEQ ID NOs: 117-119. In some embodiments, the modified endonuclease comprises a sequence having 100% identity to any one of SEQ ID NOs: 117-119.

In some embodiments, the nuclease is encoded by a nucleic acid sequence having at least 80% sequence identity with the nucleic acid sequence of SEQ ID NO: 34. In some embodiments, the nuclease is encoded by a nucleic acid sequence having at least 85% sequence identity with the nucleic acid sequence of SEQ ID NO: 34. In some embodiments, the nuclease is encoded by a nucleic acid sequence having at least 90% sequence identity with the nucleic acid sequence of SEQ ID NO: 34. In some embodiments, the nuclease is encoded by a nucleic acid sequence having at least 95% sequence identity with the nucleic acid sequence of SEQ ID NO: 34. In some embodiments, the nuclease is encoded by a nucleic acid sequence having at least 96% sequence identity with the nucleic acid sequence of SEQ ID NO: 34. In some embodiments, the nuclease is encoded by a nucleic acid sequence having at least 97% sequence identity with the nucleic acid sequence of SEQ ID NO: 34. In some embodiments, the nuclease is encoded by a nucleic acid sequence having at least 98% sequence identity with the nucleic acid sequence of SEQ ID NO: 34. In some embodiments, the nuclease is encoded by a nucleic acid sequence having at least 99% sequence identity with the nucleic acid sequence of SEQ ID NO: 34. In some embodiments, the nuclease is encoded by a nucleic acid sequence of SEQ ID NO: 34.

In some embodiments, the RuvC domain lacks nuclease activity. In some embodiments, the HNH domain lack nuclease activity. In some embodiments, the modified nuclease has a modification corresponding to position H840A in S. pyogenes Cas9. In some embodiments, the modified nuclease has a modification corresponding to position D10A in S. pyogenes Cas9. In some embodiments, the modified nuclease has a modification corresponding to position D13A in MG3-6 (SEQ ID NO: 136) termed nMG3-6 (D13A) (SEQ ID NO: 117). In some embodiments, the modified nuclease has a modification corresponding to position H586A in MG3-6 (SEQ ID NO: 136) termed nMG3-6 (H586A) (SEQ ID NO: 118). In some embodiments, the modified nuclease has a modification corresponding to position N609A in MG3-6 (SEQ ID NO: 136) termed nMG3-6 (N609A) (SEQ ID NO: 119). In some embodiments, the modified nuclease is configured to cleave one strand of a double-stranded target deoxyribonucleic acid. In some embodiments, the ribonucleic acid sequence configured to bind to the endonuclease comprises a tracr sequence.

In some embodiments, the nickase or nuclease comprises one or more nuclear localization sequences (NLSs) proximal to an N- or C-terminus of the nickase or nuclease.

In some embodiments, the NLS comprises any of the sequences in Table 1 above, or a combination thereof.

### Guide Nucleic Acids

In some embodiments, provided herein are guide nucleic acids such as guide RNAs (gRNAs) or prime editing guide RNAs (pegRNAs). In a polynucleotide when referring to a T, a T means U (Uracil) in RNA and T (Thymine) in DNA.

Prime editing enables the installation of virtually any combination of point mutations, small insertions, or small deletions in the genome of living cells. A prime editing guide RNA (pegRNA) directs the prime editor protein to the targeted locus and also encodes the desired edit.

In some embodiments, the guide RNA targets a gene in a cell. In some embodiments, the guide RNA targets a gene in a mammalian cell. In some embodiments, the target gene is TRAC, VEGFA, AAVS1, B2M, CD5, or CD38. Exemplary guide RNAs are shown in SEQ ID NOs: 6-29 and 39-70.

In some embodiments, the guide RNA is encoded by any one of the nucleic acid sequences of SEQ ID NOs: 6-29 and 39-70, a sequence having at least about 80%, 85%, 90%, 95%, 97%, 98%, or 99% sequence identity to any one of the nucleic acid sequences of SEQ ID NOs: 6-29 and 39-70, or a reverse complement thereof. In some embodiments, the guide RNA is encoded by a sequence having at least about 80% sequence identity to any one of the nucleic acid sequences of SEQ ID NOs: 6-29 and 39-70 or a reverse complement thereof. In some embodiments, the guide RNA is encoded by a sequence having at least about 85% sequence identity to any one of the nucleic acid sequences of SEQ ID NOs: 6-29 and 39-70 or a reverse complement thereof. In some embodiments, the guide RNA is encoded by a sequence having at least about 90% sequence identity to any one of the nucleic acid sequences of SEQ ID NOs: 6-29 and 39-70 or a reverse complement thereof. In some embodiments, the guide RNA is encoded by a sequence having at least about 95% sequence identity to any one of the nucleic acid sequences of SEQ ID NOs: 6-29 and 39-70 or a reverse complement thereof. In some embodiments, the guide RNA is encoded by a sequence having at least about 97% sequence identity to any one of the nucleic acid sequences of SEQ ID NOs: 6-29 and 39-70 or a reverse complement thereof. In some embodiments, the guide RNA is encoded by a sequence having at least about 98% sequence identity to any one of the nucleic acid sequences of SEQ ID NOs: 6-29 and 39-70 or a reverse complement thereof. In some embodiments, the guide RNA is encoded by a sequence having at least about 99% sequence identity to any one of the nucleic acid sequences of SEQ ID NOs: 6-29 and 39-70 or a reverse complement thereof. In some embodiments, the guide RNA is encoded by a sequence according to any one of the nucleic acid sequences of SEQ ID NOs: 6-29 and 39-70 or a reverse complement thereof.

In some embodiments, the one or more guide RNAs are encoded by a sequence comprising at least about 80%, 85%, 90%, 95%, 97%, 98%, or 99% sequence identity to any one of the nucleic acid sequences of SEQ ID NOs: 6-29 and 39-70 or a reverse complement thereof. In some embodiments, the one or more guide RNAs are encoded by a sequence comprising at least about 80% sequence identity to any one of the nucleic acid sequences of SEQ ID NOs: 6-29 and 39-70 or a reverse complement thereof. In some embodiments, the one or more guide RNAs are encoded by a sequence comprising at least about 85% sequence identity to any one of the nucleic acid sequences of SEQ ID NOs: 6-29 and 39-70 or a reverse complement thereof. In some embodiments, the one or more guide RNAs are encoded by a sequence comprising at least about 90% sequence identity to any one of the nucleic acid sequences of SEQ ID NOs: 6-29 and 39-70 or a reverse complement thereof.

In some embodiments, the one or more guide RNAs are encoded by a sequence comprising at least about 95% sequence identity to any one of the nucleic acid sequences of SEQ ID NOs: 6-29 and 39-70 or a reverse complement thereof. In some embodiments, the one or more guide RNAs are encoded by a sequence comprising at least about 97% sequence identity to any one of the nucleic acid sequences of SEQ ID NOs: 6-29 and 39-70 or a reverse complement thereof. In some embodiments, the one or more guide RNAs are encoded by a sequence comprising at least about 98% sequence identity to any one of the nucleic acid sequences of SEQ ID NOs: 6-29 and 39-70 or a reverse complement thereof. In some embodiments, the one or more guide RNAs are encoded by a sequence comprising at least about 99% sequence identity to any one of the nucleic acid sequences of SEQ ID NOs: 6-29 and 39-70 or a reverse complement thereof. In some embodiments, the guide RNA is encoded by a sequence according to any one of the nucleic acid sequences of SEQ ID NOs: 6-29 and 39-70 or a reverse complement thereof, or a reverse complement thereof.

In some embodiments, guide RNAs or pegRNAs comprise various structural elements including but not limited to: a spacer sequence which binds to the protospacer sequence (target sequence), crRNA, and an optional tracrRNA. In some embodiments, the genome editing system comprises a CRISPR guide RNA. In some embodiments, the guide RNA comprises a crRNA and a spacer sequence. In some embodiments, the guide RNA additionally comprises a tracrRNA or a modified tracrRNA.

In some embodiments, the compositions and methods provided herein comprise one or more guide RNAs. In some embodiments, the guide RNA comprises a sense sequence. In some embodiments, the guide RNA comprises an anti-sense sequence. In some embodiments, the guide RNA comprises nucleotide sequences other than the region complementary to or substantially complementary to a region of a target sequence. For example, a guide RNA is part or considered part of a crRNA, or is comprised in a crRNA, e.g., a crRNA:tracrRNA chimera.

In some embodiments, the guide RNA (e.g., gRNA) comprises synthetic nucleotides or modified nucleotides. In some embodiments, the guide RNA comprises one or more inter-nucleoside linkers modified from the natural phosphodiester. In some embodiments, all of the inter-nucleoside linkers of the guide RNA, or contiguous nucleotide sequence thereof, are modified. For example, in some embodiments, the inter nucleoside linkage comprises Sulphur (S), such as a phosphorothioate inter-nucleoside linkage.

In some embodiments, the guide RNA (e.g., gRNA) comprises modifications to a ribose sugar or nucleobase. In some embodiments, the guide RNA comprises one or more nucleosides comprising a modified sugar moiety, wherein the modified sugar moiety is a modification of the sugar moiety when compared to the ribose sugar moiety found in deoxyribose nucleic acid (DNA) and RNA. In some embodiments, the modification is within the ribose ring structure. Exemplary modifications include, but are not limited to, replacement with a hexose ring (HNA), a bicyclic ring having a biradical bridge between the C2 and C4 carbons on the ribose ring (e.g. locked nucleic acids (LNA)), or an unlinked ribose ring which typically lacks a bond between the C2 and C3 carbons (e.g. UNA). In some embodiments, the sugar-modified nucleosides comprise bicyclohexose nucleic acids or tricyclic nucleic acids. In some embodiments, the modified nucleosides comprise nucleosides where the sugar moiety is replaced with a non-sugar moiety, for example peptide nucleic acids (PNA) or morpholino nucleic acids.

In some embodiments, the guide RNA comprises one or more modified sugars. In some embodiments, the sugar modifications comprise modifications made by altering the substituent groups on the ribose ring to groups other than hydrogen, or the 2′—OH group naturally found in DNA and RNA nucleosides. In some embodiments, substituents are introduced at the 2′, 3′, 4′, 5′ positions, or combinations thereof. In some embodiments, nucleosides with modified sugar moieties comprise 2′ modified nucleosides, e.g., 2′ substituted nucleosides. A 2′ sugar modified nucleoside, in some embodiments, is a nucleoside that has a substituent other than H or —OH at the 2′ position (2′ substituted nucleoside) or comprises a 2′ linked biradical, and comprises 2′ substituted nucleosides and LNA (2′-4′ biradical bridged) nucleosides. Examples of 2′-substituted modified nucleosides comprise, but are not limited to, 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. In some embodiments, the modification in the ribose group comprises a modification at the 2′ position of the ribose group. In some embodiments, the modification at the 2′ position of the ribose group is selected from the group consisting of 2′-O-methyl, 2′-fluoro, 2′-deoxy, and 2′-O-(2-methoxyethyl).

In some embodiments, the guide RNA comprises one or more modified sugars. In some embodiments, the guide RNA comprises only modified sugars. In certain embodiments, the guide RNA comprises greater than about 10%, 25%, 50%, 75%, or 90% modified sugars. In some embodiments, the modified sugar is a bicyclic sugar. In some embodiments, the modified sugar comprises a 2′-O-methoxyethyl group. In some embodiments, the guide RNA comprises both inter-nucleoside linker modifications and nucleoside modifications.

In some embodiments, the guide RNA comprises about 15 nucleotides to about 28 nucleotides. In some embodiments, the guide RNA comprises at least about 15 nucleotides. In some embodiments, the guide RNA comprises at most about 28 nucleotides. In some embodiments, the guide RNA comprises about 15 nucleotides to about 16 nucleotides, about 15 nucleotides to about 17 nucleotides, about 15 nucleotides to about 18 nucleotides, about 15 nucleotides to about 19 nucleotides, about 15 nucleotides to about 20 nucleotides, about 15 nucleotides to about 21 nucleotides, about 15 nucleotides to about 22 nucleotides, about 15 nucleotides to about 23 nucleotides, about 15 nucleotides to about 24 nucleotides, about 15 nucleotides to about 25 nucleotides, about 15 nucleotides to about 28 nucleotides, about 16 nucleotides to about 17 nucleotides, about 16 nucleotides to about 18 nucleotides, about 16 nucleotides to about 19 nucleotides, about 16 nucleotides to about 20 nucleotides, about 16 nucleotides to about 21 nucleotides, about 16 nucleotides to about 22 nucleotides, about 16 nucleotides to about 23 nucleotides, about 16 nucleotides to about 24 nucleotides, about 16 nucleotides to about 25 nucleotides, about 16 nucleotides to about 28 nucleotides, about 17 nucleotides to about 18 nucleotides, about 17 nucleotides to about 19 nucleotides, about 17 nucleotides to about 20 nucleotides, about 17 nucleotides to about 21 nucleotides, about 17 nucleotides to about 22 nucleotides, about 17 nucleotides to about 23 nucleotides, about 17 nucleotides to about 24 nucleotides, about 17 nucleotides to about 25 nucleotides, about 17 nucleotides to about 28 nucleotides, about 18 nucleotides to about 19 nucleotides, about 18 nucleotides to about 20 nucleotides, about 18 nucleotides to about 21 nucleotides, about 18 nucleotides to about 22 nucleotides, about 18 nucleotides to about 23 nucleotides, about 18 nucleotides to about 24 nucleotides, about 18 nucleotides to about 25 nucleotides, about 18 nucleotides to about 28 nucleotides, about 19 nucleotides to about 20 nucleotides, about 19 nucleotides to about 21 nucleotides, about 19 nucleotides to about 22 nucleotides, about 19 nucleotides to about 23 nucleotides, about 19 nucleotides to about 24 nucleotides, about 19 nucleotides to about 25 nucleotides, about 19 nucleotides to about 28 nucleotides, about 20 nucleotides to about 21 nucleotides, about 20 nucleotides to about 22 nucleotides, about 20 nucleotides to about 23 nucleotides, about 20 nucleotides to about 24 nucleotides, about 20 nucleotides to about 25 nucleotides, about 20 nucleotides to about 28 nucleotides, about 21 nucleotides to about 22 nucleotides, about 21 nucleotides to about 23 nucleotides, about 21 nucleotides to about 24 nucleotides, about 21 nucleotides to about 25 nucleotides, about 21 nucleotides to about 28 nucleotides, about 22 nucleotides to about 23 nucleotides, about 22 nucleotides to about 24 nucleotides, about 22 nucleotides to about 25 nucleotides, about 22 nucleotides to about 28 nucleotides, about 23 nucleotides to about 24 nucleotides, about 23 nucleotides to about 25 nucleotides, about 23 nucleotides to about 28 nucleotides, about 24 nucleotides to about 25 nucleotides, about 24 nucleotides to about 28 nucleotides, or about 25 nucleotides to about 28 nucleotides. In some embodiments, the guide RNA comprises about 15 nucleotides, about 16 nucleotides, about 17 nucleotides, about 18 nucleotides, about 19 nucleotides, about 20 nucleotides, about 21 nucleotides, about 22 nucleotides, about 23 nucleotides, about 24 nucleotides, about 25 nucleotides, or about 28 nucleotides.

In some embodiments, the guide nucleic acid further comprises a primer binding site (PBS). In some embodiments, the primer binding site is on a 3′ of the guide nucleic acid. In some embodiments, the primer binding site comprises at least 2, 4, 6, 8, 10, 13, 16, 20, 24, 28, 32, 36, 40, 45, 50, 55, 60, or 65 nucleotides. In some embodiments, the primer binding site comprises less than 2, 4, 6, or 8, nucleotides.

In some embodiments, the guide nucleic acid further comprises a reverse transcriptase template (RTT). In some embodiments, a base in the RTT comprises a bulky modification selected from the group of complex sugars, complex amino groups, and/or other modifications compatible with RNA. In some embodiments, the RTT is fused to the guide RNA. In some embodiments, the guide nucleic acid further comprises a homology sequence that is complementary to a region in the non-edited DNA strand. In some embodiments, the guide nucleic acid comprises a nucleic acid template. In some embodiments, the RTT has a length of at least about 10, 20, 30, 40, 50, 60, 70, 80, 90, or 100 nucleotides. In some the RTT has a length of at least about 100, 200, 300, 400, 500, 600, 700, 800, 900, or 1000 nucleotides. In some embodiments, the RTT has a length of at least about 1000, 2000, 3000, 4000, or 5000 nucleotides. In some embodiments, the RTT has a length between about 10 and about 20, 30, 40, 50, 60, 70, 80, 90, 100, 120, 140, 160, 180, 200, or more than 200 nucleotides. In some embodiments, the RTT has a length between about 20 and about 30, 40, 50, 60, 70, 80, 90, 100, 120, 140, 160, 180, 200, or more than 200 nucleotides. In some embodiments, the RTT has a length between about 30 and about 40, 50, 60, 70, 80, 90, 100, 120, 140, 160, 180, 200, or more than 200 nucleotides. In some embodiments, the RTT has a length between about 40 and about 50, 60, 70, 80, 90, 100, 120, 140, 160, 180, 200, or more than 200 nucleotides. In some embodiments, the RTT has a length between about 50 and about 60, 70, 80, 90, 100, 120, 140, 160, 180, 200, or more than 200 nucleotides. In some embodiments, the RTT has a length between about 60 and about 70, 80, 90, 100, 120, 140, 160, 180, 200, or more than 200 nucleotides. In some embodiments, the RTT has a length between about 70 and about 80, 90, 100, 120, 140, 160, 180, 200, or more than 200 nucleotides. In some embodiments, the RTT has a length between about 80 and about 100, 120, 140, 160, 180, 200, or more than 200 nucleotides. In some embodiments, the RTT has a length between about 100 and about 120, 140, 160, 180, 200, or more than 200 nucleotides. In some embodiments, the RTT has a length between about 100 and about 4000 nucleotides. In some embodiments, the RTT has a length between about 100 and about 200, 300, 400, 500, 600, 700, 800, 900, 1000, 1500, 2000, 2500, 3000, 3500, or 4000 nucleotides. In some embodiments, the RTT has a length between about 500 and about 600, 700, 800, 900, 1000, 1500, 2000, 2500, 3000, 3500, or 4000 nucleotides. In some embodiments, the RTT has a length between about 1000 and about 1500, 2000, 2500, 3000, 3500, or 4000 nucleotides. In some embodiments, the RTT has a length between about 2000 and about 2500, 3000, 3500, or 4000 nucleotides. In some embodiments, the RTT has a length between about 3000 and about 3500, or 4000 nucleotides.

Methods of making guide nucleic acids are known in the art. For example, guide RNAs and pegRNAs, as well as and modified guide RNAs and pegRNAs, can be chemically synthesized. Additionally, nucleic sequences encoding guide nucleic acids can be cloned into a vector and transcribed from the vector in vitro or in vivo using RNA polymerases.

### Delivery and Vectors

Disclosed herein, in some embodiments, are nucleic acid sequences encoding a reverse transcriptase, a fusion protein, or a gene editing system described herein.

In some embodiments, the nucleic acid encoding the endonuclease system or components thereof is a DNA, for example a linear DNA, a plasmid DNA, or a minicircle DNA. In some embodiments, the nucleic acid encoding the reverse transcriptase, the fusion protein, or the gene editing system described herein is an RNA, for example a mRNA.

In some embodiments, the nucleic acid encoding the reverse transcriptase, the fusion protein, or the gene editing system described herein is delivered by a nucleic acid-based vector. In some embodiments, the nucleic acid-based vector is a plasmid (e.g., circular DNA molecules that can autonomously replicate inside a cell), cosmid (e.g., pWE or sCos vectors), artificial chromosome, human artificial chromosome (HAC), yeast artificial chromosomes (YAC), bacterial artificial chromosome (BAC), P1-derived artificial chromosomes (PAC), phagemid, phage derivative, bacmid, or virus. In some embodiments, the nucleic acid-based vector is selected from the list consisting of: pSF-CMV-NEO-NH2-PPT-3×FLAG, pSF-CMV-NEO-COOH-3×FLAG, pSF-CMV-PURO-NH2-GST-TEV, pSF-OXB20-COOH-TEV-FLAG(R)-6His, pCEP4 pDEST27, pSF-CMV-Ub-KrYFP, pSF-CMV-FMDV-daGFP, pEF1a-mCherry-N1 vector, pEF1a-tdTomato vector, pSF-CMV-FMDV-Hygro, pSF-CMV-PGK-Puro, pMCP-tag(m), pSF-CMV-PURO-NH2-CMYC, pSF-OXB20-BetaGal, pSF-OXB20-Fluc, pSF-OXB20, pSF-Tac, pRI 101-AN DNA, pCambia2301, pTYB21, pKLAC2, pAc5.1N5-His A, and pDEST8.

In some embodiments, the nucleic acid-based vector comprises a promoter. In some embodiments, the promoter is selected from the group consisting of a mini promoter, an inducible promoter, a constitutive promoter, and derivatives thereof. In some embodiments, the promoter is selected from the group consisting of CMV, CBA, EF1a, CAG, PGK, TRE, U6, UAS, T7, Sp6, lac, araBad, trp, Ptac, p5, p19, p40, Synapsin, CaMKII, GRK1, and derivatives thereof. In some embodiments the promoter is a U6 promoter. In some embodiments, the promoter is a CAG promoter.

In some embodiments, the nucleic acid-based vector is a virus. In some embodiments, the virus is an alphavirus, a parvovirus, an adenovirus, an AAV, a baculovirus, a Dengue virus, a lentivirus, a herpesvirus, a poxvirus, an anellovirus, a bocavirus, a vaccinia virus, or a retrovirus. In some embodiments, the virus is an alphavirus. In some embodiments, the virus is a parvovirus. In some embodiments, the virus is an adenovirus. In some embodiments, the virus is an AAV. In some embodiments, the virus is a baculovirus. In some embodiments, the virus is a Dengue virus. In some embodiments, the virus is a lentivirus. In some embodiments, the virus is a herpesvirus. In some embodiments, the virus is a poxvirus. In some embodiments, the virus is an anellovirus. In some embodiments, the virus is a bocavirus. In some embodiments, the virus is a vaccinia virus. In some embodiments, the virus is or a retrovirus.

In some embodiments, the AAV is AAV1, AAV2, AAV3, AAV4, AAV5, AAV6, AAV7, AAV8, AAV9, AAV10, AAV11, AAV12, AAV13, AAV14, AAV15, AAV16, AAV-rh8, AAV-rh10, AAV-rh20, AAV-rh39, AAV-rh74, AAV-rhM4-1, AAV-hu37, AAV-Anc80, AAV-Anc80L65, AAV-7m8, AAV-PHP-B, AAV-PHP-EB, AAV-2.5, AAV-2tYF, AAV-3B, AAV-LK03, AAV-HSC1, AAV-HSC2, AAV-HSC3, AAV-HSC4, AAV-HSC5, AAV-HSC6, AAV-HSC7, AAV-HSC8, AAV-HSC9, AAV-HSC10, AAV-HSC11, AAV-HSC12, AAV-HSC13, AAV-HSC14, AAV-HSC15, AAV-TT, AAV-DJ/8, AAV-Myo, AAV-NP40, AAV-NP59, AAV-NP22, AAV-NP66, AAV-HSC16, or a derivative thereof. In some embodiments, the herpesvirus is HSV type 1, HSV-2, VZV, EBV, CMV, HHV-6, HHV-7, or HHV-8.

In some embodiments, the virus is AAV1 or a derivative thereof. In some embodiments, the virus is AAV2 or a derivative thereof. In some embodiments, the virus is AAV3 or a derivative thereof. In some embodiments, the virus is AAV4 or a derivative thereof. In some embodiments, the virus is AAV5 or a derivative thereof. In some embodiments, the virus is AAV6 or a derivative thereof. In some embodiments, the virus is AAV7 or a derivative thereof. In some embodiments, the virus is AAV8 or a derivative thereof. In some embodiments, the virus is AAV9 or a derivative thereof. In some embodiments, the virus is AAV10 or a derivative thereof. In some embodiments, the virus is AAV11 or a derivative thereof. In some embodiments, the virus is AAV12 or a derivative thereof. In some embodiments, the virus is AAV13 or a derivative thereof. In some embodiments, the virus is AAV14 or a derivative thereof. In some embodiments, the virus is AAV15 or a derivative thereof. In some embodiments, the virus is AAV16 or a derivative thereof. In some embodiments, the virus is AAV-rh8 or a derivative thereof. In some embodiments, the virus is AAV-rh10 or a derivative thereof. In some embodiments, the virus is AAV-rh20 or a derivative thereof. In some embodiments, the virus is AAV-rh39 or a derivative thereof. In some embodiments, the virus is AAV-rh74 or a derivative thereof. In some embodiments, the virus is AAV-rhM4-1 or a derivative thereof. In some embodiments, the virus is AAV-hu37 or a derivative thereof. In some embodiments, the virus is AAV-Anc80 or a derivative thereof. In some embodiments, the virus is AAV-Anc80L65 or a derivative thereof. In some embodiments, the virus is AAV-7m8 or a derivative thereof. In some embodiments, the virus is AAV-PHP-B or a derivative thereof. In some embodiments, the virus is AAV-PHP-EB or a derivative thereof. In some embodiments, the virus is AAV-2.5 or a derivative thereof. In some embodiments, the virus is AAV-2tYF or a derivative thereof. In some embodiments, the virus is AAV-3B or a derivative thereof. In some embodiments, the virus is AAV-LK03 or a derivative thereof. In some embodiments, the virus is AAV-HSC1 or a derivative thereof. In some embodiments, the virus is AAV-HSC2 or a derivative thereof. In some embodiments, the virus is AAV-HSC3 or a derivative thereof. In some embodiments, the virus is AAV-HSC4 or a derivative thereof. In some embodiments, the virus is AAV-HSC5 or a derivative thereof. In some embodiments, the virus is AAV-HSC6 or a derivative thereof. In some embodiments, the virus is AAV-HSC7 or a derivative thereof. In some embodiments, the virus is AAV-HSC8 or a derivative thereof. In some embodiments, the virus is AAV-HSC9 or a derivative thereof. In some embodiments, the virus is AAV-HSC10 or a derivative thereof. In some embodiments, the virus is AAV-HSC11 or a derivative thereof. In some embodiments, the virus is AAV-HSC12 or a derivative thereof. In some embodiments, the virus is AAV-HSC13 or a derivative thereof. In some embodiments, the virus is AAV-HSC14 or a derivative thereof. In some embodiments, the virus is AAV-HSC15 or a derivative thereof. In some embodiments, the virus is AAV-TT or a derivative thereof. In some embodiments, the virus is AAV-DJ/8 or a derivative thereof. In some embodiments, the virus is AAV-Myo or a derivative thereof. In some embodiments, the virus is AAV-NP40 or a derivative thereof. In some embodiments, the virus is AAV-NP59 or a derivative thereof. In some embodiments, the virus is AAV-NP22 or a derivative thereof. In some embodiments, the virus is AAV-NP66 or a derivative thereof. In some embodiments, the virus is AAV-HSC16 or a derivative thereof.

In some embodiments, the virus is HSV-1 or a derivative thereof. In some embodiments, the virus is HSV-2 or a derivative thereof. In some embodiments, the virus is VZV or a derivative thereof. In some embodiments, the virus is EBV or a derivative thereof. In some embodiments, the virus is CMV or a derivative thereof. In some embodiments, the virus is HHV-6 or a derivative thereof. In some embodiments, the virus is HHV-7 or a derivative thereof. In some embodiments, the virus is HHV-8 or a derivative thereof.

In some embodiments, the nucleic acid encoding the reverse transcriptase, the fusion protein, or the gene editing system described herein is delivered by a non-nucleic acid-based delivery system (e.g., a non-viral delivery system). In some embodiments, the non-viral delivery system is a liposome. In some embodiments, the nucleic acid is associated with a lipid. The nucleic acid associated with a lipid, in some embodiments, is encapsulated in the aqueous interior of a liposome, interspersed within the lipid bilayer of a liposome, attached to a liposome via a linking molecule that is associated with both the liposome and the nucleic acid, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. In some embodiments, the nucleic acid is comprised in a lipid nanoparticle (LNP).

In some embodiments, the reverse transcriptase, the fusion protein, or the gene editing system described herein is introduced into the cell in any suitable way, either stably or transiently. In some embodiments, the reverse transcriptase, the fusion protein, or the gene editing system described herein is transfected into the cell. In some embodiments, the cell is transduced or transfected with a nucleic acid construct that encodes the reverse transcriptase, the fusion protein, or the gene editing system described herein. For example, a cell is transduced (e.g., with a virus encoding the reverse transcriptase, the fusion protein, or the gene editing system described herein), or transfected (e.g., with a plasmid encoding the reverse transcriptase, the fusion protein, or the gene editing system described herein) with a nucleic acid that encodes the reverse transcriptase, the fusion protein, or the gene editing system described herein, or the reverse transcriptase, the fusion protein, or the gene editing system described herein. In some embodiments, the transduction is a stable or transient transduction. In some embodiments, cells expressing the reverse transcriptase, the fusion protein, or the gene editing system described herein or containing the reverse transcriptase, the fusion protein, or the gene editing system described herein are transduced or transfected with one or more gRNA molecules, for example, when the reverse transcriptase, the fusion protein, or the gene editing system described herein comprises a CRISPR nuclease. In some embodiments, a plasmid expressing the reverse transcriptase, the fusion protein, or the gene editing system described herein is introduced into cells through electroporation, transient (e.g., lipofection) and stable genome integration (e.g., piggybac) and viral transduction (for example lentivirus or AAV) or other methods known to those of skill in the art. In some embodiments, the gene editing system is introduced into the cell as one or more polypeptides. In some embodiments, delivery is achieved through the use of RNP complexes. Delivery methods to cells for polypeptides and/or RNPs are known in the art, for example by electroporation or by cell squeezing.

Exemplary methods of delivery of nucleic acids include lipofection, nucleofection, electroporation, stable genome integration (e.g., piggybac), microinjection, biolistics, virosomes, liposomes, immunoliposomes, polycation or lipid nucleic acid conjugates, naked DNA, artificial virions, and agent-enhanced uptake of DNA. Lipofection is described in e.g., U.S. Pat. Nos. 5,049,386; 4,946,787, and 4,897,355) and lipofection reagents are sold commercially (e.g., Transfectam™, Lipofectin™ and SF Cell Line 4D-Nucleofector X Kit™ (Lonza)). Cationic and neutral lipids that are suitable for efficient receptor-recognition lipofection of polynucleotides include those of WO 91/17424 and WO 91/16024. In some embodiments, the delivery is to cells (e.g., in vitro or ex vivo administration) or target tissues (e.g., in vivo administration). In some embodiments, the nucleic acid is comprised in a liposome or a nanoparticle that specifically targets a host cell.

Additional methods for the delivery of nucleic acids to cells are known to those skilled in the art. See, for example, US 2003/0087817.

In some embodiments, the present disclosure provides a cell comprising a vector or a nucleic acid described herein. In some embodiments, the cell expresses a gene editing system or parts thereof. In some embodiments, the cell is a human cell. In some embodiments, the cell is genome edited ex vivo. In some embodiments, the cell is genome edited in vivo.

### Cells

Described herein, in certain embodiments, is a cell comprising the systems or vectors described herein.

In some embodiments, the cell is a eukaryotic cell (e.g., a plant cell, an animal cell, a protist cell, or a fungi cell), a mammalian cell (a Chinese hamster ovary (CHO) cell, baby hamster kidney (BHK), human embryo kidney (HEK), mouse myeloma (NSO), or human retinal cells), an immortalized cell (e.g., a HeLa cell, a COS cell, a HEK-293T cell, a MDCK cell, a 3T3 cell, a PC12 cell, a Huh7 cell, a HepG2 cell, a K562 cell, a N2a cell, or a SY5Y cell), an insect cell (e.g., a Spodoptera frugiperda cell, a Trichoplusia ni cell, a Drosophila melanogaster cell, a S2 cell, or a Heliothis virescens cell), a yeast cell (e.g., a Saccharomyces cerevisiae cell, a Cryptococcus cell, or a Candida cell), a plant cell (e.g., a parenchyma cell, a collenchyma cell, or a sclerenchyma cell), a fungal cell (e.g., a Saccharomyces cerevisiae cell, a Cryptococcus cell, or a Candida cell), or a prokaryotic cell (e.g., a E. coli cell, a streptococcus bacterium cell, a streptomyces soil bacteria cell, or an archaea cell). In some embodiments, the cell is a eukaryotic cell. In some embodiments, the cell is a mammalian cell. In some embodiments, the cell is an immortalized cell. In some embodiments, the cell is an insect cell. In some embodiments, the cell is a yeast cell. In some embodiments, the cell is a plant cell. In some embodiments, the cell is a fungal cell. In some embodiments, the cell is a prokaryotic cell.

In some embodiments, the cell is an A549, HEK-293, HEK-293T, BHK, CHO, HeLa, MRC5, Sf9, Cos-1, Cos-7, Vero, BSC 1, BSC 40, BMT 10, WI38, HeLa, Saos, C2C12, L cell, HT1080, HepG2, Huh7, K562, a primary cell, or derivative thereof.

### Methods of Use

Described herein, in some embodiments, are methods for modifying a double- and/or single-stranded nucleic acid, comprising a) providing a cell with a guide nucleic acid to bind to a target strand of the double-stranded nucleic acid; b) providing a cell with a nuclease or nickase to cleave the double-stranded nucleic acid at a location of binding of the guide nucleic acid; c) providing a cell with a reverse transcriptase to synthesize a modification in the target strand of the double-stranded nucleic acid at a location of cleavage by the nickase and/or double strand nuclease.

In some embodiments, the methods are used to introduce a modification in the genome of a cell. In some embodiments, the modification is an insertion, deletion, or mutation. In some embodiments, the methods are used to introduce site-directed insertions, deletions, and/or mutations in the genome of a cell (for example an insertion and a mutation). In some embodiments, the methods are used in combination with a nucleic acid template to facilitate site-directed insertions into the genome of a cell. In some embodiments, the cell is a human cell. In some embodiments, the cell genome or a vector comprised in the cell is modified. In some embodiments, the cell genome is modified ex vivo. In some embodiments, the cell genome is modified in vivo.

In some embodiments, the methods further comprise providing the cell a transposase, integrase, or homing endonuclease. In some embodiments, the methods further comprise providing the cell a retrotransposon. In some embodiments, the method further comprises providing an RNA or DNA insertion template.

In some embodiments, the methods described herein further comprise detecting the genome modifications. In some embodiments, after the cell genome is modified, the cell is cultured for a certain amount of time. In some embodiments, the DNA or RNA is extracted and sequenced, and modified sequence areas are mapped and compared with an unmodified sequence. In some embodiments, cells are stained with antibodies for protein products that are translated from the modified nucleic acid, and the resulting stained proteins or polypeptides in the cell are analyzed, for example by flow cytometry.

The methods described herein can be used, for example, for targeted SNP corrections, small insertions, or small deletions. Additionally, the methods described herein can be used for targeted insertion of large templates into the genome of a cell by using a suitable RTT.

### Kits

In some embodiments, this disclosure provides kits comprising one or more nucleic acid constructs encoding the various components of the fusion protein or genome editing system described herein, e.g., comprising a nucleotide sequence encoding the components of the fusion protein or genome editing system capable of modifying a target DNA sequence. In some embodiments, the nucleotide sequence comprises a heterologous promoter that drives expression of the RNA genome editing system components.

In some embodiments, any of the targetable reverse transcriptases or genome editing systems disclosed herein is assembled into a pharmaceutical, diagnostic, or research kit to facilitate its use in therapeutic, diagnostic, or research applications. A kit may include one or more containers housing any of the vectors disclosed herein and instructions for use.

The kit may be designed to facilitate use of the methods described herein by researchers and can take many forms. Each of the compositions of the kit, where applicable, may be provided in liquid form (e.g., in solution), or in solid form, (e.g., a dry powder). In some embodiments, the compositions are constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions, in some embodiments, are in a form prescribed by a governmental agency regulating the manufacture, use, or sale of pharmaceuticals or biological products, which instructions can also reflect approval by the agency of manufacture, use, or sale for animal administration.

### EXAMPLES

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Example 1. Bioinformatic Identification of Reverse Transcriptases

This example describes the identification of proteins with reverse transcriptase function by a bioinformatic approach.

An extensive assembly-driven metagenomic database of microbial, viral, and eukaryotic genomes was bioinformatically analyzed for proteins with putative reverse transcriptase function. The analysis uncovered millions of proteins with predicted reverse transcriptase function. The predicted RT hits were then bioinformatically filtered for complete open reading frames (ORFs) with a high quality RT domain hit covering over 70% of the reference RT domains, and containing expected catalytic residues. After filtering, 29 RTs were selected for their potential to develop gene editing tools (SEQ ID NOs: 88-116 and 138-139). For all of these identified putative RTs, the predicted active site tetrad motif is [Y/F]ADD, where the most frequent amino acid at the first position of the tetrad is tyrosine (Y, 96.6%) vs. phenylalanine (F, 3.4%). The aspartate dyad (DD) is the most conserved feature for RT activity.

### Example 2. Reverse Transcriptases (RTs) for Short Corrections, Small Insertions, and Deletions

This example describes the use of untethered reverse transcriptases in combination with pegRNAs for targeted genome editing in HEK293T cells.

Testing Reverse Transcriptase Candidates from the Nickase

Reverse transcriptase (RT) candidates from the MG153 and MG160 families were cloned into a plasmid where expression of the RT candidate is driven by the CMV promoter. The plasmid was isolated for transfection into HEK293T cells. A second plasmid containing a nickase spCas9 (H840A) where the expression was driven by a CMV promoter, and the RT-containing plasmid were co-transfected. Chemically synthesized pegRNAs (SEQ ID NOs: 6-29) containing the desired edit in the RT template were transfected. All components (plasmids and pegRNAs) were reverse transfected into 150,000 HEK293T cells in a 24 well plate. 72 hours post-transfection, cells were lysed in 100 μL of DNA extraction solution. Primers containing barcodes for next generation sequencing (NGS) (SEQ ID NOs: 30-31) were used to amplify a ˜250 bp target (SEQ ID NO: 32. PCR clean-up was then performed, and samples were NGS sequenced. FASTQ files were then processed to determine the percentage of reads with desired change.

### MG153 Family

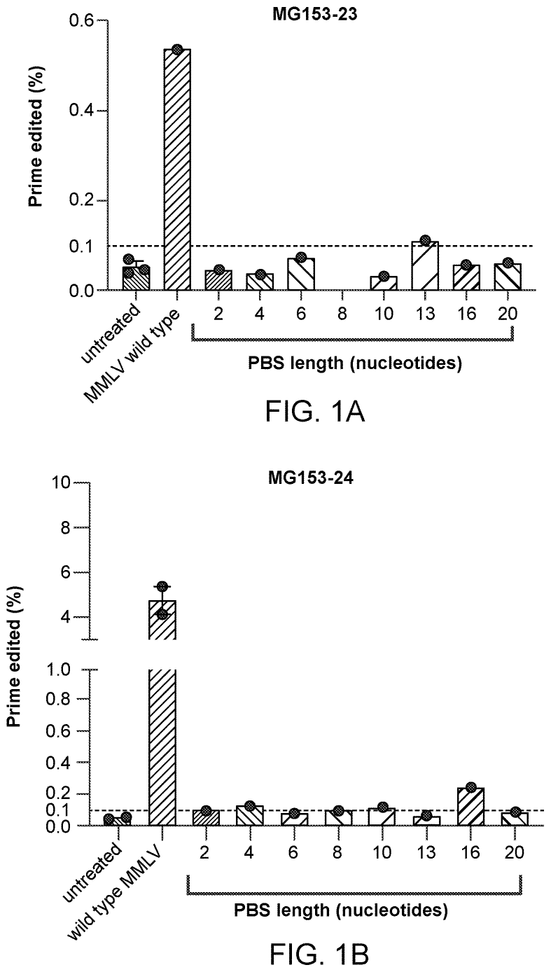

Untethered MG153 candidates MG153-23 and MG153-24 (SEQ ID NOs: 2-3) were tested for prime editing in HEK293T cells to determine percent change of desired correction. Percent editing for each RT is shown in FIG. 1 for each pegRNA with varying PBS lengths (2, 4, 6, 8, 10, 13, 16, 20 nucleotides).

### MG160 Family

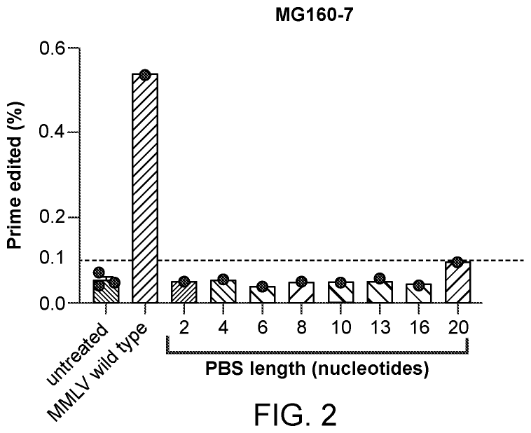

Untethered MG160 family candidate MG160-7 (SEQ ID NO: 5) was tested in mammalian cells for activity as described above. The detected activity in this assay was not above the background (FIG. 2).

### Testing Reverse Transcriptase Candidates Tethered to a Nickase

The activity of diverse RT classes with CRISPR Type II nucleases was evaluated. RT candidates were cloned into a plasmid containing the nickase spCas9(H840A) to generate an RT-nickase fusion. The CMV promoter drove the expression of the RT-Nickase fusion protein, which contained a thirty three amino acid linker (SEQ ID NO: 33) between the nickase and the RT candidate. The fusion protein was then transfected into HEK293T cells and processed for NGS as described above.

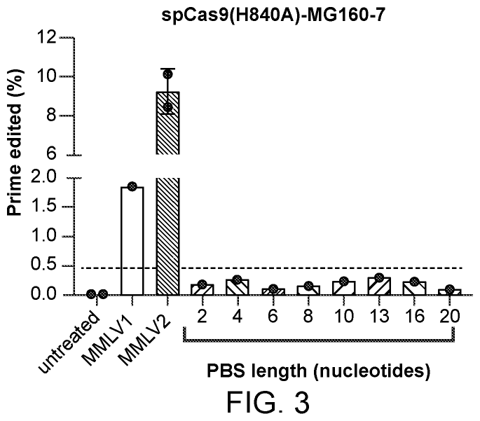

The activity of tethered MG160-7 (SEQ ID NO: 5) is shown in FIG. 3. The activity detected in this assay was not above the background.

The data above demonstrates that candidate RTs were identified that showed comparable or higher activity than MMLV WT in a prime editing context. Having activity across a broad range of families allows the identification of RT candidates for different kinds of genomic modifications (i.e., SNP corrections, insertions, or deletions). The small size of the identified RT (⅓ of MMLV WT) allows an efficient delivery using adeno-associated viruses (AAVs) and lipid nanoparticles (LNPs).

### Example 3. RTs for Short Corrections, Small Insertions, and Deletions (Prophetic)

Additional RTs from the MG153 families, including MG153-22 (SEQ ID NO: 1), or additional candidates are tested as described in Example 2 in the untethered format. This allows for the identification of additional RT candidates for small corrections, insertions, and deletions.

### REFERENCES

Anzalone A V, Gao X D, Podracky C J, Nelson A T, Koblan L W, Raguram A, Levy J M, Mercer J A M, Liu D R. Programmable deletion, replacement, integration and inversion of large DNA sequences with twin prime editing. Nat Biotechnol. 2022, 40(5):731-740. doi: 10.1038/s41587-021-01133-w. Epub 2021 Dec. 9. PMID: 34887556: PMCID: PMC9117393.
Clement K, Rees H, Canver M C, Gehrke J M, Farouni R, Hsu J Y, Cole M A, Liu D R, Joung J K, Bauer D E, Pinello L. CRISPResso2 provides accurate and rapid genome editing sequence analysis. Nat Biotechnol. 2019, 37(3):224-226. doi: 10.1038/s41587-019-0032-3. PMID: 30809026; PMCID: PMC6533916.
Gonzalez-Delgado A, Mestre M R, Martinez-Abarca F, Toro N. Prokaryotic reverse transcriptases: from retroelements to specialized defense systems. FEMS Microbiol Rev. 2021, 45(6):fuab025. doi: 10.1093/femsre/fuab025. PMID: 33983378; PMCID: PMC8632793.
He S, Corneloup A, Guynet C, Lavatine L, Caumont-Sarcos A, Siguier P, Marty B, Dyda F, Chandler M, Ton Hoang B. The IS200/IS605 Family and “Peel and Paste” Single-strand Transposition Mechanism. Microbiol Spectr. 2015, 3(4). Doi: 10.1128/microbiolspec.MDNA3-0039-2014. PMID: 26350330.

### EQUIVALENTS

The disclosure may be embodied in other specific forms without departing from the spirit or essential characteristics thereof. The foregoing embodiments are therefore to be considered in all respects illustrative rather than limiting on the disclosure described herein. Scope of the disclosure is thus indicated by the appended claims rather than by the foregoing description, and all changes that come within the meaning and range of equivalency of the claims are intended to be embraced therein.

SEQUENCE LISTING

| SEQ | | |
|---|---|---|
| ID | | |
| NO: | Description | Sequence |
| 1 | MG153-22_ | GCTAGCGCCACCATGTACCCCTATGATGTACCTGATTATGCTTCTGGCGGCAGCA |
| | cloned_ | GCGGCGGCTCAAGCGGAAGTGAAACACCTGGAACTTCCGAAAGCGCTACGCCTGA |
| | into_ | ATCTTCTGGTGGATCTTCTGGGGGATCTTCCGCATGCATGCGGCAGAAATCAGAA |
| | untethered_ | CAACTTGAATTAGCCCTGGATAATAGGGGAGAGGCTCCCACCTCCAGACGAAGTG |
| | plasmid | GCGAAGCACCGACTACAGCGCACGAAGCAGAGAGGTCCGGCGGGGGACACCGGTT |
| | | GATGGAGCAGGTCGTAGCTCGCGCTAATGCTTTGGCCGCCCTGAGGAGGGTGAAA |
| | | CAAAACCGGGGGAGCCCTGGCGTGGACGGGATGACAGTCGGCGAGCTGCCTCAAT |
| | | ATTTAGCAAAACACTGGGAAACCGTGCGGGAGCAGCTGCTGGCCGGATCTTACCA |
| | | ACCAGAGCCCGTACGTAGACAGGCCATCCCCAAACCTGGCGGAGGTACCCGAGTT |
| | | CTGGGGATCCCGACAGTGTTGGATCGCTTCATCCAGCAGTGCCTCCTGCAGGTAC |
| | | TTCAGCCAAGGTTTGACCCCAGTTTTTCAGATCACTCATACGGATTCAGGCCAGG |
| | | GAGGAACGCTCACGATGCAGTCTGTGCTGCACAGCAGTACATACAAGCCGGTCGC |
| | | AGATGGGTGGCTGACCTGGATCTTGAGAAGTTCTTTGATCGGGTGAATCATGACG |
| | | TGCTCATGGAGCGGCTCGCGCGGAGAATCGCTGATCGGAGGGTGCTGCGGTTGAT |
| | | ACGACGTTACCTTGTCGCTGGGATCCTGCATGGAGGTGTTGTTGTGGAGCGAAGA |
| | | GAAGGGACTCCGCAGGGGGGCCCACTGAGCCCTCTGCTGGCCAGTGTCCTGCTGG |
| | | ACGAAGTGGATCGCGAACTTGAGAGGCGAGGGCTGGCATTCGCAAGATATGCTGA |
| | | TGATCTCAACGTTTACTGCGGGAGTAGGCGAGCGGCCCATGACGCAATGGCCACA |
| | | TTGAAGAGGCTCTTTGCCGCCCTCCGGTTAAAAGTAAACGAATCCAAGTCCACGG |
| | | TGGCCAGAGTCTGGGAAAGGAAATTTCTGGGATATTCATTCTGGGTGGCACCCGG |
| | | TAGGGTTATCAGACCAAGGATCGCACCGGCAGCCCTGGCTGTGATGAAAGAGCGG |
| | | GTGCGCCGCATCACGCGGCGCACCGGCGGTCGAAGCTTGGAAGCTGTGGCACAGG |
| | | AGCTGCGAGAATATCTCACAGGGTGGAAAGCATACTTTCGGCTTGCCGGGAAACC |
| | | CCGGGTGTTCAGAGACCTGGATGAGTGGACCCGCCACAGGTTGCGCGCCGTCCAG |
| | | CTGAAACAATGGAAGAGAGGAAGGACCGTCTGTAGAGAGTTACTGGCCAGAGGTG |
| | | TCCCCGAGAGGGAGGCCAGAGCTGCCGCTGCCCATGCAAGGAGGTGGTGGGCGAT |
| | | GGCTGCTCACAGTGCGTTACAGACCGCCCTGCCAAACTCCCATTTCGACCAATTA |
| | | GGAGTGCCAAGGTTGGCGGGTCGTCCTAGGCCTAAGAAGAAAAGAAAGGTGGGTG |
| | | GTTCTCCTAAGAAAAAGAGAAAGGTCTGAGAATTC |
| 2 | MG153-23_ | GCTAGCGCCACCATGTACCCCTATGATGTACCTGATTATGCTTCTGGCGGCAGCA |
| | cloned_ | GCGGCGGCTCAAGCGGAAGTGAAACACCTGGAACTTCCGAAAGCGCTACGCCTGA |
| | into_ | ATCTTCTGGTGGATCTTCTGGCGGATCTTCCGCATGCATGAGGGCTGACGAGGCT |
| | untethered_ | GAGGCTCACGCCAGCGCCGCGAGCACAGGAAAGGGCGGCAGGAACCTTCCTGGTA |
| | plasmid | CTGCAGCTGGCGCTGAAGTGAGAGCTGCCGCAGGTGGGCGAACGAAACCGGAGGC |
| | | TCTGAGGCTTATGGAGGCTGCCGTTGAGAGATCTAACATGCTGGGCGCCTATGAA |
| | | AGAGTGGTTAAGAATCAGGGCGCCCCCGGGGTTGATGGCCTGACCGTGACTGAGT |
| | | TTAAACCATGGCTGCAGGCACACTGGCCCAAAATCAGACAAGTGCTGCTCGCAGG |
| | | GGAATACATGCCCGCCGCCGTGAGAAAGGTTGATATACCCAAACCGCAGGGGGGG |
| | | GTCCGGACCCTGGGCATTCCGACAGTGCTGGATCGGCTCATTCAGCAGGCCCTCC |
| | | ACCAGGTCCTGCAGCCCCTGTTCGAACCAGAGTTCAGTGAGTCTTCCTATGGCTT |
| | | TAGACCCGGTCGGAATGCCCACCAGGCCGTCGAAGCCGCGCGCTCTTACGTCGCT |
| | | GAAGGGAAACGTTGGGTGGTTGATCTGGAAAAATTTTTTGATCGAGTGAACCACG |
| | | ACGTACTTATGGCCCGGGTGGCCCGCAAGGTTAAGGATGAGAGAGTGCTGAAACT |
| | | GATCCGCAGGTATCTCGAAGCAGGACTGATGGAGGGTGGCATGACCAGTGCACGA |
| | | ACAGAGGGTACACCTCAGGGTGGCCCCTTAAGTCCCCTCTTGAGCAATACCCTGC |
| | | TTACAGACTTGGATAGAGAGCTGGAGAAACGCGGTCATCGTTTCTGTCGCTATGC |
| | | TGACGATTGTAATGTTTATGTGGGAAGCAGGAGATCTGGTCAGAGGGTCATGGCA |
| | | AAGATTACCGCGTTTCTGGAACAAAGGCTTAAGTTACAGGTTAATGCTGATAAAA |
| | | GCGCCGTGGCTAGACCATGGCAAAGGAAATTCCTTGGGTACAGCGTCACCTGGCA |
| | | CCGGAACCCCAAACTTAAAATTGCTCCATCCTCCAGACAGAGACTGGCTGAGAAG |
| | | ATACGGCAGACCCTGAGAGGGGCCCGCGGCCAGAGCCTCAGGCAGGTAATCGCAC |
| | | AGCTGAACCCTATCCTGAGGGGCTGGGTTGCTTACTTTAGACTGACCGAAGAAAA |
| | | AGGCGTCCTGGAAGAATTAGATGGGGGGTCAGGCGCAAGCTGAGAGCCTTGCTTT |
| | | GGAGACATTGGAAACGCGGGTACCCAAGAGCACAGAACTTGATGAGGGCTGGTTT |
| | | AAGGCCAGAAAGAGCTTGGCAGTCTGCGACAAATGGGCACGGGCCTTGGTGGAAT |
| | | GGGGGATCATCTCATATGAATGCTGCATGTCCGAAGAGCTGGTTCGATCACATGC |
| | | AGCTCGTGTCACTTTTGGCTACCCAGAGAAGATTTTCCCTTGTAAGCCCTAGGCC |
| | | TAAGAAGAAAAGAAAGGTGGGTGGTTCTCCTAAGAAAAAGAGAAAGGTCTGAGAA |
| | | TTC |
| 3 | MG153-24_ | GCTAGCGCCACCATGTACCCCTATGATGTACCTGATTATGCTTCTGGCGGCAGCA |
| | cloned_ | GCGGCGGCTCAAGCGGAAGTGAAACACCTGGAACTTCCGAAAGCGCTACGCCTGA |
| | into_ | ATCTTCTGGTGGATCTTCTGGCGGATCTTCCGCATGCATGAAGGGGGGAAAGCAA |
| | untethered_ | AAGATTAGCCAGGACACATGTCTCCAAGAGTCCAGAGCCGAACCAGAGGGTTATG |
| | plasmid | CTGGGGGCCAGACCTTTATCTGGATGACAGAGAACAATCTCACAAACGCTAACAA |
| | | ACCAGAATATGGGCTTCTGGAGCAAATCCTGTCCCCTACAAACCTTAATAGGGCT |
| | | TATAAAGGAGTGCGATCCAATAGGGGGTCTGGAGGTATTGATAAAATGGAGGTTG |
| | | AGTCCCTGAAGGACTATCTGGTTGACAATAAGGAGACGTTAATTCAGTCTATATT |
| | | GGACGGAAAATATAGACCAAACCCTGTGAGGAGGGTGGAGATTAGCAAGGAGAAG |
| | | GGCACACGCAAGCTCGGTATACCTACGGTAGTAGACAGGGTCATCCAGCAGGCCA |
| | | TTGCTCAGGTTTTGTCACCAACTTACGAAAGGCAGTTCAGCGAAAATTCCTATGG |
| | | GTTCCGCCCAGGCAGAAACGCCCATCAGGCTCTGAACCGCTGTAGGGATTACATC |
| | | ACAGACGGCTACATATACGCAGTAGATATGGATCTCGAGAAATTCTTTGATACCG |
| | | TGAATCAGTCAAAACTCATAGAGGTCTTGTCCAGGACAGTGAAAGATGGCCGTGT |
| | | GGTGTCACTTATTCACAAATACCTTGATGCCGGCGTTGTGATCAGAAATAAGTTC |
| | | GAAGAAACAGAGATGGGAGTTCCTCAAGGAAGCCCATTGAGCCCCGTTCTGTCTA |
| | | ACATTATGCTTAACGAACTGGACAAAGAGCTGGAAAAGAGGGGGCACCCATTCGT |
| | | GAGATATGCAGACGACCTGATTATATTCTGCAAATCTAAAAGAAGCGCCGACCGG |
| | | ACGCTGGCTAACACCGTGCCCTATATCGAAAACAAGCTGTTTCTGAAAGTGAACA |
| | | GGGAGAAAACTACTACAGCCTATGTCTCTGGAATTAAATTCCTCGGCTACTCCTT |
| | | TTATGTGTATAAAGGAGAAGGCCGGCTGCGCGTGCACCCGAAAAGCATTGCTAAG |
| | | ATGAAAGAAAGGATTAGAAAACTGACTTCTCGTTCTAATGGCTGGGGCTACGCTC |
| | | GCCGCAAGGAGGCCCTTAGACAGTACATCACAGGCTGGGTCAACTATTTCAAACT |
| | | TGCAGATATGAAAAAACTGTTAGTTAGCGTTGACGAGTGGTACAGACGACGCCTG |
| | | CGTCTCGTTATATGGAAGCAGTGGAAAAGGGTTAGGACGCGGGGGCGGTATCTGA |
| | | TGAAACTGGGGATCGTGAAGCATCAAGCATGGGAGTTTGCCAATACACGCAAAGG |
| | | TTACTGGCACACTGCCAAATCACCAATCCTTAACAGATCTGTAACGTCCAACCGG |
| | | CTCAGACAAGCCGGGTATGTATTCTTCGTGGATTACTACCGGGTAGTGAATGGAA |
| | | TAAACCCTAGGCCTAAGAAGAAAAGAAAGGTGGGTGGTTCTCCTAAGAAAAAGAG |
| | | AAAGGTCTGAGAATTC |
| 4 | MG160-7_ | GCTAGCGCCACCATGTACCCCTATGATGTACCTGATTATGCTTCTGGCGGCAGCA |
| | cloned_ | GCGGCGGCTCAAGCGGAAGTGAAACACCTGGAACTTCCGAAAGCGCTACGCCTGA |
| | into_ | ATCTTCTGGTGGATCTTCTGGGGGATCTTCCGCATGCATGCATAACTTCAAGAGT |
| | untethered_ | AGATTCGAACTCTCCAAGGGCAAATGGGTTTACATCCAGATAGAGGACCTCGCCA |
| | plasmid | ACCATGCCAAGGACCACATCAGACAGATTAGGGACCTGTGGACACCGCCAGAGTA |
| | | CTTTTTCCACCTTCAAAAGGGGGGACATATTGCAGCTCTCAGGCTTCATACGCCT |
| | | AACGAGTGGTACGGCAAGGTCGATCTGTCTAAGTTCTTTAACAACATAACCCGGC |
| | | ACAGGGTGACACGCAGCCTTAAAAGAATTGGCTATTCTTTCCGAGACGCCGAAGA |
| | | GTTCGCCGTTGCCAGCACTGTGCTTGTGAATCACGCAACCAGACGCTATGTGCTG |
| | | CCATACGGGTTTGTCCAGTCTCCCTTGTTGGCGTCCATCAGCCTCGACAAAAGTG |
| | | ATCTTGGAAACTGCCTGAGGCGCCTTCACGAAGAAACTGTCAGCGTCAGTGTATA |
| | | TGTTGATGATATCGTGGTATCCGCAGATAGCGAGCGAGACGTTGCAGAGGCCCTG |
| | | TCCAACATCTATCTGGCAGCGATTAATTCACGGTTTCCTATTAATGAAGAGAAAA |
| | | GTCGAGGTCCCAGCTCAACACTGAATGCCTTTAATATCGAACTGGACATGCACGA |
| | | GTTGGAGATCACTGCTAAACGGTACGAGAAGATGTGTGGTGAAGTCCTGATCAAC |
| | | GGGACCGGACAGGTGTCCGATGCTATTTTGAATTACGTGCAGACCGTGAATCCCG |
| | | CTCAGGCCGAACAAATGCTCCGGGATTTTCCTGGCGTGTTCGGGACACTGTCATC |
| | | TTCCGCTTCTCGCTATGACGCTTCACCTAGGCCTAAGAAGAAAAGAAAGGTGGGT |
| | | GGTTCTCCTAAGAAAAAGAGAAAGGTCTGAGAATTC |
| 5 | MG160-7_ | ATGAAGAGGACCGCTGATGGTTCCGAATTTGAATCTCCTAAGAAGAAACGAAAGG |
| | cloned_ | TTGATAAGAAATATAGTATTGGGTTGGATATTGGTACGAATAGTGTAGGATGGGG |
| | into_ | GGTTATTACTGACGAATATAAAGTCCCCTCTAAGAAGTTTAAAGTCCTCGGTAAT |
| | tethered_ | ACAGATAGGCATAGTATTAAGAAGAATCTTATTGGCGCTCTCTTATTTGATTCCG |
| | plasmid | GTGAGACTGCTGAAGCTACAAGGTTAAAGAGGACTGCTCGCCGGCGCTATACACG |
| | | AAGGAAGAATCGCATATGTTACCTTCAGGAAATATTTAGTAATGAAATGGCAAAA |
| | | GTCGATGATTCATTCTTCCATCGACTTGAGGAATCATTTCTCGTTGAGGAAGACA |
| | | AGAAGCATGAAAGGCATCCAATCTTTGGAAATATAGTCGATGAAGTAGCTTATCA |
| | | TGAGAAATATCCGACTATTTATCATTTGCGAAAGAAGCTTGTCGATTCTACAGAT |
| | | AAAGCGGATCTTAGGCTTATATACCTCGCACTCGCTCATATGATTAAATTTAGGG |
| | | GTCATTTTCTGATTGAAGGGGATCTCAATCCTGATAATTCCGATGTAGATAAGCT |
| | | CTTTATTCAACTTGTACAAACATATAATCAATTATTTGAAGAGAATCCTATTAAT |
| | | GCTTCTGGAGTCGATGCAAAAGCGATTTTGAGCGCTCGCCTTTCCAAAAGTCGCA |
| | | GACTTGAGAACTTGATTGCTCAACTCCCAGGTGAAAAGAAGAACGGACTCTTTGG |
| | | TAATTTGATCGCATTGAGTCTTGGGTTGACTCCAAATTTTAAAAGTAATTTTGAT |
| | | TTGGCTGAAGACGCAAAGCTCCAATTGTCCAAAGATACTTATGATGATGATTTGG |
| | | ATAATTTATTAGCCCAAATTGGAGATCAATATGCTGATCTCTTCCTCGCAGCTAA |
| | | GAATCTCTCAGATGCAATTTTGTTGTCTGATATACTTCGCGTAAATACTGAAATT |
| | | ACAAAAGCTCCTCTCTCCGCAAGCATGATAAAACGGTATGATGAACATCATCAAG |
| | | ATCTTACTCTTCTTAAGGCCTTAGTACGCCAACAACTTCCGGAGAAATATAAGGA |
| | | AATCTTCTTTGATCAATCAAAGAATGGGTATGCAGGGTATATAGATGGCGGCGCG |
| | | TCACAAGAGGAATTTTATAAATTTATAAAACCGATTCTTGAGAAAATGGATGGAA |
| | | CAGAAGAGCTTTTGGTCAAATTAAATAGGGAAGATTTGTTGAGGAAACAAAGAAC |
| | | CTTTGATAATGGGTCTATTCCACATCAAATACATCTTGGCGAATTGCACGCGATA |
| | | CTCAGGCGACAAGAGGACTTCTATCCTTTTCTTAAAGATAATCGCGAGAAAATTG |
| | | AAAAGATTCTCACGTTTCGGATTCCTTATTATGTTGGACCGTTAGCGCGGGGTAA |
| | | TTCACGGTTTGCTTGGATGACTAGGAAATCAGAAGAGACTATTACGCCTTGGAAT |
| | | TTTGAAGAGGTTGTCGATAAAGGAGCCTCAGCTCAATCTTTTATTGAACGAATGA |
| | | CTAATTTTGACAAGAATCTCCCTAATGAAAAGGTCTTGCCTAAACATAGTCTTCT |
| | | CTATGAATATTTTACAGTCTACAATGAATTGACAAAGGTTAAGTATGTCACAGAA |
| | | GGTATGAGGAAACCTGCATTTCTGAGTGGGGAACAAAAGAAAGCGATTGTCGATC |
| | | TCCTCTTTAAAACGAATAGAAAGGTCACGGTAAAACAATTAAAGGAAGATTATTT |
| | | TAAGAAGATTGAATGTTTTGATTCAGTCGAGATATCTGGGGTCGAGGACAGATTT |
| | | AATGCAAGCTTGGGTACCTATCATGACTTGCTCAAGATCATTAAAGATAAAGATT |
| | | TTCTCGATAACGAAGAGAATGAAGATATCCTTGAGGACATAGTCCTTACTCTCAC |
| | | CCTCTTCGAAGATCGGGAAATGATTGAAGAGCGCCTTAAGACATACGCACATCTC |
| | | TTTGATGATAAGGTTATGAAACAACTCAAACGCCGACGGTATACAGGATGGGGAA |
| | | GACTTAGTCGCAAACTCATAAATGGAATAAGGGATAAACAATCAGGAAAGACCAT |
| | | TCTTGACTTTCTTAAATCTGATGGATTTGCAAATCGCAATTTCATGCAACTCATT |
| | | CATGATGATTCCCTCACATTCAAGGAAGATATACAAAAGGCACAAGTCAGTGGGC |
| | | AAGGGGACTCCCTTCATGAACATATCGCAAACCTTGCTGGTTCTCCTGCAATCAA |
| | | GAAGGGAATACTCCAAACTGTAAAAGTCGTCGATGAACTTGTTAAGGTAATGGGA |
| | | CGCCATAAACCGGAAAATATTGTAATAGAGATGGCGCGAGAAAATCAAACGACTC |
| | | AAAAGGGCCAGAAGAATAGCAGAGAAAGGATGAAACGCATAGAGGAAGGGATAAA |
| | | GGAATTGGGGTCCCAAATTTTGAAGGAGCATCCAGTAGAGAATACTCAATTGCAA |
| | | AATGAGAAACTCTATCTTTATTATCTCCAAAACGGCAGGGACATGTATGTCGATC |
| | | AAGAGTTAGATATAAATCGCCTTAGTGATTATGACGTAGATGCAATTGTCCCCCA |
| | | ATCCTTCCTTAAAGATGATAGTATTGATAATAAAGTCTTGACTAGGAGTGATAAG |
| | | AATCGCGGGAAAAGTGATAATGTACCATCTGAGGAAGTAGTAAAGAAAATGAAGA |
| | | ATTATTGGCGACAACTCCTCAATGCAAAATTGATCACGCAACGAAAATTTGATAA |
| | | CTTGACGAAAGCTGAACGGGGGGGACTCTCCGAGCTTGACAAAGCAGGGTTTATT |
| | | AAACGCCAATTGGTCGAGACTAGACAAATTACGAAACATGTTGCTCAAATTCTTG |
| | | ATAGCAGAATGAATACCAAATATGATGAAAACGATAAATTGATTCGAGAGGTCAA |
| | | GGTAATTACACTCAAATCAAAATTAGTCTCTGACTTTAGGAAAGACTTTCAATTC |
| | | TATAAGGTAAGGGAAATTAATAATTATCATCATGCGCATGATGCATATTTGAATG |
| | | CTGTGGTCGGCACAGCGTTGATTAAGAAATATCCCAAATTAGAGTCAGAATTTGT |
| | | TTATGGAGATTATAAAGTCTATGATGTTAGGAAAATGATTGCAAAGTCTGAGCAA |
| | | GAGATTGGGAAAGCGACAGCAAAATATTTCTTTTATAGTAATATTATGAACTTCT |
| | | TTAAAACGGAAATCACTCTCGCTAATGGGGAAATTCGCAAAAGACCCCTTATTGA |
| | | AACTAATGGTGAGACAGGCGAAATTGTTTGGGACAAAGGACGTGACTTCGCTACA |
| | | GTCCGCAAGGTTCTTTCTATGCCACAGGTAAACATTGTTAAGAAAACTGAAGTCC |
| | | AAACTGGCGGTTTCTCCAAGGAAAGCATTCTCCCTAAACGCAATAGTGACAAATT |
| | | GATTGCAAGGAAGAAGGATTGGGATCCAAAGAAATATGGTGGGTTTGATTCACCG |
| | | ACAGTTGCGTACAGTGTCCTTGTTGTAGCTAAGGTAGAGAAAGGAAAATCCAAGA |
| | | AGCTTAAATCAGTTAAGGAATTATTAGGCATTACAATTATGGAGAGGTCCAGTTT |
| | | TGAGAAGAACCCGATAGATTTCTTAGAGGCTAAAGGGTACAAAGAGGTTAAGAAA |
| | | GATCTTATTATAAAACTTCCAAAATATAGTCTCTTTGAACTGGAGAATGGACGAA |
| | | AACGAATGCTTGCAAGTGCTGGAGAGTTACAAAAGGGCAATGAGCTCGCTTTACC |
| | | AAGCAAGTACGTAAATTTTCTCTATCTTGCATCCCATTACGAAAAGTTGAAAGGT |
| | | AGTCCAGAAGACAACGAACAAAAGCAATTATTCGTAGAGCAACATAAACATTATC |
| | | TTGATGAAATTATTGAACAGATTAGTGAATTTTCAAAACGCGTTATACTCGCAGA |
| | | TGCCAACTTAGATAAGGTACTCTCTGCTTATAATAAACATAGGGACAAACCTATA |
| | | CGAGAACAAGCGGAAAACATTATTCATTTGTTCACACTCACTAACCTCGGCGCTC |
| | | CAGCTGCTTTTAAATATTTCGATACTACAATAGATAGAAAACGTTATACTTCTAC |
| | | AAAGGAAGTCTTAGATGCTACACTCATTCATCAAAGTATTACAGGATTATATGAA |
| | | ACTCGTATAGATCTCTCACAACTCGGCGGGGATTCTGGCGGCAGCAGCGGCGGCT |
| | | CAAGCGGAAGTGAAACACCTGGAACTTCCGAAAGCGCTACGCCTGAATCTTCTGG |
| | | TGGATCTTCTGGCGGATCTTCCGCATGCATGCATAACTTCAAGAGTAGATTCGAA |
| | | CTCTCCAAGGGCAAATGGGTTTACATCCAGATAGAGGACCTCGCCAACCATGCCA |
| | | AGGACCACATCAGACAGATTAGGGACCTGTGGACACCGCCAGAGTACTTTTTCCA |
| | | CCTTCAAAAGGGCGGACATATTGCAGCTCTCAGGCTTCATACGCCTAACGAGTGG |
| | | TACGGCAAGGTCGATCTGTCTAAGTTCTTTAACAACATAACCCGGCACAGGGTGA |
| | | CACGCAGCCTTAAAAGAATTGGCTATTCTTTCCGAGACGCCGAAGAGTTCGCCGT |
| | | TGCCAGCACTGTGCTTGTGAATCACGCAACCAGACGCTATGTGCTGCCATACGGG |
| | | TTTGTCCAGTCTCCCTTGTTGGCGTCCATCAGCCTCGACAAAAGTGATCTTGGAA |
| | | ACTGCCTGAGGCGCCTTCACGAAGAAACTGTCAGCGTCAGTGTATATGTTGATGA |
| | | TATCGTGGTATCCGCAGATAGCGAGCGAGACGTTGCAGAGGCCCTGTCCAACATC |
| | | TATCTGGCAGCGATTAATTCACGGTTTCCTATTAATGAAGAGAAAAGTCGAGGTC |
| | | CCAGCTCAACACTGAATGCCTTTAATATCGAACTGGACATGCACGAGTTGGAGAT |
| | | CACTGCTAAACGGTACGAGAAGATGTGTGGTGAAGTCCTGATCAACGGGACCGGA |
| | | CAGGTGTCCGATGCTATTTTGAATTACGTGCAGACCGTGAATCCCGCTCAGGCCG |
| | | AACAAATGCTCCGGGATTTTCCTGGCGTGTTCGGGACACTGTCATCTTCCGCTTC |
| | | TCGCTATGACGCTTCA |
| 6 | VEGFA_ | mG*mA*mU*rUrCrUrGrCrArGrGrCrCrArGrArUrGrArGrUrUrUrUrArG |
| | G_ | rArGrCrUrArGrArArArUrArGrCrArArGrUrUrArArArArUrArArGrGr |
| | to_ | CrUrArGrUrCrCrGrUrUrArUrCrArArCrUrUrGrArArArArArGrUrGrG |
| | T_ | rGrArCrCrGrArGrUrCrGrGrUrCrCrArArUrGrUrGrCrCrArUrCrUrGr |
| | change_ | GrArGrCrArCrUrCrArUrCrUrUrU*mU*mU*mU |
| | PBS_ | |
| | 2 | |
| 7 | VEGFA_ | mG*mA*mU*rGrUrCrUrGrCrArGrGrCrCrArGrArUrGrArGrUrUrUrUrA |
| | G_ | rGrArGrCrUrArGrArArArUrArGrCrArArGrUrUrArArArArUrArArGr |
| | to_ | GrCrUrArGrUrCrCrGrUrUrArUrCrArArCrUrUrGrArArArArArGrUrG |
| | T_ | rGrCrArCrCrGrArGrUrCrGrGrUrGrCrArArUrGrUrGrCrCrArUrCrUr |
| | change_ | GrGrArGrCrArCrUrCrArUrCrUrGrUrUrU*mU*mU*mU |
| | PBS_ | |
| | 4 | |
| 8 | VEGFA_ | mG*mA*mU*rGrUrCrUrGrCrArGrGrCrCrArGrArUrGrArGrUrUrUrUrA |
| | G_ | rGrArGrCrUrArGrArArArUrArGrCrArArGrUrUrArArArArUrArArGr |
| | to_ | GrCrUrArGrUrCrCrGrUrUrArUrCrArArCrUrUrGrArArArArArGrUrG |
| | T_ | rGrCrArCrCrGrArGrUrCrGrGrUrGrCxArArUrGrUrGrCrCrArUrCrUr |
| | change_ | GrGrArGrCrArCrUrCrArUrCrUrGrGrCrUrUrU*mU*mU*mU |
| | PBS_ | |
| | 6 | |
| 9 | VEGFA_ | mG*mA*mU*rGrUrCrUrGrCrArGrGrCrCrArGrArUrGrArGrUrUrUrUrA |
| | G_ | rGrArGrCrUrArGrArArArUrArGrCrArArGrUrUrArArArArUrArArGr |
| | to_ | GrCrUrArGrUrCrCrGrUrUrArUrCrArArCrUrUrGrArArArArArGrUrG |
| | T_ | rGrCrArCrCrGrArGrUrCrGrGrUrGrCrArArUrGrUrGrCrCrArUrCrUr |
| | change_ | GrGrArGrCrArCrUrCrArUrCrUrGrGrCrCrUrUrUrU*mU*mU*mU |
| | PBS_ | |
| | 8 | |
| 10 | VEGFA_ | mG*mA*mU*rGrUrCrUrGrCrArGrGrCrCrArGrArUrGrArGrUrUrUrUrA |
| | G_ | rGrArGrCrUrArGrArArArUrArGrCrArArGrUrUrArArArArUrArArGr |
| | to_ | GrCrUrArGrUrCrCrGrUrUrArUrCrArArCrUrUrGrArArArArArGrUrG |
| | T_ | rGrCrArCrCrGrArGrUrCrGrGrUrGrCrArArUrGrUrGrCrCrArUrCrUr |
| | change_ | GrGrArGrCrArCrUrCrArUrCrUrGrGrCrCrUrGrCrUrUrU*mU*mU*mU |
| | PBS_ | |
| | 10 | |
| 11 | VEGFA_ | mG*mA*mU*rGrUrCrUrGrCrArGrGrCrCrArGrArUrGrArGrUrUrUrUrA |
| | G_ | rGrArGrCrUrArGrArArArUrArGrCrArArGrUrUrArArArArUrArArGr |
| | to_ | GrCrUrArGrUrCrCrGrUrUrArUrCrArArCrUrUrGrArArArArArGrUrG |
| | T_ | rGrCrArCrCrGrArGrUrCrGrGrUrGrCrArArUrGrUrGrCrCrArUrCrUr |
| | change_ | GrGrArGrCrArCrUrCrArUrCrUrGrGrCrCrUrGrCrArGrArUrUrU*mU* |
| | PBS_ | mU*mU |
| | 13 | |
| 12 | VEGFA_ | mG*mA*mU*rGrUrCrUrGrCrArGrGrCrCrArGrArUrGrArGrUrUrUrUrA |
| | G_ | rGrArGrCrUrArGrArArArUrArGrCrArArGrUrUrArArArArUrArArGr |
| | to_ | GrCrUrArGrUrCrCrGrUrUrArUrCrArArCrUrUrGrArArArArArGrUrG |
| | T_ | rGrGrArCrCrGrArGrUrCrGrGrUrCrCrArArUrGrUrGrCrCrArUrCrUr |
| | change_ | GrGrArGrCrArCrUrCrArUrCrUrGrGrCrCrUrGrCrArGrArCrArUrUrU |
| | PBS_ | rU*mU*mU*mU |
| | 16 | |
| 13 | VEGFA_ | mG*mA*mU*rGrUrCrUrGrCrArGrGrCrCrArGrArUrGrArGrUrUrUrUrA |
| | G_ | rGrArGrCrUrArGrArArArUrArGrCrArArGrUrUrArArArArUrArArGr |
| | to_ | GrCrUrArGrUrCrCrGrUrUrArUrCrArArCrUrUrGrArArArArArGrUrG |
| | T_ | rGrCrArCrCrGrArGrUrCrGrGrUrGrCrArArUrGrUrGrCrCrArUrCrUr |
| | change_ | GrGrArGrCrArCrUrCrArUrCrUrGrGrCrCrUrGrCrArGrArCrArUrCrA |
| | PBS_ | rArArUrUrU*mU*mU*mU |
| | 20 | |
| 14 | VEGFA_ | mG*mA*mU*rGrUrCrUrGrCrArGrGrCrCrArGrArUrGrArGrUrUrUrUrA |
| | deletion_ | rGrArGrCrUrArGrArArArUrArGrCrArArGrUrUrArArArArUrArArGr |
| | change_ | GrCrUrArGrUrCrCrGrUrUrArUrCrArArCrUrUrGrArArArArArGrUrG |
| | PBS_ | rGrCrArCrCrGrArGrUrCrGrGrUrGrCrGrUrGrUrGrUrCrCrCrUrCrUr |
| | 2 | GrArCrArArUrGrUrGrCrUrCrUrUrUrU*mU*mU*mU |
| 15 | VEGFA_ | mG*mA*mU*rGrUrCrUrGrCrArGrGrCrCrArGrArUrGrArGrUrUrUrUrA |
| | deletion_ | rGrArGrCrUrArGrArArArUrArGrCrArArGrUrUrArArArArUrArArGr |
| | change_ | GrCrUrArGrUrCrCrGrUrUrArUrCrArArCrUrUrGrArArArArArGrUrG |
| | PBS_ | rGrCrArCrCrGrArGrUrCrGrGrUrGrCrGrUrGrUrGrUrCrCrCrUrCrUr |
| | 4 | GrArCrArArUrGrUrGrCrUrCrUrGrUrUrUrU*mU*mU*mU |
| 16 | VEGFA_ | mG*mA*mU**GrUrCrUrGrCrArGrGrCrCrArGrArUrGrArGrUrUrUrUrA |
| | deletion_ | rGrArGrCrUrArGrArArArUrArGrCrArArGrUrUrArArArArUrArArGr |
| | change_ | GrCrUrArGrUrCrCrGrUrUrArUrCrArArCrUrUrGrArArArArArGrUrG |
| | PBS_ | rGrCrArCrCrGrArGrUrCrGrGrUrGrCrGrUrGrUrGrUrCrCrCrUrCrUr |
| | 6 | GrArCrArArUrGrUrGrCrUrCrUrGrGrCrUrUrUrU*mU*mU*mU |
| 17 | VEGFA_ | mG*mA*mU**GrUrCrUrGrCrArGrGrCrCrArGrArUrGrArGrUrUrUrUrA |
| | deletion_ | rGrArGrCrUrArGrArArArUrArGrCrArArGrUrUrArArArArUrArArGr |
| | change_ | GrCrUrArGrUrCrCrGrUrUrArUrCrArArCrUrUrGrArArArArArGrUrG |
| | PBS_ | rGrCrArCrCrGrArGrUrCrGrGrUrGrCrGrUrGrUrGrUrCrCrCrUrCrUr |
| | 8 | GrArCrArArUrGrUrGrCrUrCrUrGrGrCrCrUrUrUrUrU*mU*mU*mU |
| 18 | VEGFA_ | mG*mA*mU*rGrUrCrUrGrCrArGrGrCrCrArGrArUrGrArGrUrUrUrUrA |
| | deletion_ | rGrArGrCrUrArGrArArArUrArGrCrArArGrUrUrArArArArUrArArGr |
| | change_ | GrCrUrArGrUrCrCrGrUrUrArUrCrArArCrUrUrGrArArArArArGrUrG |
| | PBS_ | rGrCrArCrCrGrArGrUrCrGrGrUrGrCrGrUrGrUrGrUrCrCrCrUrCrUr |
| | 10 | GrArCrArArUrGrUrGrCrUrCrUrGrGrCrCrUrGrCrUrUrUrU*mU*mU*m |
| | | U |
| 19 | VEGFA_ | mG*mA*mU*rGrUrCrUrGrCrArGrGrCrCrArGrArUrGrArGrUrUrUrUrA |
| | deletion_ | rGrArGrCrUrArGrArArArUrArGrCrArArGrUrUrArArArArUrArArGr |
| | change_ | GrCrUrArGrUrCrCrGrUrUrArUrCrArArCrUrUrGrArArArArArGrUrG |
| | PBS_ | rGrCrArCrCrGrArGrUrCrGrGrUrGrCrGrUrGrUrGrUrCrCrCrUrCrUr |
| | 13 | GrArCrArArUrGrUrGrCrUrCrUrGrGrCrCrUrGrCrArGrArUrUrUrU*m |
| | | U*mU*mU |
| 20 | VEGFA_ | mG*mA*mU*rGrUrCrUrGrCrArGrGrCrCrArGrArUrGrArGrUrUrUrUrA |
| | deletion_ | rGrArGrCrUrArGrArArArUrArGrCrArArGrUrUrArArArArUrArArGr |
| | change_ | GrCrUrArGrUrCrCrGrUrUrArUrCrArArCrUrUrGrArArArArArGrUrG |
| | PBS_ | rGrCrArCrCrGrArGrUrCrGrGrUrGrCrGrUrGrUrGrUrCrCrCrUrCrUr |
| | 16 | GrArCrArArUrGrUrGrCrUrCrUrGrGrCrCrUrGrCrArGrArCrArUrUrU |
| | | rUrU*mU*mU*mU |
| 21 | VEGFA_ | mG*mA*mU*rGrUrCrUrGrCrArGrGrCrCrArGrArUrGrArGrUrUrUrUrA |
| | deletion_ | rGrArGrCrUrArGrArArArUrArGrCrArArGrUrUrArArArArUrArArGr |
| | change_ | GrCrUrArGrUrCrCrGrUrUrArUrCrArArCrUrUrGrArArArArArGrUrG |
| | PBS_ | rGrCrArCrCrGrArGrUrCrGrGrUrGrCrGrUrGrUrGrUrCrCrCrUrCrUr |
| | 20 | GrArCrArArUrGrUrGrCrUrCrUrGrGrCrCrUrGrCrArGrArCrArUrCrA |
| | | rArArUrUrUrU*mU*mU*mU |
| 22 | VEGFA_ | mG*mA*mU*rGrUrCrUrGrCrArGrGrCrCrArGrArUrGrArGrUrUrUrUrA |
| | insertion_ | rGrArGrCrUrArGrArArArUrArGrCrArArGrUrUrArArArArUrArArGr |
| | change_ | GrCrUrArGrUrCrCrGrUrUrArUrCrArArCrUrUrGrArArArArArGrUrG |
| | PBS_ | rGrCrArCrCrGrArGrUrCrGrGrUrGrCrArArUrGrUrGrCrCrArUrCrUr |
| | 2 | GrGrArGrCrArCrUrCrArCrUrUrArUrCrGrUrCrGrUrCrArUrCrCrUrU |
| | | rGrUrArArUrCrUrCrUrUrUrU*mU*mU*mU |
| 23 | VEGFA_ | mG*mA*mU*rGrUrCrUrGrCrArGrGrCrCrArGrArUrGrArGrUrUrUrUrA |
| | insertion_ | rGrArGrCrUrArGrArArArUrArGrCrArArGrUrUrArArArArUrArArGr |
| | change_ | GrCrUrArGrUrCrCrGrUrUrArUrCrArArCrUrUrGrArArArArArGrUrG |
| | PBS_ | rGrCrArCrCrGrArGrUrCrGrGrUrGrCrArArUrGrUrGrCrCrArUrCrUr |
| | 4 | GrGrArGrCrArCrUrCrArCrUrUrArUrCrGrUrCrGrUrCrArUrCrCrUrU |
| | | rGrUrArArUrCrUrCrUrGrUrUrUrU*mU*mU*mU |
| 24 | VEGFA_ | mG*mA*mU*rGrUrCrUrGrCrArGrGrCrCrArGrArUrGrArGrUrUrUrUrA |
| | insertion_ | rGrArGrCrUrArGrArArArUrArGrCrArArGrUrUrArArArArUrArArGr |
| | change_ | GrCrUrArGrUrCrCrGrUrUrArUrCrArArCrUrUrGrArArArArArGrUrG |
| | PBS_ | rGrCrArCrCrGrArGrUrCrGrGrUrGrCrArArUrGrUrGrCrCrArUrCrUr |
| | 6 | GrGrArGrCrArCrUrCrArCrUrUrArUrCrGrUrCrGrUrCrArUrCrCrUrU |
| | | rGrUrArArUrCrUrCrUrGrGrCrUrUrUrUmUmU*mU |
| 25 | VEGFA_ | mG*mA*mU*rGrUrCrUrGrCrArGrGrCrCrArGrArUrGrArGrUrUrUrUrA |
| | insertion_ | rGrArGrCrUrArGrArArArUrArGrCrArArGrUrUrArArArArUrArArGr |
| | change_ | GrCrUrArGrUrCrCrGrUrUrArUrCrArArCrUrUrGrArArArArArGrUrG |
| | PBS_ | rGrCrArCrCrGrArGrUrCrGrGrUrGrCrArArUrGrUrGrCrCrArUrCrUr |
| | 8 | GrGrArGrCrArCrUrCrArCrUrUrArUrCrGrUrCrGrUrCrArUrCrCrUrU |
| | | rGrUrArArUrCrUrCrUrGrGrCrCrUrUrUrUrU*mU*mU*mU |
| 26 | VEGFA_ | mG*mA*mU*rGrUrCrUrGrCrArGrGrCrCrArGrArUrGrArGrUrUrUrUrA |
| | insertion_ | rGrArGrCrUrArGrArArArUrArGrCrArArGrUrUrArArArArUrArArGr |
| | change_ | GrCrUrArGrUrCrCrGrUrUrArUrCrArArCrUrUrGrArArArArArGrUrG |
| | PBS_ | rGrCrArCrCrGrArGrUrCrGrGrUrGrCrArArUrGrUrGrCrCrArUrCrUr |
| | 10 | GrGrArGrCrArCrUrCrArCrUrUrArUrCrGrUrCrGrUrCrArUrCrCrUrU |
| | | rGrUrArArUrCrUrCrUrGrGrCrCrUrGrCrUrUrUrU*mU*mU*mU |
| 27 | VEGFA_ | mG*mA*mU*rGrUrCrUrGrCrArGrGrCrCrArGrArUrGrArGrUrUrUrUrA |
| | insertion_ | rGrArGrCrUrArGrArArArUrArGrCrArArGrUrUrArArArArUrArArGr |
| | change_ | GrCrUrArGrUrCrCrGrUrUrArUrCrArArCrUrUrGrArArArArArGrUrG |
| | PBS_ | rGrCrArCrCrGrArGrUrCrGrGrUrGrCrArArUrGrUrGrCrCrArUrCrUr |
| | 13 | GrGrArGrCrArCrUrCrArCrUrUrArUrCrGrUrCrGrUrCrArUrCrCrUrU |
| | | rGrUrArArUrCrUrCrUrGrGrCrCrUrGrCrArGrArUrUrUrUmU*mU*mU |
| 28 | VEGFA_ | mG*mA*mU*rGrUrCrUrGrCrArGrGrCrCrArGrArUrGrArGrUrUrUrUrA |
| | insertion_ | rGrArGrCrUrArGrArArArUrArGrCrArArGrUrUrArArArArUrArArGr |
| | change_ | GrCrUrArGrUrCrCrGrUrUrArUrCrArArCrUrUrGrArArArArArGrUrG |
| | PBS_ | rGrCrArCrCrGrArGrUrCrGrGrUrGrCrArArUrGrUrGrCrCrArUrCrUr |
| | 16 | GrGrArGrCrArCrUrCrArCrUrUrArUrCrGrUrCrGrUrCrArUrCrCrUrU |
| | | rGrUrArArUrCrUrCrUrGrGrCrCrUrGrCrArGrArCrArUrUrUrUrU*mU |
| | | *mU*mU |
| 29 | VEGFA_ | mG*mA*mU*rGrUrCrUrGrCrArGrGrCrCrArGrArUrGrArGrUrUrUrUrA |
| | insertion_ | rGrArGrCrUrArGrArArArUrArGrCrArArGrUrUrArArArArUrArArGr |
| | change_ | GrCrUrArGrUrCrCrGrUrUrArUrCrArArCrUrUrGrArArArArArGrUrG |
| | PBS_ | rGrCrArCrCrGrArGrUrCrGrGrUrGrCrArArUrGrUrGrCrCrArUrCrUr |
| | 20 | GrGrArGrCrArCrUrCrArCrUrUrArUrCrGrUrCrGrUrCrArUrCrCrUrU |
| | | rGrUrArArUrCrUrCrUrGrGrCrCrUrGrCrArGrArCrArUrCrArArArUr |
| | | UrUrU*mU*mU*mU |
| 30 | VEGFA_ | GCTCTTCCGATCTNNNNNAAATTCTTCTCCCCTGGGAAGC |
| | forward_ | |
| | primer | |
| 31 | VEGFA_ | GCTCTTCCGATCTNNNNNTGGACCCCCTATTTCTGACCTC |
| | reverse_ | |
| | primer | |
| 32 | VEGFA_ | AAATTCTTCTCCCCTGGGAAGCATCCCTGGACACTTCCCAAAGGACCCCAGTCAC |
| | target_ | TCCAGCCTGTTGGCTGCCGCTCACTTTGATGTCTGCAGGCCAGATGAGGGCTCCA |
| | amplified | GATGGCACATTGTCAGAGGGACACACTGTGGCCCCTGTGCCCAGCCCTGGGCTCT |
| | | CTGTACATGAAGCAACTCCAGTCCCAAATATGTAGCTGTTTGGGAGGTCAGAAAT |
| | | AGGGGGTCCA |
| 33 | linker_ | TCTGGCGGCAGCAGCGGCGGCTCAAGCGGAAGTGAAACACCTGGAACTTCCGAAA |
| | 1 | GCGCTACGCCTGAATCTTCTGGTGGATCTTCTGGCGGATCTTCCGCATGC |
| | for | |
| | tethered | |
| | RT | |
| 34 | MG3-6-mRNA- | AAAAGCCAGCTCCAGCAGGCGCTGCTCACTCCTCCCCATCCTCTCCCTCTGTCCC |
| | wild-type | TCTGTCCCTCTGACCCTGCACTGTCCCAGCACCATGGCCCCCAAGAAGAAGCGGA |
| | | AAGTTACCGGTGGCGGAGGCAGCAGCACCGACATGAAGAACTACCGCATCGGAGT |
| | | GGACGTGGGCGACAGAAGCGTGGGACTGGCCGCCATCGAATTCGACGACGACGGG |
| | | CTGCCCATCCAGAAACTGGCACTGGTGACCTTCCGGCACGACGGCGGCCTGGACC |
| | | CCACGAAGAACAAGACACCGATGAGCAGGAAAGAGACGAGGGGAATCGCACGCAG |
| | | AACAATGAGGATGAACCGAGAAAGGAAGAGGCGCCTGAGAAACCTGGACAACGTG |
| | | CTGGAAAACCTGGGCTACAGCGTGCCCGAGGGCCCCGAGCCCGAAACCTACGAAG |
| | | CATGGACCAGCCGGGCACTGCTGGCCAGCATCAAGCTGGCCAGCGCAGACGAACT |
| | | GAACGAACACCTGGTGCGAGCGGTGAGGCACATGGCACGCCACCGAGGATGGGCC |
| | | AACCCCTGGTGGAGCCTGGACCAGCTGGAAAAGGCAAGCCAGGAGCCCAGCGAAA |
| | | CATTCGAGATCATCCTGGCCCGGGCCAGGGAGCTGTTCGGGGAGAAAGTGCCCGC |
| | | AAACCCCACCCTGGGCATGCTGGGAGCACTGGCCGCGAACAACGAAGTGCTGCTG |
| | | CGCCCGAGGGACGAGAAGAAGAGGAAGACCGGATACGTGAGAGGGACACCCCTGA |
| | | TGTTCGCACAAGTGCGACAAGGCGACCAACTGGCAGAACTGAGGCGAATCTGCGA |
| | | GGTGCAAGGGATCGAAGACCAATACGAGGCACTGCGGCTGGGCGTGTTCGACCAC |
| | | AAACACCCCTACGTGCCAAAAGAGAGAGTGGGAAAAGACCCGCTGAACCCCAGCA |
| | | CAAACCGCACAATCAGGGCGAGCCTGGAGTTCCAAGAATTCAGAATCCTGGACAG |
| | | CGTGGCCAACCTGAGAGTGAGAATCGGCAGCAGGGCGAAAAGAGAGCTGACCGAG |
| | | GCCGAATACGACGCAGCCGTGGAATTCCTGATGGACTACGCGGACAAGGAGCAGC |
| | | CCAGCTGGGCCGACGTGGCCGAAAAGATCGGCGTGCCCGGAAACCGACTGGTGGC |
| | | CCCCGTGCTGGAAGACGTGCAACAGAAAACCGCACCCTACGACAGAAGCAGCGCA |
| | | GCATTCGAGAAAGCGATGGGGAAGAAGACCGAGGCAAGACAATGGTGGGAGAGCA |
| | | CCGACGACGACCAACTGAGAAGCCTGCTGATCGCATTCCTGGTGGACGCCACGAA |
| | | CGACACAGAAGAGGCCGCCGCCGAGGGGGGACTGAGCGAACTGTACAAGAGCTGG |
| | | CCCGCAGAAGAGAGAGAGGCCCTGAGCAACATCGACTTCGAGAAAGGAAGGGTGG |
| | | CATACAGCCAAGAGACCCTGAGCAAACTGAGCGAATACATGCACGAATACAGAGT |
| | | GGGACTGCACGAGGCCCGCAAAGCAGTGTTCGGGGGGACGACACCTGGAGACCAC |
| | | CACTGGACAAACTGGAGGAGCCAACAGGCCAACCCGCAGTGGACAGAGTGCTGAC |
| | | AATCCTGCGACGGTTCGTGCTGGACTGCGAGAGGCAATGGGGCCGCCCCAGGGCC |
| | | ATCACCGTGGAACACACCAGAACCGGCCTGATGGGGCCGACCCAGCGCCAGAAAA |
| | | TCCTGAACGAACAAAAGAAGAACCGGGCCGACAACGAGCGAATCAGAGACGAGCT |
| | | GAGGGAAAGCGGGGTGGACAACCCCAGCAGGGCCGAAGTGCGAAGACACCTGATC |
| | | GTGCAAGAACAAGAGTGCCAATGCCTGTACTGCGGAACCATGATCACCACAACCA |
| | | CGAGCGAGCTGGACCACATCGTGCCCCGCGCCGGAGGAGGCAGCAGCCGAAGAGA |
| | | AAACCTGGCCGCAGTGTGCAGGGCCTGCAACGCGAAGAAGAAGCGCGAGCTGTTC |
| | | TACGCCTGGGCAGGCCCGGTGAAAAGCCAAGAAACAATCGAGCGCGTGCGCCAAC |
| | | TGAAAGCATTCAAAGACAGCAAGAAGGCGAAAATGTTCAAGAACCAGATCCGGCG |
| | | ACTGAACCAAACCGAGGCAGACGAGCCAATCGACGAGAGAAGCCTGGCCAGCACC |
| | | AGCTACGCAGCCGTGGCAGTGAGGGAACGCCTGGAGCAACACTTCAACGAAGGGC |
| | | TGGCCCTGGACGACAAAAGCCGGGTGGTGCTGGACGTCTACGCGGGAGCCGTGAC |
| | | CAGGGAGAGCCGCAGAGCAGGCGGGATCGACGAAAGAATCCTGCTGCGGGGCGAG |
| | | CGCGACAAGAACAGATTCGACGTGCGGCACCACGCCGTGGACGCGGCCGTGATGA |
| | | CCCTGCTGAACCGCAGCGTGGCCCTGACCCTGGAGCAACGCAGCCAACTGCGACG |
| | | CACATTCTACGAACAAGGCCTGGACAAACTGGACCGGAACCAACTGAAACCCGAG |
| | | GAAGACTGGCGAGACTTCACAGGGCTGGCCCCCGCCAGCCAAGAGAAGTTCCTGG |
| | | AATGGCGAAAGGCGGCAACCATCCTGGGCGACCTGCTGGCCGAGGCGATCGAGGA |
| | | CGACAGCATCGCAGTGGTGAGCCCCCTGAGACTGAGGCCACAAAACGGCAGCGTG |
| | | CACCTGGAGACAATCAGCGCCGTGAAGAAACAGACACTGGGAAGCGACTGGCCCG |
| | | CCGACGCCGTGAAAAGGATCGTGGACCCAGAGATCTACCTGGCCATGAAAGACGC |
| | | ACTGGGCAAGCTGAAAGAACTGCCAGAGGACAGCGCACGGAGCCTGGAGCTGCCG |
| | | GACGGCAGGTTCGTGGAGGCCGACGACGAAGTGCTGTTCTTCCCCGAAAACGCCG |
| | | CCAGCATCCTGACCCCCAGAGGGGTGGCCGAAATCGGCGGCAGCATCCACCACGC |
| | | GCGGCTGTACGGCTGGCTGACCAAGAAAGGGGAGCTGAAAGTGGGCATGCTGAGG |
| | | GTGTACGGAGCAGAGTTCCCCTGGCTGATGAGGGAAAGCGGCAGCAGAAACGTGC |
| | | TGAGCATGCCCATCCACCGCGGCAGCCAGAGCTTCAGGGACATGCAAGACACCAC |
| | | CAGAAAAGCCGTGGAAAGCGGAGAAGCCGTGGAATTCGCCTGGATCACACAGAAC |
| | | GACGAACTGGAATTCGACCCGGACGACTACATCGCCCACGGCGGAAAAGACGAGC |
| | | TGAGACAATTCCTGGGGTTCATGCCCGAGTGCCGCTGGCGAGTGGACGGCTTCAA |
| | | GAAGAACTACCAGATCCGCATCCGACCAGCCATGCTGAGCCGCGAGCAGCTGCCC |
| | | AGCGACATCCAACGCAGGCTGGAAAGCAAAACACTGACCAAGAACGAGAGCCTGC |
| | | TGCTGAAAGCACTGGACACCGGGCTGGTGGTGGCAATCGGCGGCCTGCTGCCCCT |
| | | GGAAACACTGAAGGTGATCCGGAGAAACAACCTGGGCTTCCCACGATGGCGCGGG |
| | | AACGGGAACCTGCCGACCAGCTTCGAAGTGCGGAGCAGCGCACTGAGAGCACTGG |
| | | GCGTGGAAGGATCTGGCGGAAAAAGACCTGCCGCCACAAAGAAAGCCGGACAGGC |
| | | CAAGAAAAAGAAGTGACCACACCCCCATTCCCCCACTCCAGATAGAACTTCAGTT |
| | | ATATCTCACGTGTCTGGAGTT |
| 35 | AAVS1_ | GGACAGCATGTTTGCTGCCTCCAGGGATCCTGTGTCCCCGAGCTGGGACCACCTT |
| | target_ | ATATTCCCAGGGCCGGTTAATGTGGCTCTGGTTCTGGGTACTTTTATCTGTCCCC |
| | amplified | TCCACCCCACAGTGGGGCCACTAGGGACAGGATTGGTGACAGAAAAGCCCCCATC |
| | | CTTAGGCCTCCTCCTTCCTAGTCTCCTGATATTCGTCTAACCCCCACCTCCTGTT |
| | | AGGCAGATTCCTTATCTGGTGACACACCCCCATTTCCTGGAGCCATCTCTCTCCT |
| | | TGCCAGAACCTCTAAGGTTTGCTTACGATGGAGCCAGAGAGGATCCTGGGAGGGA |
| | | GACTTGGCAGGGGGGGGAGGGAAGGGGGGGATGCGTGACCTGCCCGGTTCTCAGT |
| | | GGCCACCCTGCGCTACCCTCTCCCAGAACCTGAGCTGCTCTGACGCGGCTGTCTG |
| | | GTGCGTTTCACTGATCCTGGTGCTGCAGCTTCCTTACACTTCCCAAGAGGAGAAG |
| | | CAGTTTGGAA |
| 36 | B2M_ | GGGCTAGTCCAGGGCTGGATCTCGGGGAAGCGGCGGGGTGGCCTGGGAGTGGGGA |
| | target_ | AGGGGGTGCGCACCCGGGACGCGCGCTACTTGCCCCTTTCGGGGGGGAGCAGGGG |
| | amplified | AGACCTTTGGCCTACGGCGACGGGAGGGTCGGGACAAAGTTTAGGGCGTCGATAA |
| | | GCGTCAGAGCGCCGAGGTTGGGGGAGGGTTTCTCTTCCGCTCTTTCGCGGGGCCT |
| | | CTGGCTCCCCCAGCGCAGCTGGAGTGGGGGACGGGTAGGCTCGTCCCAAAGGCGC |
| | | GGCGCTGAGGTTTGTGAACGCGTGGAGGGGC |
| 37 | CD5_ | GGCTGCCTGGGTCAGGGTCCTCTGGGAAGCCCCTGCAGTGCCCCAGAAGGGACGA |
| | target_ | AGCTCACAAGGGGCAAGGCAGGCAGCCCACGGGGCAGGAGGGAGCTCAACTGGGC |
| | amplified | GTCCTAGGGAGAGGGCAGTGAGGGGTGCCAGTGGGGAACCCCTCCCAGCCTGACC |
| | | CCCACCACACCTTTCTGACCCCCAGATTTCCAGGCAAGGCTCACCCGTTCCAACT |
| | | CGAAGTGCCAGGGCCAGCTGGAGGTCTACCTCAAGGACGGATGGCACATGGTTTG |
| | | CAGCCAGAGCTGGGGCCGGAGCTCCAAGCAGTGGGAGGACCCCAGTCAAGCGTCA |
| | | AAAGTCTGCCAGCGGCTGAACTGTGGGGTGCCCTTAAGCCTTGGCCCCTTCCTTG |
| | | TCACCTACACACCTCAGAGCTCAATCATCTGCTACGGACAACTGG |
| 38 | CD38_ | GGGACAAACCCTGCTGCCGGCTCTCTAGGAGAGCCCAACTCTGTCTTGGCGTCAG |
| | target_ | TATCCTGGTCCTGATCCTCGTCGTGGTGCTCGCGGTGGTCGTCCCGAGGTGGCGC |
| | amplified | CAGCAGTGGAGCGGTCCGGGCACCACCAAGCGCTTTCCCGAGACCGTCCTGGCGC |
| | | GATGCGTCAAGTACACTGAAATTCATCCTGAGATGAGGTGGGTTGGCGACTAAGG |
| | | CGCACCGGTGGGCACTGCGGGGACAGCAGGGCCCCGCGCGCAGGGAAGCCGCCCG |
| | | GATCGCCCGGAACCGGGCATCTTCCGTGGGGGGTCAGCCGAGAGCCCGCCGGGTG |
| | | GTGCTGAGTAGGGAGTCCCGGGCTCGGGGCTCCGCGGGCCGCTTTCAGGAGCAGC |
| | | TGGCCTTG |
| 39 | AAVS1_ | mG*mG*mC*rUrCrCrArUrCrGrUrArArGrCrArArArCrCrUrUrGrUrUrG |
| | PBS_ | rArGrArArUrCrGrArArArGrArUrUrCrUrUrArArUrArArGrGrCrArUr |
| | 2 | CrCrUrUrCrCrGrArUrGrCrUrGrArCrUrUrCrUrCrArCrCrGrUrCrCrG |
| | | rUrUrArUrCrCrArArUrArGrGrArGrCrGrGrGrCrGrGrUrArUrGrGrCr |
| | | CrArGrArCrCrUrUrCrUrArArGrGrUrUrUrU*mU*mU*mU |
| 40 | AAVS1_ | mG*mG*mC*rUrCrCrArUrCrGrUrArArGrCrArArArCrCrUrUrGrUrUrG |
| | PBS_ | rArGrArArUrCrGrArArArGrArUrUrCrUrUrArArUrArArGrGrCrArUr |
| | 4 | CrCrUrUrCrCrGrArUrGrCrUrGrArCrUrUrCrUrCrArCrCrGrUrCrCrG |
| | | rUrUrArUrCrCrArArUrArGrGrArGrCrGrGrGrCrGrGrUrArUrGrGrCr |
| | | CrArGrArCrCrUrUrCrUrArArGrGrUrUrUrUrUrU*mU*mU*mU |
| 41 | AAVS1_ | mG*mG*mC*rUrCrCrArUrCrGrUrArArGrCrArArArCrCrUrUrGrUrUrG |
| | PBS_ | rArGrArArUrCrGrArArArGrArUrUrCrUrUrArArUrArArGrGrCrArUr |
| | 8 | CrCrUrUrCrCrGrArUrGrCrUrGrArCrUrUrCrUrCrArCrCrGrUrCrCrG |
| | | rUrUrArUrCrCrArArUrArGrGrArGrCrGrGrGrCrGrGrUrArUrGrGrCr |
| | | CrArGrArCrCrUrUrCrUrArArGrGrUrUrUrGrCrUrUrUrUrU*mU*mU*m |
| | | U |
| 42 | AAVS1_ | mG*mG*mC*rUrCrCrArUrCrGrUrArArGrCrArArArCrCrUrUrGrUrUrG |
| | PBS_ | rArGrArArUrCrGrArArArGrArUrUrCrUrUrArArUrArArGrGrCrArUr |
| | 10 | CrCrUrUrCrCrGrArUrGrCrUrGrArCrUrUrCrUrCrArCrCrGrUrCrCrG |
| | | rUrUrArUrCrCrArArUrArGrGrArGrCrGrGrGrCrGrGrUrArUrGrGrCr |
| | | CrArGrArCrCrUrUrCrUrArArGrGrUrUrUrGrCrUrUrArCrUrUrU*mU* |
| | | mU*mU |
| 43 | AAVS1_ | mG*mG*mC*rUrCrCrArUrCrGrUrArArGrCrArArArCrCrUrUrGrUrUrG |
| | PBS_ | rArGrArArUrCrGrArArArGrArUrUrCrUrUrArArUrArArGrGrCrArUr |
| | 13 | CrCrUrUrCrCrGrArUrGrCrUrGrArCrUrUrCrUrCrArCrCrGrUrCrCrG |
| | | rUrUrArUrCrCrArArUrArGrGrArGrCrGrGrGrCrGrGrUrArUrGrGrCr |
| | | CrArGrArCrCrUrUrCrUrArArGrGrUrUrUrGrCrUrUrArCrGrArUrUrU |
| | | rU*mU*mU*mU |
| 44 | AAVS1_ | mG*mG*mC*rUrCrCrArUrCrGrUrArArGrCrArArArCrCrUrUrGrUrUrG |
| | PBS_ | rArGrArArUrCrGrArArArGrArUrUrCrUrUrArArUrArArGrGrCrArUr |
| | 16 | CrCrUrUrCrCrGrArUrGrCrUrGrArCrUrUrCrUrCrArCrCrGrUrCrCrG |
| | | rUrUrArUrCrCrArArUrArGrGrArGrCrGrGrGrCrGrGrUrArUrGrGrCr |
| | | CrArGrArCrCrUrUrCrUrArArGrGrUrUrUrGrCrUrUrArCrGrArUrGrG |
| | | rArUrUrU*mU*mU*mU |
| 45 | AAVS1_ | mG*mG*mC*rUrCrCrArUrCrGrUrArArGrCrArArArCrCrUrUrGrUrUrG |
| | PBS_ | rArGrArArUrCrGrArArArGrArUrUrCrUrUrArArUrArArGrGrCrArUr |
| | 20 | CrCrUrUrCrCrGrArUrGrCrUrGrArCrUrUrCrUrCrArCrCrGrUrCrCrG |
| | | rUrUrArUrCrCrArArUrArGrGrArGrCrGrGrGrCrGrGrUrArUrGrGrCr |
| | | CrArGrArCrCrUrUrCrUrArArGrGrUrUrUrGrCrUrUrArCrGrArUrGrG |
| | | rArGrCrCrArUrUrU*mU*mU*mU |
| 46 | AAVS1_ | mG*mG*mC*rUrCrCrArUrCrGrUrArArGrCrArArArCrCrUrUrGrUrUrG |
| | Guide | rArGrArArUrCrGrArArArGrArUrUrCrUrUrArArUrArArGrGrCrArUr |
| | | CrCrUrUrCrCrGrArUrGrCrUrGrArCrUrUrCrUrCrArCrCrGrUrCrCrG |
| | | rUrUrArUrCrCrArArUrArGrGrArGrCrGrGrGrCrGrGrUrArUrGrUrUr |
| | | U*mU*mU*mU |
| 47 | B2M_ | mC*mG*mU*rCrArGrArGrCrGrCrCrGrArGrGrUrUrGrGrGrGrGrUrUrG |
| | PBS_ | rArGrArArUrCrGrArArArGrArUrUrCrUrUrArArUrArArGrGrCrArUr |
| | | CrCrUrUrCrCrGrArUrGrCrUrGrArCrUrUrCrUrCrArCrCrGrUrCrCrG |
| | | rUrUrArUrCrCrArArUrArGrGrArGrCrGrGrGrCrGrGrUrArUrGrArGr |
| | | ArGrArArArCrArCrUrCrCrUrCrCrArUrUrU*mU*mU*mU |
| 48 | B2M_ | mC*mG*mU*rCrArGrArGrCrGrCrCrGrArGrGrUrUrGrGrGrGrGrUrUrG |
| | PBS_ | rArGrArArUrCrGrArArArGrArUrUrCrUrUrArArUrArArGrGrCrArUr |
| | 4- | CrCrUrUrCrCrGrArUrGrCrUrGrArCrUrUrCrUrCrArCrCrGrUrCrCrG |
| | | rUrUrArUrCrCrArArUrArGrGrArGrCrGrGrGrCrGrGrUrArUrGrArGr |
| | | ArGrArArArCrArCrUrCrCrUrCrCrArArCrUrUrU*mU*mU*mU |
| 49 | B2M_ | mC*mG*mU*rCrArGrArGrCrGrCrCrGrArGrGrUrUrGrGrGrGrGrUrUrG |
| | PBS_ | rArGrArArUrCrGrArArArGrArUrUrCrUrUrArArUrArArGrGrCrArUr |
| | 8 | CrCrUrUrCrCrGrArUrGrCrUrGrArCrUrUrCrUrCrArCrCrGrUrCrCrG |
| | | rUrUrArUrCrCrArArUrArGrGrArGrCrGrGrGrCrGrGrUrArUrGrArGr |
| | | ArGrArArArCrArCrUrCrCrUrCrCrArArCrCrUrCrGrUrUrU*mU*mU*m |
| | | U |
| 50 | B2M_ | mC*mG*mU*rCrArGrArGrCrGrCrCrGrArGrGrUrUrGrGrGrGrGrUrUrG |
| | PBS_ | rArGrArArUrCrGrArArArGrArUrUrCrUrUrArArUrArArGrGrCrArUr |
| | 10 | CrCrUrUrCrCrGrArUrGrCrUrGrArCrUrUrCrUrCrArCrCrGrUrCrCrG |
| | | rUrUrArUrCrCrArArUrArGrGrArGrCrGrGrGrCrGrGrUrArUrGrArGr |
| | | ArGrArArArCrArCrUrCrCrUrCrCrArArCrCrUrCrGrGrCrUrUrU*mU* |
| | | mU*mU |
| 51 | B2M_ | mC*mG*mU*rCrArGrArGrCrGrCrCrGrArGrGrUrUrGrGrGrGrGrUrUrG |
| | PBS_ | rArGrArArUrCrGrArArArGrArUrUrCrUrUrArArUrArArGrGrCrArUr |
| | 13 | CrCrUrUrCrCrGrArUrGrCrUrGrArCrUrUrCrUrCrArCrCrGrUrCrCrG |
| | | rUrUrArUrCrCrArArUrArGrGrArGrCrGrGrGrCrGrGrUrArUrGrArGr |
| | | ArGrArArArCrArCrUrCrCrUrCrCrArArCrCrUrCrGrGrCrGrCrUrUrU |
| | | rU*mU*mU*mU |
| 52 | B2M_ | mC*mG*mU*rCrArGrArGrCrGrCrCrGrArGrGrUrUrGrGrGrGrGrUrUrG |
| | PBS_ | rArGrArArUrCrGrArArArGrArUrUrCrUrUrArArUrArArGrGrCrArUr |
| | 16 | CrCrUrUrCrCrGrArUrGrCrUrGrArCrUrUrCrUrCrArCrCrGrUrCrCrG |
| | | rUrUrArUrCrCrArArUrArGrGrArGrCrGrGrGrCrGrGrUrArUrGrArGr |
| | | ArGrArArArCrArCrUrCrCrUrCrCrArArCrCrUrCrGrGrCrGrCrUrCrU |
| | | rGrUrUrU*mU*mU*mU |
| 53 | B2M_ | mC*mG*mU*rCrArGrArGrCrGrCrCrGrArGrGrUrUrGrGrGrGrGrUrUrG |
| | PBS_ | rArGrArArUrCrGrArArArGrArUrUrCrUrUrArArUrArArGrGrCrArUr |
| | 20 | CrCrUrUrCrCrGrArUrGrCrUrGrArCrUrUrCrUrCrArCrCrGrUrCrCrG |
| | | rUrUrArUrCrCrArArUrArGrGrArGrCrGrGrGrCrGrGrUrArUrGrArGr |
| | | ArGrArArArCrArCrUrCrCrUrCrCrArArCrCrUrCrGrGrCrGrCrUrCrU |
| | | rGrArCrGrCrUrUrU*mU*mU*mU |
| 54 | B2M_ | mC*mG*mU*rCrArGrArGrCrGrCrCrGrArGrGrUrUrGrGrGrGrGrUrUrG |
| | Guide | rArGrArArUrCrGrArArArGrArUrUrCrUrUrArArUrArArGrGrCrArUr |
| | | CrCrUrUrCrCrGrArUrGrCrUrGrArCrUrUrCrUrCrArCrCrGrUrCrCrG |
| | | rUrUrArUrCrCrArArUrArGrGrArGrCrGrGrGrCrGrGrUrArUrGrUrUr |
| | | U*mU*mU*mU |
| 55 | CD5_ | mG*mG*mU*rGrArGrCrCrUrUrGrCrCrUrGrGrArArArUrCrUrGrUrUrG |
| | PBS_ | rArGrArArUrCrGrArArArGrArUrUrCrUrUrArArUrArArGrGrCrArUr |
| | 2 | CrCrUrUrCrCrGrArUrGrCrUrGrArCrUrUrCrUrCrArCrCrGrUrCrCrG |
| | | rUrUrArUrCrCrArArUrArGrGrArGrCrGrGrGrCrGrGrUrArUrGrUrUr |
| | | UrCrUrGrArCrCrGrArArArGrArUrUrUrUrU*mU*mU*mU |
| 56 | CD5_ | mG*mG*mU*rGrArGrCrCrUrUrGrCrCrUrGrGrArArArUrCrUrGrUrUrG |
| | PBS_ | rArGrArArUrCrGrArArArGrArUrUrCrUrUrArArUrArArGrGrCrArUr |
| | 4 | CrCrUrUrCrCrGrArUrGrCrUrGrArCrUrUrCrUrCrArCrCrGrUrCrCrG |
| | | rUrUrArUrCrCrArArUrArGrGrArGrCrGrGrGrCrGrGrUrArUrGrUrUr |
| | | UrCrUrGrArCrCrGrArArArGrArUrUrUrCrUrUrU*mU*mU*mU |
| 57 | CD5_ | mG*mG*mU*rGrArGrCrCrUrUrGrCrCrUrGrGrArArArUrCrUrGrUrUrG |
| | PBS_ | rArGrArArUrCrGrArArArGrArUrUrCrUrUrArArUrArArGrGrCrArUr |
| | 0 | CrCrUrUrCrCrGrArUrGrCrUrGrArCrUrUrCrUrCrArCrCrGrUrCrCrG |
| | | rUrUrArUrCrCrArArUrArGrGrArGrCrGrGrGrCrGrGrUrArUrGrUrUr |
| | | UrCrUrGrArCrCrGrArArArGrArUrUrUrCrCrArGrGrUrUrU*mU*mU*m |
| | | U |
| 58 | CD5_ | mG*mG*mU*rGrArGrCrCrUrUrGrCrCrUrGrGrArArArUrCrUrGrUrUrG |
| | PBS_ | rArGrArArUrCrGrArArArGrArUrUrCrUrUrArArUrArArGrGrCrArUr |
| | 10 | CrCrUrUrCrCrGrArUrGrCrUrGrArCrUrUrCrUrCrArCrCrGrUrCrCrG |
| | | rUrUrArUrCrCrArArUrArGrGrArGrCrGrGrGrCrGrGrUrArUrGrUrUr |
| | | UrCrUrGrArCrCrGrArArArGrArUrUrUrCrCrArGrGrCrArUrUrU*mU* |
| | | mU*mU |
| 59 | CD5_ | mG*mG*mU*rGrArGrCrCrUrUrGrCrCrUrGrGrArArArUrCrUrGrUrUrG |
| | PBS_ | rArGrArArUrCrGrArArArGrArUrUrCrUrUrArArUrArArGrGrCrArUr |
| | 13 | CrCrUrUrCrCrGrArUrGrCrUrGrArCrUrUrCrUrCrArCrCrGrUrCrCrG |
| | | rUrUrArUrCrCrArArUrArGrGrArGrCrGrGrGrCrGrGrUrArUrGrUrUr |
| | | UrCrUrGrArCrCrGrArArArGrArUrUrUrCrCrArGrGrCrArArGrGrUrU |
| | | rU*mU*mU*mU |
| 60 | CD5_ | mG*mG*mU*rGrArGrCrCrUrUrGrCrCrUrGrGrArArArUrCrUrGrUrUrG |
| | PBS_ | rArGrArArUrCrGrArArArGrArUrUrCrUrUrArArUrArArGrGrCrArUr |
| | 16 | CrCrUrUrCrCrGrArUrGrCrUrGrArCrUrUrCrUrCrArCrCrGrUrCrCrG |
| | | rUrUrArUrCrCrArArUrArGrGrArGrCrGrGrGrCrGrGrUrArUrGrUrUr |
| | | UrCrUrGrArCrCrGrArArArGrArUrUrUrCrCrArGrGrCrArArGrGrCrU |
| | | rCrUrUrU*mU*mU*mU |
| 61 | CD5_ | mG*mG*mU*rGrArGrCrCrUrUrGrCrCrUrGrGrArArArUrCrUrGrUrUrG |
| | PBS_ | rArGrArArUrCrGrArArArGrArUrUrCrUrUrArArUrArArGrGrCrArUr |
| | 20 | CrCrUrUrCrCrGrArUrGrCrUrGrArCrUrUrCrUrCrArCrCrGrUrCrCrG |
| | | rUrUrArUrCrCrArArUrArGrGrArGrCrGrGrGrCrGrGrUrArUrGrUrUr |
| | | UrCrUrGrArCrCrGrArArArGrArUrUrUrCrCrArGrGrCrArArGrGrCrU |
| | | rCrArCrCrCrUrUrU*mU*mU*mU |
| 62 | CD5_ | mG*mG*mU*rGrArGrCrCrUrUrGrCrCrUrGrGrArArArUrCrUrGrUrUrG |
| | Guide | rArGrArArUrCrGrArArArGrArUrUrCrUrUrArArUrArArGrGrCrArUr |
| | | CrCrUrUrCrCrGrArUrGrCrUrGrArCrUrUrCrUrCrArCrCrGrUrCrCrG |
| | | rUrUrArUrCrCrArArUrArGrGrArGrCrGrGrGrCrGrGrUrArUrGrUrUr |
| | | U*mU*mU*mU |
| 63 | CD38_ | mC*mU*mG*rArArArUrUrCrArUrCrCrUrGrArGrArUrGrArGrGrUrUrG |
| | PBS_ | rArGrArArUrCrGrArArArGrArUrUrCrUrUrArArUrArArGrGrCrArUr |
| | 2 | CrCrUrUrCrCrGrArUrGrCrUrGrArCrUrUrCrUrCrArCrCrGrUrCrCrG |
| | | rUrUrArUrCrCrArArUrArGrGrArGrCrGrGrGrCrGrGrUrArUrGrUrCr |
| | | GrCrCrArArCrArCrArCrCrUrCrArUrUrUrU*mU*mU*mU |
| 64 | CD38_ | mC*mU*mG**ArArArUrUrCrArUrCrCrUrGrArGrArUrGrArGrGrUrUrG |
| | PBS_ | rArGrArArUrCrGrArArArGrArUrUrCrUrUrArArUrArArGrGrCrArUr |
| | 4 | CrCrUrUrCrCrGrArUrGrCrUrGrArCrUrUrCrUrCrArCrCrGrUrCrCrG |
| | | rUrUrArUrCrCrArArUrArGrGrArGrCrGrGrGrCrGrGrUrArUrGrUrCr |
| | | GrCrCrArArCrArCrArCrCrUrCrArUrCrUrUrUrU*mU*mU*mU |
| 65 | CD38_ | mC*mU*mG**ArArArUrUrCrArUrCrCrUrGrArGrArUrGrArGrGrUrUrG |
| | PBS_ | rArGrArArUrCrGrArArArGrArUrUrCrUrUrArArUrArArGrGrCrArUr |
| | 8 | CrCrUrUrCrCrGrArUrGrCrUrGrArCrUrUrCrUrCrArCrCrGrUrCrCrG |
| | | rUrUrArUrCrCrArArUrArGrGrArGrCrGrGrGrCrGrGrUrArUrGrUrCr |
| | | GrCrCrArArCrArCrArCrCrUrCrArUrCrUrCrArGrGrUrUrU*mU*mU*m |
| | | U |
| 66 | CD38_ | mC*mU*mG*rArArArUrUrCrArUrCrCrUrGrArGrArUrGrArGrGrUrUrG |
| | PBS_ | rArGrArArUrCrGrArArArGrArUrUrCrUrUrArArUrArArGrGrCrArUr |
| | 10 | CrCrUrUrCrCrGrArUrGrCrUrGrArCrUrUrCrUrCrArCrCrGrUrCrCrG |
| | | rUrUrArUrCrCrArArUrArGrGrArGrCrGrGrGrCrGrGrUrArUrGrUrCr |
| | | GrCrCrArArCrArCrArCrCrUrCrArUrCrUrCrArGrGrArUrUrUrU*mU* |
| | | mU*mU |
| 67 | CD38_ | mC*mU*mG*rArArArUrUrCrArUrCrCrUrGrArGrArUrGrArGrGrUrUrG |
| | PBS_ | rArGrArArUrCrGrArArArGrArUrUrCrUrUrArArUrArArGrGrCrArUr |
| | 13 | CrCrUrUrCrCrGrArUrGrCrUrGrArCrUrUrCrUrCrArCrCrGrUrCrCrG |
| | | rUrUrArUrCrCrArArUrArGrGrArGrCrGrGrGrCrGrGrUrArUrGrUrCr |
| | | GrCrCrArArCrArCrArCrCrUrCrArUrCrUrCrArGrGrArUrGrArArUrU |
| | | rU*mU*mU*mU |
| 68 | CD38_ | mC*mU*mG*rArArArUrUrCrArUrCrCrUrGrArGrArUrGrArGrGrUrUrG |
| | PBS_ | rArGrArArUrCrGrArArArGrArUrUrCrUrUrArArUrArArGrGrCrArUr |
| | 16 | CrCrUrUrCrCrGrArUrGrCrUrGrArCrUrUrCrUrCrArCrCrGrUrCrCrG |
| | | rUrUrArUrCrCrArArUrArGrGrArGrCrGrGrGrCrGrGrUrArUrGrUrCr |
| | | GrCrCrArArCrArCrArCrCrUrCrArUrCrUrCrArGrGrArUrGrArArUrU |
| | | rUrUrUrU*m0*mU*mU |
| 69 | CD38_ | mC*mU*mG**ArArArUrUrCrArUrCrCrUrGrArGrArUrGrArGrGrUrUrG |
| | PBS_ | rArGrArArUrCrGrArArArGrArUrUrCrUrUrArArUrArArGrGrCrArUr |
| | 20 | CrCrUrUrCrCrGrArUrGrCrUrGrArCrUrUrCrUrCrArCrCrGrUrCrCrG |
| | | rUrUrArUrCrCrArArUrArGrGrArGrCrGrGrGrCrGrGrUrArUrGrUrCr |
| | | GrCrCrArArCrArCrArCrCrUrCrArUrCrUrCrArGrGrArUrGrArArUrU |
| | | rUrCrArGrUrUrU*mU*mU*mU |
| 70 | CD38_ | mC*mU*mG*rArArArUrUrCrArUrCrCrUrGrArGrArUrGrArGrGrUrUrG |
| | Guide | rArGrArArUrCrGrArArArGrArUrUrCrUrUrArArUrArArGrGrCrArUr |
| | | CrCrUrUrCrCrGrArUrGrCrUrGrArCrUrUrCrUrCrArCrCrGrUrCrCrG |
| | | rUrUrArUrCrCrArArUrArGrGrArGrCrGrGrGrCrGrGrUrArUrGrUrUr |
| | | U*mU*mU*mU |
| 71 | AAVS1_ | GGACAGCATGTTTGCTGCCTC |
| | forward_ | |
| | primer | |
| 72 | AAVS1_ | TTCCAAACTGCTTCTCCTCT |
| | reverse_ | |
| | primer | |
| 73 | B2M_ | GGGCTAGTCCAGGGCTGGAT |
| | forward_ | |
| | primer | |
| 74 | B2M_ | GCCAAGCATTCTACAAACGTCGCG |
| | reverse_ | |
| | primer | |
| 75 | CD5_ | GGCTGCCTGGGTCAGGGTCC |
| | forward_ | |
| | primer | |
| 76 | CD5_ | CCAGTTGTCCGTAGCAGATGA |
| | reverse_ | |
| | primer | |
| 77 | CD38_ | GGGACAAACCCTGCTGCCGG |
| | forward_ | |
| | primeH | |
| 78 | CD38_ | CAAGGCCAGCTGCTCCTGAAA |
| | reverse_ | |
| | prime | |
| 79 | VEGFA_ | mC*mU*mU*rUrGrArUrGrUrCrUrGrCrArGrGrCrCrArGrArUrGrUrUrG |
| | Guide | rArGrArArUrCrGrArArArGrArUrUrCrUrUrArArUrArArGrGrCrArUr |
| | | CrCrUrUrCrCrGrArUrGrCrUrGrArCrUrUrCrUrCrArCrCrGrUrCrCrG |
| | | rUrUrArUrCrCrArArUrArGrGrArGrCrGrGrGrCrGrGrUrArUrGrUrUr |
| | | UrU*mU*mU*mU |
| 80 | MCP-RT- | GTGATGCGGTTTTGGCAGTACATCAATGGGCGTGGATAGCGGTTTGACTCACGGG |
| | GFP- | GATTTCCAAGTCTCCACCCCATTGACGTCAATGGGAGTTTGTTTTGGCACCAAAA |
| | Normal | TCAACGGGACTTTCCAAAATGTCGTAACAACTCCGCCCCATTGACGCAAATGGGC |
| | intron-MS2 | GGTAGGCGTGTACGGTGGGAGGTCTATATAAGCAGAGCTGGTTTAGTGAACCGTC |
| | | AGATCCGCTAGCTAAAACCACAAAGGCCACCATGGACTACAAAGACGATGACGAC |
| | | AAGTACCCCTATGATGTACCTGATTATGCTCCTAAGAAGAAAAGAAAGGTGGCCA |
| | | GCAATTTCACACAGTTCGTCCTTGTAGACAACGGCGGCACCGGGGATGTTACAGT |
| | | GGCCCCGAGCAATTTCGCAAACGGCGTAGCCGAGTGGATCTCCTCTAATAGCAGA |
| | | AGCCAGGCTTATAAGGTTACATGCTCTGTAAGGCAGTCTTCCGCACAAAATAGAA |
| | | AGTACACAATCAAGGTCGAAGTGCCCAAGGTTGCTACACAGACCGTTGGTGGAGA |
| | | GGAGTTGCCAGTGGCTGGGTGGCGCTCCTACCTGAATATGGAGTTAACAATCCCC |
| | | ATTTTCGCTACCAATTCAGACTGTGAACTCATTGTCAAAGCTATGCAAGGTCTTC |
| | | TTAAGGATGGTAACCCAATTCCAAGTGCAATTGCTGCTAATTCAGGCATTTATTC |
| | | TGGCGGCAGCAGCGGCGGCTCAAGCGGAAGTGAAACACCTGGAACTTCCGAAAGC |
| | | GCTACGCCTGAATCTTCTGGTGGATCTTCTGGCGGATCTTCCGCATGCGTCGACG |
| | | ATATCCCTAAGAAGAAAAGAAAGGTGTGACTAGCTGGCCGGCCGTGCGTTTTATT |
| | | CCTGTCTTTTTATTGCCGATCCCCGCGATCGCTTACTTGTACAGCTCGTCCATGC |
| | | CGAGAGTGATCCCGGCGGCGGTCACGAACTCCAGCAGGACCATGTGATCGCGCTT |
| | | CTCGTTGGGGTCTTTGCTCAGGGCGGACTGGGTGCTCAGGTAGTGGTTGTCGGGC |
| | | AGCAGCACGGGGCCGTCGCCGATGGGGGTGTTCTGCTGGTAGTGGTCGGCGAGCT |
| | | GCACGCTGCCGTCCTCGATGTTGTGGCGGATCTTGAAGTTCACCTTGATGCCGTT |
| | | CTTCTGCTTGTCGGCCATGATATAGACGTTGTGGCTGTTGTAGTTGTACTCCAGC |
| | | TTGTGCCCCAGGATGTTGCCGTCCTCCTTGAAGTCGATGCCCTTCAGCTCGATGC |
| | | GGTTCACCAGGGTGTCGCCCTCGAACTTCACCTAAGGTGAGTCCAGGAGATGTTT |
| | | CAGCACTGTTGCCTTTAGTCTCGAGGCAACTTAGACAACTGAGTATTGATCTGAG |
| | | CACAGCAGGGTGTGAGCTGTTTGAAGATACTGGGGTTGGGGGTGAAGAAACTGCA |
| | | GAGGACTAACTGGGCTGAGACCCAGTGGCAATGTTTTAGGGCCTAAGGAATGCCT |
| | | CTGAAAATCTAGATGGACAACTTTGACTTTGAGAAAAGAGAGGTGGAAATGAGGA |
| | | AAATGACTTTTCTTTATTAGATTTCGGTAGAAAGAACTTTCACCTTTCCCCTATT |
| | | TTTGTTATTCGTTTTAAAACATCTATCTGGAGGCAGGACAAGTATGGTCGTTAAA |
| | | AAGATGCAGGCAGAAGGCATATATTGGCTCAGTCAAAGTGGGGGAACTTTGGTGG |
| | | CCAAACATACATTGCTAAGGCTATTCCTATATCAGCTGGACACATATAAAATGCT |
| | | GCTAATGCTTCATTACAAACTTATATCCTTTAATTCCAGATGGGGGCAAAGTATG |
| | | TCCAGGGGTGAGGAACAATTGAAACATTTGGGCTGGAGTAGATTTTGAAAGTCAG |
| | | CTCTGTATGTGTATGTGTATGTGTATGTGTATGTGTACGCGCACGTGTGTTTGTG |
| | | TGTGTGTGAGAGCGTGTGTTTCTTTTAACGTTTTCAGCCTACAGCATACAGGGTT |
| | | CATGGTGGCAAGAAGATAACAAGATTTAAATTATGGCCAGTGACTAGTGCTGCAA |
| | | GAAGAACAACTACCTGCATTTAATGGGAAAGCAAAATCTCAGGCTTTGAGGGAAG |
| | | TTAACATAGGCTTGATTCTGGGTGGAAGCTGGGTGTGTAGTTATCTGGAGGCCAG |
| | | GCTGGAGCTCTCAGCTCACTATGGGTTCATCTTTATTGTCTCCTTTCATCTCAAC |
| | | GGCGCGGGTCTTGTAGTTGCCGTCGTCCTTGAAGAAGATGGTGCGCTCCTGGACG |
| | | TAGCCTTCGGGCATGGCGGACTTGAAGAAGTCGTGCTGCTTCATGTGGTCGGGGT |
| | | AGCGGCTGAAGCACTGCACGCCGTAGGTCAGGGTGGTCACGAGGGTGGGCCAGGG |
| | | CACGGGCAGCTTGCCGGTGGTGCAGATGAACTTCAGGGTCAGCTTGCCGTAGGTG |
| | | GCATCGCCCTCGCCCTCGCCGGACACGCTGAACTTGTGGCCGTTTACGTCGCCGT |
| | | CCAGCTCGACCAGGATGGGCACCACCCCGGTGAACAGCTCCTCGCCCTTGCTCAC |
| | | CATGGTGGCCCTGCAGGCTGTGTTCTGGCGGCAAACCCGTTGCGAAAAAGAACGT |
| | | TCACGGCGACTACTGCACTTATATACGGTTCTCCCCCACCCTCGGGAAAAAGGCG |
| | | GAGCCAGTACACGACATCACTTTCCCAGTTTACCCCGCGCCACCTTCTCTAGGCA |
| | | CCGGATCAATTGCCGACCCCTCCCCCCAACTTCTCGGGGACTGTGGGCGATGTGC |
| | | GCTCTGCCCGATGAGGGCTCCAGATGGCATTTAGAGCTAGGCCAACATGAGGATT |
| | | ACCCATGTCTGCAGGGCCTAGCAAGTTAAAATAAGGCTAGTCCGTTATCAACTTG |
| | | GCCAGGACTGAGGATTACCCAGTCTATCTGCAGGGCCAAGTAAAAAAAAAAGGAA |
| | | GGGAAAAGGGGAATTC |
| 81 | MCP-RT | GTGATGCGGTTTTGGCAGTACATCAATGGGCGTGGATAGCGGTTTGACTCACGGG |
| | GFP- | GATTTCCAAGTCTCCACCCCATTGACGTCAATGGGAGTTTGTTTTGGCACCAAAA |
| | Chimeric | TCAACGGGACTTTCCAAAATGTCGTAACAACTCCGCCCCATTGACGCAAATGGGC |
| | intron-MS2 | GGTAGGCGTGTACGGTGGGAGGTCTATATAAGCAGAGCTGGTTTAGTGAACCGTC |
| | | AGATCCGCTAGCTAAAACCACAAAGGCCACCATGGACTACAAAGACGATGACGAC |
| | | AAGTACCCCTATGATGTACCTGATTATGCTCCTAAGAAGAAAAGAAAGGTGGCCA |
| | | GCAATTTCACACAGTTCGTCCTTGTAGACAACGGCGGCACCGGGGATGTTACAGT |
| | | GGCCCCGAGCAATTTCGCAAACGGCGTAGCCGAGTGGATCTCCTCTAATAGCAGA |
| | | AGCCAGGCTTATAAGGTTACATGCTCTGTAAGGCAGTCTTCCGCACAAAATAGAA |
| | | AGTACACAATCAAGGTCGAAGTGCCCAAGGTTGCTACACAGACCGTTGGTGGAGA |
| | | GGAGTTGCCAGTGGCTGGGTGGCGCTCCTACCTGAATATGGAGTTAACAATCCCC |
| | | ATTTTCGCTACCAATTCAGACTGTGAACTCATTGTCAAAGCTATGCAAGGT |
| | | CTTCTTAAGGATGGTAACCCAATTCCAAGTGCAATTGCTGCTAATTCAGGCATTT |
| | | ATTCTGGGGGCAGCAGCGGCGGCTCAAGCGGAAGTGAAACACCTGGAACTTCCGA |
| | | AAGCGCTACGCCTGAATCTTCTGGTGGATCTTCTGGCGGATCTTCCGCATGCGTC |
| | | GACGATATCCCTAAGAAGAAAAGAAAGGTGTGACTAGCTGGCCGGCCGTGCGTTT |
| | | TATTCCTGTCTTTTTATTGCCGATCCCCGCGATCGCTTACTTGTACAGCTCGTCC |
| | | ATGCCGAGAGTGATCCCGGCGGCGGTCACGAACTCCAGCAGGACCATGTGATCGC |
| | | GCTTCTCGTTGGGGTCTTTGCTCAGGGCGGACTGGGTGCTCAGGTAGTGGTTGTC |
| | | GGGCAGCAGCACGGGGCCGTCGCCGATGGGGGTGTTCTGCTGGTAGTGGTCGGCG |
| | | AGCTGCACGCTGCCGTCCTCGATGTTGTGGCGGATCTTGAAGTTCACCTTGATGC |
| | | CGTTCTTCTGCTTGTCGGCCATGATATAGACGTTGTGGCTGTTGTAGTTGTACTC |
| | | CAGCTTGTGCCCCAGGATGTTGCCGTCCTCCTTGAAGTCGATGCCCTTCAGCTCG |
| | | ATGCGGTTCACCAGGTAAGTATCAAGGTTACAAGACAGGTTTAAGGAGACCAATA |
| | | GAAACTGGGCTTGTCGAGACAGAGAAGACTCTTGCGTTTCTGATAGGCACCTATT |
| | | GGTCTTACTGACATCCACTTTGCCTTTCTCTCCACAGGGTGTCGCCCTCGAACTT |
| | | CACCTCGGCGCGGGTCTTGTAGTTGCCGTCGTCCTTGAAGAAGATGGTGCGCTCC |
| | | TGGACGTAGCCTTCGGGCATGGCGGACTTGAAGAAGTCGTGCTGCTTCATGTGGT |
| | | CGGGGTAGCGGCTGAAGCACTGCACGCCGTAGGTCAGGGTGGTCACGAGGGTGGG |
| | | CCAGGGCACGGGCAGCTTGCCGGTGGTGCAGATGAACTTCAGGGTCAGCTTGCCG |
| | | TAGGTGGCATCGCCCTCGCCCTCGCCGGACACGCTGAACTTGTGGCCGTTTACGT |
| | | CGCCGTCCAGCTCGACCAGGATGGGCACCACCCCGGTGAACAGCTCCTCGCCCTT |
| | | GCTCACCATGGTGGCCCTGCAGGCTGTGTTCTGGGGGCAAACCCGTTGCGAAAAA |
| | | GAACGTTCACGGCGACTACTGCACTTATATACGGTTCTCCCCCACCCTCGGGAAA |
| | | AAGGGGGAGCCAGTACACGACATCACTTTCCCAGTTTACCCCGCGCCACCTTCTC |
| | | TAGGCACCGGATCAATTGCCGACCCCTCCCCCCAACTTCTCGGGGACTGTGGGCG |
| | | ATGTGCGCTCTGCCCGATGAGGGCTCCAGATGGCATTTAGAGCTAGGCCAACATG |
| | | AGGATTACCCATGTCTGCAGGGCCTAGCAAGTTAAAATAAGGCTAGTCCGTTATC |
| | | AACTTGGCCAGGACTGAGGATTACCCAGTCTATCTGCAGGGCCAAGTAAAAAAAA |
| | | AAGGAAGGGAAAAGGGGAATTC |
| 82 | linker 1 | SGGSSGGSSGSETPGTSESATPESSGGSSGGSSAC |
| | for | |
| | tethered | |
| | RT | |
| 83 | linker 2 | KLGGGAPAVGGGPK |
| | for | |
| | tethered | |
| | RT | |
| 84 | linker 3 | (GGGGS)3 |
| | for | |
| | tethered | |
| | RT | |
| 85 | linker 4 | (GGGGS)2EAAAK(GGGGS)2 |
| | for | |
| | tethered | |
| | RT | |
| 86 | linker 5 | (GGGGS)2(EAAAK)2(GGGGS)2 |
| | for | |
| | tethered | |
| | RT | |
| 87 | linker 6 | SGSETPGTSESATPES |
| | for | |
| | tethered | |
| | RT | |
| 88 | MG140-29 | MDGVFAVHNVDNLTDHEPIVIQLSLKTDVLQNSEIVHVPRVSWRKASHDQVRNYK |
| | retrotransposition | NVISSNLKSIYLPVKILLCNDIHCSNPIHFEDINRYSISIHDACISAARDTIPVT |
| | protein | CRRGSRGRIPGWTERVKPFRERSLFWHNIWVDCGRPRDGHVAAAMRRTRAAYHLQ |
| | | IKLVRKNEDAVVREKVADSVIHDRGRNFWSEIKKIRGTKTRPPGTVDGLIDTPSI |
| | | AALFADKYRDLYTSVPYDKDDMQRIIDVVDSKLSCINESGDCTFYATEVREAIGH |
| | | MKRGKNDGSNELLSDHLINANDDLIIHIACLFSTFSVHGAVPANFHSSVILPIPK |
| | | SRNASAASSNNYRGIALSSLLGKILDYIILERYRDTLSSCEYQFGFKRSSSTNLC |
| | | TMVLKVTLNYYHDNSTNVECCELDATKAFDRVNYCKLFRLLLTRDLPPHIIRLLI |
| | | NIYVNNNVCVSWCNVKSDTFRALNGVKQGAVISPILYCVYVDNLLQLLEKAGVGC |
| | | HIGFQFLGALAYADDLVLIAPTASAMRTMLSVCDNFARDEDVIENASKSKWLAVI |
| | | PSNRSEMSSMINDCLFEVGGNPIDFVDKFVHLGHIISSNLIDTADVESRRSSFIA |
| | | EVNNMSCFFSKLDVFTRESLERSYCTSYYGSELWKLNCPSIDSFYTSWRRALRTV |
| | | WRLPFRAHSELLPLISNVLPFRDEICRRSINEMRSCLFHNSYLVRFISYNSIMFG |
| | | TKSSELRHNAYYCASRLGCGLDSIIYGNVCVLAEVESNEMFADAGMLLELLLLRD |
| | | GVLHCGLNIDEISTLIIHLCIND* |
| 89 | MG140-30 | MILSIDNTSDHDPIILRLSLDIKYVSVCNRTSSSRVSWVKASDRDIRNYQYNLAS |
| | retrotransposition | NLQHVTIPSVALLCKDVNCSNFAHRCQLSRYLTDISDACLAAGEASIPHTCSRHS |
| | protein | GKRIPGWSEKVEPLRQRSLFWHSMWVECGRPRSGVVADCMRRARASYHYAIRAVR |
| | | RNEQNIIRERVADALIRDPSRDFWTEVKKIRNSKSGRAVIVDGCSDAPSISQLFA |
| | | SKYRHLYTSVPYQHNDLQSIVSDVESRISEDGDCFIGSQEVMAALSKLKLHKNDG |
| | | DLGLASDYFINSDPALSVHIALLFTGIVINGFVPSNLLSSTIVPIPKKSNVNATD |
| | | SDNYRGIALSSVLGKIFDNVILVKYSDKLSTCNLQFGFKRNSSTHMCIMVLKETI |
| | | SYYVNNNSSVECTFLDASKAFDRVHYCKLERLLLGRGLPVCILRVLIQLYVGHSI |
| | | RVTWAGLVSSCFTALNGVKQGGVLSPVLFCIYVDELLIRLAESGVGCYIGFSFAG |
| | | ALAYADDIVLIAPTPSAMRKLLAICDTYATEYNILFNAQKSNFIAFVPSSRHFLH |
| | | KAMINCVFRIGGVQIEHVESYTHLGHIITSRLDDADDILHRRSSYIGQVNNVVCY |
| | | FDSLSWTVKLGLHKSYCSSIFGCELWALDSVRDIEKFCVAWRKGLRRVISLPRAA |
| | | HSHLLPLLSNSLPVYDEICKRSAKFIVSCQCSDNILVRAVVNYAIAARSKSVLGR |
| | | NVMLLCRREHLSEDDEVSGRLLLSGDIFVSHYLNSLSEAQLQSVCFALELLCLRE |
| | | QSFKLNNNMRINADEISDYLAAVLC* |
| 90 | MG140-31 | MLLFIRSVMENSNSVIDNLQLTICSYNCRGENGFKKDYISGLLESAQIDVLLLQE |
| | retrotransposition | HWLSDAQLNALNNVGANYINEGVSGEDTSAVLGGRPYGGCAVLWRSDLLLQVQPL |
| | | VVSSRRISAVIESTDNWSLILINVYMPYEGDEIKTDEFIDLLSIIEDLVLSNSAS |
| | protein | HVIVGGDFNVEENRNRMHTALLNSFCDNTGLSPVIQHSSCNIDYTYNFNMSRENI |
| | | LDHFLLSGTLFDVCVTSAYVLHDIDNTSHHDPIILRLSLDIKYVSVCNRISSSRV |
| | | SWVKASDRDIRNYQYNLASNLQHVTIPSVALLCKDVNCSNFAHRCQLSRYLTDIS |
| | | DACLAAGEASIPHTCSRHSGKRIPGWSEKVEPLRQRSLFWHSMWVECGRPRSGVV |
| | | ADCMRRARASYHYAIRAVRRNEQNIIRERVADALLRDPSRDFWTEVKKIRNSKSG |
| | | RAVIVDGCSDAPSISQLFASKYRHLYTSVPYQHNDLQSIVSDVESRISEDGDCFI |
| | | GSQEVMAALSKLKLHKNDGDLGLASDYFINSDPALSVHIALLFTGIVINGFVPSN |
| | | LLSSTIVPIPKKSNVNATDSDNYRGIALSSVIGKIFDNVILVKYSDKLSTCNLQF |
| | | GFKRNSSTHMCTMVLKETISYYVNNNSSVECTFLDASKAFDRVHYCKLERLLLGR |
| | | GLPVCILRVLIQLYVGHSIRVTWAGLVSSCFTALNGVKQGGVLSPVLFCIYVDEL |
| | | LIRLAESGVGCYIGESFAGALAYADDIVLIAPTPSAMRKLLAICDTYATEYNILF |
| | | NAQKSNFIAFVPSSRHELHKAMINCVFRIGGVQIEHVESYTHLGHIITSRLDDAD |
| | | DILHRRSSYIGQVNNVVCYFDSLSWTVKLGLHKSYCSSIFGCELWALDSVRDIEK |
| | | FCVAWRKGLRRVLSLPRAAHSHLLPLLSNSLPVYDEICKRSAKFIVSCQCSDNIL |
| | | VRAVVNYAIAARSKSVLGRNVMLLCRRFHLSEDDEVSGRILLSGDIEVSHYLNSL |
| | | SEAQLQSVCFALELLCLREQSFKLNNNMRLNADEISDYLAAVLC* |
| 91 | MG140-32 | MVQNTELADCINITLQINDSRFIVISYNMHGFNQGLEGTKEMINKLYPDVIALQE |
| | retrotransposition | HWLTPINLDRLGEVSNDYFFIGSSSMNDVVTAGPLFGRPFGGTAILINNKLANAT |
| | protein | VNVACNDKFTAVLISECLVISAYMPCAGTCDRMSLYSAIISEIQALIEFYPQYKL |
| | | ILCADINVELDAPSSVADLVNNFICRNNIRRTELANPTASRFTYMHESLQAASYI |
| | | DYIISSDALDSIAENVLDLDINLSDHLPIMCVFMCKMSACYSDPSQSVKSKSHVD |
| | | DVSYFRWDHAALHLYYEQTRVMMEPILDKLNICVNSVCDEDAFIDHSKLEDIYSS |
| | | VVAALITSANLCIPKIKHNFFKEWWNQELNELKAAAVISARAWQQVGKPKHGSVY |
| | | HKYQKDKLLYKKCIRENQKQESVSFTNELHDALLRKTGKDFWKTWNAKVGVKRKC |
| | | IEQVDGIVDGATIACNFAKHFEKICQPATATENTIMKAKFEEMRSMYYTPFCDKT |
| | | VEFDVQLIGKIISNMSCGKAAGLDELSAEHLKHCHPIVIIILCKLENLFVHVGYL |
| | | PLSFGTSYTVPIPKQNGRSHALTVNDERGISISPVISKIFEHAIFAREGDYESTS |
| | | DHQFGFKESLSCSHAIYCVRNVIDHYVKRGSTVNICTVDISKAFDTVNHEVLFIK |
| | | LMERKLPVQLLDLEVLWFSMSETCVRWGAHDSYFFKLKAGVRQGGVLSPYFFAVS |
| | | VDDVVDKIAACNAGCYINNLCSAIFLYADDIILLSPSVCGLQRLLGICEAAITEL |
| | | DMKINASKTVCTRVGPREDSACVGPNLTSGGQLNWVKTCRYLGVHFAAGRSENCS |
| | | FEEAKKKEYFSENSVYSKLGREASEEVILNLLNVKCVSAMLYATEACPVLSRHKH |
| | | SLDFVITRVEMKILRTGSPQVVAECQKYFGFLPVSYRIDIRSARFLERFSSSVNS |
| | | FCIAFGDRARKQLSDIVCKYNISDGLSSGVLRTVINRIYFQ* |
| 92 | MG140-33 | MTIINDAAQLVVISYNLHGLNQGLPGIREFMTELKPDVIMVQEHWLTSDNLNKLS |
| | retrotransposition | DISDEYFVIGSSAMDARVSAGPLEGRPFGGTAVFIKNKYINVTVNLVISERYVVI |
| | protein | QLCDWLLINVYLPCIGTSNRILLYSDTICELQSIIRAHPECNCLVGGDENTDIND |
| | | TRSHTANTVVNGFIAGCNLHRCDCLFPTRLKHTYVNDSMNCYSTIDYMLSSNPEK |
| | | IVAFNVLDIDLNLSDHLPIISICVEDCNCKMNNPKPTSTENVTHERWDHAPLQDY |
| | | YEHTRLGLQPILADLDELIGNKLSYSNVDELCQVDCIYNRVVSVLQQCSHAYVPK |
| | | HKKNFYKEWWNQELDELKDKAIASCKMWKDAGKPRHGAIHAKYRQDKLLYKKRIR |
| | | QERVQETSSFTNELHDALLINKSGRDFWKKWNSKFENKSNKILQVGGTSDTATIL |
| | | NNFAKHFEQVCVPFNATRNEELKSRYKEMRLNYSESSVINDTTVEDVQLVENLLT |
| | | NMKNGKAGGLDELTSEHLKFCHPVVVIILCKLENLFVINGHIPDSFGVSYTVPIP |
| | | KSDGRSRSMTVDDERGISISPVISKLFELCVLDRYSDYLQTSDHQFGFKKQLGCR |
| | | HAIFSVRSVIEQYISNGSTVNICALDLSKAFDRMNQYALFIKLMERREPVKILTI |
| | | LEQWESIAETCVRWGSEFSYFFSLLAGVRQGGVLSPVLFAIFIDGIVNRVNATNV |
| | | GCYNSTVCVSIFLYADDILLLSPTVTGLQTLLTVCENELCELDMRLNVNKSVCMR |
| | | EGARFKAHCANLVSVQGGALQWASSCRYLGVYEVSGRVERCCFHSAKQNFERAFN |
| | | SVYSKIGCCASEEVILSLIKSKCLPYILYGVEACPVLQRDKHSEDFTLTRTIMKL |
| | | EMTSSPVIVNECQTQENLLPLRYQIDIRTVKFLNQYIISRNSICMLYKSHAQSVL |
| | | DTIFGVYGNNVSSLHDLHNIINEHFYDENPSTLGYN* |
| 93 | MG140-34 | MYNLRITSYNCRSLSALKRDFVKTLIVSCDILFLQEHWLSDDQLTLLGGLDEGFT |
| | retrotransposition | FTGVSGEGNKEILSGRPYGGCAILWRSALKFSVDFLSVNSRRVSAIKLSNDCCNL |
| | protein | LLINVYMPFEDSDCNVNEFFDVLFDIEYLLVNNVDCHYLIGGDENVDLSRNTVHT |
| | | ALLRSESDNNGLLYASDFDGANFDFTYQFNMSRFSLIDNFMLSSFLFHNMLQRAD |
| | | VVHDVNNLSDHEPVTIELCMSVTHVDIQRSTVPPLQKVSWTTAAESHVIDYREEL |
| | | VDRLKTVVMPIDSLTCCDRRCSVAKHRSDIAEYANNIADVCIKAGLITIPLMKPG |
| | | HRATPGFSEHVKPARDKSMFWHQLWLECGRPRTGHVADCMRRTRAAYHYALRSVK |
| | | RRRDEITQERFATALLHNNSRNEWSEVKKIRANRMTYNGVVDGHSDATDIVRIFG |
| | | ERYRDLYTSVPYDECNMHEINDTIDEHISDDDDAAFVVSLRDVSDAIAHIKHYKN |
| | | DVDNMLTSDHFINAPSVLHVHISILFSAMLMHGCVPNLLMYSSIRPIPKGHNIST |
| | | CDSNNYRGIAISSIFNKIEDNVVLIKYRHLLSTCDLQFGFKKKHSTQMCTMVLKE |
| | | TLSYYLSNRSNVFCTFLDATKAFDRINYCKLFNLLLCRSLPYCIIRVLLSLYTNN |
| | | YVYVSWVGSNSSSFRACNGVKQGGVLSPVLFCLYMDGLINKLSHAGVGCYMGEMF |
| | | VGALAYADDIVLISPTPSAMNNMLSICDEFSIEYNVLENPLKSKCMYFYPKSRST |
| | | LLLHRYNVCDLQFSINGKAIEFVDSYKHLGHVICSDMTDDIDISEKRGVEIGQAN |
| | | NIICYFAKLSSAIKYRLFTSYCTSFFGCELWRISNDSLDSMCTAWRRAIRRIWSL |
| | | PYTAHGRFLPVLCNSYNIFDQFCVRILNFIRRCLSDQSSLLVRSIATQALMFHHA |
| | | RSPLGYNFIYCARRYCESLTDEVNYNNEVSNVEKLNCTQNDDDTIANCRLLRELI |
| | | DLRDGVLHLSDDVFLINSELSFMINCVSTL* |
| 94 | MG140-35 | MYNLRITSYNCRSLSALKRDEVKILLVSCDILFLQEHWLSDDQLTLLGGLDEGFT |
| | retrotransposition | FTGVSGFGNKEILSGRPYGGCAILWRSALKFSVDFLSVNSRRVSAIKLSNDCCNL |
| | protein | LLINVYMPFEDSDCNVNEFFDVLFDIEYLLVNNVDCHYLIGGDENVDLSRNTVHT |
| | | ALLRSESDNNGLLYASDEDGANFDFTYQFNMSRFSLIDNFMLSSFLFHNMLQRAD |
| | | VVHDVNNLSDHEPVTIELCMSVTHVDIQRSTVPPLQKVSWTTAAESHVIDYREEL |
| | | VDRLKTVVMPIDSLTCCDRRCSVARHRSDIAEYANNIADVCIKAGLITIPLMKPG |
| | | HRATPGESEHVKPARDKSMFWHQLWLECGRPRTGHVADCMRRTRAAYHYALRSVK |
| | | RRRDEITQERFATALLHNNSRNEWSEVKKIRANRMTYNGVVDGHSDATDIVRIFG |
| | | ERYRDLYTSVPYDECNMHEINDTIDEHISDDDDAAFVVSLRDVSDAIAHIKHYKN |
| | | DVDNMLTSDHFINAPSVLHVHISILFSAMLMHGCVPNLLMYSSIRPIPKGHNLST |
| | | CDSNNYRGIAISSIENKIFDNVVLIKYRHLISTCDLQFGFKKKHSTQMCTMVLKE |
| | | TLSYYLSNRSNVECTELDATKAFDRINYCKLFNLLLCRSLPYCIIRVLLSLYTNN |
| | | YVYVSWVGSNSSSFRACNGVKQGGVLSPVLFCLYMDGLLNKLSHAGVGCYMGEMF |
| | | VGALAYADDIVLISPTPSAMNNMLSICDEFSIEYNVLENPLKSKCMYFYPKSRST |
| | | LLLHRYNVCDLQFSINGKAIEFVDSYKHLGHVICSDMTDDIDISEKRGVFIGQAN |
| | | NIICYFAKLSSAIKYRLFTSYCTSFFGCELWRISNDSLDSMCTAWRRAIRRIWSL |
| | | PYTAHGRFLPVLCNSYNIEDQECVRILNFIRRCLSDQSSLLVRSIATQALMEHHA |
| | | RSPLGYNFIYCARRYCFSLTDFVNYNNEVSNVEKLNCTQNDDDTIANCRLIRELI |
| | | DLRDGVLHLSDDVFLINSELSEMINCVSTL* |
| 95 | MG140-36 | MVQNTELADCINITLQINDSRFIVTSYNMHGENQGLEGTKEMINKLYPDVIALQE |
| | retrotransposition | HWLTPTNLDRIGEVSNDYFFIGSSSMNDVVTAGPLFGRPEGGTAILINNKLANAT |
| | protein | VNVACNDKFTAVLISECLVISAYMPCAGTCDRMSLYSAIISEIQALIEFYPQYKL |
| | | ILCADLNVELDAPSSVADLVNNFICRNNLRRTELANPTASRFTYMHESLQAASYI |
| | | DYIISSDALDSIAFNVLDLDINLSDHIPIMCVFMCKMSACYSDPSQSVKSKSHVD |
| | | DVSYFRWDRAALHLYYEQTRVMMEPILDKLNICVNSVCDEDAFIDHSKLEDIYSS |
| | | VVAALTTSANLCIPKIKHNFFKEWWNQELNELKAAAVISARAWQQVGKPKHGSVY |
| | | HKYQKDKLLYKKCIRENQKQESVSEINELHDALLRKTGKDEWKTWNAKVGVKRKC |
| | | IEQVDGIVDGATIACNFAKHFEKICQPATATENTTMKAKFEEMRSMYYTPFCDKT |
| | | VEFDVQLIGKIISNMSCGKAAGLDELSAEHLKHCHPIVIIILCKLENLFVHVGYL |
| | | PLSFGTSYTVPIPKQNGRSHALTVNDERGISISPVISKIFEHAIFARFGDYFSTS |
| | | DHQFGFKESLSCSHAIYCVRNVIDHYVKRGSTVNICTVDISKAFDTVNHFVLFIK |
| | | IMERKLPVQLLDLFVIWESMSETCVRWGAHDSYFFKLKAGVRQGGVLSPYFFAVS |
| | | VDDVVDKIAACNAGCYINNLCSAIFLYADDIILLSPSVCGLQRLLGICEAAITEL |
| | | DMKINASKTVCTRVGPREDSACVGPNLTSGGQLNWVKTCRYLGVHFAAGRSFNCS |
| | | FEEAKKKFYFSENSVYSKLGRFASEEVILNLLNVKCVSAMLYATEACPVLSRHKH |
| | | SLDFVITRVEMKILRTGSPQVVAECQKYFGFLPVSYRIDIRSARFLERFSSSVNS |
| | | FCIAFGDRARKQLSDILCKYNIPDGLSSGVLRTIINRIFFNS* |
| 96 | MG140-37 | MTIINDAAQLVVISYNLHGLNQGLPGIREFMTELKPDVIMVQEHWLTSDNINKLS |
| | retrotransposition | DISDEYFVIGSSAMDARVSAGPLEGRPFGGTAVFIKNKYINVTVNLVISERYVVI |
| | protein | QLCDWLLINVYLPCIGTSNRILLYSDTLCELQSIIRAHPECNCLVGGDENTDLND |
| | | TRSHTANTVVNGFIAGCNLHRCDCLFPTRLKHTYVNDSMNCYSTIDYMLSSNPEK |
| | | IVAFNVLDIDLNLSDHLPIITVCVEDSNCKLNNPKPTSTEDVTHFRWDHAPLQDY |
| | | YEHTRLGLQPILADLDELIGNKLYSNVDELCQVDCIYNRVVSVLQQCSHAYVPKH |
| | | KKNFYKFWWNQELDELKDKAIASCKMWKDAGKPRHGAIHAKYRQDKLLYKKRIRQ |
| | | ERVQETSSFTNELHDALLNKSGRDFWKKWNSKFENKSNKILQVGGTSDTATILNN |
| | | FAKHFEQVCVPFNATRNEELKSRYKEMRINYSESSVINDTTVEDVQLVENLLINM |
| | | KNGKAGGLDELTSEHLKECHPVVVIILCKLENLFVINGHIPDSFGVSYTVPIPKS |
| | | DGRSRSMTVDDERGISISPVISKLFELCVLDRYSDYLQTSDHQFGFKKQLGCRHA |
| | | IFSVRSVIEQYISNGSTVNLCALDLSKAFDRMNQYALFIKLMERRFPVKILTILE |
| | | QWESIAETCVRWGSEFSYFFSLLAGVRQGGVLSPVLFAIFIDGIVNRVNATNVGC |
| | | YNSTVCVSIFLYADDILLLSPTVTGLQTLLTVCENELCELDMRLNVNKSVCMRFG |
| | | ARFKAHCANLVSVQGGALQWASSCRYLGVYFVSGRVFRCCFHSAKQNFERAFNSV |
| | | YSKIGCCASEEVILSLLKSKCLPYILYGVEACPVLQRDKHSFDFILTRTLMKLFM |
| | | TSSPVIVNECQTQFNLLPLRYQIDIRTVKELNQYIISRNSICMLYKSHAQSVLDT |
| | | IFGVYGNNVSSLHDLHNIINEHFYDENPSTLGYN* |
| 97 | MG140-38 | MVILLLMASYTNAILSIASENCRGENQIKKQYICNLLGKCNFLFLQEHWLSERQL |
| | retrotransposition | GILGNIQSGVLYSGISGEDSSEVLAGRPFGGCAILWHSDVLINVTSVETNSNRIC |
| | protein | AVLVTTESWKLILINVYLPHEGDDIKSDEFVHCLYLVEDIIGKHSDCHVVVGGDF |
| | | NVDENRNWNHTKIINSFCDSNELLSACRSNSNIDYTYNESMERFSILDHFLLSST |
| | | LHNTCIEKISVMHDVDNISDHDPIFLKLKLDVKYIGFSSRIFAPRISWVKATEND |
| | | LNRFRHILSDNLKCITTPTSLLLCHDMKCTDACHHNAIAEYATAISEACLLAAGI |
| | | CIPHISNRCTDRRVPGWTERVEPLREKSIFWHKLWMECGRPRDGHVADCMRRTRA |
| | | GYHYAIRQVRKDEELIVKQRIAEALARDPSRDEWTEIKRIRGNKAGLSRIVDGCI |
| | | DEASISKLFADKYKCLYASVPYDIVEMQNIQDIVDSQLAASDFEDDSYIINYQDV |
| | | SDAIFKLNAHKNDGNLGISTDHFLHGGSDLHMHIAFLLTSIVVHGSVPSEFVSST |
| | | VIPIPKKPNVNATQSDNYRGIALSSCFCKILDNIILAKNADRLSTSDLQFGFKRK |
| | | SSTHMCTMVLKETLSYYVSNNSCTFCTFLDATKAFDRVNYCKLFHLLIKRGLPAS |
| | | VIRILIVMYTGHCVRVAWAGLASSFFSAVNGVKQGGVLSPILFCIYMDDLLVKLH |
| | | KSGVGCYIGTAFVGALAYADDIVLLAPSPSAMRKLLSVCEFYALDEDISFNAGKS |
| | | NFILVMPNSNQRLRTQMNNCTFSIGGAPVVKVNSYLHLGHIINNQLNDDDDVMYR |
| | | RNCFIGQANNVLCYENKLDMCVRIKLEKNECSSMYGCELWSTDNVEVECVAWRKA |
| | | LRRVINLPYDTHSYLLPLLIDTLPVFEEICRRSAKFILKCENSSSTLVKYVTRHA |
| | | IDIARYNSNVGKNALFCCNYEKWQLYDFVNGSVSLNRNSFLTFCLNRLSGCEVDN |
| | | AGSLYEALLVREGNLFVESFTRDDIELIIDAMSRC* |
| 98 | MG140-39 | MANCNGTIISYNMHGENQGSELLKSYCANSSVDELLIQEHWLSPDALHKIDDIAQ |
| | retrotransposition | DYFCFSVSSMTAVLESGPLRGRPFGGLSILVKNMHRQFCSVVALNERYIAVQYND |
| | protein | ILIIDVYFPCVSSINHKDETIDLIVQLDTLVNQSVVKDIIIGGDENCNLEIESWS |
| | | SKVICDEMEDNSLSSCNKLAVNVIGAEYTYSNEVLGHYSYLDYFVVSNSLTSSVM |
| | | SLEICSDDENLSDHSPVCIEVCNIISDCETTVLCSNKGGKSTKIPDTCYQNRWDH |
| | | ADIVAYYELTRLGLQPILAYLNDCSNSFPPQSAIDYKRYMRACIDLAYNDAVSVL |
| | | VDAAKNTVPREKANALKHWWDQELSELKEKAFASHKLWIEAGKPRNGCIFDIRKA |
| | | DKYKYKCLIKRKQLEVRDSIINDLHDALLLKDADHEWKVWKSKFPAKRPNKYALI |
| | | EGNSDPNIISNTFCNYFSEICTAHPSVNASSNNIFLNREDGYIGDLNQSTKNIIS |
| | | IDLIEDFILSMKRGKAAGLDSLTIEHIQFAHPAIICILKLLFNYMLEFGIVPEGE |
| | | RNGLVIPLPKEDSIKKNVKLENERCITISPVISKIFEHCLMRLFAKYLNSDDAQL |
| | | GFKKKCGCSHAIYCVKQVVDYYVRGGSTVNVCTLDISKAFDKVNLFVLICKLMDR |
| | | NIPNYVINVLYDWFSNNYITVKWLNILSSRCPVNSGVRQGGVLSPVLFAIYVDDI |
| | | LVKLRKSRLGCTIQGLSVNAYMYADDLIILSGSVTDLQKLITLSIEELKCIHLSI |
| | | NPKKCFCMRVGKRFKVNCNNVVVDNYSIQWSSEIRYLGVYLTAGHVLKENLDYGK |
| | | KSSIAA* |
| 99 | MG140-40 | MHGFNQGFTYVNEICNDQAYDIIFIQEHWLYPSTLHKILEISDNYIGFGTSAMQS |
| | retrotransposition | ELEARFLRGRPYGGTAILINKKEKTYCIDSCVFKRVVSVLLGDELFINVYLPCND |
| | protein | GSAANVNTLGEILANISNIIDAHDVNCIIFGGDINTDITKVSLHSNMILNEMKDY |
| | | DLTVCMKHIFGTCDVQFVDTFVCENINASSCIDFLCMSNSLSDNVSRYKVIDAYN |
| | | NHSDHLPVSIDVCL.PVKCVLYACMNTVCDDVQVHSEHASKCTRSKAKDSRLRWD |
| | | LGNIQMYYDLICAHLYPIYECITQVESNMYMYDEQDRSSRIDELYNKLVSTLHNC |
| | | TKPAIPLVKANTLKHWWTTELNQLKSKSLTSHNIWLDAGKPLRGEIYQSKRHDKL |
| | | SYKLAIKTAKKEADAAVSDALHTNLISKSSVNFWKTWKSKVCNKVKTCVSVEGCN |
| | | TDEQAAAKFSDYESRATSPNSQEYSSAKQNEFNRKLLQYRKVEYANDITAELVAL |
| | | AVAKMQDGKAAGFDNIYVEHIRHSHPLIFSLLAKLENLMLRAGIVPTLFGNGVVI |
| | | PIPKENTHKKTHPVDNERGITLSPIISKVFEHCLLQLMSKYLATSENQFGEKSNT |
| | | GCTQAVYAVRKVTEYYVANESTMNLCELDISKGEDKVNNYELMMKLMKRRTPSYF |
| | | IQILNHWFSISQCTVRWNGVLSAMYRLEAGVRQGGVLSPVLESVYVDDMLCKLKS |
| | | MGCSYKALHLSAFMYADDLVLLSPSVSAMQTMLNVCNCELKLLDLKLNVKKSKAV |
| | | RIGKRFKNNTVNLNIEGQAICWSSEAKYLGVVIVSAPRFKCSFDAAKAKFYRSAN |
| | | SILAKVGHKQNVTVTLHLIATIALPTLTYCIEALHINKSEVSSLSHPWQSCLFKL |
| | | FRTFDAEIIKNCCELLNYKTISELYNERVAKFARNLKICNNHVLPLL* |
| 100 | MG140-41 | MHGFNQGHSVISDECNPKNVNSMDIICIQEHWISSTNLSKLANFSNSYFMYGISA |
| | retrotransposition | MENAEKAGLIKGRPYGGVCILLRKELCKCVKFSKCLERLVCLVIDGYIFINCYFP |
| | protein | SIRSDDDLNTVYLLFSEIDDILRDFPGNRVILGGDFNVNIVNKAKYADIFIKKLQ |
| | | NINIMECNDIYNSAINFTYCHESLQNYSYIDYFAVSNEMIKDIIDEDILDIGNNI |
| | | SDHNPIVIKVKCQLSDVNVSSKASNYKEFFRWDHANISNYCNLTRTYLQSILEYV |
| | | NDVSDYLEMHACCYSCSDILKAGKERAYADLNLSASYQTEPCKSCSGNYDIARRC |
| | | IDSSYNKIVNVINESAEKTVPVRKANFYKEWWDDELSQLKYNSIQAHETWVKLNK |
| | | PKQGIAEDNKVKAKTEYKLAIRQKQSLERDGIINDLHESLLAKSINVFWKSWNSK |
| | | FGQKSKVPLVIESSQDNQVISDKFAGYFASINSSNEPSNKNRHKDVELCKFKDYK |
| | | GDNWSYRDKINTFMLDYCIKESKLGKSPGADGITIEHLLYSHPMLICLLSKLFNL |
| | | MLRFSYVPDKFAECIVIPVPKLQN |
| | | VSKIMKCEDERPISLSPIISKIFEKCILKLVEEYEVTSDRQFGEKKRIGCTHAIS |
| | | AVKSTVDFFTENGSTVNMCALDISKAEDKINHFILENKLMKSNMPLRILLLIICW |
| | | YSKLVNVVRWNGSLSNVFKTANGIRQGGILSPFLENIYVDDILESLKRSKLGCYV |
| | | GNMCCNSFMYADDLLLLAISVTQLKQMIIMCTNLFEEIDMRLNITKINCIRVGNR |
| | | HLSTVIPMVIGDIVVNWKQEMRYLGVCFVSAKKLKLNIIFAKQKFEKSLNSIFSK |
| | | VGSNASPEVILSLENSFCVPVLLYGLGPENLSLNLKTRNSLIKAYDSVFYKTEKT |
| | | FDNEVTRYCQLMFRVMPLTFKLDLLTINEMLSSQKCDNEIIKFFSHRSFKINMDK |
| | | LLYKYNIVIDDMYKAKEALEDNEKVKVESNIALGLV* |
| 101 | MG140-42 | MTETSHAPVLDQTCTRYQRHNVHEQAMQFVPPNRVVFSFFGPCDIVCHRGAFLSV |
| | retrotransposition | TSYCKTLSLLQRAHFTRHRNKATGQPFLLISVYKQQDLQTLTQQGLVPETQTCNE |
| | protein | ALSIRGCRVAPSGRVALPYMMYPQGDHRNQPVHNQETHTPLTPGLDANPFAALSV |
| | | EQQTCGASTSRTSLIVGSMNVNGLFTQTMSSKHQDVCAFVSSHTVDICCLQETWC |
| | | DMSETEYSSMLASCGYTSFGVSGYSTRTRTGGGIGILVATSISHLAHMLHWESQK |
| | | HTQTTWIKLNSTQYKARKTAVGSVYLRPVSHTRPGDAERYVTELIALRDDITYLS |
| | | EHNFDVVLCGDFNARIGNSQVSPHVPQHNEQTRNTQGDRLVILLEQCNMFVMHNH |
| | | TSESPTCMHAGGSSVVDYCITNSSCRARCSDAAINFAGPCVSDHALLTMSLSHAT |
| | | RTRHRKTRRAHAKLWNRRAMHDEQTIAQFHATLETPLTQLSDFIDASTLSPQEAT |
| | | DTETSKLTAALRDAGATCFGTHQVRPHRAQWWNKDYANIRHECWNLYETHRRSGS |
| | | EADRAAYEEKRAAKNKMKRNLKRAYVKAQAGHVSKLCTPGLVSKTSWGAAKRLLK |
| | | LVTRGSSATEHTLPTVIHDGAPTDDLEAVMQVESSHYHTEMNPVPSATFCEATDT |
| | | LVKDKLHAWRQAEPEHINNMDDEFTITELDRALGSMHNWKAADHDGMIIELLRAG |
| | | GPALQLVVLKILNFCWTHETIPTNWKIGTIISLYKAGKKENPSNYRGITLLSVVR |
| | | KLFCTLIRSRLQDNISLHESQAAFRANRGCMDHVHTLARIVRAANRKDIPVYAFF |
| | | LDIRKAYDTVWRDGLIYKLLQKGVTGRLGRVISQVLIDTQSRVRFQHRESAYFPL |
| | | TLGVGQGDPLSTILFDVFIDDLLEELHARPVHHCIPVDSPHLDRIADITYADDVN |
| | | ALSLIPEGLQGHIDTIDTWLERWRSQPNVSKSKIMVENPPQGVPPTVETMRGSNL |
| | | DTVQCFKYLGVFFQSNGSWTDHTQHVRTQMNKAVGMWRPVLRCHYLPVAARLRII |
| | | YAFVYAPALYGAEVWIAPKSELEKLDTICKSAIRTVFGLHQFDCREEILFADTGL |
| | | LPVSSLINAAKLCWFTKLLNMPESRFPTAVEAVTLPGDTQRGRVAGGDFGTRIAD |
| | | ITTEIRRYAHMKIVMQDPLEHAPHRRRRPIRVSKRISHTKPDAHLYETITYSSLT |
| | | RERVKALLWKVYMEKCFERRGRKDGVTGEWMRSVIKHELGSLAPFLHSVECGLVR |
| | | VLMSARSRLSPVLCRPPQDKHREYSQAAGAFTTYISRAPIGAVLPSPPVAHCVRH |
| | | VSAVAELVRELLAQPGPYGCGSSVSWSDIAVVLVVGIDGVGGAPGVLGVVARPSP |
| | | LSELARVVAFCGASPRLRHHVAWMSRSVPVAGVVNVPVDMAMLQRTKAGRQVAPQ |
| | | GKHGAHSS* |
| 102 | MG140-43 | MAISYTSNNSVRICSFNMHGENNGVSMTKILCNSEDVILLQEHWLLPSNLSKLGD |
| | retrotransposition | ISHDFTHHSVSAMTTKISEGILYGRPEGGVSILYRNSLIKNISIIDADKEEGRYV |
| | protein | TIKLKCVNNEFITVINVYFPTICAYSDYIVNTSSIMAYLDNLFANEVSCHHVVAG |
| | | DENFEYRNDNIGFDLFRSLAVDNNLICCDDLHLNQNIKFTYKHETLPQQSWLDHF |
| | | FVSAGLTSSIVYCDTIDNGSNLSDHLPICCTINVSLSDSNCTPKLSKVYRDRWDK |
| | | ADLIKYYYQSGIHLQSLTAPPHVTQCSTHCQKAQHLQDINAYYERIIQALKASAI |
| | | GCVPRMPVNCLKHWWNDDLTRLKNLSIDMHNLWRQVGSPRNGIINEARLKAKLDY |
| | | KQAIRQAMLDCENKDADIINNKENQKDSRNFWKCWGAKYRKKVNNTACIDGCIDN |
| | | STIANKFKAYFQNTYVDSTCDVNACMEFDKLMSDHSHLQHNDIVEEIRIEDIEKC |
| | | IELLKPLKSAGHDDVAPEHLIHSHPSLCMHLKLLFSMMINHNYVPDSFGIGIIIP |
| | | VVKDKRGNLNSVENYRPITLSPIISKVFESFVLNRFAKFMTCDPLQFGFQRSVGC |
| | | NNALFAIRQVIQYENDRDSNVMVASLDACKAFDRVNHFKLESTLLQRKLPLHIIK |
| | | VLINWYCKLMVQVRWNNSLSDLFHVKSGVRQGGILSPALENVYIDCVINKIRASQ |
| | | LGCHIGSLYIAVVLFADDILLLSSSEMELQRMIDLCVESGDEIGLKFNAAKSHCM |
| | | IIGPHKVLVKPDMMMGNSPTAWSETIKYLGVYIQSDKKFTVDLSLVRRKEFASVN |
| | | CILRNAAFTSDIVKLELVEKQCFPILLYGLQSEDLKSSVIANVNAWVNSVYRKIF |
| | | GYHKWESVKECIYLLGRIDVFHTIKLRRINFLKNIQKCNNEVARSLENYVLHTRE |
| | | FQSCCTLPNNSVINISKSCDNVRKAVENSFQSKVVGH* |
| 103 | MG140-44 | MASQTLDTQTANNVCNSVSVMSYNMHGENQGYSYLNDICMKQTYQVILIQEHWLY |
| | retrotransposition | PATLHKLANISEHYSFYGASAMQTALDAGFIRGRPYGGTGILLHKDIAKYCVESC |
| | protein | AFERVVGVLLGDFLFVNAYFPCWDGSVINLDAANELLANLSNVLDSYQAKHVVEG |
| | | GDFNANLTKNSVYSNMILEFMSEHQLDICRKVIFGSSNVIFSDTYVNEALNASSC |
| | | IDFICISSGLSNNVAQYDVHDVENNHADHLPVSLKLCLPVSSILYNCIASGSSAC |
| | | MENEAKSRDTQSCDSKGNKLRWDKGNTQLYSDLTYAQLFPIYETLHSIDVESSAY |
| | | TAQEHRSLINETYTQVVSALHYAANVSIPAMASHTLKHWWCSDLSELKKKSMLSH |
| | | NEWINAGKPHAGRIHTAKQQSKLQYKRAIKHAKASAENSVSDELHRNICSRNNVK |
| | | EWKTWKNKIKRPGTEKLYVEGCNTDAKAAELLADYENKATSPNSNEYNNSKRVEF |
| | | EKSFASYAIQANDIDISAGIVESAALKMTAGKAAGIDNISIEHVHYCHIVIYSLL |
| | | AKLENLMLCFSCVPDAFGYGVTTPIPKEESHKKIHPVENFRGITLSPVLSKLFEH |
| | | CLLSVESDFLQTSDNQFGFKRSTGCTHAIYALRKVTEFFICNESTVNMCFLDISK |
| | | GFDKVNNFELLLKLMKRKAPSCFIKLLHDWESISYGSVKWNTSRSDWYKIGAGVR |
| | | QGGVISPILEAVYVDAMLEKVKTMGCQYKSLCTGAFMYADDLVLLSPSIYELQHM |
| | | IALCRNELHSLDLKLNVKKSKALRIGKRYKCKALPLMIDGQAVHWSNEARYLGIV |
| | | IRSACKFKCNFDPAKVKFYKAANTILAKLGNKCNVTVTLYLVAAVALPPLIYGIE |
| | | ALTLNSSELNSLNHPWNNCLGKMENTEDKELIKNCCDILGYESCQNVYVKKVEKE |
| | | LRNMKFIDNAILSALNTEQVNV* |
| 104 | MG148-12 | MANCNGTIISYNMHGENQGSELLKSYCANSSVDELLIQEHWLSPDALHKIDDIAQ |
| | reverse | DYFCESVSSMTAVLESGPLRGRPEGGLSILVKNMHRQFCSVVALNERYIAVQYND |
| | transcriptase | ILIIDVYFPCVSSLNHKDETIDLLVQLDTLVNQSVVKDIIIGGDENCNLEIESWS |
| | | SKVICDEMEDNSLSSCNKLAVNVIGAEYTYSNEVLGHYSYLDYFVVSNSLISSVM |
| | | SLEICSDDENISDHSPVCIEVCNIISDCETTVLCSNKGGKSTKIPDTCYQNRWDH |
| | | ADIVAYYELTRLGLQPILAYLNDCSNSFPPQSAIDYKRYMRACIDLAYNDAVSVL |
| | | VDAAKNTVPRFKANALKHWWDQELSELKEKAFASHKLWIEAGKPRNGCIFDIRKA |
| | | DKYKYKCLIKRKQLEVRDSITNDLHDALLLKDADHEWKVWKSKEPAKRPNKYALI |
| | | EGNSDPNIISNTFCNYFSEICTAHPSVNASSNNIFLNREDGYIGDLNQSTKNIIS |
| | | IDLIEDFILSMKRGKAAGLDSLTIEHIQFAHPAIICILKLLFNYMLEFGIVPEGE |
| | | RNGLVIPLPKEDSIKKNVKLENERCITISPVISKIFEHCLMRLFAKYLNSDDAQL |
| | | GFKKKCGCSHAIYCVKQVVDYYVRGGSTVNVCTLDISKAFDKVNLFVLLCKLMDR |
| | | NIPNYVINVLYDWESNNYITVKWLNILSSRCPVNSGVRQGGVLSPVLFAIYVDDI |
| | | LVKLRKSRLGCTIQGLSVNAYMYADDLIILSGSVTDLQKLITLSIEELKCIHLSI |
| | | NPKKCFCMRVGKREKVNCNNVVVDNYSIQWSSEIRYLGVYLTAGHVLKENIDYGK |
| | | KSSIAALNSIMSKTGNKAIDIVISLTQSYCIPILLYAVESMCLTTTERQRLGSSE |
| | | NKLYNKLFSTEDTQTIAYCQYYTGYLPLDYVIDLRCWNFLQKLSISNNLVINKLE |
| | | RLNGNNTIDSLCLKYNCEFKNYPSLKVSMWEKEKEVYSLCP |
| 105 | MG148-38 | MRGFFQGLVVVNDLISGYQSPDVILLQEHWLTPANMNLFDEKITTHEVVGKSAMS |
| | reverse | DRVSAGPLVGRPYGGAAILIRNELRADTECVECSDRFAVVRICNLLIMSVYLPCA |
| | transcriptase | ATAARMFIVEDLLQEIWSLRLKYSECSVVIGGDENADLNKQNDVSNFINSELTTC |
| | | ILIRCDSKELSRQQSTYVNESIGQSSTIDFFVCDVVDDIIDYCVIDPDVNFSDHL |
| | | PVAVRCKWSRTDNIKRSKSSNSSKVKHLRWDHGDLLSYYSSTMSRLYPLYEYLIR |
| | | IEGWLVEPSNAVQRETVIRLVDHVYDQLVVALRESANSYIPKHAKKEFKEWWNQE |
| | | LDALKHNAIASSSVWKNAGKPRGGALYLQYKRDKLLYKKRLREEQKAEAACYSND |
| | | LHDALLRKSGQDFWKCWNSKFERRSNKVVQVDGITDSAVIADKFAEYFESVCRPF |
| | | NADRNNLIKSKYNELRSTYTGTPIVEEQWENVELLSKLVDSMSKGKAAGLDELSS |
| | | EHLKYSHPVVVCILSKLENLEVYYSHIPASFGRSFTVPIPKHDGRTHALHVDDER |
| | | GISISPVISKLFEMAILDRESVEF |
| | | STSDHQFGFKKNISCRHAIYCVRSVIDNEVLHGSTVNVCALDISKAFDRMNHYAL |
| | | FIKLMERSFPCELLAILETWEYISVSCVKWNDNLSSFFVLTAGVRQGGVLSPYLF |
| | | AIFIDDLVYKVKSLNVGCYISLICAAIFMYADDILLLSPTVDGLQQLLHVCENEL |
| | | EQLDMKLNVNKSVCIRVGPRFNADCAELRSRNGAVLKWTDSCRYLGVYFVRGRTL |
| | | KCSFSNAKSRFFRAFNAVEGKVGRAASEETVIELIRAKCIPILLYATEACPFFSR |
| | | DKQSLEFTVTRLFMKIFQTGSPAVVRDCQRSENELPIEMQVIIRTSRFLQAFVAT |
| | | QNSLCSLFQRSASCQLNDIFSKYDRVQTASQLANRIHHTFAMCDSV* |
| 106 | MG148-40 | MHGFTPDCMLLSTVVPIQKGKNVNVIDSANYRGISLSSIFAKLEDLLILQRYSDC |
| | reverse | LCLSDQQFGFRAKRSTDMCSMVLKESISYYVNNGSSVYCTFIDATKAFDRVEYCK |
| | transcriptase | LFRQLLSRGLPPVIIRIMLNLYVGHVTRVEWNGIRSRNFSVENGVKQGGIVSPIL |
| | | ECIYLDGLLQSLATSGVGCYIGTIFVGAMAYADDLVLLAPSANAMRLMLRNCDAF |
| | | ANEYNIRENANKSKCLFCSARRHSRCNIGAQPVFYIGGNPIEIVDHWPHLGHIIS |
| | | SHLDDEQDIIQRRNAMAGQINNVLNYFVGLDCFVKQKLLTTYCYSLYGSVLWDLR |
| | | HLCIDSVCTTWRRGLRRVWGLPHNTHSNLLPLLSCSLPVYDELCKRFVLFAQKCL |
| | | MSDSSLVSSVATYAFIYGRSDSVFGRNVSKCCLRENVANDDFLSINKKEMENNYC |
| | | SNLEQDTMSTVNLLLELICVRDGLFIMPMEADAAELIEIIRLLCTV* |
| 107 | MG148-41 | MTIINDAAQLVVISYNLHGLNQGLPGIREFMTELKPDVIMVQEHWLTSDNINKLS |
| | reverse | DISDEYFVIGSSAMDARVSAGPLEGRPEGGTAVFIKNKYINVTVNLVISERYVVI |
| | transcriptase | QLCDWLLINVYLPCIGTSNRILLYSDTLCELQSIIRAHPECNCLVGGDENTDLND |
| | | TRSHTANTVVNGFIAGCNLNLSDHLPIITVCVEDSNCKINNPKPTSTEDVTHFRW |
| | | DHAPLQDYYEHTRLGLQPILADLDELIGNKLSYSNVDFICQVDCIYNRVVSVIQQ |
| | | CSHAYVPKHKKNFYKFWWNQELDELKDKAIASCKMWKDAGKPRHGAIHAKYRQDK |
| | | LLYKKRIRQERVQETSSFTNELHDALLNKSGRDEWKKWNSKFENKSNKILQVGGT |
| | | SDTATILNNFAKHFEQVCVPFNATRNEELKSRYKEMRLNYSESSVINDTTVEDVQ |
| | | LVENLLTNMKNGKAGGLDELTSEHLKFCHPVVVIILCKLENLFVINGHIPDSFGV |
| | | SYTVPIPKSDGRSRSMTVDDERGISISPVISKIFELCVLDRYSDYLQTSDHQFGE |
| | | KKQLGCRHAIFSVRSVIEQYISNGSTVNLCALDLSKAFDRMNQYALFIKLMERRF |
| | | PVKILTILEQWFSIAETCVRWGSEFSYFFSLLAGVRQGGVLSPVLFAIFIDGIVN |
| | | RVNATNVGCYNSTVCVSIFLYADDILLISPTVTGLQTLLTVCENELCELDMRLNV |
| | | NKSVCMREGARFKSHCANLVSVQGGALQWASSCRYLGVYFVSGRVFRCCFHSAKC |
| | | NFFRAENSVYSKIGCCASEEVILSLLKSKCLP |
| 108 | MG148-42 | MRGFFQGLVVVNDLISGYQSPDVILLQEHWLTPANMNLFDEKITTHEVVGKSAMS |
| | reverse | DRVSAGPLVGRPYGGAAILIRNELRADTECVECSDRFAVVRICNLLIMSVYLPCA |
| | transcriptase | GTADRMFIVEDLLQEIWSLRLKYSECSVVIGGDFNADLNKQNDVSNFINSELTTC |
| | | LLIRCDSKELSRQQSTYVNESLGQSSTIDFFVCDVVDDIIDYCVIDPDVNESDHL |
| | | PVAVRCKWSRTDNLKRSKSSNSSKVKHLRWDHGDLLSYYSSTMSRLYPLYEYLIK |
| | | EEGELVEPTNAVQRETVIRLVDHVYDQLVVALRESANSYIPKHAKKFEKEWWNQE |
| | | LDALKHNAIASSSVWKNAGKPRGGALYLQYKRDKLLYKKRLREEQKAEAACYSND |
| | | LHDALLRKSGQDFWKCWNSKFERRSNKVVQVDGITDSAVIADKFAEYFESVCRPF |
| | | NADRNNLIKSKYNELRSTYTGTPIVEEQWENVELLSKLVDSMSKGKAAGLDELSS |
| | | EHLKYSHPVVVCILSKLENLFVYYSHIPASFGRSFTVPIPKHDGRTHALHVDDER |
| | | GISISPVISKLFEMAILDRESVEFSTSDHQFGFKKNLSCRHAIYCVRSVIDNFVL |
| | | HGSTVNVCALDISKAFDRMNHYALFIKLMERSFPCELLAILETWFYISVSCVKWN |
| | | DNISSFFVLTAGVRQGGVLSPYLFAIFIDDLVYKVKSINVGCYISLICAAIEMYA |
| | | DDILLLSPTVDGLQQLLHVCENELEQLDMKLNVNKSVCIRVGPRENADCAELRSR |
| | | NGAVLKWTDSCRYLGVYFVRGRTLKCSFSNAKSRFFRAFNAVEGKVGRAASEETV |
| | | IELIRAKCIPILLYATEACPFFSRDKQSLEFTVTRLEMKIFQTGSPAVVRDCQRS |
| | | ENELPIEMQVIIRTSRELQAFVATQNSLCSLFQRSASCQLNDIFSKYDRVQTASQ |
| | | LANRIHHTFAMCDSV* |
| 109 | MG148-43 | MESAQIDVLLLQEHWISDAQLNALNNVGANYLNFGVSGFDTSAVLGGRPYGGCAV |
| | reverse | IWRSDLLLQVQPLVVSSRRLSAVIFSTDNWSLILINVYMPYEGDEIKTDEFIDLL |
| | transcriptase | SIIEDLVLSNSASHVIVGGDENVEENRNRMHTALLNSECDNTGLSPVIQHSSCNI |
| | | DYTYNFNMSRFNILDHFLLSGTLFDVCVTSAYVVHDIDNTSDHDPIILRLSLDIK |
| | | YVSVCNRTSSSRVSWVKASDRDIRNYQYNLASNIQHVTIPSVALLCKDVNCSNFA |
| | | HRCQLSRYLTDISDACLAAGEASIPHTCSRHSGKRIPGWSEKVEPLRQRSLFWHS |
| | | MWVECGRPRSGVVADCMRRARASYHYAIRAVRRNEQNIIRERVADALLRDPSRDE |
| | | WTEVKKIRNSKSGRAVIVDGCSDAPSISQLFASKYRHLYTSVPYQHNDLQSIVSD |
| | | VESRISEDGDCFIGSQEVMAALSKIKLHKNDGDLGLASDYFINSDPALSVHIALL |
| | | FTGIVIHGFVPSNLLSSTIVPIPKKSNVNATDSDNYRGIALSSVIGKIFDNVILV |
| | | KYSDKLSTCNLQFGEKRNSSTHMCTMVLKETISYYVNNNSSVFCTELDASKAFDR |
| | | VHYCKLFRLLLGRGLPVCILRVLIQLYVGHSIRVTWAGLVSSCFTALNGVKQGGV |
| | | ISPVLECIYVDELLIRLAESGVGCYIGESEAGALAYADDIVLIAPTPSAMRKLLA |
| | | ICDTYATEYNILENAQKSNFIAFVPSSRHFLHKAMINCVFRIGGVQIEHVESYTH |
| | | IGHIITSRLDDADDILHRRSSYIGQVNNVVCYEDSLS |
| 110 | MG148-44 | GSARCPSIGQPTSRVEPWRLTREGNPTASNVVDGCTTPDSISQLFAAKYKELYTS |
| | reverse | VPYSADDMSSLNSDINALMSSGFCAGCFVNANEVFAAVKELKLHKNDGNTGLTSD |
| | transcriptase | HVKCALPDLSVHIALLLSGVLSHGSIPKDLLLSTVIPIPKNKNGNLADSSNYRGI |
| | | ALSSVECKIFDKILLARYNDKFVISELQFGFKAGRSTHMCSMLIKESMLYYKNNN |
| | | SLVYCAFLDATKAFDRVNYCKLERLLIKRGLPPPILRALLNFYVGHTVRVSWEGS |
| | | VSSYFLALNGMKQGGVISPLLFCVYIDDLLCALEKSGVGCYIGLHETGALAYADD |
| | | IVLLAPSPTALRRLLSICDVYAAEYCI |
| 111 | MG148-45 | MGFQAQTAGGMNNTAIMTSDQNCTSLSALTENMHGENQGSVLLKEVCEKQIYDLI |
| | reverse | FIQEHWLYPSNISKLLEISPHYIGHGISAMESAVDAGFIKGRPYGGCAILVNNKY |
| | transcriptase | KNMHADVCTFERVVAILLGDVLLINVYCPCENGSTEGFDALNEILANIANILDTY |
| | | KAQYVVIGGDENTDISKDTTHSVLIKEELDEYGIKYCKSEMEGSVNIQFERTFAC |
| | | ETTGAGSCIDEFCVSDNICNNVMRYKVLDMYSNHSDHRPILCQFCVPVQSSLHQY |
| | | VSSGCVNEVNERANANVHASKQSAESEQALRWDLGNVQLYNDLTYQYLYPIYDRI |
| | | MNYDNMHSVNETNINEGKEAACVFIEQSYNDLINALHHCAANSIPRVQRNAIKHW |
| | | WNSELTELKKCSITSHNNWLESGKPRSGAVFALKQQDKLRYKQAIKKVKNEADAA |
| | | ISDELHAKLCAKKTTSFWKTWKNKVSNKAKKQISVEGCNTDEMAVCSLAEHERKA |
| | | TVPNCEIFNNEKKTEFVHKLSEYSVNSSPTAVTAEIVYISLSKVHKGKAPGIDNI |
| | | TAEHIGNSHVVIYSLLAKLFNMMLEYGHVPVSFINGIIVPIPKDENNKNKYPVDN |
| | | ERGITLSPVLSKIFEHCLINTEGDYLLTCDNQFGFKTKVGCSHAVYAVRKIIDYF |
| | | VSGESTVNLCFLDISKGFDKVNKYELLLKLMNRRIPVCFIELLNYWLSMSISTVK |
| | | WNGFFSQPYSLEAGVRQGGVLSPILFAVYVNDMLCKLRKMGCSYKWLHFGAIMYA |
| | | DDLVLLAPSVYELQCMIRVCQEQLKLLDLRLNFKKSKALRIGKRYKHKAAQLQLN |
| | | GEKIPWGTEAKYLGMVIQSAARFKCSFDQAKVKFYRGANAILSKLSNKSNVTLTL |
| | | YLVSTIALPTLTYGIECLSLNKTEIKSLDYPWYKCFCRVFQTFDAGIIQLCQAIL |
| | | GYKSIKELYENRCQMFLEKIKIYGNEMLREASEL* |
| 112 | MG148-46 | MNSYSSTILSAAESSCTTLSITTENMHGENQGSTLLIDVCYKETYDVIFIQEHWL |
| | reverse | YPHNIRKLLDISSKYTGYGISAMQSVVDSGFVKGRPFGGCAILIHNKYINLHVDC |
| | transcriptase | SVFERVVAVNVGDFLLINVYFPCENGSAEGFDTLNEILANVSNILDTYKAPYAII |
| | | GGDENTNLTKRSIHANTVNDELNAHNMSYCKAKVFGSVEISFEDTFACDKTGASS |
| | | CIDFVCVSNCLMHNVSKYEVIDVYNNHSDHKPLFCQLCVPVSSTLHQFVASGCRK |
| | | LMNGNAGDSKCSNDSNCALRWDLGNVQLYTTLTSERLYPIYEKLLNYRYVVDEHN |
| | | PHESKQLASQRIDKLYNELILALHECAATCIPQVKRNAIKHWWNSELNELKTCSI |
| | | KSHKSWLEAHKPKSGALYLAKQQAKLRYKQAIKRTRLAADASISDALHVNLITSS |
| | | TEKFWKTWKTKVCKKNKQNMLLEGCSTDDIAVCKLADFFSKITAPNSDEYNV |
| | | KKKSEFLEQITDYSGQTNYDTINAELVYNSMSKLKKGKSPGIDNITVEHICNSHV |
| | | VIFALLAKLENLMLEVRYVPAIFGNGIIVPIPKEDTGKKVHPVDNERGITLSPVL |
| | | SKLFEHCLKCIFAEFMSTCDNQFAFKNNMGCSHAIYSVRKVTEYFVMGDSTVNLC |
| | | FLDISKGFDKVNKYELLLKLIKRNVPVCFIELMHHWFDISKSTVKWNGHFSQSYE |
| | | LKAGVRQGGVLSPLLFAVYVDDMLRKISKMGCTYNWIHLGAIMYADDLVLIASSV |
| | | YELQCMLRVCQAELNLIDLKLNVKKSKAIRIGKRSKLKAASLKLSEGHIAWSSEA |
| | | KYLGIVVQSAARFKCNFDHVKIKFYRGANAILSKLSSKANVTVTLHLISSIALPS |
| | | LTYGLECLALNKTELKSLHYPWHKCLCRIFQTEDEQIINICQAILGYKSVKDLYE |
| | | IRACMELSRVKKCDNFLLRALAA* |
| 113 | MG153-22 | MRQKSEQLELALDNRGEAPTSRRSGEAPTTAHEAERSGGGHRLMEQVVARANALA |
| | reverse | ALRRVKQNRGSPGVDGMTVGELPQYLAKHWETVREQLLAGSYQPEPVRRQAIPKP |
| | transcriptase | GGGTRVLGIPTVLDRFIQQCLLQVLQPREDPSFSDHSYGERPGRNAHDAVCAAQQ |
| | | YIQAGRRWVADLDLEKFFDRVNHDVLMERLARRIADRRVLRLIRRYLVAGILHGG |
| | | VVVERREGTPQGGPLSPLLASVLLDEVDRELERRGLAFARYADDINVYCGSRRAA |
| | | HDAMATIKRLFAALRIKVNESKSTVARVWERKFLGYSEWVAPGRVIRPRIAPAAL |
| | | AVMKERVRRITRRTGGRSLEAVAQELREYLTGWKAYERLAGKPRVERDLDEWTRH |
| | | RLRAVQLKQWKRGRTVCRELLARGVPEREARAAAAHARRWWAMAAHSALQTALPN |
| | | SHFDQLGVPRLAGR |
| 114 | MG153-23 | MRADEAEAHASAASTGKGGRNLPGTAAGAEVRAAAGGRTKPEALRLMEAAVERSN |
| | reverse | MLGAYERVVKNQGAPGVDGLTVTEEKPWLQAHWPKIRQVLLAGEYMPAAVRKVDI |
| | transcriptase | PKPQGGVRTLGIPTVLDRLIQQALHQVLQPLFEPEFSESSYGERPGRNAHQAVEA |
| | | ARSYVAEGKRWVVDLEKFFDRVNHDVLMARVARKVKDERVLKLIRRYLEAGLMEG |
| | | GMTSARTEGTPQGGPLSPLLSNTLLTDLDRELEKRGHRFCRYADDCNVYVGSRRS |
| | | GQRVMAKITAFLEQRLKLQVNADKSAVARPWQRKFLGYSVTWHRNPKLKIAPSSR |
| | | QRLAEKIRQTLRGARGQSLRQVIAQLNPILRGWVAYFRITEEKGVLEELDGWVRR |
| | | KLRALLWRHWKRGYPRAQNLMRAGLRPERAWQSATNGHGPWWNGGSSHMNAACPK |
| | | SWFDHMQLVSLLATQRRESLVS |
| 115 | MG153-24 | MKGGKQKISQDTCLQESRAEPEGYAGGQTFIWMTENNLTNANKPEYGLLEQILSP |
| | reverse | TNLNRAYKGVRSNRGSGGIDKMEVESLKDYLVDNKETLIQSILDGKYRPNPVRRV |
| | transcriptase | EISKEKGTRKLGIPTVVDRVIQQAIAQVLSPTYERQFSENSYGERPGRNAHQALN |
| | | RCRDYITDGYIYAVDMDLEKFFDTVNQSKLIEVLSRTVKDGRVVSLIHKYLDAGV |
| | | VIRNKFEETEMGVPQGSPLSPVLSNIMLNELDKELEKRGHPFVRYADDLIIFCKS |
| | | KRSADRILANTVPYIENKLELKVNREKTTTAYVSGIKFLGYSFYVYKGEGRLRVH |
| | | PKSIAKMKERIRKLTSRSNGWGYARRKEALRQYITGWVNYFKLADMKKLLVSVDE |
| | | WYRRRLRLVIWKQWKRVRTRGRYLMKLGIVKHQAWEFANTRKGYWHTAKSPILNR |
| | | SVTSNRLRQAGYVEFVDYYRVVNGIN |
| 116 | MG160-7 | MHNEKSRFELSKGKWVYIQIEDLANHAKDHIRQIRDLWTPPEYFFHLQKGGHIAA |
| | reverse | LRLHTPNEWYGKVDLSKFENNITRHRVTRSLKRIGYSERDAEEFAVASTVLVNHA |
| | transcriptase | TRRYVLPYGFVQSPLLASISLDKSDLGNCLRRLHEETVSVSVYVDDIVVSADSER |
| | | DVAEALSNIYLAAINSREPINEEKSRGPSSTINAFNIELDMHELEITAKRYEKMC |
| | | GEVLINGTGQVSDAILNYVQTVNPAQAEQMLRDFPGVFGTLSSSASRYDAS |
| 117 | nMG3- | MSTDMKNYRIGVAVGDRSVGLAAIEFDDDGLPIQKLALVTFRHDGGLDPTKNKTP |
| | 6 D13A | MSRKETRGIARRTMRMNRERKRRLRNLDNVLENLGYSVPEGPEPETYEAWTSRAL |
| | | LASIKLASADELNEHLVRAVRHMARHRGWANPWWSLDQLEKASQEPSETFEIILA |
| | | RARELFGEKVPANPTLGMLGALAANNEVLLRPRDEKKRKTGYVRGTPLMFAQVRQ |
| | | GDQLAELRRICEVQGIEDQYEALRIGVEDHKHPYVPKERVGKDPINPSINRTIRA |
| | | SLEFQEFRILDSVANLRVRIGSRAKRELTEAEYDAAVEFLMDYADKEQPSWADVA |
| | | EKIGVPGNRLVAPVLEDVQQKTAPYDRSSAAFEKAMGKKTEARQWWESTDDDQLR |
| | | SLLIAFLVDATNDTEEAAAEAGLSE |
| | | LYKSWPAEEREALSNIDEEKGRVAYSQETLSKLSEYMHEYRVGLHEARKAVFGVD |
| | | DTWRPPLDKLEEPTGQPAVDRVLTILRREVLDCERQWGRPRAITVEHTRIGLMGP |
| | | TQRQKILNEQKKNRADNERIRDELRESGVDNPSRAEVRRHLIVQEQECQCLYCGT |
| | | MITTTTSELDHIVPRAGGGSSRRENLAAVCRACNAKKKRELFYAWAGPVKSQETI |
| | | ERVRQLKAFKDSKKAKMFKNQIRRLNQTEADEPIDERSLASTSYAAVAVRERLEQ |
| | | HENEGLALDDKSRVVLDVYAGAVTRESRRAGGIDERILLRGERDKNRFDVRHHAV |
| | | DAAVMILLNRSVALTLEQRSQLRRIFYEQGLDKLDRNQLKPEEDWRDFTGLAPAS |
| | | QEKFLEWRKAATILGDLLAEAIEDDSIAVVSPLRLRPQNGSVHLETISAVKKQTL |
| | | GSDWPADAVKRIVDPEIYLAMKDALGKLKELPEDSARSLELPDGREVEADDEVLF |
| | | FPENAASILIPRGVAEIGGSIHHARLYGWLTKKGELKVGMLRVYGAEFPWLMRES |
| | | GSRNVLSMPIHRGSQSERDMQDTTRKAVESGEAVEFAWITQNDELEFDPDDYIAH |
| | | GGKDELRQFLGEMPECRWRVDGFKKNYQIRIRPAMLSREQLPSDIQRRLESKTLT |
| | | KNESLLLKALDTGLVVAIGGLIPLETLKVIRRNNLGFPRWRGNGNLPTSFEVRSS |
| | | ALRALGVEG |
| 118 | nMG3- | MSTDMKNYRIGVDVGDRSVGLAAIEFDDDGLPIQKLALVTERHDGGLDPTKNKTP |
| | 6 H586A | MSRKETRGIARRTMRMNRERKRRLRNLDNVLENLGYSVPEGPEPETYEAWTSRAL |
| | | LASIKLASADELNEHLVRAVRHMARHRGWANPWWSLDQLEKASQEPSETFEIILA |
| | | RARELFGEKVPANPTLGMLGALAANNEVLLRPRDEKKRKTGYVRGTPLMFAQVRQ |
| | | GDQLAELRRICEVQGIEDQYEALRIGVFDHKHPYVPKERVGKDPINPSINRTIRA |
| | | SLEFQEFRILDSVANIRVRIGSRAKRELTEAEYDAAVEFLMDYADKEQPSWADVA |
| | | EKIGVPGNRLVAPVLEDVQQKTAPYDRSSAAFEKAMGKKTEARQWWESTDDDQLR |
| | | SLLIAFLVDATNDTEEAAAEAGLSELYKSWPAEEREALSNIDFEKGRVAYSQETL |
| | | SKLSEYMHEYRVGLHEARKAVFGVDDTWRPPLDKLEEPTGQPAVDRVLTILRRFV |
| | | IDCERQWGRPRAITVEHTRTGLMGPTQRQKILNEQKKNRADNERIRDELRESGVD |
| | | NPSRAEVRRHLIVQEQECQCLYCGTMITTTTSELDAIVPRAGGGSSRRENLAAVC |
| | | RACNAKKKRELFYAWAGPVKSQETIERVRQLKAFKDSKKAKMFKNQIRRLNQTEA |
| | | DEPIDERSLASTSYAAVAVRERLEQHENEGLALDDKSRVVLDVYAGAVTRESRRA |
| | | GGIDERILLRGERDKNREDVRHHAVDAAVMTLLNRSVALTLEQRSQLRRTFYEQG |
| | | LDKLDRNQLKPEEDWRDFTGLAPASQEKFLEWRKAATILGDLLAEAIEDDSIAVV |
| | | SPLRLRPQNGSVHLETISAVKKQTLGSDWPADAVKRIVDPEIYLAMKDALGKLKE |
| | | LPEDSARSLELPDGREVEADDEVIFFPENAASILIPRGVAEIGGSIHHARLYGWL |
| | | TKKGELKVGMIRVYGAEFPWLMRESGSRNVLSMPIHRGSQSFRDMQDTTRKAVES |
| | | GEAVEFAWITQNDELEFDPDDYIAHGGKDELRQFLGEMPECRWRVDGFKKNYQIR |
| | | IRPAMLSREQLPSDIQRRLESKILTKNESLLLKALDTGLVVAIGGLLPLETLKVI |
| | | RRNNIGEPRWRGNGNLPTSFEVRSSALRALGVEG |
| 119 | nMG3- | MSTDMKNYRIGVDVGDRSVGLAAIEFDDDGLPIQKLALVTERHDGGLDPTKNKTP |
| | 6 N609A | MSRKETRGIARRTMRMNRERKRRLRNLDNVLENLGYSVPEGPEPETYEAWTSRAL |
| | | LASIKLASADELNEHLVRAVRHMARHRGWANPWWSLDQLEKASQEPSETFEIILA |
| | | RARELFGEKVPANPTLGMLGALAANNEVLLRPRDEKKRKTGYVRGTPLMFAQVRQ |
| | | GDQLAELRRICEVQGIEDQYEALRIGVEDHKHPYVPKERVGKDPLNPSTNRTIRA |
| | | SLEFQEFRILDSVANLRVRIGSRAKRELTEAEYDAAVEFLMDYADKEQPSWADVA |
| | | EKIGVPGNRLVAPVLEDVQQKTAPYDRSSAAFEKAMGKKTEARQWWESTDDDQLR |
| | | SLLIAFLVDATNDTEEAAAEAGLSELYKSWPAEEREALSNIDFEKGRVAYSQETI |
| | | SKLSEYMHEYRVGLHEARKAVFGVDDTWRPPLDKLEEPTGQPAVDRVITILRRFV |
| | | IDCERQWGRPRAITVEHTRTGLMGPTQRQKILNEQKKNRADNERIRDELRESGVD |
| | | NPSRAEVRRHLIVQEQECQCLYCGTMITTTTSELDHIVPRAGGGSSRRENLAAVC |
| | | RACAAKKKRELFYAWAGPVKSQETIERVRQLKAFKDSKKAKMFKNQIRRLNQTEA |
| | | DEPIDERSLASTSYAAVAVRERLEQHENEGLALDDKSRVVLDVYAGAVTRESRRA |
| | | GGIDERILLRGERDKNREDVRHHAVDAAVMTLLNRSVALTLEQRSQLRRTFYEQG |
| | | LDKLDRNQLKPEEDWRDFTGLAPASQEKFLEWRKAATILGDLLAEAIEDDSIAVV |
| | | SPLRLRPQNGSVHLETISAVKKQTLGSDWPADAVKRIVDPEIYLAMKDALGKLKE |
| | | LPEDSARSLELPDGREVEADDEVIFFPENAASILTPRGVAEIGGSIHHARLYGWL |
| | | TKKGELKVGMLRVYGAEFPWLMRESGSRNVLSMPIHRGSQSERDMQDTTRKAVES |
| | | GEAVEFAWITQNDELEEDPDDYIAHGGKDELRQFLGEMPECRWRVDGFKKNYQIR |
| | | IRPAMLSREQLPSDIQRRLESKILTKNESLLLKALDTGLVVAIGGLLPLETLKVI |
| | | RRNNIGEPRWRGNGNLPTSFEVRSSALRALGVEG |
| 120 | SV40 | PKKKRKV |
| 122 | C-myc NLS | PAAKRVKLD |
| 123 | c-myc NLS | RQRRNELKRSP |
| 124 | hRNPA1 M9 | NQSSNFGPMKGGNFGGRSSGPYGGGGQYFAKPRNQGGY |
| | NLS | |
| 125 | Importin- | RMRIZFKNKGKDTAELRRRRVEVSVELRKAKKDEQILKRRNV |
| | alpha IBB | |
| | domain | |
| 126 | Myoma T | VSRKRPRP |
| | protein | |
| 127 | Myoma T | PPKKARED |
| | protein | |
| 128 | p53 | PQPKKKPL |
| 129 | mouse | SALIKKKKKMAP |
| | c-abl IV | |
| 130 | influenza | DRLRR |
| | virus NS1 | |
| 131 | influenza | PKQKKRK |
| | virus NS1 | |
| 132 | Hepatitis | RKLKKKIKKL |
| | virus | |
| | delta | |
| | antigen | |
| 133 | mouse Mx1 | REKKKELKRR |
| | protein | |
| 134 | human | KRKGDEVDGVDEVAKKKSKK |
| | poly (ADP- | |
| | ribose) | |
| | polymerase | |
| 135 | steroid | RKCLQAGMNLEARKTKK |
| | hormone | |
| | receptor | |
| | (human) | |
| | glucocorticoid | |
| 136 | MG3-6 | MSTDMKNYRIGVDVGDRSVGLAAIEFDDDGLPIQKLALVTFRHDGGLDPTKNKTP |
| | | MSRKETRGIARRTMRMNRERKRRIRNLDNVLENLGYSVPEGPEPETYEAWTSRAL |
| | | LASIKLASADELNEHLVRAVRHMARHRGWANPWWSLDQLEKASQEPSETFEIILA |
| | | RARELFGEKVPANPTLGMLGALAANNEVLLRPRDEKKRKTGYVRGTPLMFAQVRQ |
| | | GDQLAELRRICEVQGIEDQYEALRIGVFDHKHPYVPKERVGKDPLNPSTNRTIRA |
| | | SLEFQEFRILDSVANLRVRIGSRAKRELTEAEYDAAVEFLMDYADKEQPSWADVA |
| | | EKIGVPGNRLVAPVLEDVQQKTAPYDRSSAAFEKAMGKKTEARQWWESTDDDQLR |
| | | SLLIAFLVDATNDTEEAAAEAGLSELYKSWPAEEREALSNIDEEKGRVAYSQETL |
| | | SKLSEYMHEYRVGLHEARKAVFGVDDTWRPPLDKLEEPTGQPAVDRVITILRRFV |
| | | LDCERQWGRPRAITVEHTRTGLMGPTQRQKILNEQKKNRADNERIRDELRESGVD |
| | | NPSRAEVRRHLIVQEQECQCLYCGIMITTTTSELDHIVPRAGGGSSRRENLAAVC |
| | | RACNAKKKRELFYAWAGPVKSQETIERVRQLKAFKDSKKAKMEKNQIRRINQTEA |
| | | DEPIDERSLASTSYAAVAVRERLEQHENEGLALDDKSRVVLDVYAGAVTRESRRA |
| | | GGIDERILLRGERDKNREDVRHHAVDAAVMTLLNRSVALTLEQRSQLRRTFYEQG |
| | | LDKLDRNQLKPEEDWRDFTGLAPASQEKFLEWRKAATILGDLLAEAIEDDSIAVV |
| | | SPIRLRPQNGSVHLETISAVKKQTLGSDWPADAVKRIVDPEIYLAMKDALGKLKE |
| | | LPEDSARSLELPDGREVEADDEVIFFPENAASILTPRGVAEIGGSIHHARLYGWL |
| | | TKKGELKVGMLRVYGAEFPWLMRESGSRNVLSMPIHRGSQSERDMQDTTRKAVES |
| | | GEAVEFAWITQNDELEFDPDDYIAHGGKDELRQFLGFMPECRWRVDGFKKNYQIR |
| | | IRPAMLSREQLPSDIQRRLESKILTKNESLLLKALDTGLVVAIGGLIPLETLKVI |
| | | RRNNLGFPRWRGNGNLPTSFEVRSSALRALGVEG |
| 137 | MG29-1 | MENNFIKKYSLQKTLRFELKPVGETADYIEDFKSEYLKDIVIKDEQRAKDYQEIK |
| | | TLIDDYHREYIEECLREPVDKKTGEILDETQDLEDAFSYYQKLKENPTENRVGWE |
| | | KEQESLRKKLVISFVGNDGLEKKEFITRDLPEWLQKKGLWGEYKDTVENFKKFTT |
| | | YFSGFHENRKNMYTAEAQSTAIANRLMNDNLPKFENNYLAYQTIKEKHPDLVERL |
| | | DDALLQAAGVEHLDEAFQPRYFSRLFAQSGITAFNELIGGRITENGEKIQGLNEQ |
| | | INLYRQQNPEKAKGFPREMPLFKQILSDRETHSFLPDAFENDKELLQALRDYVDA |
| | | ATSEEGMISQLNKAMNQFVTADLKRVYIKSAALTSLSQELFHEFGVISDAIAWYA |
| | | EKRLSPKKAQESFLKQEVYAIEELNQAVVGYIDQLEDQSELQQLLVDLPDPQKPV |
| | | SSFILTHWQKSQEPLQAVIAKVEPLFELEELSKNKRAPKHDKDQGGEGFQQVDAI |
| | | KNMLDAFMEVSHAIKPLYLVKGRKAIDMPDVDTGFYADFAEAYSAYEQVTVSLYN |
| | | KTRNHLSKKPFSKDKIKINEDAPTLLNGWDLNKESDNKSIILRKDGNFYLAIMHP |
| | | KHTKVEDCYSASEAAGKCYEKMNYKLLSGANKMLPKVFFSKKGIETFSPPQEILD |
| | | LYKNNEHKKGATFKLESCHKLIDFFKRNIPKYKVHPTDNFGWDVEGFHFSPTSSY |
| | | GDLSGFYREVEAQGYKLWFSDVSEAYINKCVEEGKLELFQIYNKDESPNSTGKPN |
| | | LHTLYWKGLFEPENLKDVVLKLNGEAEIFYRKHSIKHEDKTIHRAKDPIANKNAD |
| | | NPKKQSVEDYDIIKDKRYTQDKFFFHVPISLNFKSQGVVRENDKINGLLAAQDDV |
| | | HVIGIDRGERHLLYYTVVNGKGEVVEQGSLNQVATDQGYVVDYQQKLHAKEKERD |
| | | QARKNWSTIENIKELKAGYLSQVVHKLAQLIVKHNAIVCLEDLNFGEKRGREKVE |
| | | KQVYQKFEKALIDKLNYLVEKERGATQAGGYLNAYQLAAPFESFEKLGKQTGILY |
| | | YVRSDYTSKIDPATGFVDELKPKYESMAKSKVFFESFERIQWNQAKGYFEFEFDY |
| | | KKMCPSRKFGDYRTRWVVCTEGDTRYQNRRNKSSGQWETETIDVTAQLKALFAAY |
| | | GITYNQEDNIKDAIAAVKYTKFYKQLYWLLRLTLSLRHSVIGTDEDFILSPVADE |
| | | NGVFFDSRKATDKQPKDADANGAYHIALKGLWNLQQIRQHDWNVEKPKKLNLAMK |
| | | NEEWFGFAQKKKFRA |
| 138 | MG160-62 | MAVRESTEDYKYQKNAKWYFAPSKKGEEIGGDLKRQIEAAHREDTTYFHLHKGGH |
| | reverse | VAALHEHRSHRLFCRVDIENFFYRIARNRVARALRNIGIARAEHYAKWSTVKDPF |
| | transcriptase | GEENYVLPYGFVQSPILATLVLDQSGVGDFIRAIPPAIGRSIYMDDIVLSGDDEV |
| | | QLQQYFKGLTNELERSNFRLNPSKIRAPAKTMDVENCDLETGQSVVQTKRKEKFF |
| | | AKARSPASIEAFEEYCERVEHGNSI* |
| 139 | MG160-366 | MPSDDGRAVGYELKALVEAKFTPDPELYHLAKGGHVAAIHIHRRQKYFSRLDFAN |
| | reverse | FFYSIARNRVARALREIGLARAEYYAKWSCVKNPYERPSYALPYGFVQSPLLASL |
| | transcriptase | VLAKSEVGTFLRELSERIVVSVYVDDIALSGNNRRVLQRVHRELRRIAVTSNFTI |
| | | NEAKSVELGETVELENCDLLHQKTIVTEARRREFYAIARSAASETAFENYCAAIA |
| | | VGNVA* |

1. A fusion protein comprising a nickase linked to a reverse transcriptase using a linker, wherein the reverse transcriptase comprises at least about 80% sequence identity to any one of SEQ ID NOs: 88-116 and 138-139.

2. A fusion protein comprising a nuclease linked to a reverse transcriptase using a linker, wherein the reverse transcriptase comprises at least about 80% sequence identity to any one of SEQ ID NOs: 88-116 and 138-139.

3. A fusion protein comprising a catalytically dead nuclease linked to a reverse transcriptase using a linker, wherein the reverse transcriptase comprises at least about 80% sequence identity to any one of SEQ ID NOs: 88-116 and 138-139.

4. A gene editing system, comprising:
a) a nickase;
b) a guide nucleic acid configured to form a complex with the nickase and to hybridize to a target nucleic acid sequence; and
c) a reverse transcriptase comprising at least about 80% sequence identity to any one of SEQ ID NOs: 88-116 and 138-139 and configured to form a complex with the nickase.

5. The gene editing system of claim 4, wherein the nickase is a modified endonuclease.

6. The gene editing system of claim 5, wherein the modified endonuclease is a Type II CRISPR endonuclease.

7. The gene editing system of claim 5, wherein the modified endonuclease is a Type V CRISPR endonuclease.

8. The gene editing system of any one of claims 6-7, wherein the Type II CRISPR endonuclease or the Type V CRISPR endonuclease has nickase activity.

9. The gene editing system of claim 5, wherein the modified endonuclease is selected from the group consisting of: spCas9 (H840A), spCas9 (D10A), nMG3-6 (D13A), nMG3-6 (H586A), nMG3-6 (N609A), Cas12a, and MG29-1.

10. The gene editing system of claim 5, wherein the modified endonuclease comprises at least about 80% sequence identity to any one of SEQ ID NOs: 117-119.

11. The gene editing system of any one of claims 4-10, wherein the nickase and the reverse transcriptase are fused.

12. The gene editing system of any one of claims 4-10, wherein the nickase and the reverse transcriptase are linked by a linker.

13. The gene editing system of claim 11, wherein the linker comprises at least 10, 20, or 30 amino acids.

14. The gene editing system of claim 11, wherein the linker comprises about 30-35 amino acids.

15. The gene editing system of claim 11, wherein the linker comprises about 30 amino acids.

16. The gene editing system of claim 11, wherein the linker comprises at least 809% sequence identity to SEQ ID NO: 33.

17. The gene editing system of claim 11, wherein the linker comprises at least 809% sequence identity to any one of SEQ ID NOs: 82-87.

18. The gene editing system of any one of claims 4-17, wherein the guide nucleic acid comprises a spacer sequence and a crRNA.

19. The gene editing system of any one of claims 4-18, wherein the guide nucleic acid further comprises a reverse transcriptase template (RTT).

20. The gene editing system of claim 19, wherein a base in the RTT comprises a bulky modification selected from the group of complex sugars, or complex amino groups, and/or other modifications compatible with RNA.

21. The gene editing system of any one of claims 4-20, wherein the guide nucleic acid further comprises a primer binding site.

22. The gene editing system of claim 21, wherein the primer binding site is on a 3′ end of the guide nucleic acid.

23. The gene editing system of any one of claims 21-22, wherein the primer binding site comprises at least 2, 4, 6, 8, 10, 13, 16, 20, 24, 28, 32, 36, 40, 45, 50, 55, 60, or 65 nucleotides.

24. The gene editing system of any one of claims 4-23, further comprising a transposase, integrase, or homing endonuclease.

25. The gene editing system of any one of claims 4-23, further comprising a retrotransposon.

26. The gene editing system of any one of claims 4-25, wherein the reverse transcriptase comprises a processivity of at least about 2-fold more than Moloney Murine Leukemia Virus (MMLV) reverse transcriptase.

27. The gene editing system of any one of claims 4-25, wherein the reverse transcriptase comprises a processivity of at least about 2-fold less than Moloney Murine Leukemia Virus (MMLV) reverse transcriptase.

28. The gene editing system of any one of claims 4-27, wherein the reverse transcriptase comprises an error rate of less than about 2.5%, 2.0%, 1.5%, 1%, 0.5%, 0.25%, 0.10%, or 0.05%.

29. The gene editing system of any one of claims 4-28, wherein the reverse transcriptase comprises an error rate of less than about 2.5%, 2.0%, 1.5%, 1%, 0.5%, 0.25%, 0.10%, or 0.05% as compared to Moloney Murine Leukemia Virus (MMLV) reverse transcriptase.

30. A gene editing system, comprising:
a) a nuclease;
b) a guide nucleic acid configured to form a complex with the nuclease and to hybridize to a target nucleic acid sequence; and
c) a reverse transcriptase having at least about 80% sequence identity to any one of SEQ ID NOs: 88-116 and 138-139 and configured to form a complex with the nuclease.

31. The gene editing system of claim 30, wherein the nuclease is a double strand nuclease.

32. The gene editing system of any one of claims 30-31, wherein the nuclease is a Type H CRISPR endonuclease.

33. The gene editing system of claim 32, wherein the CRISPR endonuclease is Cas9.

34. The gene editing system of claim 33, wherein the Cas9 is catalytically dead Cas9 (dCas9).

35. The gene editing system of any one of claims 30-34, wherein the nuclease and the reverse transcriptase are fused.

36. The gene editing system of claim 35, wherein the nuclease and the reverse transcriptase are linked by a linker.

37. The gene editing system of claim 36, wherein the linker comprises at least 10, 20, or 30 amino acids.

38. The gene editing system of claim 36, wherein the linker comprises about 30-35 amino acids.

39. The gene editing system of claim 36, wherein the linker comprises about 30 amino acids.

40. The gene editing system of claim 36, wherein the linker comprises at least 80% sequence identity to SEQ ID NO: 33.

41. The gene editing system of claim 36, wherein the linker comprises at least 80% sequence identity to any one of SEQ ID NOs: 82-87.

42. The gene editing system of any one of claims 30-34, wherein the nuclease and the reverse transcriptase are not linked.

43. The gene editing system of any one of claims 30-42, wherein the guide nucleic acid further comprises a primer binding site.

44. The gene editing system of claim 43, wherein the primer binding site is on a 3′ end of the guide nucleic acid.

45. The gene editing system of any one of claims 43-44, wherein the primer binding site comprises at least 2, 4, 6, 8, 10, 13, 16, 20, 24, 28, 32, 36, 40, 45, 50, 55, 60, or 65 nucleotides.

46. The gene editing system of any one of claims 30-45, further comprising a transposase, integrase, or homing endonuclease.

47. The gene editing system of any one of claims 30-46, further comprising a retrotransposon.

48. The gene editing system of any one of claims 30-47, wherein the reverse transcriptase comprises a processivity of at least about 2-fold more than Moloney Murine Leukemia Virus (MMLV) reverse transcriptase.

49. The gene editing system of any one of claims 30-47, wherein the reverse transcriptase comprises a processivity of at least about 2-fold less than Moloney Murine Leukemia Virus (MMLV) reverse transcriptase.

50. The gene editing system of any one of claims 30-49, wherein the reverse transcriptase comprises an error rate of less than about 2.5%, 2.0%, 1.5%, 1%, 0.5%, 0.25%, 0.10%, or 0.05%.

51. The gene editing system of any one of claims 30-49, wherein the reverse transcriptase comprises an error rate of less than about 2.5%, 2.0%, 1.5%, 1%, 0.5%, 0.25%, 0.10%, or 0.05% as compared to Moloney Murine Leukemia Virus (MMLV) reverse transcriptase.

52. A gene editing system, comprising:
a) a nickase
b) a guide nucleic acid configured to form a complex with the nickase and to hybridize to a target nucleic acid sequence; and
c) a reverse transcriptase configured to form a complex with the nickase, the reverse transcriptase having a X₁X₂DD motif, wherein X₁ is F or Y, and wherein when X₁ is Y, X₂ is A, R, N, D, C, E, Q, G, H, I, L, K, M, F, P, S, T, V, W, or Y.

53. The gene editing system of claim 52, wherein the X₂ is A or I.

54. The gene editing system of claim 52, wherein the X₁X₂DD motif is YADD (SEQ ID NO: 140) or YIDD (SEQ ID NO: 141).

55. The gene editing system of claim 52, wherein the X₁X₂DD motif is FADD (SEQ ID NO: 142), FVDD (SEQ ID NO: 143), FIDD (SEQ ID NO: 144), or FLDD (SEQ ID NO: 145).

56. The gene editing system of any one of claims 52-55, wherein the reverse transcriptase has at least about 80% sequence identity to any one of SEQ ID NOs: 88-116 and 138-139.

57. A gene editing system, comprising:
a) a nuclease;
b) a guide nucleic acid configured to form a complex with the nuclease and to hybridize to a target nucleic acid sequence; and
c) a reverse transcriptase configured to form a complex with the nuclease, the reverse transcriptase having a X₁X₂DD motif, wherein X₁ is F or Y, and wherein when X₁ is Y, X₂ is A, R, N, D, C, E, Q, G, H, I, L, K, M, F, P, S, T, V, W, or Y.

58. The gene editing system of claim 57, wherein the X₂ is A or I.

59. The gene editing system of claim 57, wherein the X₁X₂DD motif is YADD (SEQ ID NO: 140) or YIDD (SEQ ID NO: 141).

60. The gene editing system of claim 57, wherein the X₁X₂DD motif is FADD (SEQ ID NO: 142), FVDD (SEQ ID NO: 143), FIDD (SEQ ID NO: 144), or FLDD (SEQ ID NO: 145).

61. The gene editing system of any one of claims 57-60, wherein the reverse transcriptase has at least about 80% sequence identity to any one of SEQ ID NOs: 88-116 and 138-139.

62. An isolated reverse transcriptase having at least about 80% sequence identity to any one of SEQ ID NOs: 88-116 and 138-139.

63. A nucleic acid encoding for a fusion protein of any one of claims 1 to 3 or a gene editing system of any one of claims 4-61.

64. The nucleic acid of claim 63, wherein the nucleic acid is a DNA or an RNA.

65. The nucleic acid of claim 64, wherein the RNA is an mRNA.

66. A vector comprising the nucleic acid of any one of claims 63-65.

67. An adeno-associated virus or a lipid nanoparticle comprising the nucleic acid of any one of claims 63-65 or the vector of claim 66.

68. A cell comprising the nucleic acid of any one of claims 63-65 or the vector of claim 66.

69. The cell of claim 68, wherein the cell is a human cell.

70. A method for modifying a double- and/or single-stranded nucleic acid, comprising contacting a cell using the fusion protein of any one of claims 1-3, or the gene editing system of any one of claims 4-61.

71. A method for modifying a double- and/or single-stranded nucleic acid, comprising:
a) providing a cell with a guide nucleic acid that hybridizes to a target nucleic acid sequence;
b) providing the cell with a nuclease or nickase to cleave the nucleic acid at a location of binding of the guide nucleic acid;
c) providing the cell with a reverse transcriptase to synthesize a modification in the target strand of the nucleic acid at a location of cleavage by the nickase and/or nuclease.

72. The method of claim 71, wherein the reverse transcriptase has at least about 800% sequence identity to any one of SEQ ID NOs: 88-116 and 138-139.

73. The method of claim 71, wherein the modification is an insertion, deletion, or mutation.

74. The method of claim 71, further comprising providing an RNA or DNA template.

75. The method of claim 71, wherein the nucleic acid is a genome or a vector.

76. The method of claim 71, further comprising providing the cell with a transposase, integrase, or homing endonuclease.

77. The method of claim 71, further comprising providing the cell with a retrotransposon.
